(19) United States
(12) Patent Application Publication
(10) Pub. No.: US 2011/0312930 A1
(43) Pub. Date: Dec. 22, 2011

(54) Aryl compounds with aminoalkyl substituents and their use

(75) Inventors: Michael Härter, Leverkusen (DE); Hartmut Beck, Wuppertal (DE); Peter Ellinghaus, Melle (DE); Kerstin Berhörster, Essen (DE); Susanne Greschat, Wagenfeld (DE); Karl-Heinz Thierauch, Berlin (DE)

(73) Assignee: BAYER SCHERING PHARMA AKTIENGESELLSCHAFT, Berlin (DE)

(21) Appl. No.: 13/129,404

(22) PCT Filed: Oct. 31, 2009
(86) PCT No.: PCT/EP09/07805

§ 371 Date: May 16, 2011

(30) Foreign Application Priority Data
Nov. 14, 2008 (DE) 10 2008 057 344.2

(51) Int. Cl. A61K 31/4439; C07D 417/14; C07D 413/04; A61K 31/4545; A61K 31/5377; A61K 31/541; A61K 31/496; A61K 31/4245; A61K 31/454; A61P 35/00; A61P 9/10; A61P 9/00; A61P 9/06; A61P 25/00; A61P 9/12; A61P 13/12; A61P 11/00; A61P 17/06; A61P 3/10; A61P 27/02; A61P 29/00; A61P 7/00; C07D 413/14

(52) U.S. Cl. 514/210.2; 546/269.1; 546/194; 544/124; 544/60; 544/364; 548/131; 546/209; 544/138; 514/341; 514/318; 514/236.2; 514/227.8; 514/253.1; 514/343; 514/364; 514/326

(57) ABSTRACT

The present application relates to novel aryl compounds with aminoalkyl substituents, to processes for their preparation, to their use for treatment and/or prevention of diseases and to their use for the preparation of medicaments for treatment and/or prevention of diseases, in particular for treatment and/or prevention of hyperproliferative and angiogenic diseases and those diseases which arise from metabolic adaptation to hypoxic states. Such treatments can be carried out as monotherapy or also in combination with other medicaments or further therapeutic measures.

The present application relates to novel aryl compounds with aminoalkyl substituents, to processes for their preparation, to their use for treatment and/or prevention of diseases and to their use for the preparation of medicaments for treatment and/or prevention of diseases, in particular for treatment and/or prevention of hyperproliferative and angiogenic diseases and those diseases which arise from metabolic adaptation to hypoxic states. Such treatments can be carried out as monotherapy or also in combination with other medicaments or further therapeutic measures.

Cancer diseases are the consequence of uncontrolled cell growth of the most diverse tissue. In many cases the new cells penetrate into existing tissue (invasive growth), or they metastase into remote organs. Cancer diseases occur in the most diverse organs and often have tissue-specific courses of the disease. The term cancer disease as a generic term therefore describes a large group of defined diseases of various organs, tissue and cell types.

In the year 2002 4.4 million people worldwide were diagnosed with tumour diseases of the breast, intestine, ovaries, lung or prostate. In the same year, approx. 2.5 million deaths were assumed to be a consequence of these diseases (Globocan 2002 Report). In the USA alone, for the year 2005 over 1.25 million new cases and over 500,000 deaths were predicted from cancer diseases. The majority of these new cases concern cancer diseases of the intestine (˜100,000), lung (˜170,000), breast (˜210,000) and prostate (˜230,000). A further increase in cancer diseases of approx. 15% over the next 10 years is assumed (American Cancer Society, Cancer Facts and Figures 2005).

Tumours in early stages can possibly be removed by surgical and radiotherapy measures. Metastased tumours as a rule can only be treated palliatively by chemotherapeutics. The aim here is to achieve the optimum combination of an improvement in the quality of life and prolonging of life.

Chemotherapies are often composed of combinations of cytotoxic medicaments. The majority of these substances have as their action mechanism bonding to tubulin, or they are compounds which interact with the formation and processing of nucleic acids. More recently these also include enzyme inhibitors, which interfere with epigenetic DNA modification or cell cycle progression (e.g. histone deacetylase inhibitors, aurora kinase inhibitors). Since such therapies are toxic, more recently the focus has increasingly been on targeted therapies in which specific processes in the cell are blocked without there being a high toxic load. These include in particular inhibitors of kinases which inhibit the phosphorylation of receptors and signal transmission molecules. An example of these is imatinib, which is employed very successfully for treatment of chronic myeloid leukaemia (CML) and gastrointestinal stromal tumours (GIST). Further examples are substances which block EGFR kinase and HER2, such as erlotinib, and VEGFR kinase inhibitors, such as sorafenib and sunitinib, which are employed on kidney cell carcinomas, liver carcinomas and advanced stages of GIST.

The life expectancy of colorectal carcinoma patients has been successfully prolonged with an antibody directed against VEGF. Bevacizumab inhibits growth of blood vessels, which obstructs rapid expansion of tumours since this requires connection to the blood vessel system for a continuously functioning supply and disposal.

One stimulus of angiogenesis is hypoxia, which occurs again and again with solid tumours since the blood supply is inadequate because of the unregulated growth. If there is a lack of oxygen, cells switch their metabolism from oxidative phosphorylation to glycolysis so that the ATP level in the cell is stabilized. This process is controlled by a transcription factor, which is regulated upwards depending on the oxygen content in the cell. This transcription factor, called “hypoxia-induced factor” (HIF) is normally removed posttranslationally by rapid degradation and prevented from transportation into the cell nucleus. This is effected by hydroxylation of two proline units in the oxygen degradable domain (ODD) and an asparagine unit in the vicinity of the C terminus by the enzymes prolyl dehydrogenase and FIH (“factor inhibiting HIF”). After the modification of the proline units, HIF can be degraded with mediation by the Hippel-Lindau protein (part of a ubiquitin-E3-ligase complex) via the proteasome apparatus (Maxwell, Wiesener et al, 1999). In the event of oxygen deficiency, the degradation does not take place and the protein is regulated upwards and leads to transcription or blockade of the transcription of numerous (more than 100) other proteins (Semenza and Wang, 1992; Wang and Semenza, 1995).

The transcription factor HIF is formed by the regulated α-subunit and a constitutively present β-subunit (ARNT, aryl hydrocarbon receptor nuclear translocator). There are three different species of the α-subunit, 1α, 2α and 3α, the last of these being rather to be assumed as a suppressor (Makino, Cao et al, 2001). The HIF subunits are bHLH (basic helix loop helix) proteins, which dimerize via their HLH and PAS (Per-Arnt-Sim) domain, which starts their transactivation activity (Jiang, Rue et al., 1996).

In the most important tumour entities, overexpression of the HIF1α protein is correlated with increasing density of blood vessels and enhanced VEGF expression (Hirota and Semenza, 2006). At the same time glucose metabolism is changed to glycolysis, and the Krebs cycle is reduced in favour of the production of cell units. This also implies a change in fat metabolism. Such changes appear to guarantee the survival of the tumours. On the other hand, if the activity of HIF is now inhibited, the development of tumours could consequently be suppressed. This has already been observed in various experimental models (Chen, Zhao et al., 2003; Stoeltzing, McCarty et al., 2004; Li, Lin et al., 2005; Mizukami, Jo et al., 2005; Li, Shi et al., 2006). Specific inhibitors of the metabolism controlled by HIF should therefore be suitable as tumour therapeutics.

The object of the present invention was therefore to provide novel compounds which act as inhibitors of the transactivating action of the transcription factor HIF and can be employed as such for treatment and/or prevention of diseases, in particular of hyperproliferative and angiogenic diseases, such as cancer diseases.

Substituted multicyclic heteroaryl compounds with pyrrole, pyrazole and/or oxadiazole partial structures and the use of these compounds for treatment of diverse diseases are described in numerous forms in the patent literature, thus inter alia EP 0 908 456-A1, WO 97/36881-A1, WO 01/12627-A1, WO 01/85723-A1, WO 02/100826-A2, WO 2004/014370-A2, WO 2004/014881-A2, WO 2004/014902-A2, WO 2004/035566-A1, WO 2004/058176-A2, WO 2004/089303-A2, WO 2004/089308-A2, WO 2005/070925-A1, WO 2006/114313-A1, WO 2007/002559-A1, WO 2007/034279-A2, WO 2008/004096-A1, WO 2008/024390-A2 and WO 2008/114157-A1. WO 2005/030121-A2 and WO 2007/065010-A2 claim the use of certain pyrazole derivatives for inhibition of the expression of HIF and HIF-regulated genes in tumour cells. WO 2008/141731-A2 describes heteroaryl-substituted N-benzylpyrazoles as inhibitors of the HIF regulation pathway for treatment of cancer diseases. Heteroaryl-substituted 5-(1H-pyrazol-3-yl)-1,2,4-oxadiazoles as cannabinoid receptor modulators for treatment of diverse diseases are disclosed in US 2008/0255211-A1. Further diaryl-substituted isoxazole and 1,2,4-oxadiazole derivatives are described in WO 2009/029632-A1 as inhibitors of monoamine oxidase B for treatment of psychiatric diseases.

The present invention provides compounds of the general formula (I)

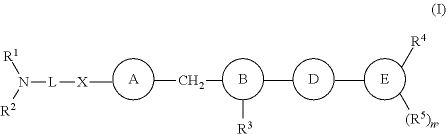

in which
the ring

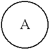

represents a phenyl or pyridyl ring,
the ring

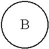

with the substituent R³ represents a heteroaryl ring of the formula

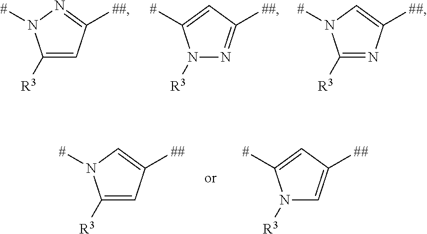

wherein
# designates the linkage point with the adjacent CH₂ group
and
## designates the linkage point with the ring

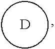

the ring

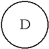

represents a heteroaryl ring of the formula

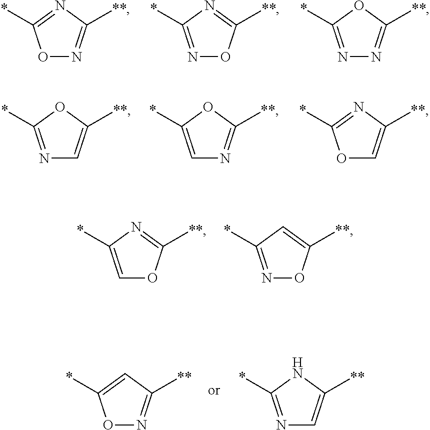

wherein
* designates the linkage point with the ring

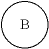

and
** designates the linkage point with the ring

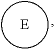

the ring

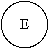

represents a phenyl or pyridyl ring,
X represents a bond or represents —N(R⁶)—, —O—, —S—, —S(═O)₂—, ♦-C(═O)—N(R⁶)-♦♦ or ♦-N(R⁶)—C(═O)-♦♦, wherein
   ♦ designates the linkage point with the group L
   and
   ♦♦designates the linkage point with the ring

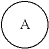

and
R⁶ denotes hydrogen, (C₁-C₆)-alkyl or (C₃-C₆)-cycloalkyl,
   where (C₁-C₆)-alkyl and (C₃-C₆)-cycloalkyl can each be substituted by hydroxyl or (C₁-C₄)-alkoxy,
L represents straight-chain (C₁-C₄)-alkanediyl if X denotes a bond or the group —S(═O)₂— or ♦-C(═O)—N(R⁶)-♦♦,
   and
   represents straight-chain (C₂-C₄)-alkanediyl if X denotes the group —N(R⁶)—, —O—, —S— or ♦-N(R⁶)—C(═O)-♦♦,
R¹ represents hydrogen, (C₁-C₆)-alkyl, (C₁-C₆)-alkylcarbonyl, (C₁-C₆)-alkoxycarbonyl, (C₁-C₆)-alkylsulphonyl or (C₃-C₆)-cycloalkyl,
   where the alkyl group in (C₁-C₆)-alkyl, (C₁-C₆)-alkylcarbonyl, (C₁-C₆)-alkoxycarbonyl and (C₁-C₆)-alkylsulphonyl can be substituted up to three times by fluorine and up to two times by identical or different radicals selected from the group consisting of hydroxyl, (C₁-C₄)-alkoxy, amino, mono-(C₁-C₄)-alkylamino and di-(C₁-C₄)-alkylamino
   and
   (C₃-C₆)-cycloalkyl can be substituted up to two times by identical or different radicals selected from the group consisting of fluorine, (C₁-C₄)-alkyl, hydroxyl, (C₁-C₄)-alkoxy, amino, mono-(C₁-C₄)-alkylamino and di-(C₁-C₄)-alkylamino,
R² represents hydrogen, (C₁-C₆)-alkyl or (C₃-C₆)-cycloalkyl,
   where (C₁-C₆)-alkyl can be substituted up to three times by fluorine and up to two times by identical or different radicals selected from the group consisting of hydroxyl, (C₁-C₄)-alkoxy, amino, mono-(C₁-C₄)-alkylamino and di-(C₁-C₄)-alkylamino
   and
   (C₃-C₆)-cycloalkyl can be substituted up to two times by identical or different radicals selected from the group consisting of fluorine, (C₁-C₄)-alkyl, hydroxyl, (C₁-C₄)-alkoxy, amino, mono-(C₁-C₄)-alkylamino and di-(C₁-C₄)-alkylamino,
or
R¹ and R² together with the nitrogen atom to which they are attached form a saturated 4- to 7-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl, (C₁-C₄)-alkylsulphonyl and (C₃-C₆)-cycloalkyl,
   where (C₁-C₄)-alkyl and (C₃-C₆)-cycloalkyl for their part can be substituted by hydroxyl or (C₁-C₄)-alkoxy,
R³ represents methyl, ethyl or trifluoromethyl,
R⁴ represents hydrogen or a substituent selected from the group consisting of halogen, cyano, pentafluorothio, (C₁-C₆)-alkyl, tri-(C₁-C₄)-alkylsilyl, —OR⁷, —NR⁷R⁸, —N(R⁷)—C(═O)—R⁸, —N(R⁷)—C(═O)—OR⁸, —N(R⁷)—S(═O)₂—R⁸, —C(═O)—OR⁷, —C(═O)—NR⁷R⁸, —SR⁷, —S(═O)—R⁷, —S(═O)₂—R⁷, —S(═O)₂—NR⁷R⁸, (C₃-C₆)-cycloalkyl, 4- to 6-membered heterocyclyl and 5- or 6-membered heteroaryl,
   where (C₁-C₆)-alkyl for its part can be substituted up to three times by fluorine and up to two times by identical or different radicals selected from the group consisting of —OR⁷, —NR⁷R⁸, —N(R⁷)—C(═O)—R⁸, —N(R⁷)—C(═O)—OR⁸, —C(═O)—OR⁷, —C(═O)—NR⁷R⁸, (C₃-C₆)-cycloalkyl, 4- to 6-membered heterocyclyl and 5- or 6-membered heteroaryl
   and where
   the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo, amino, mono-(C₁-C₄)-alkylamino, di-(C₁-C₄)-alkylamino, (C₁-C₄)-alkylcarbonylamino, (C₁-C₄)-alkoxycarbonylamino, (C₁-C₄)-alkylcarbonyl and (C₁-C₄)-alkoxycarbonyl
   and
   the heteroaryl groups mentioned for their part can be substituted up to two times by identical or different radicals selected from the group consisting of fluorine, chlorine, cyano, (C₁-C₄)-alkyl, trifluoromethyl, (C₁-C₄)-alkoxy and trifluoromethoxy,
   and wherein
   R⁷ and R⁸ independently of each other for each individual occurrence denote hydrogen, (C₁-C₆)-alkyl, (C₃-C₆)-cycloalkyl or 4- to 6-membered heterocyclyl,
      where (C₁-C₆)-alkyl can be substituted up to three times by fluorine and up to two times by identical or different radicals selected from the group consisting of hydroxyl, (C₁-C₄)-alkoxy, trifluoromethoxy, amino, mono-(C₁-C₄)-alkylamino, di-(C₁-C₄)-alkylamino, (C₁-C₄)-alkoxycarbonyl, (C₃-C₆)-cycloalkyl and 4- to 6-membered heterocyclyl
      and
      the cycloalkyl and heterocyclyl groups mentioned can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
   or
   R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
R⁵ represents a substituent selected from the group consisting of fluorine, chlorine, cyano, methyl, trifluoromethyl and hydroxyl
and
n represents the number 0, 1 or 2,
   where, if the substituent R⁵ occurs twice, its meanings can be identical or different, and their salts, solvates and solvates of the salts.

Compounds according to the invention are the compounds of the formula (I) and their salts, solvates and solvates of the salts, the compounds included in the formula (I) of the formulae mentioned in the following and their salts, solvates and solvates of the salts, and the compounds included in the formula (I) and mentioned in the following as embodiment examples and their salts, solvates and solvates of the salts, where the compounds included in the formula (I) and mentioned in the following are not already salts, solvates and solvates of the salts.

The compounds according to the invention can exist in stereoisomeric forms (enantiomers, diastereomers), depending on their structure. The invention therefore includes the enantiomers or diastereomers and their particular mixtures. The stereoisomerically uniform constituents can be isolated from such mixtures of enantiomers and/or diastereomers in a known manner; chromatography processes are preferably used for this, in particular HPLC chromatography on an achiral or chiral phase.

Where the compounds according to the invention can occur in tautomeric forms, the present invention includes all the tautomeric forms.

Preferred salts in the context of the present invention are physiologically acceptable salts of the compounds according to the invention. Salts which are not themselves suitable for pharmaceutical uses but can be used, for example, for isolation or purification of the compounds according to the invention are also included.

Physiologically acceptable salts of the compounds according to the invention include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates in the context of the invention are described as those forms of the compounds according to the invention which form a complex in the solid or liquid state by coordination with solvent molecules. Hydrates are a specific form of solvates, in which the coordination takes place with water. Hydrates are preferred solvates in the context of the present invention.

The N-oxides of pyridyl rings and tertiary cyclic amine groupings contained in compounds according to the invention are similarly included in the present invention.

The present invention moreover also includes prodrugs of the compounds according to the invention. The term “prodrugs” here designates compounds which themselves can be biologically active or inactive, but are converted (for example metabolically or hydrolytically) into compounds according to the invention during their dwell time in the body.

In the context of the present invention, the substituents have the following meaning, unless specified otherwise:

(C₁-C₆)-Alkyl and C₁-C₄)-alkyl in the context of the invention represent a straight-chain or branched alkyl radical having 1 to 6 or, respectively, 1 to 4 carbon atoms. A straight-chain or branched alkyl radical having 1 to 4 carbon atoms is preferred. There may be mentioned by way of example and preferably: methyl, ethyl, n-propyl, isopropyl, n-butyl, iso-butyl, sec-butyl, tert-butyl, n-pentyl, 2-pentyl, 3-pentyl, neopentyl, n-hexyl, 2-hexyl and 3-hexyl.

(C₁-C₄)-Alkanediyl and (C₂-C₄)-alkanediyl in the context of the invention represent a straight-chain divalent alkyl radical having 1 to 4 and 2 to 4 carbon atoms, respectively. There may be mentioned by way of example and preferably: methylene, ethane-1,2-diyl (1,2-ethylene), propane-1,3-diyl (1,3-propylene) and butane-1,4-diyl (1,4-butylene).

(C₁-C₆)-Alkylcarbonyl and (C₁-C₄)-alkylcarbonyl in the context of the invention represent a straight-chain or branched alkyl radical having 1 to 6 and 1 to 4 carbon atoms, respectively, which is linked via a carbonyl group [—C(═O)—]. A straight-chain or branched alkylcarbonyl group having 1 to 4 carbon atoms in the alkyl radical is preferred. There may be mentioned by way of example and preferably: acetyl, propionyl, n-butyryl, isobutyryl, n-pentanoyl, pivaloyl, n-hexanoyl and n-heptanoyl.

(C₁-C₆)-Alkylsulphonyl and (C₁-C₄)-alkylsulphonyl in the context of the invention represent a straight-chain or branched alkyl radical having 1 to 6 and 1 to 4 carbon atoms, respectively, which is linked via a sulphonyl group [—S(═O)₂—]. A straight-chain or branched alkylsulphonyl group having 1 to 4 carbon atoms in the alkyl radical is preferred. There may be mentioned by way of example and preferably: methylsulphonyl, ethylsulphonyl, n-propylsulphonyl, isopropylsulphonyl, n-butylsulphonyl, tert-butylsulphonyl, n-pentylsulphonyl and n-hexylsulphonyl.

Tri-(C₁-C₄)-alkylsilyl in the context of the invention represents a silyl group having three identical or different straight-chain or branched alkyl substituents, each of which contains 1 to 4 carbon atoms. There may be mentioned by way of example and preferably: trimethylsilyl, tert-butyldimethylsilyl and triisopropylsilyl.

(C₁-C₆)-Alkoxy and C₁-C₄)-alkoxy in the context of the invention represent a straight-chain or branched alkoxy radical having 1 to 6 and 1 to 4 carbon atoms, respectively. A straight-chain or branched alkoxy radical having 1 to 4 carbon atoms is preferred. There may be mentioned by way of example and preferably: methoxy, ethoxy, n-propoxy, isopropoxy, n-butoxy, isobutoxy, sec-butoxy, tert-butoxy, n-pentoxy, 2-pentoxy, 3-pentoxy, neopentoxy, n-hexoxy, 2-hexoxy and 3-hexoxy.

(C₁-C₆)-Alkoxycarbonyl and (C₁-C₄)-alkoxycarbonyl in the context of the invention represent a straight-chain alkoxy radical having 1 to 6 and 1 to 4 carbon atoms, respectively, which is linked via a carbonyl group [—C(═O)—]. A straight-chain or branched alkoxycarbonyl group having 1 to 4 carbon atoms in the alkoxy radical is preferred. There may be mentioned by way of example and preferably: methoxycarbonyl, ethoxycarbonyl, n-propoxycarbonyl, isopropoxycarbonyl, n-butoxycarbonyl, tert-butoxycarbonyl, n-pentoxycarbonyl and n-hexoxycarbonyl.

Mono-(C₁-C₄)-alkylamino in the context of the invention represents an amino group having a straight-chain or branched alkyl substituent which contains 1 to 4 carbon atoms. There may be mentioned by way of example and preferably: methylamino, ethylamino, n-propylamino, isopropylamino, n-butylamino and tert-butylamino.

Di-(C₁-C₄)-alkylamino in the context of the invention represents an amino group having two identical or different straight-chain or branched alkyl substituents, each of which contains 1 to 4 carbon atoms. There may be mentioned by way of example and preferably: N,N-dimethylamino, N,N-diethylamino, N-ethyl-N-methylamino, N-methyl-N-n-propylamino, N-isopropyl-N-methylamino, N-isopropyl-N-n-propylamino, N,N-diisopropylamino, N-n-butyl-N-methylamino and N-tert-butyl-N-methylamino.

(C₁-C₄)-Alkylcarbonylamino in the context of the invention represents an amino group having a straight-chain or branched alkylcarbonyl substituent which contains 1 to 4 carbon atoms in the alkyl radical and is linked to the nitrogen atom via the carbonyl group. There may be mentioned by way of example and preferably: acetylamino, propionylamino, n-butyrylamino, iso-butyrylamino, n-pentanoylamino and pivaloylamino.

(C₁-C₄)-Alkoxycarbonylamino in the context of the invention represents an amino group having a straight-chain or branched alkoxycarbonyl substituent which contains 1 to 4 carbon atoms in the alkoxy radical and is linked to the nitrogen atom via the carbonyl group. There may be mentioned by way of example and preferably: methoxycarbonylamino, ethoxycarbonylamino, n-propoxy-carbonylamino, isopropoxycarbonylamino, n-butoxycarbonylamino and tert-butoxycarbonylamino.

(C₃-C₆)-Cycloalkyl represents in the context of the invention a monocyclic saturated cycloalkyl group having 3 to 6 ring carbon atoms. There may be mentioned by way of example and preferably: cyclopropyl, cyclobutyl, cyclopentyl and cyclohexyl.

4- to 7-membered heterocyclyl and 4- to 6-membered heterocyclyl in the context of the invention represent a monocyclic, saturated heterocycle having 4 to 7 and 4 to 6 ring atoms, respectively, in total, which contains one or two ring heteroatoms from the group consisting of N, O, S and S(O)₂ and is linked via a ring carbon atom or optionally via a ring nitrogen atom. Preference is given to 4- to 6-membered heterocyclyl having one or two ring heteroatoms from the group consisting of N, O and S. There may be mentioned by way of example: azetidinyl, oxetanyl, thietanyl, pyrrolidinyl, pyrazolidinyl, tetrahydrofuranyl, thiolanyl, 1,1-dioxidothiolanyl, 1,3-oxazolidinyl, 1,3-thiazoli-dinyl, piperidinyl, piperazinyl, tetrahydropyranyl, tetrahydrothiopyranyl, 1,3-dioxanyl, 1,4-dioxanyl, morpholinyl, thiomorpholinyl, 1,1-dioxidothiomorpholinyl, hexahydroazepinyl and hexa-hydro-1,4-diazepinyl. Azetidinyl, oxetanyl, pyrrolidinyl, tetrahydrofuranyl, piperidinyl, piperazinyl, tetrahydropyranyl, morpholinyl and thiomorpholinyl are preferred.

5- or 6-membered heteroaryl in the context of the invention represents an aromatic heterocycle (heteroaromatics) having 5 or 6 ring atoms, respectively, in total, which contains up to three identical or different ring heteroatoms from the group consisting of N, O and S and is linked via a ring carbon atom or optionally via a ring nitrogen atom. Those which may be mentioned by way of example are: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl and triazinyl. 5- or 6-membered heteroaryl radicals having up to two ring heteroatoms from the group consisting of N, O and S, such as, for example, furyl, pyrrolyl, thienyl, thiazolyl, oxazolyl, isothiazolyl, isoxazolyl, pyrazolyl, imidazolyl, pyridyl, pyrimidinyl, pyridazinyl and pyrazinyl, are preferred.

Halogen in the context of the invention includes fluorine, chlorine, bromine and iodine. Chlorine, fluorine or bromine are preferred, and fluorine or chlorine are particularly preferred.

An oxo substituent in the context of the invention represents an oxygen atom, which is bonded to a carbon atom via a double bond.

If radicals in the compounds according to the invention are substituted, the radicals can be mono- or polysubstituted, unless specified otherwise. In the context of the present invention, for all the radicals which occur several times, the meaning thereof is independent of each other. Substitution by one or by two or three identical or different substituents is preferred. Substitution by one or by two identical or different substituents is particularly preferred.

The present invention provides in particular those compounds of the general formula (I) in which the ring

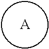

represents a phenyl or pyridyl ring and the adjacent groups X and CH₂ are bonded to ring carbon atoms of

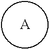

in 1, 3 or 1,4 relation to one another
and
the ring

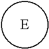

with the substituents R⁴ and R⁵ represents a phenyl ring of the formula

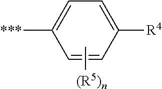

wherein
*** designates the linkage point with the ring

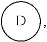

and their salts, solvates and solvates of the salts.

Compounds of the formula (I) which are preferred in the context of the present invention are those in which the ring

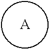

represents a pyridyl ring and the adjacent groups X and CH₂ are bonded to ring carbon atoms of this pyridyl ring in 1, 3 or 1,4 relation to one another
and
the ring

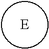

with the substituents R⁴ and R⁵ represents a phenyl ring of the formula

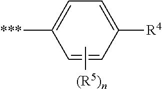

wherein
*** designates the linkage point with the ring

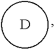

and their salts, solvates and solvates of the salts.

Preference is also given to compounds of the formula (I) in which the ring

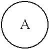

represents a phenyl ring and the adjacent groups X and CH₂ are bonded to this phenyl ring in 1, 3 or 1,4 relation to one another,
the ring

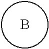

with the substituent R³ represents a heteroaryl ring of the formula

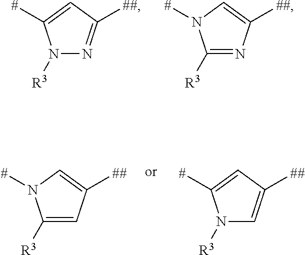

wherein
# designates the linkage point with the adjacent CH₂ group
and
## designates the linkage point with the ring

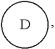

and
the ring

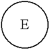

with the substituents R⁴ and R⁵ represents a phenyl ring of the formula

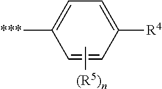

wherein
*** designates the linkage point with the ring

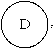

and their salts, solvates and solvates of the salts.

Preference is likewise given to compounds of the formula (I) in which the ring

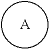

represents a phenyl ring and the adjacent groups X and CH₂ are bonded to this phenyl ring in 1, 3 or 1,4 relation to one another,
the ring

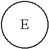

with the substituents R⁴ and R⁵ represents a phenyl ring of the formula

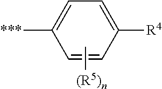

wherein
*** designates the linkage point with the ring

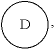

and
R¹ and R² together with the nitrogen atom to which they are attached form a saturated 4- to 7-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl, (C₁-C₄)-alkylsulphonyl and (C₃-C₆)-cycloalkyl,
   where (C₁-C₄)-alkyl and (C₃-C₆)-cycloalkyl for their part can be substituted by hydroxyl or (C₁-C₄)-alkoxy,
      and their salts, solvates and solvates of the salts.

Particular preference in the context of the present invention is given to compounds of the formula (I) in which

the ring

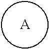

represents a pyridyl ring and the adjacent groups X and CH₂ are bonded to ring carbon atoms of this pyridyl ring in 1, 3 or 1,4 relation to one another,
the ring

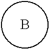

with the substituent R³ represents a heteroaryl ring of the formula

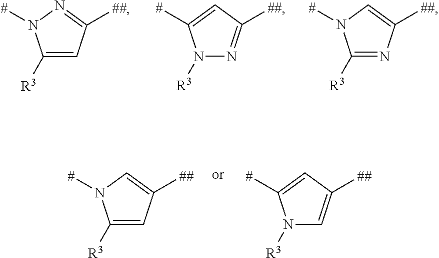

wherein
# designates the linkage point with the adjacent CH₂ group
and
## designates the linkage point with the ring

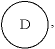

the ring

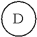

represents a heteroaryl ring of the formula

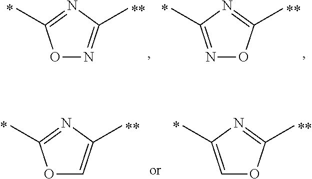

wherein
* designates the linkage point with the ring

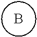

and
** designates the linkage point with the ring

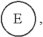

the ring

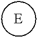

with the substituents R⁴ and R⁵ represents a phenyl ring of the formula

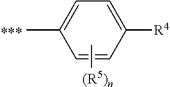

wherein
*** designates the linkage point with the ring

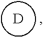

X represents a bond or represents —N(R⁶)—, —O—, —S—, ♦-C(═O)—N(R⁶)-♦♦ or ♦-N(R⁶)—C(═O)-♦♦, wherein
   ♦ designates the linkage point with the group L
   and
   ♦♦ designates the linkage point with the ring

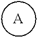

and
R⁶ denotes hydrogen, (C₁-C₄)-alkyl or (C₃-C₆)-cycloalkyl,
L represents straight-chain (C₁-C₄)-alkanediyl if X denotes a bond or the group ♦-C(═O)—N(R⁶)—♦♦,
   and
   represents straight-chain (C₂-C₄)-alkanediyl if X denotes the group —N(R⁶)—, —O—, —S— or ♦-N(R⁶)—C(═O)-♦♦,
R¹ represents hydrogen, (C₁-C₄)-alkyl, (C₁-C₄)-alkylcarbonyl, (C₁-C₄)-alkylsulphonyl or (C₃-C₆)-cycloalkyl,
   where the alkyl group in (C₁-C₄)-alkyl, (C₁-C₄)-alkylcarbonyl and (C₁-C₄)-alkylsulphonyl can be substituted up to three times by fluorine and up to two times by identical or different radicals selected from the group consisting of hydroxyl and (C₁-C₄)-alkoxy
   and
   (C₃-C₆)-cycloalkyl can be substituted up to two times by identical or different radicals selected from the group consisting of fluorine, (C₁-C₄)-alkyl, hydroxyl and (C₁-C₄)-alkoxy,
R² represents hydrogen, (C₁-C₄)-alkyl or (C₃-C₆)-cycloalkyl,
   where (C₁-C₄)-alkyl may be substituted up to three times by fluorine
   and
   (C₃-C₆)-cycloalkyl can be substituted up to two times by identical or different radicals selected from the group consisting of fluorine and (C₁-C₄)-alkyl,
or
R¹ and R² together with the nitrogen atom to which they are attached form a saturated 4- to 6-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl, (C₁-C₄)-alkylsulphonyl and (C₃-C₆)-cycloalkyl,
R³ represents methyl, ethyl or trifluoromethyl,
R⁴ represents a substituent selected from the group consisting of fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, (C₃-C₆)-cycloalkyl and 4- to 6-membered heterocyclyl, where (C₁-C₆)-alkyl for its part can be substituted up to three times by fluorine and up to two times by identical or different radicals selected from the group consisting of —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
   and where
   the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
   and
   the heteroaryl group mentioned for its part can be substituted up to two times by identical or different radicals selected from the group consisting of fluorine, chlorine, cyano, (C₁-C₄)-alkyl, trifluoromethyl, (C₁-C₄)-alkoxy and trifluoromethoxy,
   and wherein
   R⁷ and R⁸ independently of each other for each individual occurrence denote hydrogen, (C₁-C₄)-alkyl, (C₃-C₆)-cycloalkyl or 4- to 6-membered heterocyclyl,
      where (C₁-C₄)-alkyl can be substituted up to three times by fluorine and up to two times by identical or different radicals selected from the group consisting of hydroxyl, (C₁-C₄)-alkoxy, trifluoromethoxy, (C₃-C₆)-cycloalkyl and 4- to 6-membered heterocyclyl
      and
      the cycloalkyl and heterocyclyl groups mentioned can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
   or
   R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents a substituent selected from the group consisting of fluorine, chlorine and methyl
and
n represents the number 0 or 1,
and their salts, solvates and solvates of the salts.

Particular preference is also given to compounds of the formula (I) in which

the ring

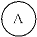

represents a phenyl ring and the adjacent groups X and CH₂ are bonded to this phenyl ring in 1, 3 or 1,4 relation to one another,
the ring

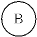

with the substituent R³ represents a heteroaryl ring of the formula

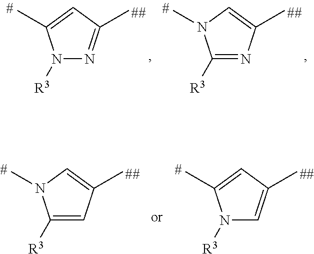

wherein
# designates the linkage point with the adjacent CH₂ group
and
## designates the linkage point with the ring

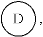

the ring

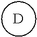

represents a heteroaryl ring of the formula

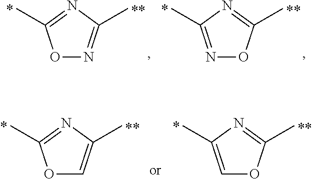

wherein
* designates the linkage point with the ring

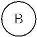

and
** designates the linkage point with the ring

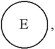

the ring

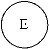

with the substituents R⁴ and R⁵ represents a phenyl ring of the formula

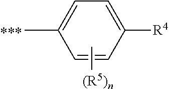

wherein
*** designates the linkage point with the ring

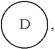

X represents a bond or represents —N(R⁶)—, —O—, —S—, ♦-C(═O)—N(R⁶)-♦♦ or ♦-N(R⁶)—C(═O)-♦♦, wherein
   ♦ designates the linkage point with the group L
   and
   ♦♦ designates the linkage point with the ring

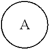

and
R⁶ denotes hydrogen, (C₁-C₄)-alkyl or (C₃-C₆)-cycloalkyl,
L represents straight-chain (C₁-C₄)-alkanediyl if X denotes a bond or the group ♦-C(═O)—N(R⁶)-♦♦,
   and
   represents straight-chain (C₂-C₄)-alkanediyl if X denotes the group —N(R⁶)—, —O—, —S— or ♦-N(R⁶)—C(═O)-♦♦,
R¹ represents hydrogen, (C₁-C₄)-alkyl, (C₁-C₄)-alkylcarbonyl, (C₁-C₄)-alkylsulphonyl or (C₃-C₆)-cycloalkyl,
   where the alkyl group in (C₁-C₄)-alkyl, (C₁-C₄)-alkylcarbonyl and (C₁-C₄)-alkylsulphonyl can be substituted up to three times by fluorine and up to two times by identical or different radicals selected from the group consisting of hydroxyl and (C₁-C₄)-alkoxy
   and
   (C₃-C₆)-cycloalkyl can be substituted up to two times by identical or different radicals selected from the group consisting of fluorine, (C₁-C₄)-alkyl, hydroxyl and (C₁-C₄)-alkoxy,
R² represents hydrogen, (C₁-C₄)-alkyl or (C₃-C₆)-cycloalkyl,
   where (C₁-C₄)-alkyl may be substituted up to three times by fluorine
   and
   (C₃-C₆)-cycloalkyl can be substituted up to two times by identical or different radicals selected from the group consisting of fluorine and (C₁-C₄)-alkyl,
R³ represents methyl, ethyl or trifluoromethyl,
R⁴ represents a substituent selected from the group consisting of fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, (C₃-C₆)-cycloalkyl and 4- to 6-membered heterocyclyl,
   where (C₁-C₆)-alkyl for its part can be substituted up to three times by fluorine and up to two times by identical or different radicals selected from the group consisting of —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
   and where
   the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
   and
   the heteroaryl group mentioned for its part can be substituted up to two times by identical or different radicals selected from the group consisting of fluorine, chlorine, cyano, (C₁-C₄)-alkyl, trifluoromethyl, (C₁-C₄)-alkoxy and trifluoromethoxy,
   and wherein
   R⁷ and R⁸ independently of each other for each individual occurrence denote hydrogen, (C₁-C₄)-alkyl, (C₃-C₆)-cycloalkyl or 4- to 6-membered heterocyclyl,
      where (C₁-C₄)-alkyl can be substituted up to three times by fluorine and up to two times by identical or different radicals selected from the group consisting of hydroxyl, (C₁-C₄)-alkoxy, trifluoromethoxy, (C₃-C₆)-cycloalkyl and 4- to 6-membered heterocyclyl
      and
      the cycloalkyl and heterocyclyl groups mentioned can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
   or
   R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents a substituent selected from the group consisting of fluorine, chlorine and methyl
and
n represents the number 0 or 1,
and their salts, solvates and solvates of the salts.

Particular preference is likewise given to compounds of the formula (I) in which

the ring

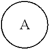

represents a phenyl ring and the adjacent groups X and CH₂ are bonded to this phenyl ring in 1, 3 or 1,4 relation to one another,
the ring

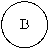

with the substituent R³ represents a heteroaryl ring of the formula

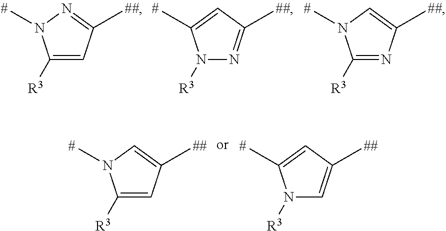

wherein
# designates the linkage point with the adjacent CH₂ group
and
## designates the linkage point with the ring

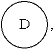

the ring

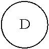

represents a heteroaryl ring of the formula

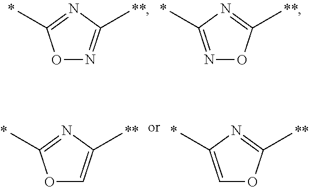

wherein
* designates the linkage point with the ring

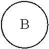

and
** designates the linkage point with the ring

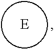

the ring

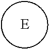

with the substituents R⁴ and R⁵ represents a phenyl ring of the formula

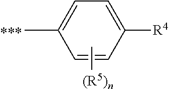

wherein
*** designates the linkage point with the ring

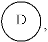

X represents a bond or represents —N(R⁶)—, —O—, —S—, ♦-C(═O)—N(R⁶)-♦♦ or ♦-N(R⁶)—C(═O)-♦♦, wherein
   ♦ designates the linkage point with the group L
   and
   ♦♦ designates the linkage point with the ring

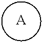

and
R⁶ denotes hydrogen, (C₁-C₄)-alkyl or (C₃-C₆)-cycloalkyl,
L represents straight-chain (C₁-C₄)-alkanediyl if X denotes a bond or the group ♦-C(═O)—N(R⁶)-♦♦,
   and
   represents straight-chain (C₂-C₄)-alkanediyl if X denotes the group —N(R⁶)—, —O—, —S— or ♦-N(R⁶)—C(═O)-♦♦,
R¹ and R² together with the nitrogen atom to which they are attached form a saturated 4- to 6-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl, (C₁-C₄)-alkylsulphonyl and (C₃-C₆)-cycloalkyl,
R³ represents methyl, ethyl or trifluoromethyl,
R⁴ represents a substituent selected from the group consisting of fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, (C₃-C₆)-cycloalkyl and 4- to 6-membered heterocyclyl,
   where (C₁-C₆)-alkyl for its part can be substituted up to three times by fluorine and up to two times by identical or different radicals selected from the group consisting of —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
   and where
   the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
   and
   the heteroaryl group mentioned for its part can be substituted up to two times by identical or different radicals selected from the group consisting of fluorine, chlorine, cyano, (C₁-C₄)-alkyl, trifluoromethyl, (C₁-C₄)-alkoxy and trifluoromethoxy,
   and wherein
   R⁷ and R⁸ independently of each other for each individual occurrence denote hydrogen, (C₁-C₄)-alkyl, (C₃-C₆)-cycloalkyl or 4- to 6-membered heterocyclyl,
      where (C₁-C₄)-alkyl can be substituted up to three times by fluorine and up to two times by identical or different radicals selected from the group consisting of hydroxyl, (C₁-C₄)-alkoxy, trifluoromethoxy, (C₃-C₆)-cycloalkyl and 4- to 6-membered heterocyclyl
      and
      the cycloalkyl and heterocyclyl groups mentioned can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
   or
   R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents a substituent selected from the group consisting of fluorine, chlorine and methyl
and
n represents the number 0 or 1,
and their salts, solvates and solvates of the salts.

In a particularly preferred embodiment, the present invention also comprises compounds of the formula (I) in which

the ring

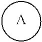

represents a phenyl ring and the adjacent groups X and CH₂ are bonded to this phenyl ring in 1, 3 or 1,4 relation to one another,
the ring

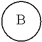

with the substituent R³ represents a heteroaryl ring of the formula

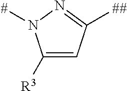

wherein
# designates the linkage point with the adjacent CH₂ group
and
## designates the linkage point with the ring

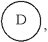

the ring

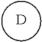

represents a heteroaryl ring of the formula

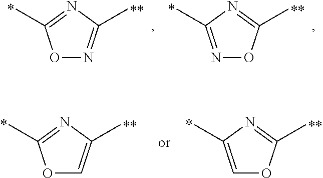

wherein
* designates the linkage point with the ring

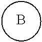

and
** designates the linkage point with the ring

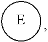

the ring

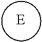

with the substituents R⁴ and R⁵ represents a phenyl ring of the formula

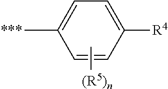

wherein
*** designates the linkage point with the ring

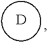

X represents a bond or represents —N(R⁶)—, —O—, —S—, ♦-C(═O)—N(R⁶)-♦♦ or ♦-N(R⁶)—C(═O)-♦♦, wherein
   ♦ designates the linkage point with the group L
   and
   ♦♦ designates the linkage point with the ring

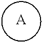

and
R⁶ denotes hydrogen, (C₁-C₄)-alkyl or (C₃-C₆)-cycloalkyl,
L represents straight-chain (C₁-C₄)-alkanediyl if X denotes a bond or the group ♦-C(═O)—N(R⁶)-♦♦,
   and
   represents straight-chain (C₂-C₄)-alkanediyl if X denotes the group —N(R⁶)—, —O—, —S— or ♦-N(R⁶)—C(═O)-♦♦,
R¹ represents hydrogen, (C₁-C₄)-alkyl or (C₃-C₆)-cycloalkyl,
   where (C₁-C₄)-alkyl can be substituted up to three times by fluorine and up to two times by identical or different radicals selected from the group consisting of hydroxyl and (C₁-C₄)-alkoxy,
R² represents hydrogen, (C₁-C₄)-alkyl or (C₃-C₆)-cycloalkyl,
R³ represents methyl, ethyl or trifluoromethyl,
R⁴ represents a substituent selected from the group consisting of fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, (C₃-C₆)-cycloalkyl and 4- to 6-membered heterocyclyl,
   where (C₁-C₆)-alkyl for its part can be substituted up to three times by fluorine and up to two times by identical or different radicals selected from the group consisting of —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
   and where
   the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
   and
   the heteroaryl group mentioned for its part can be substituted up to two times by identical or different radicals selected from the group consisting of fluorine, chlorine, cyano, (C₁-C₄)-alkyl, trifluoromethyl, (C₁-C₄)-alkoxy and trifluoromethoxy,
   and wherein
   R⁷ and R⁸ independently of each other for each individual occurrence denote hydrogen, (C₁-C₄)-alkyl, (C₃-C₆)-cycloalkyl or 4- to 6-membered heterocyclyl,
      where (C₁-C₄)-alkyl can be substituted up to three times by fluorine and up to two times by identical or different radicals selected from the group consisting of hydroxyl, (C₁-C₄)-alkoxy, trifluoromethoxy, (C₃-C₆)-cycloalkyl and 4- to 6-membered heterocyclyl
      and
      the cycloalkyl and heterocyclyl groups mentioned can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
   or
   R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents a substituent selected from the group consisting of fluorine, chlorine and methyl
and
n represents the number 0 or 1,
and their salts, solvates and solvates of the salts.

This last-described embodiment of the invention is distinguished by good solubility of the compounds in aqueous or physiological media, resulting in an easier absorption of the compounds after oral administration.

Very particular preference in the context of the present invention is given to compounds of the formula (I) in which

the ring

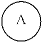

represents a pyridyl ring of the formula

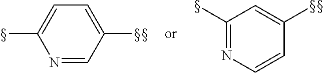

wherein
§ designates the linkage point with the adjacent group X
and
§§ designates the linkage point with the adjacent CH₂ group,
   the ring

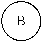

with the substituent R³ represents a heteroaryl ring of the formula

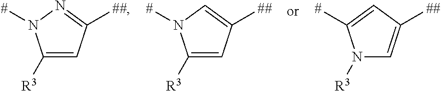

wherein
# designates the linkage point with the adjacent CH₂ group
and
## designates the linkage point with the ring

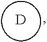

the ring

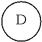

represents a heteroaryl ring of the formula

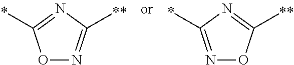

wherein
* designates the linkage point with the ring

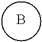

and
** designates the linkage point with the ring

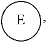

the ring

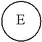

with the substituents R⁴ and R⁵ represents a phenyl ring of the formula

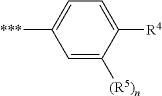

wherein
*** designates the linkage point with the ring

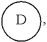

X represents —N(R⁶)—, —O—, —S— or ♦-N(R⁶)—C(═O)-♦♦ wherein
   ♦ designates the linkage point with the group L
   and
   ♦♦ designates the linkage point with the ring

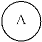

and
R⁶ denotes hydrogen, methyl, ethyl, isopropyl, cyclopropyl or cyclobutyl,
L represents ethane-1,2-diyl or propane-1,3-diyl,
R¹ represents hydrogen, (C₁-C₄)-alkyl, (C₁-C₄)-alkylcarbonyl, (C₁-C₄)-alkylsulphonyl or (C₃-C₆)-cycloalkyl,
   where the alkyl group in (C₁-C₄)-alkyl, (C₁-C₄)-alkylcarbonyl and (C₁-C₄)-alkylsulphonyl may be substituted by hydroxyl or (C₁-C₄)-alkoxy or up to three times by fluorine
      and
      (C₃-C₆)-cycloalkyl can be substituted up to two times by identical or different radicals selected from the group consisting of fluorine, (C₁-C₄)-alkyl, hydroxyl and (C₁-C₄)-alkoxy,
R² represents hydrogen, (C₁-C₄)-alkyl or cyclopropyl,
   where (C₁-C₄)-alkyl may be substituted up to three times by fluorine,
or
R¹ and R² together with the nitrogen atom to which they are attached form a saturated 4- to 6-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₃-C₆)-cycloalkyl,
R³ represents methyl,
R⁴ represents a substituent selected from the group consisting of chlorine, (C₁-C₆)-alkyl, trimethylsilyl, —OR⁷, —SR⁷, —S(═O)—R⁷, —S(═O)₂—R⁷, (C₃-C₆)-cycloalkyl and 4- to 6-membered heterocyclyl,
   where (C₁-C₆)-alkyl for its part can be substituted by a radical selected from the group consisting of —OR⁷, —NR⁷R⁸, —C(═O)—NR⁷R⁸, (C₃-C₆)-cycloalkyl and 4- to 6-membered heterocyclyl and up to three times by fluorine
   and
   the cycloalkyl and heterocyclyl groups mentioned can for their part be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
   and wherein
   R⁷ and R⁸ independently of each other for each individual occurrence denote hydrogen, (C₁-C₄)-alkyl or (C₃-C₆)-cycloalkyl,
      where (C₁-C₄)-alkyl can be substituted by hydroxyl, (C₁-C₄)-alkoxy, trifluoromethoxy or (C₃-C₆)-cycloalkyl and up to three times by fluorine
      and
      the cycloalkyl groups mentioned can be substituted up to two times by identical or different radicals selected from the group consisting of fluorine, (C₁-C₄)-alkyl, trifluoromethyl, hydroxyl, (C₁-C₄)-alkoxy and trifluoromethoxy,
   or
   R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
and
n represents the number 0 or 1,
and their salts, solvates and solvates of the salts.

Very particular preference is also given to compounds of the formula (I) in which

the ring

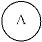

represents a phenyl ring and the adjacent groups X and CH₂ are bonded to this phenyl ring in 1, 3 or 1,4 relation to one another,
the ring

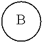

with the substituent R³ represents a heteroaryl ring of the formula

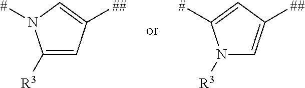

wherein
# designates the linkage point with the adjacent CH₂ group
and
## designates the linkage point with the ring

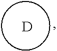

the ring

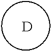

represents a heteroaryl ring of the formula

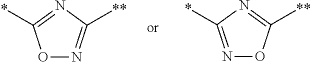

wherein
* designates the linkage point with the ring

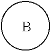

and
** designates the linkage point with the ring

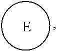

the ring

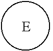

with the substituents R⁴ and R⁵ represents a phenyl ring of the formula

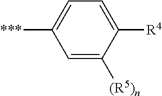

wherein
*** designates the linkage point with the ring

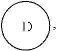

X represents —N(R⁶)—, —O—, —S— or ♦-N(R⁶)—C(═O)-♦♦ wherein
   ♦ designates the linkage point with the group L
   and
   ♦♦ designates the linkage point with the ring

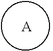

and
R⁶ denotes hydrogen, methyl, ethyl, isopropyl, cyclopropyl or cyclobutyl,
L represents ethane-1,2-diyl or propane-1,3-diyl,
R¹ represents hydrogen, (C₁-C₄)-alkyl, (C₁-C₄)-alkylcarbonyl, (C₁-C₄)-alkylsulphonyl or (C₃-C₆)-cycloalkyl,
   where the alkyl group in (C₁-C₄)-alkyl, (C₁-C₄)-alkylcarbonyl and (C₁-C₄)-alkylsulphonyl may be substituted by hydroxyl or (C₁-C₄)-alkoxy or up to three times by fluorine
   and
   (C₃-C₆)-cycloalkyl can be substituted up to two times by identical or different radicals selected from the group consisting of fluorine, (C₁-C₄)-alkyl, hydroxyl and (C₁-C₄)-alkoxy,
R² represents hydrogen, (C₁-C₄)-alkyl or cyclopropyl,
   where (C₁-C₄)-alkyl may be substituted up to three times by fluorine,
R³ represents methyl,
R⁴ represents a substituent selected from the group consisting of chlorine, (C₁-C₆)-alkyl, trimethylsilyl, —OR⁷, —SR⁷, —S(═O)—R⁷, —S(═O)₂—R⁷, (C₃-C₆)-cycloalkyl and 4- to 6-membered heterocyclyl,
   where (C₁-C₆)-alkyl for its part can be substituted by a radical selected from the group consisting of —OR⁷, —NR⁷R⁸, —C(═O)—NR⁷R⁸, (C₃-C₆)-cycloalkyl and 4- to 6-membered heterocyclyl and up to three times by fluorine
   and
   the cycloalkyl and heterocyclyl groups mentioned can for their part be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (Cᵣ C₄)-alkylcarbonyl
   and wherein
   R⁷ and R⁸ independently of each other for each individual occurrence denote hydrogen, (C₁-C₄)-alkyl or (C₃-C₆)-cycloalkyl,
      where (C₁-C₄)-alkyl can be substituted by hydroxyl, (C₁-C₄)-alkoxy, trifluoromethoxy or (C₃-C₆)-cycloalkyl and up to three times by fluorine
      and
      the cycloalkyl groups mentioned can be substituted up to two times by identical or different radicals selected from the group consisting of fluorine, (C₁-C₄)-alkyl, trifluoromethyl, hydroxyl, (C₁-C₄)-alkoxy and trifluoromethoxy,
   or
   R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
and
n represents the number 0 or 1,
and their salts, solvates and solvates of the salts.

Very particular preference is likewise given to compounds of the formula (I) in which

the ring

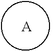

represents a phenyl ring and the adjacent groups X and CH₂ are bonded to this phenyl ring in 1, 3 or 1,4 relation to one another,
the ring

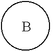

with the substituent R³ represents a heteroaryl ring of the formula

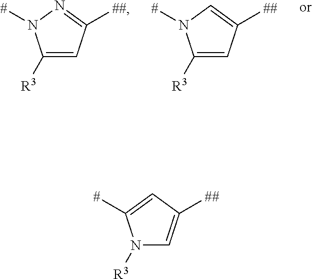

wherein
# designates the linkage point with the adjacent CH₂ group
and
## designates the linkage point with the ring

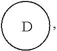

the ring

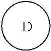

represents a heteroaryl ring of the formula

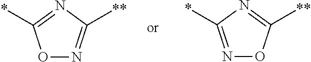

wherein
* designates the linkage point with the ring

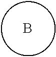

and
** designates the linkage point with the ring

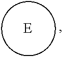

the ring

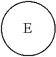

with the substituents R⁴ and R⁵ represents a phenyl ring of the formula

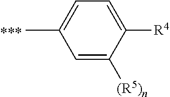

wherein
*** designates the linkage point with the ring

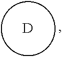

X represents —N(R⁶)—, —O—, —S— or ♦-N(R⁶)—C(═O)-♦♦ wherein
   ♦ designates the linkage point with the group L
   and
   ♦♦ designates the linkage point with the ring

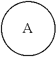

and
R⁶ denotes hydrogen, methyl, ethyl, isopropyl, cyclopropyl or cyclobutyl,
L represents ethane-1,2-diyl or propane-1,3-diyl,
R¹ and R² together with the nitrogen atom to which they are attached form a saturated 4- to 6-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₃-C₆)-cycloalkyl,
R³ represents methyl,
R⁴ represents a substituent selected from the group consisting of chlorine, (C₁-C₆)-alkyl, trimethylsilyl, —OR⁷, —SR⁷, —S(═O)—R⁷, —S(═O)₂—R⁷, (C₃-C₆)-cycloalkyl and 4- to 6-membered heterocyclyl,
   where (C₁-C₆)-alkyl for its part can be substituted by a radical selected from the group consisting of —OR⁷, —NR⁷R⁸, —C(═O)—NR⁷R⁸, (C₃-C₆)-cycloalkyl and 4- to 6-membered heterocyclyl and up to three times by fluorine
   and
   the cycloalkyl and heterocyclyl groups mentioned can for their part be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
   and wherein
   R⁷ and R⁸ independently of each other for each individual occurrence denote hydrogen, (C₁-C₄)-alkyl or (C₃-C₆)-cycloalkyl,
      where (C₁-C₄)-alkyl can be substituted by hydroxyl, (C₁-C₄)-alkoxy, trifluoromethoxy or (C₃-C₆)-cycloalkyl and up to three times by fluorine
      and
      the cycloalkyl groups mentioned can be substituted up to two times by identical or different radicals selected from the group consisting of fluorine, (C₁-C₄)-alkyl, trifluoromethyl, hydroxyl, (C₁-C₄)-alkoxy and trifluoromethoxy,
   or
   R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
and
n represents the number 0 or 1,
and their salts, solvates and solvates of the salts.

In a very particularly preferred embodiment, the present invention also comprises compounds of the formula (I) in which

the ring

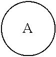

represents a phenyl ring and the adjacent groups X and CH₂ are bonded to this phenyl ring in 1, 3 or 1,4 relation to one another,
the ring

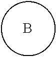

with the substituent R³ represents a heteroaryl ring of the formula

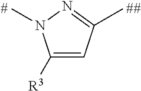

wherein
# designates the linkage point with the adjacent CH₂ group
and
## designates the linkage point with the ring

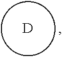

the ring

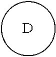

represents a heteroaryl ring of the formula

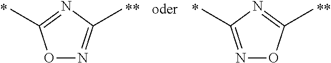

wherein
* designates the linkage point with the ring

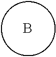

and
** designates the linkage point with the ring

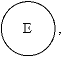

the ring

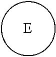

with the substituents R⁴ and R⁵ represents a phenyl ring of the formula

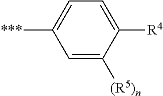

wherein
*** designates the linkage point with the ring

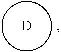

X represents —N(R⁶)—, —O—, —S— or ♦-N(R⁶)—C(═O)-♦♦ wherein
   ♦ designates the linkage point with the group L
   and
   ♦♦ designates the linkage point with the ring

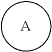

and
R⁶ denotes hydrogen, methyl, ethyl, isopropyl, cyclopropyl or cyclobutyl,
L represents ethane-1,2-diyl or propane-1,3-diyl,
R¹ represents hydrogen, methyl, ethyl, 2-hydroxyethyl, 2-methoxyethyl, isopropyl, cyclopropyl or cyclobutyl,
R² represents hydrogen, methyl or cyclopropyl,
R³ represents methyl,
R⁴ represents a substituent selected from the group consisting of chlorine, (C₁-C₆)-alkyl, trimethylsilyl, —OR⁷, —SR⁷, —S(═O)—R⁷, —S(═O)₂—R⁷, (C₃-C₆)-cycloalkyl and 4- to 6-membered heterocyclyl,
   where (C₁-C₆)-alkyl for its part can be substituted by a radical selected from the group consisting of —OR⁷, —NR⁷R⁸, —C(═O)—NR⁷R⁸, (C₃-C₆)-cycloalkyl and 4- to 6-membered heterocyclyl and up to three times by fluorine
   and
   the cycloalkyl and heterocyclyl groups mentioned can for their part be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
   and wherein
   R⁷ and R⁸ independently of each other for each individual occurrence denote hydrogen, (C₁-C₄)-alkyl or (C₃-C₆)-cycloalkyl,
      where (C₁-C₄)-alkyl can be substituted by hydroxyl, (C₁-C₄)-alkoxy, trifluoromethoxy or (C₃-C₆)-cycloalkyl and up to three times by fluorine
      and
      the cycloalkyl groups mentioned can be substituted up to two times by identical or different radicals selected from the group consisting of fluorine, (C₁-C₄)-alkyl, trifluoromethyl, hydroxyl, (C₁-C₄)-alkoxy and trifluoromethoxy,
   or
   R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
and
n represents the number 0 or 1,
and their salts, solvates and solvates of the salts.

This last-described embodiment of the invention is distinguished, in particular, by good solubility of the compounds in aqueous or physiological media, resulting in an easier absorption of the compounds after oral administration.

The radical definitions given in detail in the particular combinations or preferred combinations of radicals are also replaced as desired by radical definitions of other combinations, independently of the particular combinations of radicals given.

Combinations of two or more of the abovementioned preferred ranges are very particularly preferred.

The compounds according to the invention can be prepared in various ways. The main methods which are called process A, B, C and D in the following and can be carried out in various variants were used here in particular.

Process A (with variants A.1, A.2, A.3 and A.4; see Equations 1-4) is characterized in that compounds of the formula (VI) in which B, D, E, R³, R⁴, R⁵ and n have the meanings described above and in which the hydrogen atoms indicated is attached to a nitrogen atom of ring B are reacted with a compound of the formula (II), (III) or (IV) in which A, L, X, R¹ and R² have the meanings described above and in which Y quite generally represents an atom or a group from which or with the aid of which the radical R¹R²N-L-X can be constructed and in which Z represents a leaving group. Examples of Y are chlorine, bromine, iodine, cyano, nitro, hydroxyl, formyl, carboxyl and alkoxycarbonyl; examples of Z are chlorine, bromine, iodine, methanesulphonate (mesylate), trifluoromethanesulphonate (triflate) and 4-methylbenzenesulphonate (tosylate).

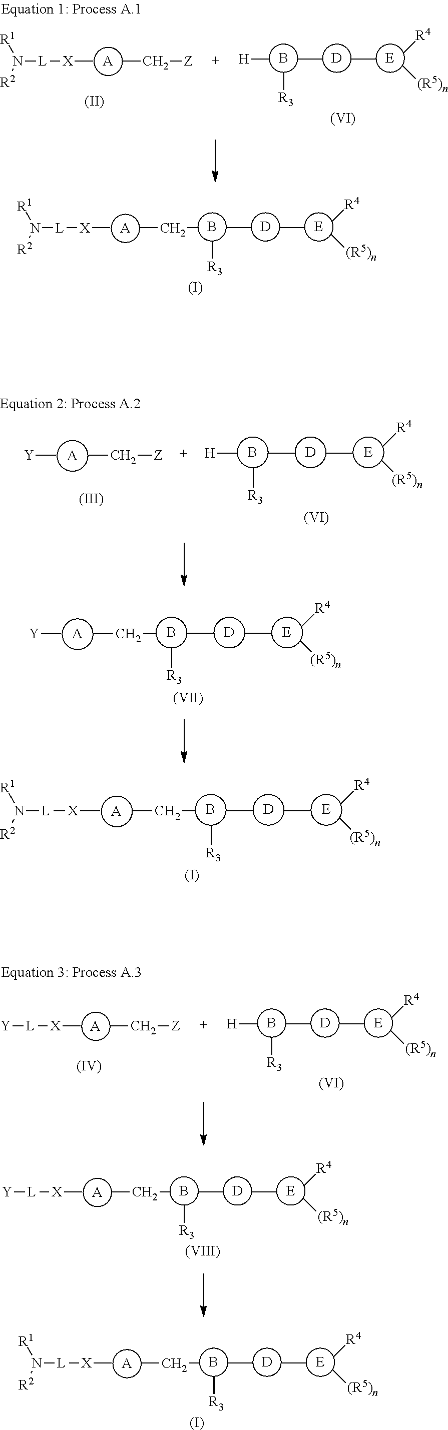

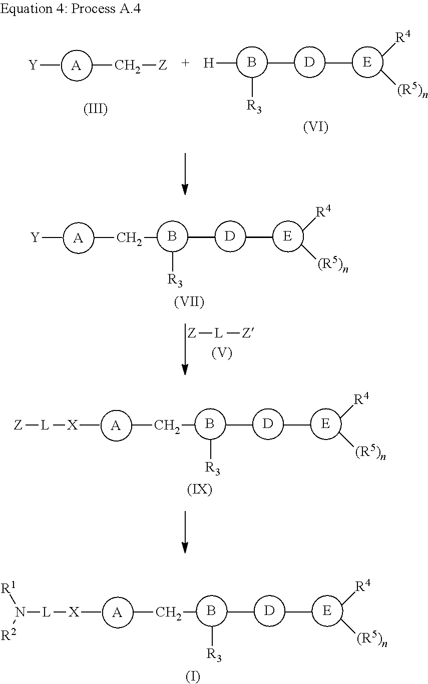

[Z and Z′ in formula (V) are leaving groups as described in the text above which may be identical or may differ from one another; furthermore, the leaving groups Z in formula (III) and in formula (V) may be identical or may differ from one another].

The reaction of the compounds of the formula (II), (III) or (IV) with the compounds of the formula (VI) is carried out in the presence of a strong base, such as, for example and preferably, potassium tert-butoxide, in a suitable solvent, such as, for example and preferably, tetrahydrofuran, in a temperature range of between −10° C. and +50° C., preferably between 0° C. and room temperature. The subsequent reaction of the intermediates of the formulae (VII), (VIII) and (IX) to give the products of the formula (I) varies and depends in particular on the nature of group X and ring A. These subsequent reactions are described below.

In process B ring D is built up, ring D representing a 1,2,4-oxadiazole here. Process B is also used in various modifications. The variants of process B (variants B.1, B.2, B.3 and B.4) are similar to the various variants of process A with respect to the starting materials used and the part-reactions which follow the ring closure to the oxadiazole. Only variant B.1 is therefore to be described in detail in the following (Equation 5). Compounds of the formula (X) in which A, B, L, X, R′, R² and R³ have the meanings described above, are reacted here with hydroxyamidines of the formula (XI) in which E, R⁴, R⁵ and n have the meanings given above, to give the products of the formula (I-A).

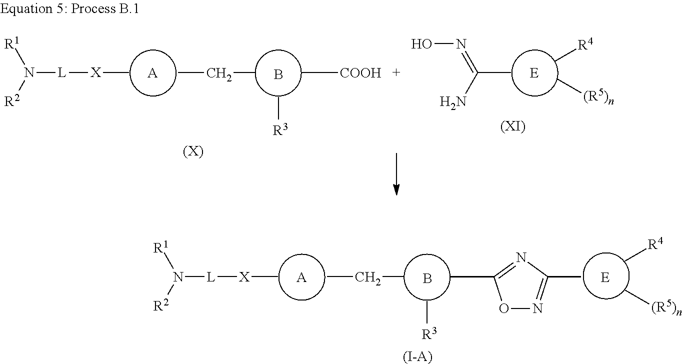

The reaction of the compounds of the formula (X) with the compounds of the formula (XI) is carried out in the presence of coupling reagents, such as, for example, 1H-benzotriazol-1-ol and N-[3-(dimethylamino)propyl]-N-ethylcarbodiimide hydrochloride, in the presence of tertiary amine bases, such as, for example, triethylamine, in suitable solvents, such as, for example, N,N-dimethylformamide. The reaction partners are first reacted with one another at room temperature for some time, before the mixture is then heated to temperatures in the range of from +80° C. to +140° C. Alternatively, the compounds of the formula (X) can first be converted into the corresponding carboxylic acid chlorides. Chlorinating reagents, such as, for example, oxalyl chloride or thionyl chloride, in inert solvents, such as, for example, dichloromethane or chloroform, are employed for this. The reaction is preferably carried out at room temperature and in the presence of a catalytic amount of N,N-dimethylformamide. The acid chloride obtained in this way is then reacted with the compounds of the formula (XI). The product of this reaction is then heated to temperatures in the range of from +80° C. to +140° C. in inert solvents, such as, for example, dimethylsulphoxide or N,N-dimethylformamide.

In the remaining variants of process B, instead of compounds of the formula (X), carboxylic acids of the formula (XII) (processes B.2 and B.4) or (XIII) (process B.3) in which A, B, L, X, Y and R³ in each case have the meanings described above, are employed.

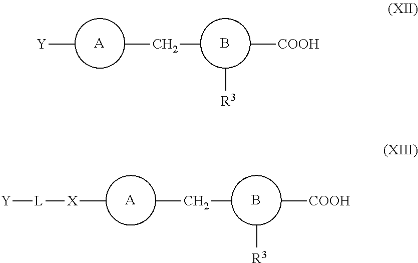

If the ring D represents a 1,3-oxazole, process C can be used, which can be carried out analogously to processes A and B in various variants C.1, C.2, C.3 and C.4. As is the case for process B, only variant C.1 is explained in more detail in the following (Equation 6). In process C.1 compounds of the formula (X) are reacted with compounds of the formula (XIV) to give intermediates of the formula (XV), which, after cyclization, are oxidized to the products of the formula (I-B). A, B, E, L, X, R¹, R², R³, R⁴, R⁵ and n each have the meanings described above.

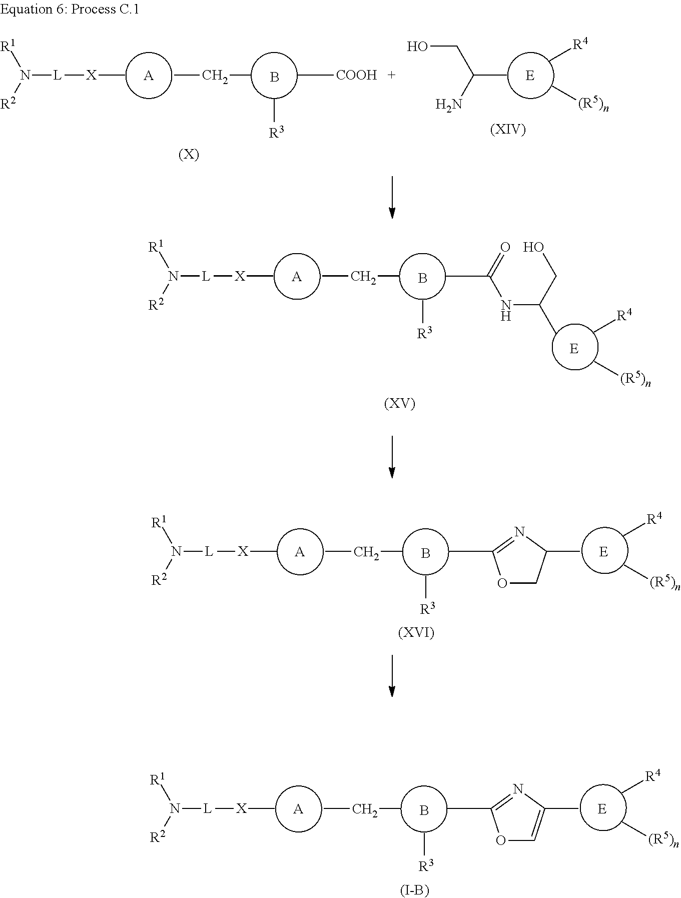

The compounds of the formula (X) are reacted with the amino alcohols of the formula (XIV) in the presence of coupling reagents, such as, for example, O-(7-azabenzotriazol-1-yl)-N,N,N′,N′-tetramethyluronium hexafluorophosphate. The reaction is carried out at room temperature in the presence of tertiary amine bases, such as, for example, triethylamine, in polar aprotic solvents, such as, for example, N,N-dimethylformamide. Subsequent cyclization to give the compounds of the formula (XVI) is achieved with the aid of a cyclizing reagent, such as, for example and preferably, with Burgess reagent (carbomethoxysulphamoyltriethylammonium hydroxide). The reaction is carried out in suitable solvents, such as, for example, tetrahydrofuran, at the boiling point of the solvent. The final oxidation can be carried out with various oxidizing agents. Oxidation with activated manganese dioxide in tetrahydrofuran at the boiling point of the solvent is preferred.

In the other variants of process C, the 1,3-oxazole ring is built up in the same manner Instead of compounds of the formula (X), carboxylic acids of the formula (XII) (processes C.2 and C.4) or (XIII) (process C.3) in which A, B, L, X, Y and R³ have the meanings described above, are employed.

Process D describes the preparation of compounds of the formula (I) in which the ring D represents a 1,2,4-oxadiazole which, in contrast to the oxadiazole derivatives described in process B, is linked to the adjacent groups in a manner in which the sides are switched. Analogously to processes A, B and C, process D can be carried out in the various variants D.1, D.2, D.3 and D.4; as is the case for processes B and C, only variant D.1 is explained in more detail in the following (Equation 7). The carboxylic acids of the formula (X) are first converted here into the primary amides of the formula (XVII), from which the nitriles of the formula (XVIII) are then prepared. By reaction with hydroxylamine, these are converted into the hydroxyamidines of the formula (XIX), from which the products of the formula (I-C) are obtained by coupling with the acid chlorides of the formula (XX) and subsequent cyclization. A, B, E, L, X, R¹, R², R³, R⁴, R⁵ and n each have the meanings described above.

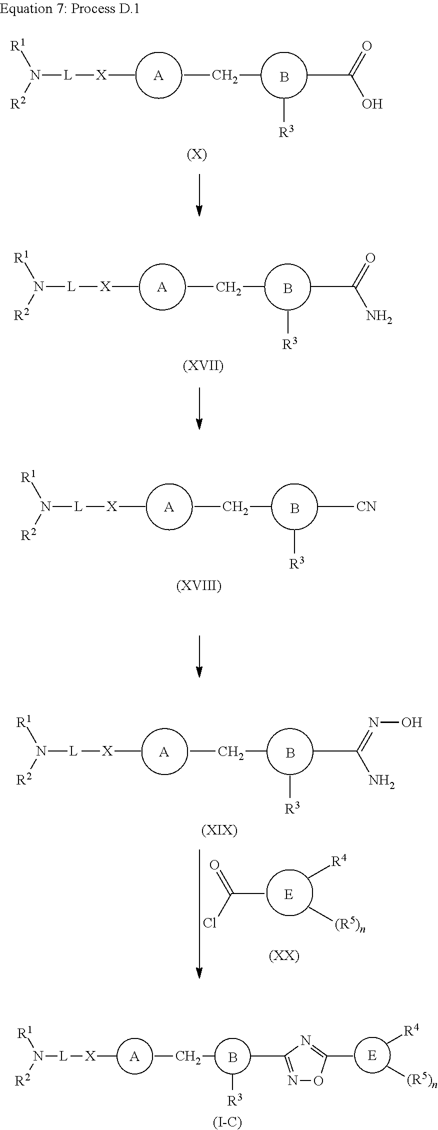

The reaction of the carboxylic acids of the formula (X) to give the amides of the formula (XVII) is carried out in two stages: First by reaction with chlorinating reagents, such as, for example, oxalyl chloride or thionyl chloride, in inert solvents, such as, for example, dichloromethane or chloroform, and then by reaction of the carboxylic acid chlorides obtained in this way with solutions of ammonia in methanol or water in a suitable co-solvent, such as, for example, tetrahydrofuran or 1,4-dioxane. The dehydration of the primary amides of the formula (XVII) to give the nitriles of the formula (XVIII) is carried out by reaction with anhydrides or chlorides of strong acids, such as, for example and preferably, of trifluoromethanesulphonic acid or trifluoroacetic acid, in the presence of an excess of a base, such as, for example, triethylamine or N,N-diisopropylethylamine, in inert solvents, such as, for example, dichloromethane. The reaction is preferably carried out in the temperature range of between 0° C. and room temperature. The subsequent reaction with hydroxylamine is preferably carried out in alcoholic solvents, such as, for example, ethanol, at the boiling point of the solvent. The hydroxyamidines of the formula (XIX) obtained in this way are reacted with the acid chlorides of the formula (XX) in the presence of bases, such as, for example, triethylamine or N,N-diisopropylethylamine, in inert solvents, such as, for example, dichloromethane or ethyl acetate, at temperatures of between −10° C. and room temperature. The intermediate products thereby obtained are cyclized to the products of the formula (I-C) in inert solvents, such as, for example, dimethylsulphoxide or N,N-dimethylformamide, at temperatures of between +80° C. and +160° C.

The reactions which lead from the intermediates of the formula (VII) (process A.2, Equation 2) to the products of the formula (I), depending on the group X and the nature of the ring A, are described in the following. These reactions are also used correspondingly in processes B.2, C.2 and D.2.

a) If X represents NR⁶, O or S where R⁶ has the meaning described above, and the ring A represents a pyridine ring, and the group Y is bonded to a carbon atom of this pyridine ring which is in the direct neighbourhood of the pyridine nitrogen atom, and Y represents halogen or a sulphonate, according to Equation 8 compounds of the formula (VII) are reacted with corresponding compounds of the formula (XXI). The reaction is carried out in the presence of an excess of the compound of the formula (XXI), and, if X represents O or S, additionally in the presence of a base, such as, for example, sodium hydride. The reaction takes place in solvents, such as diethylene glycol dimethyl ether or N-methylpyrrolidinone, or the compounds of the formula (XXI) themselves serve as solvents. The reaction is carried out at elevated temperature, preferably in a temperature range of between +80° C. and +200° C. Reactions in the upper region of the temperature interval mentioned are preferably carried out in closed pressure vessels in a microwave apparatus.

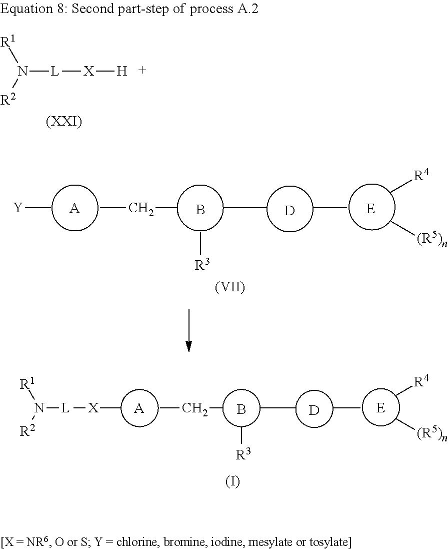

b) If X represents NR⁶, O or S and the group Y represents halogen or a sulphonate and is bonded to a carbon atom of a pyridine ring A which is in any desired position in relation to the pyridine nitrogen atom, or ring A is a phenyl ring, the compounds of the formula (VII) and the compounds of the formula (XXI) are reacted with one another according to Equation 8 in the presence of palladium catalysts. Suitable palladium sources are, for example, palladium(II) acetate or tris(dibenzylidene-acetone)dipalladium(0). Ligands which can be used are, for example, 2,2′-bis(diphenylphosphino)-1,1′-binaphthyl, 1-[2-(dicyclohexylphosphino)ferrocenyl]ethyldi-tert-butylphosphine or bis(diphenylphosphino)ferrocene. The reactions are carried out in the presence of bases, such as, for example, triethylamine or sodium tert-butoxide. Suitable solvents are, for example, toluene, N-methylpyrrolidinone or 1,2-dimethoxyethane. The reactions are usually carried out in the temperature interval of between +60° C. and the particular boiling point of the solvent.
c) If X represents ♦-NR⁶—C(═O)-♦♦, wherein R⁶, ♦ and ♦♦ have the meanings described above, compounds of the formula (VII) in which Y represents an alkoxycarbonyl group or cyano are first converted into the corresponding carboxylic acids by treatment with aqueous alkali, and these are then reacted with compounds of the formula (XXII) to give the products of the formula (I) (see Equation 9). This reaction is carried out either directly from the carboxylic acid in the presence of coupling reagents, such as, for example, 1H-benzotriazol-1-ol and N-[3-(dimethylamino)propyl]-N-ethylcarbodiimide hydrochloride, or by converting the carboxylic acid into the corresponding acid chloride, for example with the aid of thionyl chloride or oxalyl chloride, and then reacting this with the amine component (XXII).

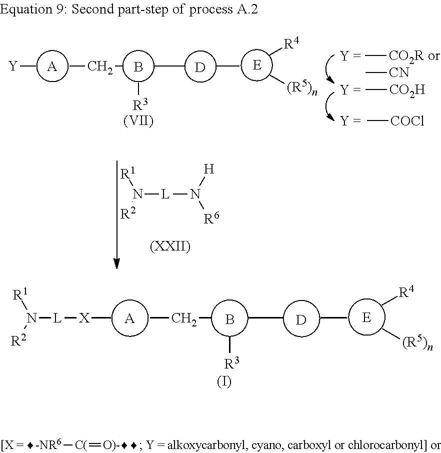

The hydrolysis of the esters (VII) [Y=alkoxycarbonyl] is preferably carried out with aqueous solutions of lithium hydroxide, sodium hydroxide or potassium hydroxide in the presence of water-miscible inert solvents, such as, for example, methanol, ethanol or tetrahydrofuran. The reaction is in general carried out in the temperature interval of between room temperature and +60° C., preferably at room temperature. The hydrolysis of the nitriles (VII) [Y=cyano] is likewise carried out with aqueous alkali, preferably with aqueous potassium hydroxide, in ethanol at the boiling point of the solvent. The subsequent conversion of the carboxylic acids obtained in this way into the corresponding acid chlorides is carried out with chlorinating reagents, such as, for example and preferably, oxalyl chloride or thionyl chloride, in inert solvents, such as, for example, dichloromethane. The reaction is carried out in the temperature range of between 0° C. and the boiling point of the solvent, preferably at room temperature. The final reaction of the amines of the formula (XXII) with the acid chlorides of the formula (VII) [Y=chlorocarbonyl] is carried out in the presence of bases, such as, for example, triethylamine, N,N-diisopropylethylamine or potassium carbonate, in inert solvents, such as, for example, dichloromethane or ethyl acetate. The reaction is carried out in the temperature range of from 0° C. to room temperature. The reaction of the amines of the formula (XXII) with the carboxylic acids of the formula (VII) [Y=carboxyl] is carried out with the aid of conventional coupling reagents, such as, for example, 1H-benzotriazol-1-ol and N-[3-(dimethylamino)propyl]-N-ethylcarbodiimide hydrochloride, in suitable solvents, such as, for example, N,N-dimethylformamide, and in the presence of tertiary amine bases, such as, for example, triethylamine. The reaction is preferably carried out at room temperature.

In cases where one or both of the radicals R′ and R² in the compounds of the formula (XXII) represent(s) hydrogen, it may be expedient or necessary in the reactions described in Equation 9 to use, instead of one of these hydrogen atoms, an amino-protective group which is then removed again at the end of the reaction sequence to give the target compounds of the formula (I). Such amino-protective groups are known per se to the person skilled in the art; the introduction and removal of these protective groups are likewise carried out by processes known to the person skilled in the art. Examples of such amino-protective groups are tert-butoxycarbonyl and benzyloxycarbonyl. Detailed descriptions of such protective group operations are to be found in the Experimental Part in the experimental procedures for the preparation of the starting materials and intermediates and the working examples.

d) If X represents ♦-C(═O)—NH-♦♦, wherein ♦ and ♦♦ have the meanings described above, compounds of the formula (VII) in which Y represents a nitro group are first reduced to the corresponding amines [Y═NH₂] and these are then reacted with compounds of the formula (XXIII) or (XXIV) to give the products of the formula (I) (see Equation 10). This reaction is carried out in the presence of conventional coupling reagents, such as, for example, 1H-benzotriazol-1-ol and N-[3-(dimethylamino)propyl]-N′-ethylcarbodiimide hydrochloride, in the case of the carboxylic acids (XXIII), and in the case of the acid chlorides (XXIV) directly in the presence of tertiary amine bases, such as triethylamine or N,N-diisopropylethylamine. Here, too, it may optionally be expedient or required to introduce a temporary amino-protective group into the compounds of the formulae (XXIII) and (XXIV).

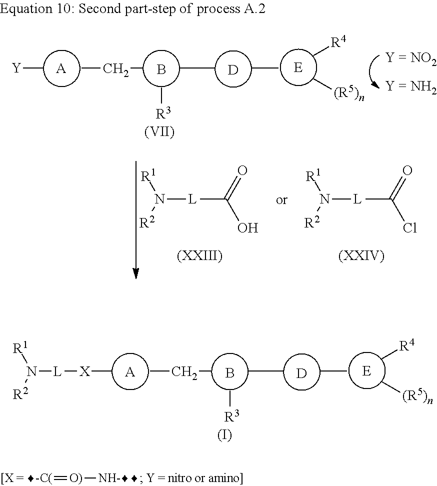

The reduction of the nitro group is achieved, for example, by catalytic hydrogenation with the aid of noble metal catalysts, such as, for example, palladium on charcoal, in inert solvents, such as, for example, ethanol, in the presence of hydrogen under a pressure of from 1 to 50 bar, preferably from 1 to 5 bar. The reaction is typically carried out at room temperature. The subsequent reaction with the carboxylic acids (XXIII) or acid chlorides (XXIV) is carried out either with the aid of coupling reagents or directly in the presence of tertiary amine bases, as has already been described above.

e) If X represents oxygen, compounds of the formula (XXV), in which Z represents a leaving group, such as, for example, chlorine, bromine or methanesulphonate, and compounds of the formula (VII) in which Y represents hydroxyl can alternatively also be reacted with one another. The latter are obtainable, for example, via corresponding silyl ethers (see Equation 11).

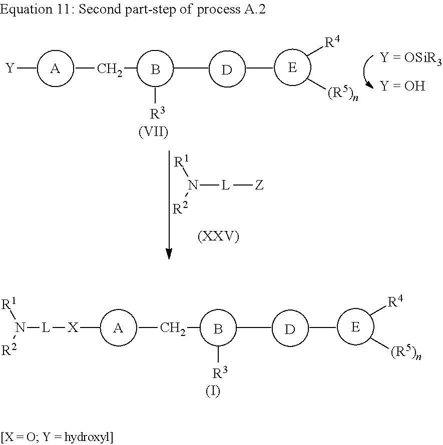

The reaction of the compounds of the formula (VII) in which Y represents a silyl ether to give the free hydroxy compounds of the formula (VII) [Y═OH] is carried out, for example, by treatment with a source of fluoride, such as tetra-n-butylammonium fluoride, in solvents, such as tetrahydrofuran, at temperatures preferably of between 0° C. and room temperature. The subsequent reaction with the compounds of the formula (XXV) is carried out in inert solvents, such as, for example and preferably, N,N-dimethylformamide, in the presence of bases, such as, for example, sodium hydride or caesium carbonate, at temperatures of between room temperature and +140° C.

f) A process similar to that described under e) can be used if X represents NH. The compounds of the formula (VII) shown in Equation 10 in which Y represents NH₂ are first converted into the corresponding carbamates, for example with di-tert-butyl dicarbonate or with benzyloxycarbonyl chloride, which are then reacted with compounds of the formula (XXV) (see Equation 11), in which Z represents a leaving group, such as chlorine, bromine or methanesulphonate. In the final reaction, the carbamate protective group is removed again in order to obtain the products of the formula (I) in which X represents NH. The processes for introducing and removing the carbamate protective groups have been described in the chemical literature and are known to the person skilled in the art. The reaction of the compounds of the formula (XXV) with the carbamates derived from compounds of the formula (VII) [Y═NH₂] is carried out under similar conditions as described under e).
g) If X represents a bond and L represents methylene, compounds of the formula (VII) in which Y represents cyano are initially reduced to give the aldehydes of the formula (XXVI) which are then reacted in a reductive amination with amines of the formula (XXVII) to give the corresponding products of the formula (I) (see Equation 12).

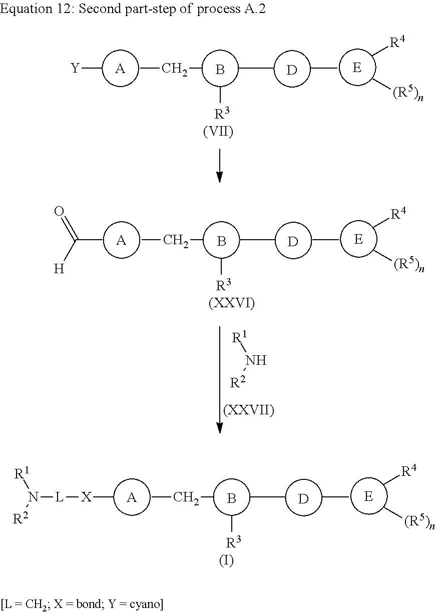

The reduction of the nitriles of the formula (VII) [Y=cyano] is advantageously carried out with diisobutylaluminium hydride in suitable solvent mixtures which preferably consist of tetrahydrofuran in combination with toluene, hexane, heptane or cyclohexane. The reaction is carried out at low temperature, preferably at about −78° C. Subsequent reductive amination with the amines of the formula (XXVII) is carried out in the presence of alkali metal borohydrides such as, for example sodium triacetoxyborohydride, sodium cyanoborohydride or sodium borohydride, in inert solvents such as, preferably, 1,2-dichloroethane or ethanol, at temperatures between 0° C. and room temperature.

The reactions which lead from the intermediates (VIII) or (IX) (process A.3, Equation 3 and process A.4, Equation 4) to the products of the formula (I), depending on the nature of the groups Y and Z, are described below. These reactions are also used correspondingly in processes B.3, C.3 and D.3 and B.4, C.4 and D.4, respectively.

h) The compounds of the formula (VIII) (Equation 3) in which Y represents hydroxyl are initially converted into compounds of the formula (IX) (Equation 4) in which Z represents a leaving group such as, for example, chlorine, bromine or methanesulphonate, and then reacted with amines of the formula (XXVII) to give the products of the formula (I) (see Equation 13).

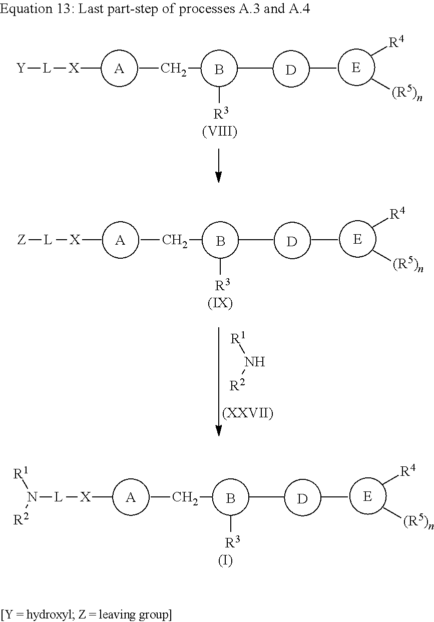

The compounds of the formula (VIII) in which Y represents hydroxyl are converted into compounds of the formula (IX) by reacting them, for example, with bromine in the presence of triphenylphosphine in suitable solvents, such as, for example, tetrahydrofuran, at room temperature to give the corresponding bromides (IX) [Z═Br]. The conversion can also be carried out, for example and preferably, with the aid of trifluoromethanesulphonic anhydride or methanesulphonic anhydride in the presence of bases, such as, for example, triethylamine or 2,6-dimethylpyridine. These reactions are preferably carried out in dichloromethane or tetrahydrofuran at low temperatures of approx. −78° C. Compounds of the formula (IX) in which Z represents trifluoromethanesulphonate (triflate) or methanesulphonate (mesylate) are obtained in this way. The compounds of the formula (IX) are then reacted with amines of the formula (XXVII) to give the products of the formula (I) by reacting the reactants, for example, in dichloromethane or tetrahydrofuran in the presence of tertiary amine bases, such as, for example, triethylamine or 2,6-dimethylpyridine, at temperatures of between −78° C. and room temperature. If Z represents trifluoromethanesulphonate or methanesulphonate, the reaction sequence can also be carried out as a one-pot process starting from compounds of the formula (VIII) [Y═OH].

The starting compounds of the formulae (II), (III), (IV), (V), (VI), (X), (XI), (XII), (XIII), (XIV), (XX), (XXI), (XXII), (XXIII), (XXIV), (XXV) and (XXVII) are either commercially obtainable or described as such in the literature, or they can be prepared by routes evident to the person skilled in the art analogously to methods published in the literature. Thus, for example, compounds of the formula (VI) in which ring D represents a 1,2,4-oxadiazole or a 1,3-oxazole can be prepared analogously to process methods B, C and D described above, and compounds of the formulae (II), (X), (XII) and (XIII) can be obtained analogously to process variants A.1, A.2, A.3 and A.4 with the part-steps described in Equations 8-13.

For example, compounds of the formula (I-D) according to the invention

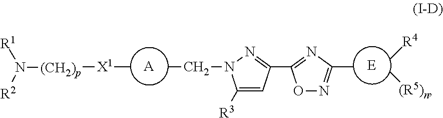

in which rings A and E and also R′, R², R³, R⁴, R⁵ and n each have the meanings given above,
X¹ represents NH or O,
and
p represents the number 2, 3 or 4,
can be prepared by initially condensing an N′-hydroxyamidine of the formula (XI)

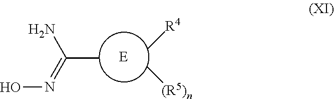

in which ring E and also R⁴, R⁵ and n have the meanings given above,
with a pyrazolecarboxylic acid of the formula (XXVIII)

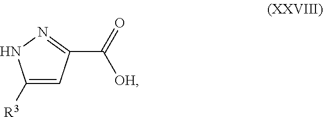

in which R³ has the meaning given above
to give a 1,2,4-oxadiazole derivative of the formula (XXIX)

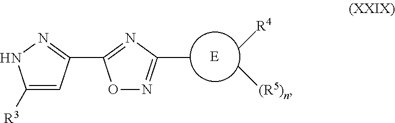

in which ring E and also R³, R⁴, R⁵ and n have the meanings given above,
and then alkylating the compound (XXIX) in the presence of a base either
[A] with a compound of the formula (XXX)

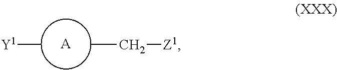

in which ring A has the meaning given above
Y¹ represents chlorine, bromine or iodine
and
Z¹ represents a leaving group such as, for example, chlorine, bromine, iodine, mesylate, triflate or tosylate,
to give a compound of the formula (XXXI)

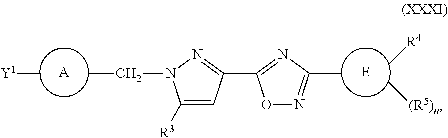

in which rings A and E and R³, R⁴, R⁵, n and Y¹ each have the meanings given above,
and then optionally reacting in the presence of a palladium catalyst and/or a base with a compound of the formula (XXXII)

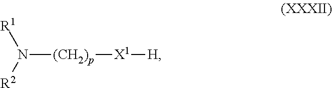

in which R¹, R², p and X¹ have the meanings given above,
to give the compound of the formula (I-D)
or
[B] in an alternative, if in compound (I-D) X¹ represents O and ring A represents a phenyl ring, alkylating with a compound of the formula (XXXIII)

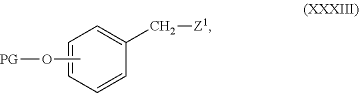

in which
PG represents a silyl protective group such as, for example trimethylsilyl, triisopropylsilyl or tert-butyldimethylsilyl
and
Z¹ represents a leaving group such as, for example, chlorine, bromine, iodine, mesylate, triflate or tosylate,
to give a compound of the formula (XXXIV)

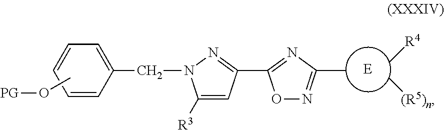

in which ring E and also R³, R⁴, R⁵, n and PG have the meanings given above,
and, after removal of the silyl protective group PG, reacting the resulting compound of the formula (XXXV)

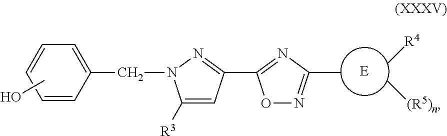

in which ring E and also R³, R⁴, R⁵ and n have the meanings given above,
if appropriate in the presence of a base with a compound of the formula (XXXVI)

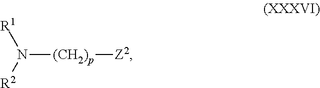

in which R¹, R² and p have the meanings given above,
and
Z² represents a leaving group such as, for example, chlorine, bromine, iodine, hydroxyl, mesylate, triflate or tosylate,
to give the compound of the formula (I-D′)

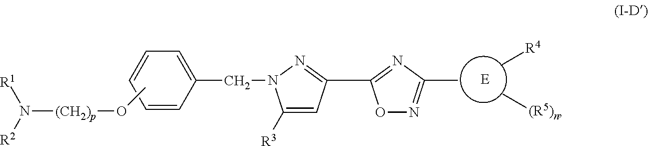

in which ring E and R¹, R², R³, R⁴, R⁵, n and p each have the meanings given above
   (cf. the processes A.2 and B.1 described above in combination with the variants shown in Equations 8 and 11 of the second part-step of process A.2 and the reaction parameters stated therein in each case).

Numerous detailed instructions and literature information for the preparation of the starting materials are also to be found in the experimental part in the section for the preparation of the starting compounds and intermediates.

The compounds according to the invention have valuable pharmacological properties and can be used for prevention and treatment of diseases in humans and animals.

The compounds according to the invention are highly potent inhibitors of the HIF regulation pathway and have a satisfactory solubility in aqueous or physiological media.

On the basis of their action profile, the compounds according to the invention are suitable in particular for treatment of hyperproliferative diseases in humans and in mammals generally. The compounds can inhibit, block, reduce or lower cell proliferation and cell division and on the other hand increase apoptosis.

The hyperproliferative diseases for the treatment of which the compounds according to the invention can be employed include, inter alia, psoriasis, keloids, formation of scars and other proliferative diseases of the skin, benign diseases, such as benign prostate hyperplasia (BPH), and in particular the group of tumour diseases. In the context of the present invention, these are understood as meaning, in particular, the following diseases, but without being limited to them: mammary carcinomas and mammary tumours (ductal and lobular forms, also in situ), tumours of the respiratory tract (parvicellular and non-parvicellular carcinoma, bronchial carcinoma), cerebral tumours (e.g. of the brain stem and of the hypothalamus, astrocytoma, medulloblastoma, ependymoma and neuro-ectodermal and pineal tumours), tumours of the digestive organs (oesophagus, stomach, gall bladder, small intestine, large intestine, rectum), liver tumours (inter alia hepatocellular carcinoma, cholangiocellular carcinoma and mixed hepatocellular and cholangiocellular carcinoma), tumours of the head and neck region (larynx, hypopharynx, nasopharynx, oropharynx, lips and oral cavity), skin tumours (squamous epithelial carcinoma, Kaposi sarcoma, malignant melanoma, Merkel cell skin cancer and nonmelanomatous skin cancer), tumours of soft tissue (inter alia soft tissue sarcomas, osteosarcomas, malignant fibrous histiocytomas, lymphosarcomas and rhabdomyosarcomas), tumours of the eyes (inter alia intraocular melanoma and retinoblastoma), tumours of the endocrine and exocrine glands (e.g. thyroid and parathyroid glands, pancreas and salivary gland), tumours of the urinary tract (tumours of the bladder, penis, kidney, renal pelvis and ureter) and tumours of the reproductive organs (carcinomas of the endometrium, cervix, ovary, vagina, vulva and uterus in women and carcinomas of the prostate and testicles in men). These also include proliferative blood diseases in solid form and as circulating blood cells, such as lymphomas, leukaemias and myeloproliferative diseases, e.g. acute myeloid, acute lymphoblastic, chronic lymphocytic, chronic myelogenic and hair cell leukaemia, and AIDS-correlated lymphomas, Hodgkin's lymphomas, non-Hodgkin's lymphomas, cutaneous T cell lymphomas, Burkitt's lymphomas and lymphomas in the central nervous system.

These well-described diseases in humans can also occur with a comparable aetiology in other mammals and can be treated there with the compounds of the present invention.

In the context of this invention the term “treatment” or “treat” is used in the conventional sense and means attending to, caring for and nursing a patient with the aim of combating, reducing, attenuating or alleviating a disease or health abnormality and improving the living conditions impaired by this disease, such as, for example, with a cancer disease.

The compounds according to the invention act as modulators of the HIF regulation pathway and are therefore also suitable for treatment of diseases associated with a harmful expression of the HIF transcription factor. This applies in particular to the transcription factors HIF-1α and HIF-2α. The term “harmful expression of HIF” here means a non-normal physiological presence of HIF protein. This can be due to excessive synthesis of the protein (mRNA- or translation-related), reduced degradation or inadequate counter-regulation in the functioning of the transcription factor.

HIF-1α and HIF-2α regulate more than 100 genes. This applies to proteins which play a role in angiogenesis and are therefore directly relevant to tumours, and also those which influence glucose, amino acid and lipid metabolism as well as cell migration, metastasis and DNA repair, or improve the survival of tumour cells by suppressing apoptosis. Others act more indirectly via inhibition of the immune reaction and upwards regulation of angiogenic factors in inflammation cells. HIF also plays an important role in stem cells, and here in particular tumour stem cells, which are reported to have increased HIF levels. By the inhibition of the HIF regulation pathway by the compounds of the present invention, tumour stem cells, which do not have a high proliferation rate and therefore are affected only inadequately by cytotoxic substances, are therefore also influenced therapeutically (cf. Semenza, 2007; Weidemann and Johnson, 2008).

Changes in cell metabolism by HIF are not exclusive to tumours, but also occur with other hypoxic pathophysiological processes, whether chronic or transient. HIF inhibitors—such as the compounds of the present invention—are therapeutically helpful in those connections in which, for example, additional damage arises from adaptation of cells to hypoxic situations, since damaged cells can cause further damage if they do not function as intended. One example of this is the formation of epileptic foci in partly destroyed tissue following strokes. A similar situation is found with cardiovascular diseases if ischaemic processes occur in the heart or in the brain as a consequence of thromboembolic events, inflammations, wounds, intoxications or other causes. These can lead to damage such as a locally retarded action potential, which in turn can bring about arrhythmias or chronic heart failure. In a transient form, e.g. due to apnoea, under certain circumstances an essential hypertension may occur, which can lead to known secondary diseases, such as, for example, stroke and cardiac infarction.

Inhibition of the HIF regulation pathway such as is achieved by the compounds according to the invention can therefore also be helpful for diseases such as cardiac insufficiency, arrhythmia, cardiac infarction, apnoea-induced hypertension, pulmonary hypertension, transplant ischaemia, reperfusion damage, stroke and macular degeneration, as well as for recovery of nerve function after traumatic damage or severance.

Since HIF is one of the factors which control the transition from an epithelial to a mesenchymal cell type, which is of importance specifically for the lung and kidney, the compounds according to the invention can also be employed for preventing or controlling fibroses of the lung and kidney associated with HIF.

Further diseases for the treatment of which the compounds according to the invention can be used are inflammatory joint diseases, such as various forms of arthritis, and inflammatory intestinal diseases, such as, for example, Crohn's disease.

Chugwash polycythaemia is mediated by HIF-2α activity during erythropoiesis inter alia in the spleen. The compounds according to the invention, as inhibitors of the HIF regulation pathway, are therefore also suitable here for suppressing excessive erythrocyte formation and therefore for alleviating the effects of this disease.

The compounds of the present invention can furthermore be used for treatment of diseases associated with excessive or abnormal angiogenesis. These include, inter alia, diabetic retinopathy, ischaemic retinal vein occlusion and retinopathy in premature babies (cf. Aiello et al., 1994; Peer et al., 1995), age-related macular degeneration (AMD; cf. Lopez et al., 1996), neovascular glaucoma, psoriasis, retrolental fibroplasia, angiofibroma, inflammation, rheumatic arthritis (RA), restenosis, in-stent restenosis and restenosis following vessel implantation.

An increased blood supply is furthermore associated with cancerous, neoplastic tissue and leads here to an accelerated tumour growth. The growth of new blood and lymph vessels moreover facilitates the formation of metastases and therefore the spread of the tumour. New lymph and blood vessels are also harmful for allografts in immunoprivileged tissues, such as the eye, which, for example, increases the susceptibility to rejection reactions. Compounds of the present invention can therefore also be employed for therapy of one of the abovementioned diseases, e.g. by an inhibition of the growth or a reduction in the number of blood vessels. This can be achieved via inhibition of endothelial cell proliferation or other mechanisms for preventing or lessening the formation of vessels and via a reduction of neoplastic cells by apoptosis.

The present invention furthermore provides the use of the compounds according to the invention for treatment and/or prevention of diseases, in particular the abovementioned diseases.

The present invention furthermore provides the use of the compounds according to the invention for the preparation of a medicament for treatment and/or prevention of diseases, in particular the abovementioned diseases.

The present invention furthermore provides the use of the compounds according to the invention in a method for treatment and/or prevention of diseases, in particular the abovementioned diseases.

The present invention furthermore provides a method for treatment and/or prevention of diseases, in particular the abovementioned diseases, using an active amount of at least one of the compounds according to the invention.

The compounds according to the invention can be employed by themselves or, if required, in combination with one or more other pharmacologically active substances, as long as this combination does not lead to undesirable and unacceptable side effects. The present invention furthermore therefore provides medicaments containing at least one of the compounds according to the invention and one or more further active compounds, in particular for treatment and/or prevention of the abovementioned diseases.

For example, the compounds of the present invention can be combined with known antihyperproliferative, cytostatic or cytotoxic substances for treatment of cancer diseases. The combination of the compounds according to the invention with other substances customary for cancer therapy or also with radiotherapy is therefore indicated in particular, since hypoxic regions of a tumour respond only weakly to the conventional therapies mentioned, whereas the compounds of the present invention display their activity there in particular.

Suitable active compounds in the combination which may be mentioned by way of example are:

aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulphate, broxuridine, bortezomib, busulfan, calcitonin, campath, capecitabine, carboplatin, casodex, cefesone, celmoleukin, cerubidin, chlorambucil, cisplatin, cladribin, clodronic acid, cyclophosphamide, cytarabine, dacarbazine, dactinomycin, daunoxome, decadron, decadron phosphate, delestrogen, denileukin diftitox, depomedrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin-alfa, epogen, eptaplatin, ergamisol, estrace, estradiol, estramustine sodium phosphate, ethinylestradiol, ethyol, etidronic acid, etopophos, etoposide, fadrozole, farstone, filgrastim, finasteride, fligrastim, floxuridine, fluconazole, fludarabin, 5-fluorodeoxyuridine monophosphate, 5-fluoruracil (5-FU), fluoxymesterone, flutamide, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, kytril, lentinan sulphate, letrozole, leucovorin, leuprolide, leuprolide acetate, levamisole, levofolic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, picibanil, pilocarpine hydrochloride, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofuran, sobuzoxane, solu-medrol, streptozocin, strontium-89 chloride, synthroid, tamoxifen, tamsulosin, tasonermin, tastolactone, taxoter, teceleukin, temozolomide, teniposide, testosterone propionate, testred, thioguanine, thiotepa, thyrotropin, tiludronic acid, topotecan, toremifen, tositumomab, tastuzumab, teosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin-stimalamer, zofran; ABI-007, acolbifen, actimmune, affinitak, aminopterin, arzoxifen, asoprisnil, atamestane, atrasentan, avastin, BAY 43-9006 (sorafenib), CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon-gamma, intron-PEG, ixabepilone, keyhole limpet hemocyanine, L-651582, lanreotide, lasofoxifen, libra, lonafarnib, miproxifen, minodronate, MS-209, liposomal MTP-PE, MX-6, nafarelin, nemorubicin, neovastat, nolatrexed, oblimersen, onko-TCS, osidem, paclitaxel polyglutamate, pamidronate disodium, PN-401, QS-21, quazepam, R-1549, raloxifen, ranpirnas, 13-cis-retic acid, satraplatin, seocalcitol, T-138067, tarceva, taxoprexin, thymosin-alpha-1, tiazofurin, tipifarnib, tirapazamine, TLK-286, toremifen, transMID-107R, valspodar, vapreotide, vatalanib, verteporfin, vinflunin, Z-100, zoledronic acid and combinations of these.

In a preferred embodiment, the compounds of the present invention can be combined with antihyperproliferative agents, which can be, by way of example—without this list being conclusive:

aminoglutethimide, L-asparaginase, azathioprine, 5-azacytidine, bleomycin, busulfan, camptothecin, carboplatin, carmustine, chlorambucil, cisplatin, colaspase, cyclophosphamide, cytarabine, dacarbazine, dactinomycin, daunorubicin, diethylstilbestrol, 2′,2′-difluorodeoxycytidine, docetaxel, doxorubicin (adriamycin), epirubicin, epothilone and its derivatives, erythro-hydroxynonyladenin, ethinylestradiol, etoposide, fludarabin phosphate, 5-fluorodeoxyuridine, 5-fluoro deoxyuridine monophosphate, 5-fluorouracil, fluoxymesterone, flutamide, hexamethylmelamine, hydroxyurea, hydroxyprogesterone caproate, idarubicin, ifosfamide, interferon, irinotecan, leucovorin, lomustine, mechlorethamine, medroxyprogesterone acetate, megestrol acetate, melphalan, 6-mercaptopurine, mesna, methotrexate, mitomycin C, mitotane, mitoxantrone, paclitaxel, pentostatin, N-phosphonoacetyl L-aspartate (PALA), plicamycin, prednisolone, prednisone, procarbazine, raloxifen, semustine, streptozocin, tamoxifen, teniposide, testosterone propionate, thioguanine, thiotepa, topotecan, trimethylmelamine, uridine, vinblastine, vincristine, vindesine and vinorelbine.

The compounds according to the invention can also be combined in a very promising manner with biological therapeutics, such as antibodies (e.g. avastin, rituxan, erbitux, herceptin) and recombinant proteins, which additively or synergistically intensify the effects of inhibition of the HIF signal pathway transmission.

Inhibitors of the HIF regulation pathway, such as the compounds according to the invention, can also achieve positive effects in combination with other therapies directed against angiogenesis, such as, for example, with avastin, axitinib, DAST, recentin, sorafenib or sunitinib. Combinations with inhibitors of the proteasome and of mTOR and antihormones and steroidal metabolic enzyme inhibitors are particularly suitable because of their favourable profile of side effects.

Generally, the following aims can be pursued with the combination of compounds of the present invention with other agents having a cytostatic or cytotoxic action:
an improved activity in slowing down the growth of a tumour, in reducing its size or even in its complete elimination compared with treatment with an individual active compound;
the possibility of employing the chemotherapeutics used in a lower dosage than in monotherapy;
the possibility of a more tolerable therapy with fewer side effects compared with individual administration;
the possibility of treatment of a broader spectrum of tumour diseases;
achievement of a higher rate of response to the therapy;
a longer survival time of the patient compared with present-day standard therapy.

The compounds according to the invention can moreover also be employed in combination with radiotherapy and/or surgical intervention.

The present invention furthermore provides medicaments which comprise at least one compound according to the invention, conventionally together with one or more inert, non-toxic, pharmaceutically suitable auxiliary substances, and the use thereof for the above-mentioned purposes.

The compounds according to the invention can act systemically and/or locally. They can be administered in a suitable manner for this purpose, such as e.g. orally, parenterally, pulmonally, nasally, sublingually, lingually, buccally, rectally, dermally, transdermally, conjunctivally, otically or as an implant or stent.

The compounds according to the invention can be administered in suitable administration forms for these administration routes.

Administration forms which function according to the prior art, release the compounds according to the invention rapidly and/or in a modified manner and contain the compounds according to the invention in crystalline and/or amorphized and/or dissolved form are suitable for oral administration, such as e.g. tablets (non-coated or coated tablets, for example with coatings which are resistant to gastric juice or dissolve in a delayed manner or are insoluble and control the release of the compound according to the invention), tablets or films/oblates, films/lyophilisates or capsules which disintegrate rapidly in the oral cavity (for example hard or soft gelatine capsules), sugar-coated tablets, granules, pellets, powders, emulsions, suspensions, aerosols or solutions.

Parenteral administration can be effected with bypassing of an absorption step (e.g. intravenously, intraarterially, intracardially, intraspinally or intralumbally) or with inclusion of an absorption (e.g. intramuscularly, subcutaneously, intracutaneously, percutaneously or intraperitoneally). Administration forms which are suitable for parenteral administration are, inter alia, injection and infusion formulations in the form of solutions, suspensions, emulsions, lyophilisates or sterile powders.

For the other administration routes e.g. inhalation medicament forms (inter alia powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents are suitable.

Oral or parenteral administration is preferred, in particular oral and intravenous administration.

The compounds according to the invention can be converted into the administration forms mentioned. This can be effected in a manner known per se by mixing with inert, non-toxic, pharmaceutically suitable auxiliary substances. These auxiliary substances include inter alia carrier substances (for example microcrystalline cellulose, lactose, mannitol), solvents (e.g. liquid polyethylene glycols), emulsifiers and dispersing or wetting agents (for example sodium dodecyl sulphate, polyoxysorbitan oleate), binders (for example polyvinylpyrrolidone), synthetic and natural polymers (for example albumin), stabilizers (e.g. antioxidants, such as, for example, ascorbic acid), dyestuffs (e.g. inorganic pigments, such as, for example, iron oxides) and flavour and/or smell correctants.

In general, it has proven advantageous in the case of parenteral administration to administer amounts of from about 0.001 to 1 mg/kg, preferably about 0.01 to 0.5 mg/kg of body weight to achieve effective results. In the case of oral administration the dosage is about 0.01 to 100 mg/kg, preferably about 0.01 to 20 mg/kg and very particularly preferably 0.1 to 10 mg/kg of body weight.

Nevertheless it may be necessary to deviate from the amounts mentioned, and in particular depending on the body weight, administration route, individual behaviour towards the active compound, nature of the formulation and point in time or interval at which administration takes place. Thus in some cases it may be sufficient to manage with less than the abovementioned minimum amount, while in other cases the upper limit mentioned must be exceeded. In the case where relatively large amounts are administered, it may be advisable to spread these into several individual doses over the day.

The following embodiment examples illustrate the invention. The inventions is not limited to the examples.

The percentage data in the following tests and examples are percentages by weight, unless stated otherwise; parts are parts by weight. The solvent ratios, dilution ratios and concentration data of liquid/liquid solutions in each case relate to the volume.

### A. EXAMPLES

### Abbreviations and Acronyms

abs. absolute
aq. aqueous
Boc tert-butoxycarbonyl
Ex. Example
Bu butyl
approx. circa, approximately
CI chemical ionization (in MS)
d doublet (in NMR)
d day(s)
TLC thin layer chromatography
DCI direct chemical ionization (in MS)
dd doublet of doublet (in NMR)
DMAP 4-N,N-dimethylaminopyridine
DME 1,2-dimethoxyethane
DMF dimethylformamide
DMSO dimethyl sulphoxide
dt doublet of triplet (in NMR)
EDC N′-(3-dimethylaminopropyl)-N-ethylcarbodiimide hydrochloride
EI electron impact ionization (in MS)
eq. equivalent(s)
ESI electrospray ionization (in MS)
Et ethyl
GC gas chromatography
h hour(s)
HOBt 1-hydroxy-1H-benzotriazole hydrate
HPLC high pressure, high performance liquid chromatography
ⁱPr isopropyl
LC-MS liquid chromatography-coupled mass spectrometry
m multiplet (in NMR)
min minute(s)
MPLC medium pressure liquid chromatography (on silica gel; also called “flash chromatography”)
MS mass spectrometry
NMP N-methyl-2-pyrrolidinone
NMR nuclear magnetic resonance spectrometry
Pd/C palladium on activated carbon
Pr propyl
quart quartet (in NMR)
quint quintet (in NMR)
R_{f} retention index (in TLC)
RT room temperature
Rₜ retention time (in HPLC)
singlet (in NMR)
sept septet (in NMR)
t triplet (in NMR)
^{t}Bu tert-butyl
TFA trifluoroacetic acid
THF tetrahydrofuran
UV ultraviolet spectrometry
v/v volume to volume ratio (of a solution)
tog. together

### HPLC, LC/MS and GC/MS Methods:

### Method A (Analytical HPLC):

Instrument: HP 1100 with DAD detection; column: Kromasil 100 RP-18, 60 mm×2.1 mm, 3.5 μm; eluent A: 5 ml of perchloric acid (70% strength)/1 of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

### Method B (Analytical HPLC):

Instrument: HP 1100 with DAD detection; column: Kromasil 100 RP-18, 60 mm×2.1 mm, 3.5 μm; eluent A: 5 ml of perchloric acid (70% strength)/1 of water, mobile phase B: acetonitrile; gradient: 0 min 2% B→0.5 min 2% B→4.5 min 90% B→9 min 90% B→9.2 min 2% B→10 min 2% B; flow rate: 0.75 ml/min; column temperature: 30° C.; UV detection: 210 nm.

### Method C (LC/MS):

MS instrument type: Micromass ZQ; HPLC instrument type: HP 1100 series; UV DAD; column: Phenomenex Gemini 3μ, 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method D (LC/MS):

MS instrument type: Waters Micromass Quattro Micro; HPLC instrument type: Agilent 1100 series; column: Thermo Hypersil GOLD 3μ, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A→4.01 min 100% A→5.00 min 100% A; oven: 50° C.; flow rate: 2 ml/min UV detection: 210 nm.

### Method E (LC/MS):

MS instrument type: Micromass ZQ; HPLC instrument type: Waters Alliance 2795; column: Phenomenex Synergi 2.5μ MAX-RP 100A Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A 4.0 min 5% A→4.01 min 90% A; flow rate: 2 ml/min; oven: 50° C.; UV detection: 210 nm.

### Method F (LC/MS):

Instrument: Micromass Quattro Premier with Waters HPLC Acquity; column: Thermo Hypersil GOLD 1.9μ, 50 mm×1 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→1.5 min 10% A→2.2 min 10% A; flow rate: 0.33 ml/min; oven: 50° C.; UV detection: 210 nm.

### Method G (LC/MS):

Instrument: Micromass Platform LCZ with HPLC Agilent Series 1100; column: Thermo Hypersil GOLD 3μ, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method H (LC/MS):

Instrument: Micromass Quattro LCZ with HPLC Agilent Series 1100; column: Phenomenex Syn-ergi 2.5μ MAX-RP 100A Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.1 min 90% A; flow rate: 2 ml/min; oven: 50° C.; UV detection: 208-400 nm.

### Method I (GC/MS):

Instrument: Micromass GCT, GC 6890; column: Restek RTX-35, 15 m×200 μm×0.33 μm; constant helium flow rate: 0.88 ml/min; oven: 70° C.; inlet: 250° C.; gradient: 70° C., 30° C./min→310° C. (maintained for 3 min).

### Method J (GC/MS):

Instrument: Micromass GCT, GC 6890; column: Restek RTX-35, 15 m×200 μm×0.33 μm; constant helium flow rate: 0.88 ml/min; oven: 70° C.; inlet: 250° C.; gradient: 70° C., 30° C./min→310° C. (maintained for 12 min).

### Method K (Preparative HPLC):

Column: GROM-SIL 120 ODS-4 HE, 10 μm, 250 mm×30 mm; mobile phase and gradient programme: acetonitrile/0.1% aq. formic acid 10:90 (0-3 min), acetonitrile/0.1% aq. formic acid 10:90→95:5 (3-27 min), acetonitrile/0.1% aq. formic acid 95:5 (27-34 min), acetonitrile/0.1% aq. formic acid 10:90 (34-38 min); flow rate: 50 ml/min; temperature: 22° C.; UV detection: 254 nm.

### Method L (Preparative HPLC):

Column: Reprosil C18, 10 μm, 250 mm×30 mm; mobile phase and gradient programme: acetonitrile/0.1% aq. trifluoroacetic acid 10:90 (0-2 min), acetonitrile/0.1% aq. trifluoroacetic acid 10:90→90:10 (2-23 min), acetonitrile/0.1% aq. trifluoroacetic acid 90:10 (23-28 min), acetonitrile/0.1% aq. trifluoroacetic acid 10:90 (28-30 min); flow rate: 50 ml/min; temperature: 22° C.; UV detection: 210 nm.

### Method M (LC/MS):

Instrument: Waters Acquity SQD HPLC System; column: Waters Acquity HPLC HSS T3 1.8 μm, 50 mm×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; flow rate: 0.40 ml/min; oven: 50° C.; UV detection: 210-400 nm.

### Method N (LC/MS):

MS instrument: Waters ZQ 2000; HPLC instrument: Agilent 1100, 2-column circuit; autosampler: HTC PAL; column: YMC-ODS-AQ, 50 mm×4.6 mm, 3.0 μm; mobile phase A: water+0.1% formic acid, mobile phase B: acetonitrile+0.1% formic acid; gradient: 0.0 min 100% A→0.2 min 95% A→1.8 min 25% A→1.9 min 10% A→2.0 min 5% A→3.2 min 5% A→3.21 min 100% A→3.35 min 100% A; oven: 40° C.; flow rate: 3.0 ml/min; UV detection: 210 nm.

### Method O (LC/MS):

MS instrument: Waters SQD; HPLC instrument: Waters HPLC; column: Zorbax SB-Aq (Agilent), 50 mm×2.1 mm, 1.8 μm; mobile phase A: water+0.025% formic acid, mobile phase B: acetonitrile+0.025% formic acid; gradient: 0.0 min 98% A→0.9 min 25% A 1.0 min 5% A→1.4 min 5% A→1.41 min 98% A→1.5 min 98% A; oven: 40° C.; flow rate: 0.60 ml/min; UV detection: DAD, 210 nm.

### Method P (Analytical HPLC):

column: Kromasil C18, 4 mm×250 mm, 5 μm; mobile phase A: 0.2% aq. perchloric acid, mobile phase B: acetonitrile; gradient: 0 min 10% B→3.0 min 90% B→3.1 min 10% B; flow rate: 1.0 ml/min; column temperature: 30° C.; UV detection: 210 nm.

For all the reactants or reagents for which the preparation is not described explicitly in the following, they were obtained commercially from generally accessible sources. For all the other reactants or reagents for which the preparation likewise is not described in the following and which were not commercially obtainable or were obtained from sources which are not generally accessible, reference is made to the published literature in which their preparation is described.

### Starting Compounds and Intermediates

### Example 1A

### N′-Hydroxy-4-(1,1,1-trifluoro-2-methylpropan-2-yl)benzenecarboximidamide

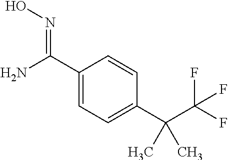

### Step 1: 2-(4-Bromophenyl)-1,1,1-trifluoropropan-2-ol

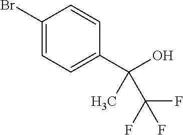

A suspension of dichloro(dimethyl)titanium in a heptane/dichloromethane mixture was first prepared as follows: 100 ml (100 mmol) of a 1 M solution of titanium tetrachloride in dichloromethane were cooled to −30° C., 100 ml (100 mmol) of a 1 M solution of dimethylzinc in heptane were added dropwise and the mixture was subsequently stirred at −30° C. for 30 min. This suspension was then cooled to −40° C. and a solution of 10 g (39.5 mmol) of 1-(4-bromophenyl)-2,2,2-trifluoroethanone in 50 ml of dichloromethane was added. The mixture was subsequently stirred at −40° C. for 5 min, the temperature was then allowed to come to RT and the mixture was stirred at RT for a further 2 h. 50 ml of water were slowly added dropwise, while cooling with ice, and the mixture was then diluted with a further 300 ml of water. It was extracted twice with dichloromethane, the combined dichloromethane phases were washed once with water, dried over anhydrous magnesium sulphate and filtered and the solvent was removed on a rotary evaporator. The residue was purified by column chromatography on silica gel (mobile phase: cyclohexane/ethyl acetate 85:15). 10.5 g (100% of theory) of the title compound were obtained which, according to ¹H-NMR, still contained residues of solvent.

¹H-NMR (400 MHz, CDCl₃, δ/ppm): 7.52 (d, 2H), 7.47 (d, 2H), 1.76 (s, 3H).

LC/MS (method C, ESIpos): Rₜ=2.27 min, m/z=268 [M+H]⁺.

### Step 2: 2-(4-Bromophenyl)-1,1,1-trifluoropropan-2-yl methanesulphonate

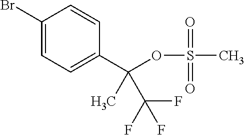

3.12 g (78.05 mmol, 60% strength in mineral oil) of sodium hydride were initially introduced into 45 ml of THF under argon and a solution of 10.5 g (39.03 mmol) of the compound obtained in Example 1A/step 1 in 20 ml of THF was added dropwise at RT. After the mixture had been stirred at RT for 1 h and at 40° C. for 30 min, a solution of 8.94 g (78.05 mmol) of methanesulphonyl chloride in 45 ml of THF was added dropwise and the reaction mixture was stirred at 40° C. for a further 60 min. 50 ml of water were then slowly added dropwise to the mixture and the mixture was diluted with saturated aqueous sodium bicarbonate solution and extracted twice with ethyl acetate. The combined ethyl acetate phases were dried over anhydrous magnesium sulphate and filtered and the solvent was removed on a rotary evaporator. The residue was stirred in hexane and the solid obtained was filtered off and dried under reduced pressure. 12.4 g (92% of theory) of the title compound were obtained.

¹H-NMR (400 MHz, CDCl₃, δ/ppm): 7.58 (d, 2H), 7.43 (d, 2H), 3.16 (s, 3H), 2.28 (s, 3H).

LC/MS (method D, ESIpos): Rₜ=2.32 min, m/z=364 [M+NH₄]⁺.

### Step 3: 1-Bromo-4-(1,1,1-trifluoro-2-methylpropan-2-yl)benzene

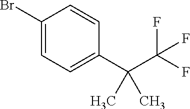

12.4 g (35.72 mmol) of the compound obtained in Example 1A/step 2 were initially introduced into 250 ml of dichloromethane and the mixture was cooled to 0° C. 35.7 ml (71.44 mmol) of a 2 M solution of trimethylaluminium were then slowly added dropwise at 0° C., while stirring, and the mixture was then allowed to come to RT and was subsequently stirred at RT for a further 1.5 h. 120 ml of a saturated aqueous sodium bicarbonate solution were slowly added dropwise to the mixture, followed by 40 ml of a saturated aqueous sodium chloride solution. The mixture was filtered over kieselguhr and the kieselguhr was rinsed twice with dichloromethane. The combined dichloromethane phases were washed once with saturated aqueous sodium chloride solution and dried over anhydrous magnesium sulphate and the solvent was removed on a rotary evaporator. 8.69 g (87% of theory) of the title compound were obtained in a purity of 95%.

¹H-NMR (400 MHz, CDCl₃, δ/ppm): 7.49 (d, 2H), 7.33 (d, 2H), 1.55 (s, 6H).

LC/MS (method E, ESIpos): Rₜ=2.54 min, no ionization.

GC/MS (method I, EI): Rₜ=3.48 min, m/z=266 [M]⁺.

### Step 4: 4-(1,1,1-Trifluoro-2-methylpropan-2-yl)benzenecarbonitrile

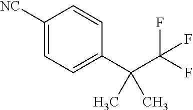

3.34 g (12.50 mmol) of the compound obtained in Example 1A/step 3 were initially introduced into 2.5 ml of degassed DMF under argon, 881 mg (7.50 mmol) of zinc cyanide and 867 mg (0.75 mmol) of tetrakis(triphenylphosphine)palladium(0) were added and the mixture was stirred at 80° C. overnight. After cooling to RT, the reaction mixture was diluted with ethyl acetate and solid constituents were filtered off. The filtrate was washed twice with 2 N aqueous ammonia solution and once with saturated aqueous sodium chloride solution, dried over anhydrous magnesium sulphate and freed from the solvent on a rotary evaporator. The residue was purified by column chromatography on silica gel (mobile phase: cyclohexane/ethyl acetate 85:15). 2.08 g (78% of theory) of the title compound were obtained.

¹H-NMR (400 MHz, CDCl₃, δ/ppm): 7.68 (d, 2H), 7.62 (d, 2H), 1.60 (s, 6H).

GC/MS (method I, EI): Rₜ=3.83 min, m/z=213 [M]⁺.

### Step 5: N′-Hydroxy-4-(1,1,1-trifluoro-2-methylpropan-2-yl)benzenecarboximidamide

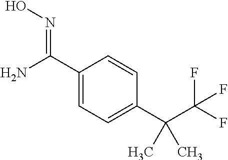

A mixture of 2.40 g (11.26 mmol) of the compound from Example 1A/step 4, 1.72 g (24.77 mmol) of hydroxylamine hydrochloride and 3.45 ml (24.77 mmol) of triethylamine in 60 ml of ethanol was stirred under reflux for 1 h. After cooling to RT, the solvent was removed on a rotary evaporator. Ethyl acetate was added to the residue and the solid present was filtered off. The ethyl acetate solution was washed successively with water and saturated aqueous sodium chloride solution, dried over anhydrous magnesium sulphate and filtered. After removal of the solvent, the oil obtained was triturated with petroleum ether. After the resulting solid had been filtered off with suction and dried under high vacuum, 2.65 g (96% of theory) of the title compound were obtained.

¹H-NMR (400 MHz, CDCl₃, δ/ppm): 8.0 (s, broad, 1H), 7.62 (d, 2H), 7.52 (d, 2H), 4.88 (s, broad, 2H), 1.60 (s, 6H).

LC/MS (method D, ESIpos): Rₜ=1.34 min, m/z=247 [M+H]⁺.

### Example 2A

### 4-(2-Fluoropropan-2-yl)-N′-hydroxybenzenecarboximidamide

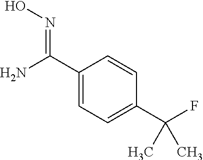

### Step 1: 4-(2-Fluoropropan-2-yl)benzenecarbonitrile

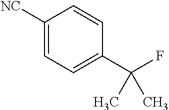

1.20 g (7.44 mmol) of diethylaminosulphur trifluoride (DAST) were added to a solution of 1.00 g (6.20 mmol) of 4-(2-hydroxypropan-2-yl)benzenecarbonitrile [obtained from 4-(propan-2-yl)benzenecarbonitrile in accordance with J. L. Tucker et al., Synth. Comm. 2006, 36 (15), 2145-2155] in 20 ml of dichloromethane at a temperature of 0° C. The reaction mixture was stirred at RT for 2 h and then diluted with water and extracted with dichloromethane. The organic phase was washed with water, dried over anhydrous magnesium sulphate and filtered. After removal of the solvent on a rotary evaporator, the residue was purified by MPLC (silica gel, mobile phase: cyclohexane/ethyl acetate 95:5). 675 mg (67% of theory) of the title compound were obtained.

¹H-NMR (400 MHz, CDCl₃, δ/ppm): 7.57 (d, 2H), 7.48 (d, 2H), 1.72 (s, 3H), 1.68 (s, 3H).

LC/MS (method D, ESIpos): Rₜ=2.12 min, m/z=163 [M+H]⁺.

### Step 2: 4-(2-Fluoropropan-2-yl)-N′-hydroxybenzenecarboximidamide

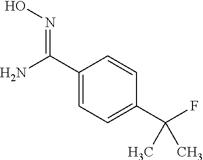

By the process described in Example 1A/step 5, 756 mg (93% of theory) of the title compound were obtained from 675 mg (4.14 mmol) of the compound from Example 2A/step 1.

¹H-NMR (400 MHz, CDCl₃, δ/ppm): 7.62 (d, 2H), 7.41 (d, 2H), 4.89 (s, broad, 2H), 1.72 (s, 3H), 1.68 (s, 3H).

LC/MS (method D, ESIpos): Rₜ=1.04 min, m/z=197 [M+H]⁺.

### Example 3A

### N′-Hydroxy-4-[(trifluoromethyl)sulphonyl]benzenecarboximidamide

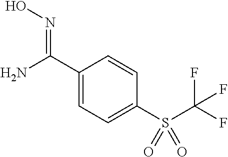

By the process described in Example 1A/step 5, 5.08 g (97% of theory) of the title compound were obtained from 4.60 g (19.56 mmol) of 4-[(trifluoromethyl)sulphonyl]benzenecarbonitrile [W. Su, Tetrahedron. Lett. 1994, 35 (28), 4955-4958].

¹H-NMR (400 MHz, DMSO-d₆, δ/ppm): 10.26 (s, 1H), 8.13 (dd, 4H), 6.12 (s, 2H).

LC/MS (method D, ESIpos): Rₜ=1.57 min, m/z=269 [M+H]⁺.

### Example 4A

### N′-Hydroxy-4-(3-methyloxetan-3-yl)benzenecarboximidamide

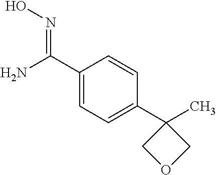

### Step 1: [4-(Dibenzylamino)phenyl]boronic acid

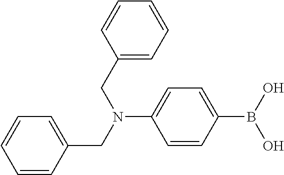

A solution of 6.0 g (17.03 mmol) of N,N-dibenzyl-4-bromoaniline [T. Saitoh et al., J. Am. Chem. Soc. 2005, 127 (27), 9696-9697] was initially introduced into a mixture of 75 ml of anhydrous diethyl ether and 75 ml of anhydrous THF under inert conditions. 13.9 ml (22.14 mmol) of a 1.6 M solution of n-butyllithium in hexane were added dropwise to this solution at −78° C. When the addition had ended, the mixture was stirred at −78° C. for 60 min, before 6.3 ml (27.25 mmol) of boric acid triisopropyl ester were added dropwise at the same temperature. After a further 15 min at −78° C., the reaction mixture was allowed to come to RT. After stirring at RT for 3 h, 18 ml of 2 M hydrochloric acid were added and the resulting mixture was stirred intensively at RT for 20 min. After dilution with approx. 200 ml of water, the mixture was extracted three times with approx. 200 ml of ethyl acetate each time. The combined organic extracts were washed successively with water and saturated sodium chloride solution. After drying over anhydrous magnesium sulphate, the mixture was filtered and the solvent was removed on a rotary evaporator. The oily residue obtained was triturated with a mixture of 50 ml of tert-butyl methyl ether and 50 ml of pentane. After the resulting solid had been filtered off with suction and dried under high vacuum, 3.91 g (72% of theory, purity 90%) of the title compound were obtained, this being employed in the next stage without further purification.

¹H-NMR (400 MHz, DMSO-d₆, δ/ppm): 7.58 (d, 2H), 7.32-7.30 (m, 4H), 7.27-7.23 (m, 6H), 6.66 (d, 2H), 4.70 (s, 4H).

HPLC (method A): Rₜ=4.35 min.

MS (ESIpos): m/z=318 [M+H]⁺.

### Step 2: Ethyl {3-[4-(dibenzylamino)phenyl]oxetan-3-yl}acetate

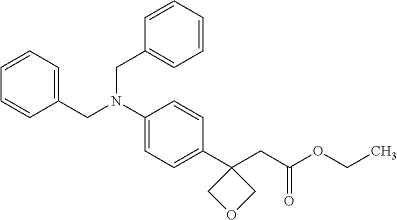

10.7 ml (16.0 mmol) of a 1.5 M potassium hydroxide solution were added to a solution of 304 mg (0.616 mmol) of (1,5-cyclooctadiene)rhodium(I) chloride dimer in 30 ml of 1,4-dioxane. Solutions of 1.75 g (12.31 mmol) of ethyl oxetan-3-ylideneacetate [G. Wuitschik et al., Angew. Chem. Int. Ed. Engl. 2006, 45 (46), 7736-7739] in 1 ml of 1,4-dioxane and 3.91 g (12.31 mmol) of the compound from Example 4A/step 1 in 60 ml of 1,4-dioxane were then added successively. The reaction mixture was stirred at RT for 6 h. It was then diluted with approx. 200 ml of water and extracted three times with approx. 200 ml of ethyl acetate each time. The combined organic extracts were washed successively with water and saturated sodium chloride solution. After drying over anhydrous magnesium sulphate, the mixture was filtered and the solvent was removed on a rotary evaporator. The crude product obtained was purified by MPLC (silica gel, mobile phase: cyclohexane/ethyl acetate 20:1→5:1). 3.51 g (67% of theory) of the title compound were obtained.

¹H-NMR (400 MHz, CDCl₃, δ/ppm): 7.33-7.30 (m, 4H), 7.27-7.23 (m, 6H), 6.97 (d, 2H), 6.69 (d, 2H), 4.94 (d, 2H), 4.81 (d, 2H), 4.62 (s, 4H), 4.00 (quart, 2H), 3.04 (s, 2H), 1.11 (t, 3H).

LC/MS (method E, ESIpos): Rₜ=2.57 min, m/z=416 [M+H]⁺.

### Step 3: 2-{3-[4-(Dibenzylamino)phenyl]oxetan-3-yl}ethanol

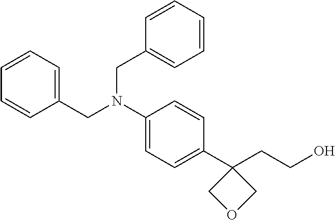

4.9 ml (4.88 mmol) of a 1 M solution of lithium aluminium hydride in THF were added dropwise to a solution of 2.90 g (6.98 mmol) of the compound from Example 4A/step 2 in 145 ml of anhydrous THF under inert conditions and at a temperature of 0° C. When the dropwise addition had ended, the reaction mixture was stirred at 0° C. for 1.5 h. 2 g of kieselguhr and 2 ml of water were then cautiously added. The heterogeneous mixture was filtered with suction over a paper filter. The filtrate was diluted with approx. 250 ml of water and extracted three times with approx. 250 ml of ethyl acetate each time. The combined organic extracts were washed successively with water and saturated sodium chloride solution. After drying over anhydrous magnesium sulphate, the mixture was filtered and the solvent was removed on a rotary evaporator. The crude product obtained was purified by MPLC (silica gel, mobile phase: cyclohexane/ethyl acetate 4:1). 2.34 g (87% of theory) of the title compound were obtained.

¹H-NMR (400 MHz, CDCl₃, δ/ppm): 7.36-7.31 (m, 4H), 7.27-7.22 (m, 6H), 6.88 (d, 2H), 6.71 (d, 2H), 4.93 (d, 2H), 4.71 (d, 2H), 4.63 (s, 4H), 3.55 (quart, 2H), 2.29 (t, 2H), 1.12 (t, 1H).

HPLC (method B): Rₜ=3.98 min.

MS (DCI, NH₃): m/z=374 [M+H]⁺.

LC/MS (method E, ESIpos): Rₜ=2.15 min, m/z=374 [M+H]⁺.

### Step 4: {3-[4-(Dibenzylamino)phenyl]oxetan-3-yl}acetaldehyde

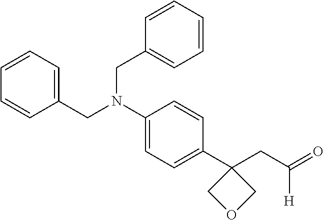

807 μl of anhydrous DMSO were added dropwise to a solution of 496 μl (5.68 mmol) of oxalyl chloride in 5 ml of anhydrous dichloromethane at −78° C. under inert conditions. After 20 min, a solution of 1.93 g (5.17 mmol) of the compound from Example 4A/step 3 in 5 ml of anhydrous dichloromethane was slowly added dropwise at the same temperature. After stirring at −78° C. for 60 min, 3.7 ml (26.87 mmol) of anhydrous triethylamine were added dropwise. After a further 10 min at this temperature, the reaction mixture was allowed to warm to RT. The mixture was then introduced into a suction filter filled with silica gel and elution was carried out first with cyclohexane and then with cyclohexane/ethyl acetate 7:1→1:1. The product fractions were combined and evaporated to dryness and the residue was taken up in ethyl acetate. Washing was carried out successively with saturated sodium bicarbonate solution, water and saturated sodium chloride solution. After drying over anhydrous magnesium sulphate, the mixture was filtered and the solvent was removed on a rotary evaporator. 1.81 g (92% of theory) of the title compound were obtained.

¹H-NMR (400 MHz, CDCl₃, δ/ppm): 9.69 (t, 1H), 7.34-7.31 (m, 4H), 7.28-7.23 (m, 6H), 6.97 (d, 2H), 6.70 (d, 2H), 5.00 (d, 2H), 4.72 (d, 2H), 4.63 (s, 4H), 3.18 (d, 2H).

HPLC (method B): Rₜ=4.61 min.

MS (DCI, NH₃): m/z=372 [M+H]⁺.

LC/MS (method F, ESIpos): Rₜ=1.43 min, m/z=372 [M+H]⁺.

### Step 5: N,N-Dibenzyl-4-(3-methyloxetan-3-yl)aniline

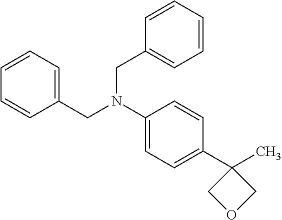

A solution of 1.81 g (4.87 mmol) of the compound from Example 4A/step 4 and 13.57 g (14.62 mmol) of tris(triphenylphosphine)rhodium(I) chloride in 240 ml of toluene was heated under reflux under inert conditions for one hour. After cooling to RT, insoluble constituents were filtered off. The solvent was removed on a rotary evaporator and the residue was purified by MPLC (silica gel, cyclohexane/ethyl acetate 20:1→5:1). 1.36 g (73% of theory, purity approx. 90%) of the title compound were obtained.

¹H-NMR (400 MHz, CDCl₃, δ/ppm): 7.35-7.31 (m, 4H), 7.27-7.24 (m, 6H), 7.07 (d, 2H), 6.72 (d, 2H), 4.90 (d, 2H), 4.64 (s, 4H), 4.55 (d, 2H), 1.96 (s, 3H).

LC/MS (method F, ESIpos): Rₜ=1.55 min, m/z=344 [M+H]⁺.

### Step 6: 4-(3-Methyloxetan-3-yl)aniline

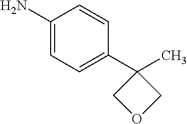

A solution of 1.35 g (3.93 mmol) of the compound from Example 4A/step 5 in 135 ml of ethanol was hydrogenated in a flow-through hydrogenation apparatus (“H-Cube” from ThalesNano, Budapest, Hungary) (conditions: 10% Pd/C catalyst, “full H₂” mode, 1 ml/min, 50° C.). After removal of the solvent on a rotary evaporator, the crude product was purified by MPLC (silica gel, cyclohexane/ethyl acetate 4:1→2:1). 386 mg (60% of theory) of the title compound were obtained.

¹H-NMR (400 MHz, CDCl₃, δ/ppm): 7.03 (d, 2H), 6.69 (d, 2H), 4.92 (d, 2H), 4.58 (d, 2H), 3.63 (s, broad, 2H), 1.69 (s, 3H).

LC/MS (method D, ESIpos): Rₜ=0.77 min, m/z=164 [M+H]⁺.

### Step 7: 4-(3-Methyloxetan-3-yl)benzenecarbonitrile

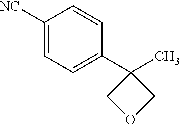

First 1.7 ml (20.7 mmol) of concentrated hydrochloric acid and then, dropwise, a solution of 159 mg (2.30 mmol) of sodium nitrite in 5 ml of water were added to a solution of 375 mg (2.30 mmol) of the compound from Example 4A/step 6 in 17 ml of water at 0° C. The mixture was stirred at 0° C. for 30 min, before 1.1 g (10.3 mmol) of solid sodium carbonate were added in portions. The solution obtained in this way was added dropwise to a solution of 257 mg (2.87 mmol) of copper(I) cyanide and 464 mg (7.12 mmol) of potassium cyanide in 16 ml of toluene/water (2:1) at 0° C. The reaction mixture was stirred at 0° C. for 1 h. The mixture was then allowed to warm to RT. The organic phase was then separated off and washed successively with water and saturated sodium chloride solution. After the solvent had been separated off on a rotary evaporator, the crude product was purified by MPLC (silica gel, cyclohexane/ethyl acetate 10:1→2:1). 390 mg (83% of theory, purity 84%) of the title compound were obtained.

¹H-NMR (400 MHz, CDCl₃, δ/ppm): 7.66 (d, 2H), 7.31 (d, 2H), 4.92 (d, 2H), 4.68 (d, 2H), 1.73 (s, 3H).

GC/MS (method I, EIpos): Rₜ=5.45 min, m/z=173 [M]⁺.

### Step 8: N′-Hydroxy-4-(3-methyloxetan-3-yl)benzenecarboximidamide

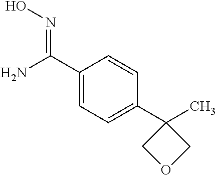

By the process described in Example 1A/step 5, 297 mg (74% of theory) of the title compound were obtained from 375 mg (1.83 mmol) of the compound from Example 4A/step 7.

¹H-NMR (400 MHz, DMSO-d₆, δ/ppm): 9.59 (s, 1H), 7.64 (d, 2H), 7.23 (d, 2H), 5.79 (s, broad, 2H), 4.80 (d, 2H), 4.53 (d, 2H), 1.62 (s, 3H).

HPLC (method A): Rₜ=2.74 min.

MS (DCI, NH₃): m/z=207 [M+H]⁺.

### Example 5A

### 4-(3-Fluorooxetan-3-yl)-N′-hydroxybenzenecarboximidamide

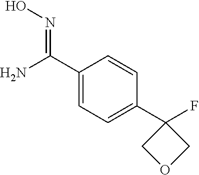

### Step 1: 4-(3-Hydroxyoxetan-3-yl)benzenecarbonitrile

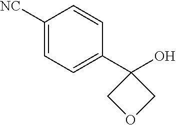

11 ml (21.8 mmol) of a 2 M solution of isopropylmagnesium chloride in diethyl ether were added dropwise to a solution of 5.0 g (21.8 mmol) of 4-iodobenzonitrile in 100 ml of anhydrous THF at −40° C. under inert conditions. After the mixture had been stirred at the same temperature for 1.5 h, it was cooled down to −78° C. and was slowly added to a solution, likewise cooled to −78° C., of 2.95 g (32.7 mmol, 80% in dichloromethane) of 3-oxooxetane [G. Wuitschik et al., Angew. Chem. Int. Ed. Engl. 2006, 45 (46), 7736-7739] in 100 ml of anhydrous THF with the aid of a cannula. When the addition had ended, the reaction mixture was stirred first at −78° C. for 10 min, then at 0° C. for 2 h and finally at RT for 30 min A few ml of saturated aqueous ammonium chloride solution were then added. The solvent was then largely removed on a rotary evaporator. The residue obtained was diluted with 200 ml of water and extracted three times with approx. 200 ml of ethyl acetate each time. The combined organic extracts were washed successively with water and saturated sodium chloride solution. After drying over anhydrous magnesium sulphate, the mixture was filtered and the solvent was removed on a rotary evaporator. The crude product obtained was purified by crystallization from cyclohexane/ethyl acetate 10:1. 2.42 g (63% of theory) of the title compound were obtained.

¹H-NMR (400 MHz, DMSO-d₆, δ/ppm): 7.88 (d, 2H), 7.80 (d, 2H), 6.63 (s, 1H), 4.79 (d, 2H), 4.65 (d, 2H).

HPLC (method A): Rₜ=3.09 min.

MS (DCI, NH₃): m/z=193 [M+NH₄]⁺.

### Step 2: 4-(3-Fluorooxetan-3-yl)benzenecarbonitrile

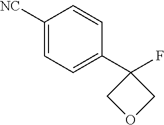

A solution of 662 mg (4.11 mmol) of diethylaminosulphur trifluoride (DAST) in 5 ml of dichloromethane was added dropwise to a suspension of 600 mg (3.43 mmol) of the compound from Example 5A/step 1 in 55 ml of dichloromethane at −78° C. under inert conditions. After 30 min at −78° C., the reaction mixture was warmed very rapidly to −20° C. with the aid of an ice/water bath. After approx. 30 seconds, 20 ml of 1 M sodium hydroxide solution were added and the mixture was allowed to warm to RT. After dilution with 150 ml of water, the mixture was extracted three times with approx. 50 ml of diethyl ether each time. The combined organic extracts were dried over anhydrous magnesium sulphate. After filtration, the solvent was removed on a rotary evaporator. The crude product was purified by MPLC (silica gel, cyclohexane/ethyl acetate 8:1). 495 mg (82% of theory) of the title compound were obtained.

¹H-NMR (400 MHz, CDCl₃, δ/ppm): 7.76 (d, 2H), 7.73 (d, 2H), 5.15 (dd, 2H), 4.81 (dd, 2H).

LC/MS (method D, ESIpos): Rₜ=1.59 min, m/z=178 [M+H]⁺.

### Step 3: 4-(3-Fluorooxetan-3-yl)-N′-hydroxybenzenecarboximidamide

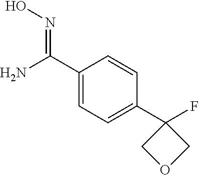

By the process described in Example 1A/step 5, 470 mg (86% of theory) of the title compound were obtained from 450 mg (2.54 mmol) of the compound from Example 5A/step 2.

¹H-NMR (400 MHz, DMSO-d₆, δ/ppm): 9.71 (s, 1H), 7.77 (d, 2H), 7.54 (d, 2H), 5.87 (broad s, 2H), 4.97 (dd, 2H), 4.91 (dd, 2H).

HPLC (method A): Rₜ=2.64 min.

MS (DCI, NH₃): m/z=211 [M+H]⁺.

LC/MS (method D, ESIpos): Rₜ=0.80 min, m/z=211 [M+H]⁺.

### Example 6A

### N′-Hydroxy-4-(3-methoxyoxetan-3-yl)benzenecarboximidamide

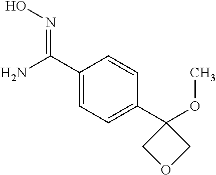

### Step 1: 4-(3-Methoxyoxetan-3-yl)benzenecarbonitrile

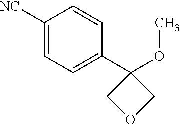

151 mg (3.77 mmol) of a 60% strength dispersion of sodium hydride in mineral oil were added to a solution of 600 mg (3.43 mmol) of the compound from Example 5A/step 1 in 12.5 ml of anhydrous DMF at 5° C. The mixture was stirred at 5° C. for 1 h, before 256 μl (4.11 mmol) of methyl iodide were added. The reaction mixture was then allowed to come to RT. After stirring for 15 h, 150 ml of water were added and the mixture was extracted twice with approx. 150 ml of diethyl ether each time. The combined organic extracts were dried over anhydrous magnesium sulphate. After filtration and removal of the solvent on a rotary evaporator, the residue obtained was purified by MPLC (silica gel, cyclohexane/ethyl acetate 20:1→4:1). 566 mg (87% of theory) of the title compound were obtained.

¹H-NMR (400 MHz, DMSO-d₆, δ/ppm): 7.92 (d, 2H), 7.68 (d, 2H), 4.81 (d, 2H), 4.74 (d, 2H), 3.07 (s, 3H).

HPLC (method A): Rₜ=3.63 min.

MS (DCI, NH₃): m/z=207 [M+NH₄]⁺.

LC/MS (method D, ESIpos): Rₜ=1.50 min, m/z=190 [M+H]⁺.

### Step 2: N′-Hydroxy-4-(3-methoxyoxetan-3-yl)benzenecarboximidamide

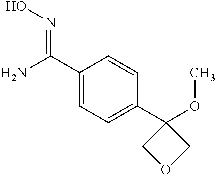

By the process described in Example 1A/step 5, 520 mg (89% of theory) of the title compound were obtained from 500 mg (2.64 mmol) of the compound from Example 6A/step 1.

¹H-NMR (400 MHz, DMSO-d₆, δ/ppm): 9.67 (s, 1H), 7.73 (d, 2H), 7.43 (d, 2H), 5.83 (broad s, 2H), 4.77 (m, 4H), 3.03 (s, 3H).

HPLC (method A): Rₜ=2.54 min.

MS (DCI, NH₃): m/z=223 [M+H]⁺.

### Example 7A

### 4-(4-Fluorotetrahydro-2H-pyran-4-yl)-N′-hydroxybenzenecarboximidamide

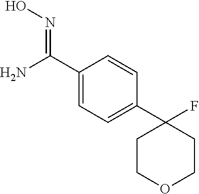

### Step 1: 4-(4-Hydroxytetrahydro-2H-pyran-4-yl)benzenecarbonitrile

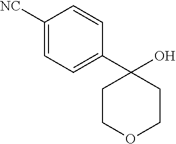

By the process described in Example 5A/step 1, 25.0 g (109 mmol) of 4-iodobenzonitrile were reacted with 16.4 g (164 mmol) of tetrahydro-4H-pyran-4-one to give 7.56 g (34% of theory) of the title compound.

¹H-NMR (400 MHz, DMSO-d₆, δ/ppm): 7.80 (d, 2H), 7.70 (d, 2H), 5.30 (s, 1H), 3.81-3.70 (m, 4H), 2.02-1.94 (m, 2H), 1.51-1.48 (m, 2H).

HPLC (method A): Rₜ=3.35 min.

MS (DCI, NH₃): m/z=204 [M+H]⁺, 221 [M+NH₄]⁺.

### Step 2: 4-(4-Fluorotetrahydro-2H-pyran-4-yl)benzenecarbonitrile

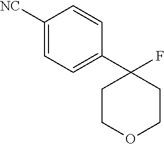

By the process described in Example 5A/step 2, 6.5 g (31.98 mmol) of the compound from Example 7A/step 1 were reacted to give 3.73 g (57% of theory) of the title compound.

¹H-NMR (400 MHz, CDCl₃, δ/ppm): 7.68 (d, 2H), 7.50 (d, 2H), 3.98-3.83 (m, 4H), 2.23-2.05 (m, 2H), 1.91-1.85 (m, 2H).

HPLC (method A): Rₜ=4.04 min.

MS (DCI, NH₃): m/z=223 [M+NH₄]⁺.

### Step 3: 4-(4-Fluorotetrahydro-2H-pyran-4-yl)-N′-hydroxybenzenecarboximidamide

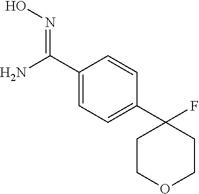

By the process described in Example 1A/step 5, 3.57 g (88% of theory) of the title compound were obtained from 3.5 g (17.05 mmol) of the compound from Example 7A/step 2.

¹H-NMR (500 MHz, DMSO-d₆, δ/ppm): 9.64 (s, 1H), 7.70 (d, 2H), 7.44 (d, 2H), 5.81 (s, 2H), 3.88-3.83 (m, 2H), 3.73-3.67 (m, 2H), 2.23-2.06 (m, 2H), 1.87-1.81 (m, 2H).

HPLC (method A): Rₜ=3.06 min.

MS (DCI, NH₃): m/z=239 [M+H]⁺.

LC/MS (method F, ESIpos): Rₜ=0.40 min, m/z=239 [M+H]⁺.

### Example 8A

### N′-Hydroxy-4-(4-methoxytetrahydro-2H-pyran-4-yl)benzenecarboximidamide

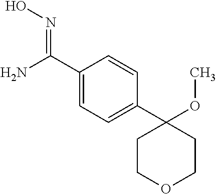

### Step 1: 4-(4-Methoxytetrahydro-2H-pyran-4-yl)benzenecarbonitrile

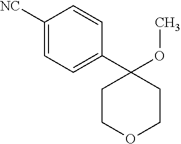

By the process described in Example 6A/step 1, 238 mg (74% of theory) of the title compound were obtained from 300 mg (1.48 mmol) of the compound from Example 7A/step 1 and 111 μl (1.77 mmol) of methyl iodide.

¹H-NMR (500 MHz, CDCl₃, δ/ppm): 7.68 (d, 2H), 7.51 (d, 2H), 3.89-3.82 (m, 4H), 2.99 (s, 3H), 2.03-1.98 (m, 2H), 1.94-1.91 (m, 2H).

HPLC (method A): Rₜ=3.99 min.

MS (DCI, NH₃): m/z=235 [M+NH₄]⁺.

GC/MS (method I, EIpos): Rₜ=6.57 min, m/z=217 [M]⁺.

### Step 2: N′-Hydroxy-4-(4-methoxytetrahydro-2H-pyran-4-yl)benzenecarboximidamide

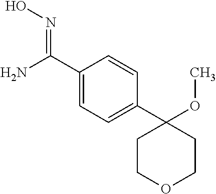

By the process described in Example 1A/step 5, 229 mg (99% of theory) of the title compound were obtained from 200 mg (0.921 mmol) of the compound from Example 8A/step 1.

¹H-NMR (400 MHz, DMSO-d₆, δ/ppm): 9.63 (s, 1H), 7.68 (d, 2H), 7.39 (d, 2H), 5.80 (s, 2H), 3.71-3.67 (m, 4H), 2.88 (m, 2H), 1.93-1.89 (m, 4H).

HPLC (method B): Rₜ=2.95 min.

MS (DCI, NH₃): m/z=251 [M+H]⁺.

LC/MS (method D, ESIpos): Rₜ=0.93 min, m/z=251 [M+H]⁺.

Analogously to the process described in Example 1A/step 5, the N′-hydroxybenzenecarboximidamides listed in the following table were prepared from the corresponding commercially obtainable benzonitriles. The benzonitriles which are not commercially obtainable were prepared in accordance with the following instructions in the literature: 4-cyclohexylbenzenecarbonitrile [E. Riguet et al., J. Organomet. Chem. 2001, 624 (1-2), 376-379], 4-(piperidin-1-yl)benzenecarbonitrile [A.-H. Kuthier et al., J. Org. Chem. 1987, 52 (9), 1710-1713], 4-(pentafluoro-λ⁶-sulphanyl)benzenecarbonitrile [P. J. Crowley et al., Chimia 2004, 58 (3), 138-142].

| | | HPLC: | MS: m/z | LC/MS |
|---|---|---|---|---|
| Example | Structure | Rₜ [min] | [M + H]⁺ | method |
| 9A | 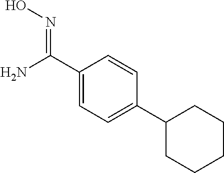 | 1.24 | 219 | H |
| | ¹H-NMR (400 MHz, DMSO-d₆, δ/ppm): 9.51 (s, 1 H), 7.56 (d, 2 H), 7.20 (d, 2 H), | | | |
| | 5.72 (s, broad, 2 H), 2.52-2.48 (m, 1 H), 1.81-1.74 (m, 4 H), 1.73-1.67 (m, 1 H), | | | |
| | 1.45-1.31 (m, 4 H), 1.28-1.19 (m, 1 H). | | | |
| 10A | 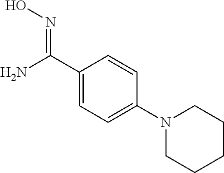 | 1.11 | 220 | D |
| | ¹H-NMR (400 MHz, CDCl₃, δ/ppm): 7.50 (d, 2 H), 6.90 (d, 2 H), 4.80 (s, broad, 2 H), | | | |
| | 3.23-3.20 (m, 4 H), 1.71-1.65 (m, 4 H), 1.63-1.57 (m, 2 H). | | | |
| 11A | 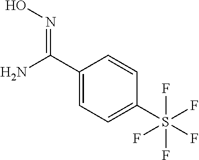 | 1.49 | 263 | D |
| | ¹H-NMR (400 MHz, DMSO-d₆, δ/ppm): 9.99 (s, 1 H), 7.94-7.85 (m, 4 H), 6.00 (s, 2 H). | | | |
| 12A | 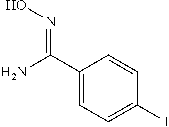 | 1.98 | 263 | G |
| | ¹H-NMR (400 MHz, DMSO-d₆, δ/ppm): 9.71 (s, 1 H), 7.73 (d, 2 H), 7.47 (d, 2 H), | | | |
| | 5.84 (s, broad, 2 H). | | | |
| 13A | 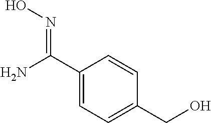 | 0.24 | 167 | D |
| | ¹H-NMR (400 MHz, DMSO-d₆, δ/ppm): 9.55 (s, 1 H), 7.62 (d, 2 H), 7.29 (d, 2 H), | | | |
| | 5.78 (s, 2 H), 5.20 (t, 1 H), 4.50 (d, 2 H). | | | |
| 14A | 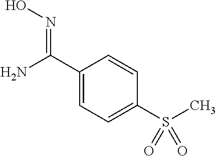 | 0.21 | 215 | F |
| | ¹H-NMR (400 MHz, DMSO-d₆, δ/ppm): 9.98 (s, 1 H), 7.92 (s, 4 H), 6.00 (s, broad, 2 H), | | | |
| | 3.23 (s, 3 H). | | | |
| 15A | 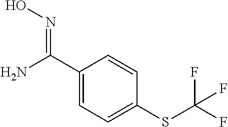 | 1.42 | 237 | D |
| | ¹H-NMR (400 MHz, DMSO-d₆, δ/ppm): 9.90 (s, 1 H), 7.80 (d, 2 H), 7.72 (d, 2 H), | | | |
| | 5.94 (s, 2 H). | | | |
| 16A | 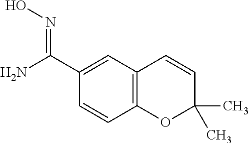 | 0.65 | 219 | F |

¹H-NMR (400 MHz, CDCl₃, δ/ppm): 11.2 (very broad, 1 H), 7.35 (dd, 1 H), 7.26 (d, 1 H),
6.78 (d, 1 H), 6.31 (d, 1 H), 5.63 (d, 1 H), 4.82 (broad, 2 H), 1.43 (s, 6 H).

### Example 17A

### 5-(5-Methyl-1H-pyrazol-3-yl)-3-[4-(trifluoromethoxy)phenyl]-1,2,4-oxadiazole

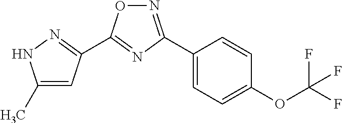

23.3 g (0.121 mol) of EDC, 16.4 g (0.121 mol) of HOBt and 26.7 g (0.121 mol) of N′-hydroxy-4-(trifluoromethoxy)benzenecarboximidamide were added successively to a solution of 15.3 g (0.121 mol) of 5-methyl-1H-pyrazole-3-carboxylic acid in 600 ml of anhydrous DMF at RT. The mixture was stirred first at RT for 2 h and then at 140° C. for 5 h. After cooling, the mixture was diluted with 2 litres of water and extracted three times with 1 litre of ethyl acetate each time. The combined organic extracts were washed successively with water and saturated sodium chloride solution. After drying over anhydrous magnesium sulphate, the mixture was filtered and the solvent was removed on a rotary evaporator. The crude product obtained was purified by filtration with suction over a suction filter filled with silica gel (eluent: cyclohexane/ethyl acetate 5:1→1:1). The product fractions were combined and the solvent was removed on a rotary evaporator to such an extent that the product just started to precipitate out. The precipitation was brought to completion at RT. By filtration and further concentration of the mother liquor, two fractions of solid were obtained, which were combined and dried under high vacuum. 19.7 g (52% of theory) of the title compound were obtained in total in this way.

¹H-NMR (400 MHz, CDCl₃, δ/ppm): 10.75 (broad, 1H), 8.24 (d, 2H), 7.34 (d, 2H), 6.81 (s, 1H), 2.46 (s, 3H).

HPLC (method A): Rₜ=4.72 min.

MS (DCI, NH₃): m/z=311 [M+H]⁺.

LC/MS (method F, ESIpos): Rₜ=1.27 min, m/z=311 [M+H]⁺.

The compounds listed in the following table were prepared by the process described in Example 17A from 5-methyl-1H-pyrazole-3-carboxylic acid, 5-(trifluoromethyl)-1H-pyrazole-3-carboxylic acid, 5-nitro-1H-pyrazole-3-carboxylic acid or 2-methyl-1H-imidazole-4-carboxylic acid hydrate and the corresponding N′-hydroxybenzenecarboximidamides. The reaction time during which stirring was initially carried out at RT was 0.5 to 4 h, depending on the size of the batch. The mixture was then heated at 140° C. for 1 to 15 h. Depending on the polarity of the product obtained, this already precipitated out on addition of water after the reaction had ended, and it was then washed and dried under high vacuum. Alternatively, as described above, the mixture was worked up by extraction and the product was then purified by chromatography over silica gel; various mobile phases were used for the chromatography. In some cases it was possible to omit the chromatography and to purify the product directly by extraction by stirring in dichloromethane, ethyl acetate, acetonitrile or tert-butyl methyl ether. The compound in Example 27A was purified by preparative HPLC (method K).

| | | HPLC: | MS: m/z | LC/MS |
|---|---|---|---|---|
| Example | Structure | Rₜ [min] | [M + H]⁺ | method |
| 18A | 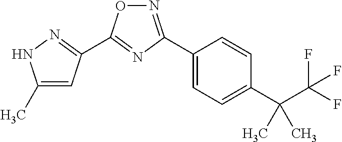 | 1.34 | 337 | F |
| | ¹H-NMR (400 MHz, DMSO-d₆, δ/ppm): 11.80 (s, broad, 1 H), 8.17 (d, 2 H), | | | |
| | 7.63 (d, 2 H), 6.83 (s, 1 H), 2.46 (s, 3 H), 1.63 (s, 6 H). | | | |
| 19A | 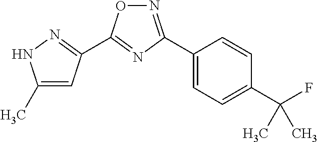 | 2.19 | 287 | D |
| | ¹H-NMR (400 MHz, DMSO-d₆, δ/ppm): 13.54 (s, broad, 1 H), 8.08 (d, 2 H), | | | |
| | 7.62 (d, 2 H), 6.81 (s, 1 H), 2.33 (s, 3 H), 1.72 (s, 3 H), 1.68 (s, 3 H). | | | |
| 20A | 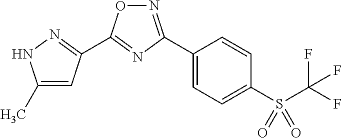 | 1.25 | 359 | F |
| | ¹H-NMR (400 MHz, DMSO-d₆, δ/ppm): 13.62 (s, broad, 1 H), 8.49 (d, 2 H), | | | |
| | 8.38 (d, 2 H), 6.83 (s, 1 H), 2.34 (s, 3 H). | | | |
| 21A | 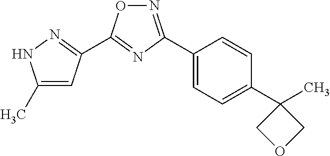 | 1.98 | 297 | C |
| | ¹H-NMR (400 MHz, CDCl₃, δ/ppm): 8.17 (d, 2 H), 7.33 (d, 2 H), 6.82 (s, 1 H), | | | |
| | 5.00 (d, 2 H), 4.68 (d, 2 H), 2.45 (s, 3 H), 1.77 (s, 3 H). | | | |
| 22A | 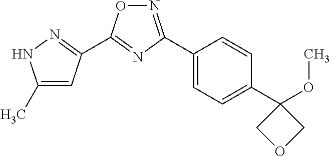 | 0.99 | 313 | F |
| | ¹H-NMR (400 MHz, DMSO-d₆, δ/ppm): 13.54 (s, broad, 1 H), 8.14 (d, 2 H), | | | |
| | 7.69 (d, 2 H), 6.80 (s, 1 H), 4.82 (d, 2 H), 4.78 (d, 2 H), 3.08 (s, 3 H), 2.37 (s, 3 H). | | | |
| 23A | 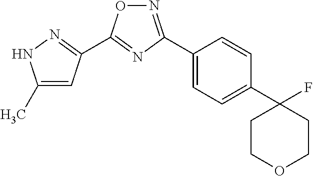 | 4.24 | 329 | C |
| | ¹H-NMR (400 MHz, CDCl₃, δ/ppm): 10.73 (broad, 1 H), 8.20 (d, 2 H), 7.52 (d, 2 H), | | | |
| | 6.81 (s, 1 H), 4.00-3.88 (m, 4 H), 2.45 (s, 3 H), 2.30-2.11 (m, 2 H), 1.98-1.91 (m, 2 H). | | | |
| 24A | 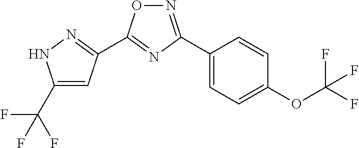 | 2.41 | 365 | E |
| | ¹H-NMR (400 MHz, CDCl₃, δ/ppm): 11.73 (broad, 1 H), 8.19 (d, 2 H), 7.38 (d, 2 H), | | | |
| | 7.37 (s, 1 H). | | | |
| 25A | 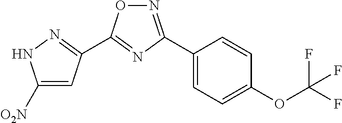 | 2.18 | 342 | E |
| | ¹H-NMR (500 MHz, DMSO-d₆, δ/ppm): 8.20 (d, 2 H), 7.58 (d, 2 H), 7.34 (s, 1 H). | | | |
| 26A | 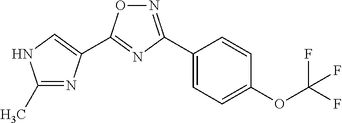 | 1.08 | 311 | F |
| | ¹H-NMR (400 MHz, CDCl₃, δ/ppm): 8.23 (d, 2 H), 7.82 (d, 2 H), 7.33 (s, 1 H), | | | |
| | 2.56 (s, 3 H). | | | |
| 27A | 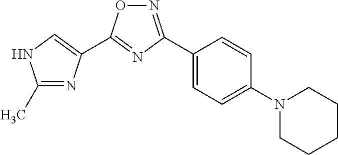 | 0.97 | 310 | F |

¹H-NMR (400 MHz, CDCl₃, δ/ppm): 9.61 (broad, 1 H), 8.02 (d, 2 H), 7.79 (s, 1 H),
6.96 (d, 2 H), 3.31-3.27 (m, 4 H), 2.54 (s, 3 H), 1.73-1.61 (m, 6 H).

### Example 28A

### 3-{3-[4-(Trifluoromethoxy)phenyl]-1,2,4-oxadiazol-5-yl}-1H-pyrazole-5-amine

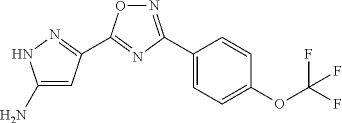

A solution of 342 mg (1.0 mmol) of the compound from Example 25A in 43 ml of ethyl acetate was hydrogenated in a flow-through hydrogenation apparatus (“H-Cube” from ThalesNano, Budapest, Hungary) (conditions: 10% Pd/C catalyst, 1 bar of H₂, 25° C., 1 ml/min). After removal of the solvent on a rotary evaporator, the crude product was purified by MPLC (silica gel, cyclohexane/ethyl acetate 1:1). 322 mg (93% of theory) of the title compound were obtained.

¹H-NMR (400 MHz, DMSO-d₆, δ/ppm): 12.49 (s, 1H), 8.19 (d, 2H), 7.49 (d, 2H), 5.93 (s, 1H), 5.44 (s, 2H).

MS (DCI, NH₃): m/z=312 [M+H]⁺.

LC/MS (method E, ESIpos): Rₜ=1.76 min, m/z=312 [M+H]⁺.

### Example 29A

### 2-Chloro-4-(chloromethyl)pyridine

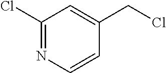

1.00 g (6.97 mmol) of (2-chloropyridin-4-yl)methanol was dissolved in 40 ml of dichloromethane, 10 ml of thionyl chloride were slowly added at RT and the mixture was stirred at RT overnight. The mixture was then concentrated on a rotary evaporator and the residue was stirred in a mixture of dichloromethane and aqueous sodium bicarbonate solution. The phases were separated and the dichloromethane phase was dried over anhydrous magnesium sulphate, filtered and concentrated on a rotary evaporator. 1.10 g (97% of theory) of the title compound were obtained.

¹H-NMR (400 MHz, CDCl₃, δ/ppm): 8.49 (d, 1H), 7.38 (s, 1H), 7.27-7.22 (m, 1H), 4.52 (s, 2H).

LC/MS (method E, ESIpos): Rₜ=1.43 min, m/z=162 [M+H]⁺.

### Example 30A

### 2-(Chloromethyl)-5-iodopyridine

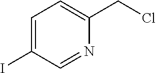

### Step 1: 2-(Hydroxymethyl)-5-iodopyridine

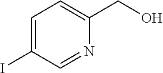

5.7 ml (9.07 mmol) of a 1.6 M solution of n-butyllithium in hexane were added dropwise to a solution of 2.50 g (7.56 mmol) of 2,5-diiodopyridine in 90 ml of toluene under inert conditions and at a temperature of −78° C. The mixture was stirred at −78° C. for 2.5 h and 756 μl of anhydrous DMF were then added at the same temperature. After a further 60 min at −78° C., the reaction mixture was allowed to warm to −10° C., 572 mg (15.11 mmol) of solid sodium borohydride were added and stirring was continued at 0° C. for 30 min. 25 ml of saturated aqueous ammonium chloride solution were then added and the mixture was warmed to RT. The organic phase was separated off and the solvent was removed on a rotary evaporator. The residue was purified by preparative HPLC. 890 mg (50% of theory) of the title compound (for the analytical data see below) and 243 mg (14% of theory) of the isomeric 5-(hydroxymethyl)-2-iodopyridine were obtained [preparative HPLC conditions: column: Sunfire C18 OBD 5 μm, 19 mm×150 mm; temperature: 40° C.; mobile phase: water/acetonitrile/1% strength aqueous TFA 76:5:19; flow rate: 25 ml/min; 1.3 g of crude product were dissolved in a mixture of 8 ml of 1% strength aqueous TFA and 4 ml of acetonitrile; injection volume: 1 ml].

¹H-NMR (400 MHz, CDCl₃, δ/ppm): 8.87 (d, 1H), 8.30 (dd, 1H), 7.38 (d, 1H), 5.43 (broad, 1H), 4.85 (s, 2H).

HPLC (method A): Rₜ=0.87 min.

MS (DCI, NH₃): m/z=236 [M+H]⁺.

LC/MS (method E, ESIpos): Rₜ=0.85 min, m/z=236 [M+H]⁺.

### Step 2: 2-(Chloromethyl)-5-iodopyridine

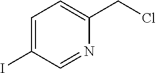

357 μl (4.88 mmol) of thionyl chloride were added dropwise to a solution of 765 mg (3.26 mmol) of the compound from Example 30A/step 1 in 12 ml of anhydrous dichloromethane at 0° C. The reaction mixture was then stirred at RT for 15 h. Approx. 50 ml of saturated aqueous sodium bicarbonate solution was then added and the mixture was extracted three times with approx. 50 ml of dichloromethane each time. The combined organic extracts were washed with saturated sodium chloride solution and dried over anhydrous magnesium sulphate. After filtration, the solvent was removed on a rotary evaporator. 541 mg (66% of theory) of the title compound were obtained.

¹H-NMR (400 MHz, CDCl₃, δ/ppm): 8.79 (d, 1H), 8.03 (dd, 1H), 7.29 (d, 1H), 4.61 (s, 2H).

MS (ESIpos): m/z=254/256 (³⁵Cl/³⁷Cl) [M+H]⁺.

LC/MS (method D, ESIpos): Rₜ=1.87 min, m/z=254/256 (³⁵Cl/³⁷Cl) [M+H]⁺.

### Example 31A

### 5-(Chloromethyl)pyridine-2-carbonitrile hydrochloride

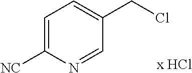

272 μl (3.73 mmol) of thionyl chloride were added to a solution of 250 mg (1.86 mmol) of 5-(hydroxymethyl)pyridine-2-carbonitrile [A. Ashimori et al., Chem. Pharm. Bull. 1990, 38 (9), 2446-2458] in 5 ml of anhydrous dichloromethane at 0° C. The reaction mixture was then stirred at RT for 6 h. All the volatile constituents were then removed on a rotary evaporator and the residue obtained in this way was dried under high vacuum. 263 mg (75% of theory) of the title compound were obtained.

¹H-NMR (400 MHz, CDCl₃, δ/ppm): 8.73 (d, 1H), 7.90 (dd, 1H), 7.72 (d, 1H), 4.63 (s, 2H).

MS (ESIpos): m/z=153/155 (³⁵Cl/³⁷Cl) [M+H]⁺.

LC/MS (method F, ESIpos): Rₜ=0.75 min, m/z=153/155 (³⁵Cl/³⁷Cl) [M+H]⁺.

### Example 32A (6-Cyanopyridin-3-yl)methyl methanesulphonate

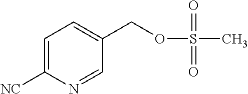

3.51 ml (27.14 mmol) of N,N-diisopropylethylamine and 2.87 ml (25.05 mmol) of methanesulphonic acid chloride were added successively to a solution of 2.8 g (20.87 mmol) of 5-(hydroxymethyl)pyridine-2-carbonitrile [A. Ashimori et al., Chem. Pharm. Bull. 1990, 38 (9), 2446-2458] in 50 ml of anhydrous dichloromethane at 0° C. The reaction mixture was then stirred at RT for 1 h. 10 ml of water were then added, the phases were separated and the aqueous phase was extracted twice with approx. 10 ml of dichloromethane each time. The combined organic extracts were washed with saturated sodium chloride solution, dried over anhydrous magnesium sulphate, filtered and freed from the solvent on a rotary evaporator. The residue obtained was separated into its components by MPLC (silica gel, cyclohexane/ethyl acetate 1:1). 2.12 g (48% of theory) of the title compound (for the analytical data see below) and 1.51 g (47% of theory) of the compound described in Example 31A were obtained.

¹H-NMR (400 MHz, CDCl₃, δ/ppm): 8.76 (d, 1H), 7.93 (dd, 1H), 7.78 (d, 1H), 5.32 (s, 2H), 3.10 (s, 3H).

MS (DCI, NH₃): m/z=213 [M+H]⁺, 230 [M+NH₄]⁺.

LC/MS (method F, ESIpos): Rₜ=0.57 min, m/z=213 [M+H]⁺.

### Example 33A

### [3-(Bromomethyl)phenoxy](tripropan-2-yl)silane

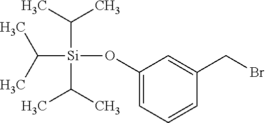

### Step 1: Ethyl 3-[(tripropan-2-ylsilyl)oxy]benzenecarboxylate

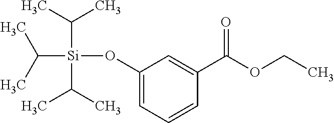

5.98 g (30.99 mmol) of triisopropylsilyl chloride were added dropwise to a solution of 5.0 g (30.09 mmol) of 3-hydroxybenzoic acid ethyl ester and 2.41 g (35.35 mmol) of imidazole in 20 ml of anhydrous DMF at 0° C. After the reaction mixture had been stirred at RT for 15 h, approx. 100 ml of water were added and the mixture was extracted three times with approx. 100 ml of diethyl ether each time. The combined organic extracts were washed successively with water and saturated sodium chloride solution. After drying over anhydrous magnesium sulphate and filtration, the solvent was removed on a rotary evaporator. The residue obtained was purified by filtration with suction over silica gel with cyclohexane/ethyl acetate 10:1→1:1 as the mobile phase. 9.70 g (100% of theory) of the title compound were obtained.

¹H-NMR (400 MHz, CDCl₃, δ/ppm): 7.62 (dd, 1H), 7.53 (m, 1H), 7.28 (dd, 1H), 7.06 (dd, 1H), 4.37 (quart, 2H), 1.39 (t, 3H), 1.28 (sept, 3H), 1.10 (d, 18H).

GC/MS (method I, EI): Rₜ=6.62 min, m/z=322 [M]⁺, 279 [M−C₃H₇]⁺.

### Step 2: {3-[(Tripropan-2-ylsilyl)oxy]phenyl}methanol

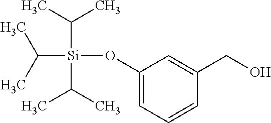

Under inert conditions, 50 ml (49.61 mmol) of a 1 M solution of lithium aluminium hydride in THF were diluted with 50 ml of anhydrous diethyl ether, and a solution of 8.0 g (24.80 mmol) of the compound from Example 33A/step 1 in 50 ml of anhydrous diethyl ether was then added dropwise at 0° C. The reaction mixture was stirred at RT for 1 h. A few ml of methanol were then first added in order to solvolyse excess hydride, and then approx. 150 ml of 0.1 M hydrochloric acid. The organic phase was separated off rapidly and the aqueous phase was extracted twice with approx. 50 ml of diethyl ether each time. The combined organic extracts were washed successively with water and saturated sodium chloride solution. After drying over anhydrous magnesium sulphate and subsequent filtration, the solvent was removed on a rotary evaporator. The residue obtained was purified by filtration with suction over silica gel with cyclohexane/ethyl acetate 5:1→1:1 as the mobile phase. 6.69 g (96% of theory) of the title compound were obtained.

¹H-NMR (400 MHz, CDCl₃, δ/ppm): 7.20 (dd, 1H), 6.93-6.90 (m, 2H), 6.80 (dd, 1H), 4.64 (d, 2H), 1.61 (t, 3H), 1.26 (sept, 3H), 1.09 (d, 18H).

GC/MS (method I, EI): Rₜ=6.38 min, m/z=280 [M]⁺, 237 [M−C₃H₇]⁺.

### Step 3: [3-(Bromomethyl)phenoxy](tripropan-2-yl)silane

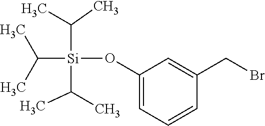

1.0 g (3.57 mmol) of the compound from Example 33A/step 2 was dissolved in 20 ml of anhydrous THF and 1.12 g (4.28 mmol) of triphenylphosphine were added. After this had dissolved, 1.42 g (4.28 mmol) of tetrabromomethane were added. The mixture was then stirred at RT for 20 h. The precipitate which had precipitated out was then filtered off and the filtrate was freed from the solvent on a rotary evaporator. The crude product was purified by MPLC (silica gel, cyclohexane/ethyl acetate 50:1). 1.10 g (90% of theory, purity approx. 90%) of the title compound were obtained, this being used without further purification.

¹H-NMR (400 MHz, CDCl₃, δ/ppm): 7.18 (dd, 1H), 6.95 (dd, 1H), 6.91 (m, 1H), 6.80 (dd, 1H), 4.43 (s, 2H), 1.25 (sept, 3H), 1.10 (d, 18H).

HPLC (method B): Rₜ=6.17 min.

GC/MS (method I, EI): Rₜ=6.56 min, m/z=342/344 (⁷⁹Br/⁸¹Br) [M]⁺.

### Example 34A

### Ethyl (4-{[(methylsulphonyl)oxy]methyl}phenyl)acetate

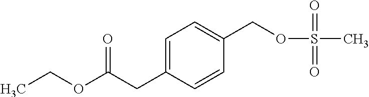

A solution of 1.1 g (5.66 mmol) of [4-(hydroxymethyl)phenyl]acetic acid ethyl ester [G. Biagi et al., Farmaco Ed. Sci. 1988, 43 (7/8), 597-612] and 1.03 ml (7.36 mmol) of triethylamine in 10 ml of anhydrous THF was cooled to 0° C. A solution of 526 μl (6.80 mmol) of methanesulphonic acid chloride in 5 ml of anhydrous THF was then added dropwise. After 15 min at 0° C., the mixture was warmed to RT. After a further hour, approx. 60 ml of water were added and the mixture was extracted twice with approx. 50 ml of ethyl acetate each time. The combined organic extracts were washed with saturated sodium chloride solution. After drying over anhydrous magnesium sulphate and filtration, the solvent was removed on a rotary evaporator. The crude product was purified by MPLC (silica gel, cyclohexane/ethyl acetate 7:3). 1.19 g (56% of theory, purity approx. 73%) of the title compound were obtained, this being used without further purification.

MS (DCI, NH₃): m/z=290 [M+NH₄]⁺.

LC/MS (method C, ESIpos): Rₜ=1.96 min, m/z=177 [M−CH₃SO₂O]⁺.

### Example 35A

### 1-[4-(Chloromethyl)benzyl]pyrrolidine hydrochloride

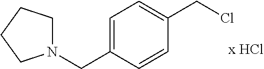

At RT, 80 μl (1.10 mmol) of thionyl chloride were added to a solution of 70 mg (0.366 mmol) of 1-[4-(hydroxymethyl)benzyl]pyrrolidine [S. Gemma et al., Bioorg. Med. Chem. Lett. 2006, 16 (20), 5384-5388] in 2.8 ml of anhydrous dichloromethane, and the mixture was stirred at RT for 3 h. The solvent was then removed on a rotary evaporator. The residue was taken up in 2-3 ml of chloroform and once more the solvent was removed on a rotary evaporator. After the residue had been dried under high vacuum, 90 mg (100% of theory) of the title compound were obtained.

¹H-NMR (400 MHz, CDCl₃, δ/ppm): 12.79 (broad, 1H), 7.69 (d, 2H), 7.47 (d, 2H), 4.58 (s, 2H), 4.20 (d, 2H), 3.68-3.60 (m, 2H), 2.87-2.80 (m, 2H), 2.31-2.20 (m, 2H), 2.10-2.00 (m, 2H).

HPLC (method A): Rₜ=3.30 min.

LC/MS (method F, ESIpos): Rₜ=0.44 min, m/z=210 [M+H]⁺.

### Example 36A

### 1-[(6-Chloropyridin-3-yl)methyl]-5-methyl-1H-pyrazole-3-carboxylic acid

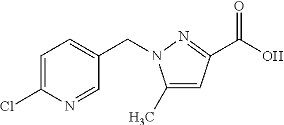

### Step 1: Ethyl 1-[(6-chloropyridin-3-yl)methyl]-5-methyl-1H-pyrazole-3-carboxylate

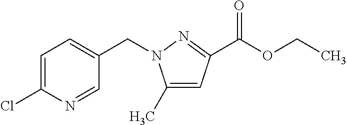

At 0° C., 9.46 g (84.3 mmol) of potassium tert-butylate were added to a solution of 10.0 g (64.9 mmol) of ethyl 3-methyl-1H-pyrazole-5-carboxylate and 13.66 g (84.3 mmol) of 2-chloro-5-(chloromethyl)pyridine in 162 ml of anhydrous THF. The mixture was allowed to come to RT and was stirred at RT for a further 18 h. It was then diluted with 200 ml of ethyl acetate and 350 ml of water, the phases were mixed thoroughly and the aqueous phase, which was separated off, was extracted twice more with 200 ml of ethyl acetate each time. The combined organic phases were dried over anhydrous sodium sulphate, filtered and concentrated on a rotary evaporator. The residue was purified by column chromatography on silica gel (mobile phase: cyclohexane/ethyl acetate 4:1→2:1). After drying under reduced pressure, 12.4 g (65% of theory) of the title compound were obtained in a purity of 95%.

¹H-NMR (400 MHz, DMSO-d₆, δ/ppm): 8.30 (d, 1H), 7.58 (dd, 1H), 7.52 (d, 1H), 6.60 (s, 1H), 5.45 (s, 2H), 4.24 (quart, 2H), 2.28 (s, 3H), 1.27 (t, 3H).

LC/MS (method C, ESIpos): Rₜ=1.88 min, m/z=280 [M+H]⁺.

### Step 2: 1-[(6-Chloropyridin-3-yl)methyl]-5-methyl-1H-pyrazole-3-carboxylic acid

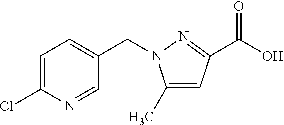

3.39 g (84.7 mmol) of sodium hydroxide, dissolved in 100 ml of water, were added to a solution of 11.85 g (42.36 mmol) of the compound from Example 36A/step 1 in 100 ml of THF and the mixture was stirred at RT for 5 h. The mixture was then diluted with 150 ml of water and washed once with 100 ml of ethyl acetate. The aqueous phase was adjusted to a pH of approx. 3 with 1 N hydrochloric acid and extracted three times with 150 ml of ethyl acetate each time. The latter ethyl acetate phases were combined, dried over anhydrous sodium sulphate, filtered and concentrated on a rotary evaporator. After the residue had been dried under reduced pressure, 9.72 g (91% of theory) of the title compound were obtained.

¹H-NMR (400 MHz, DMSO-d₆, δ/ppm): 12.60 (s, broad, 1H), 8.31 (d, 1H), 7.60 (dd, 1H), 7.52 (d, 1H), 6.53 (s, 1H), 5.42 (s, 2H), 2.28 (s, 3H).

LC/MS (method F, ESIpos): Rₜ=0.75 min, m/z=252 [M+H]⁺.

### Example 37A

### 1-[(6-Chloropyridin-3-yl)methyl]-5-methyl-1H-pyrrole-3-carboxylic acid

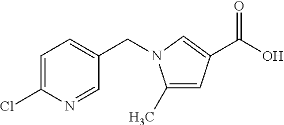

### Step 1: Methyl 2-(hydroxymethylidene)-4-oxopentanoate

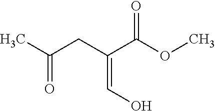

7.63 g (190.7 mmol) of a 60% strength suspension of sodium hydride in mineral oil were deoiled with pentane under inert conditions. 150 ml of anhydrous diethyl ether and, at 0° C., 138 μl (3.4 mmol) of methanol were then added. After stirring at RT for 10 min, the mixture was cooled to 0° C. again and a mixture of 12.6 ml (204.3 mmol) of formic acid methyl ester and 30.0 g (170.2 mmol) of methyl 4,4-dimethoxypentanoate [C. Meister et al., Liebigs Ann. Chem. 1983 (6), 913-921] was slowly added. The reaction mixture was stirred at RT for 16 h. Approx. 60 ml of ice-water were then added and the mixture was extracted with 100 ml of diethyl ether. The organic extract was discarded and the aqueous phase was brought to a pH of 2-3 with 3 M hydrochloric acid. It was extracted four times with approx. 50 ml of tert-butyl methyl ether each time. The combined organic extracts were dried over anhydrous magnesium sulphate, filtered and freed from the solvent on a rotary evaporator. 4.2 g (13% of theory, purity 85%) of the title compound were obtained, this being employed in the next stage without further purification.

GC/MS (method I, EI): Rₜ=3.33 min, m/z=158 [M]⁺, 140 [M−H₂O]⁺.

### Step 2: Methyl 1-[(6-chloropyridin-3-yl)methyl]-5-methyl-1H-pyrrole-3-carboxylate

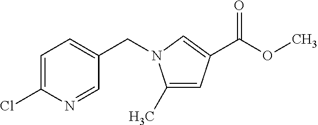

A mixture of 4.20 g (22.73 mmol, purity 85%) of the compound from Example 37A/step 1 and 3.24 g (22.73 mmol) of 5-(aminomethyl)-2-chloropyridine in 42 ml of methanol was stirred at RT for three days. The solvent was then removed on a rotary evaporator and the crude product was purified by MPLC (silica gel, cyclohexane/ethyl acetate 2:1). 3.37 g (56% of theory) of the title compound were obtained.

¹H-NMR (400 MHz, CDCl₃, δ/ppm): 8.19 (d, 1H), 7.30-7.20 (m, 3H), 6.38 (d, 1H), 5.03 (s, 2H), 3.79 (s, 3H), 2.12 (s, 3H).

HPLC (method A): Rₜ=4.10 min.

MS (DCI, NH₃): m/z=265 [M+H]⁺.

### Step 3: 1-[(6-Chloropyridin-3-yl)methyl]-5-methyl-1H-pyrrole-3-carboxylic acid

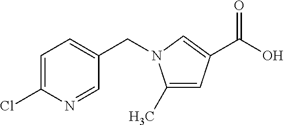

14.5 ml (14.5 mmol) of 1 M sodium hydroxide solution were added to a solution of 1.93 g (7.29 mmol) of the compound from Example 37A/step 2 in 38 ml of methanol. The reaction mixture was heated under reflux for 15 h. After cooling to RT, the methanol was mostly removed on a rotary evaporator. The residue was first diluted with 100 ml of water and then acidified with 2 M hydrochloric acid. The precipitate which had precipitated out was filtered off, rinsed with water and dried under high vacuum. 1.41 g (76% of theory) of the title compound were obtained.

¹H-NMR (400 MHz, DMSO-d₆, δ/ppm): 11.67 (s, 1H), 8.23 (s, 1H), 7.51 (d, 2H), 7.45 (d, 2H), 6.18 (d, 1H), 5.19 (s, 2H), 2.07 (s, 3H).

HPLC (method A): Rₜ=3.59 min.

MS (ESIpos): m/z=251 [M+H]⁺.

### Example 38A

### 2-Chloro-5-[(2-methyl-4-{3-[4-(trifluoromethoxy)phenyl]-1,2,4-oxadiazol-5-yl}-1H-pyrrol-1-yl)-methyl]pyridine

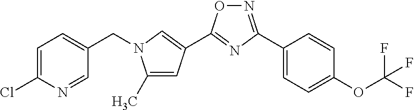

418 μl (4.79 mmol) of oxalyl chloride were added to a solution of 400 mg (1.60 mmol) of the compound from Example 37A in 20 ml of anhydrous dichloromethane at 0° C. under inert conditions. The reaction mixture was stirred at RT for 2 h. All the volatile constituents were then removed on a rotary evaporator and the residue obtained in this way was dried under high vacuum for 20 min. The residue was subsequently dissolved again in 4 ml of dichloromethane and this solution was added dropwise to a solution of 527 mg (2.39 mmol) of 4-(trifluoromethoxy)-N′-hydroxybenzenecarboximidamide and 445 μl (3.19 mmol) of triethylamine in 16 ml of dichloromethane at 0° C. After the reaction mixture had been stirred at RT for 16 h, all the volatile constituents were again removed on a rotary evaporator and the residue obtained was dissolved in 30 ml of DMSO. This solution was then heated at 140° C. in a microwave oven for 30 min (CEM Discover, initial irradiation power 250 W). After cooling to RT, the reaction mixture was purified by preparative HPLC (method K). 196 mg (28% of theory) of the title compound were obtained.

¹H-NMR (400 MHz, CDCl₃, δ/ppm): 8.24 (d, 1H), 8.17 (d, 2H), 7.47 (d, 1H), 7.32-7.27 (m, 4H), 6.60 (d, 1H), 5.10 (s, 2H), 2.20 (s, 3H).

LC/MS (method C, ESIpos): Rₜ=3.01 min, m/z=435 [M+H]⁺.

### Example 39A

### 2-Chloro-5-[(3-{3-[4-(2-fluoropropan-2-yl)phenyl]-1,2,4-oxadiazol-5-yl}-5-methyl-1H-pyrazol-1-yl)methyl]pyridine

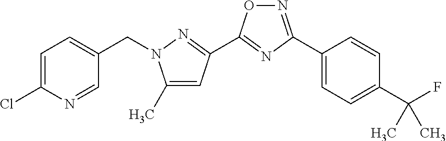

508 mg (2.65 mmol) of EDC and 358 mg (2.65 mmol) of HOBt were added to a solution of 667 mg (2.65 mmol) of the compound from Example 36A in 10 ml of anhydrous DMF at RT. After 30 min, 520 mg (2.65 mmol) of the compound from Example 2A, dissolved in 5 ml of DMF, were added. The mixture was stirred first at RT for 1 h and then at 140° C. for 1 h. After cooling, the majority of the solvent was removed on a rotary evaporator. 50 ml each of water and ethyl acetate were added. After separation of the phases, the organic phase was washed successively with 50 ml each of 10% strength aqueous citric acid, saturated sodium bicarbonate solution and saturated sodium chloride solution. After drying over anhydrous sodium sulphate, the mixture was filtered and the solvent was removed on a rotary evaporator. The crude product obtained was purified by MPLC (silica gel, cyclohexane/ethyl acetate 2:1). 418 mg (36% of theory, purity 93%) of the title compound were obtained, this being employed without further purification.

¹H-NMR (400 MHz, DMSO-d₆, δ/ppm): 8.39 (d, 1H), 8.08 (d, 2H), 7.68 (dd, 1H), 7.62 (d, 2H), 7.52 (d, 1H), 6.93 (s, 1H), 5.56 (s, 2H), 2.39 (s, 3H), 1.72 (s, 3H), 1.86 (s, 3H).

LC/MS (method F, ESIpos): Rₜ=1.43 min, m/z=412 [M+H]⁺.

The compounds in the following table were prepared from the corresponding precursors analogously to the process described in Example 39A.

| | | HPLC: | MS: m/z | LC/MS |
|---|---|---|---|---|
| Example | Structure | Rₜ [min] | [M + H]⁺ | method |
| 40A | 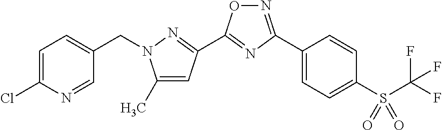 | 2.39 | 484 | E |
| | ¹H-NMR (400 MHz, DMSO-d₆, δ/ppm): 8.50 (d, 2 H), 8.42-8.33 (m, 3 H), 7.70 (dd, 1 H), | | | |
| | 7.53 (d, 2 H), 6.98 (s, 1 H), 5.56 (s, 2 H), 2.39 (s, 3 H). | | | |
| 41A | 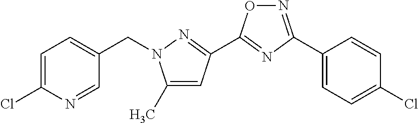 | 1.42 | 386 | F |

¹H-NMR (400 MHz, CDCl₃, δ/ppm): 8.32 (d, 2 H), 8.14 (d, 2 H), 7.51 (dd, 1 H), 7.48 (d, 2 H),
7.31 (d, 1 H), 6.82 (s, 1 H), 5.43 (s, 2 H), 2.32 (s, 3 H).

### Example 42A

### 2-Bromo-6-[(5-methyl-3-{3-[4-(trifluoromethoxy)phenyl]-1,2,4-oxadiazol-5-yl}-1H-pyrazol-1-yl)-methyl]pyridine

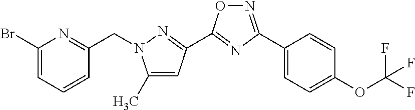

0.73 g (6.49 mmol) of solid potassium tert-butylate was added to a solution of 1.83 g (5.90 mmol) of the compound from Example 17A and 2.04 g (7.67 mmol) of (6-bromopyridin-2-yl)methyl methanesulphonate [T. Kawano et al., Bull. Chem. Soc. Jpn. 2003, 76 (4), 709-720] in 50 ml of anhydrous THF at 0° C. The reaction mixture was subsequently allowed to come to RT. After 1.5 h, approx. 100 ml of water were added and the mixture was extracted three times with approx. 100 ml of ethyl acetate each time. The combined organic extracts were dried over anhydrous sodium sulphate and, after filtration, the solvent was removed on a rotary evaporator. The residue obtained was stirred with 30 ml of dichloromethane. After filtration and drying of the residue on the filter, a first amount of 1.21 g (43% of theory) of the title compound was obtained. The mother liquor was freed from the solvent on a rotary evaporator and the residue was purified by MPLC (silica gel, cyclohexane/ethyl acetate 4:1→1:1). A further 0.42 g (16% of theory) of the title compound were obtained in this manner.

¹H-NMR (400 MHz, DMSO-d₆, δ/ppm): 8.20 (d, 2H), 7.78 (t, 1H), 7.63-7.58 (m, 3H), 7.18 (d, 1H), 6.96 (s, 1H), 5.60 (s, 2H), 2.39 (s, 3H).

LC/MS (method F, ESIpos): Rₜ=1.53 min, m/z=480/482 (⁷⁹Br/⁸¹Br) [M+H]⁺.

### Example 43A

### 5-Iodo-2-[(5-methyl-3-{3-[4-(trifluoromethoxy)phenyl]-1,2,4-oxadiazol-5-yl}-1H-pyrazol-1-yl)-methyl]pyridine

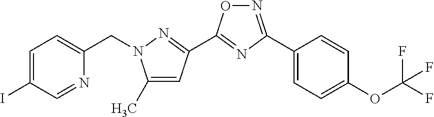

219 mg (1.95 mmol) of solid potassium tert-butylate were added to a solution of 504 mg (1.62 mmol) of the compound from Example 17A and 535 mg (2.11 mmol) of the compound from Example 30A in 20 ml of anhydrous THF at 0° C. The reaction mixture was subsequently allowed to come to RT. After 15 h, approx. 100 ml of water were added and the mixture was extracted three times with approx. 100 ml of ethyl acetate each time. The combined organic extracts were washed with saturated sodium chloride solution and dried over anhydrous magnesium sulphate. After filtration, the solvent was removed on a rotary evaporator. The title compound was isolated by preparative HPLC (method K). 657 mg (77% of theory) were obtained.

¹H-NMR (400 MHz, CDCl₃, δ/ppm): 8.79 (d, 1H), 8.24 (d, 2H), 7.97 (dd, 1H), 7.33 (d, 2H), 6.86 (d, 1H), 6.83 (s, 1H), 5.50 (s, 2H), 2.36 (s, 3H).

HPLC (method B): Rₜ=5.25 min.

MS (ESIpos): m/z=528 [M+H]⁺.

The compounds in the following table were prepared from the corresponding educts analogously to the processes described in Examples 42A and 43A. Depending on the polarity of the compounds, they were isolated either by extraction by stirring from dichloromethane, ethyl acetate, acetonitrile or diethyl ether, by preparative HPLC or by MPLC over silica gel with cyclohexane/ethyl acetate mixtures as the mobile phase. The arylmethyl chlorides, bromides or methanesulphonates used as educts were either commercially obtainable, or they were prepared as described above or their preparation is described in the literature: (6-chloropyridin-3-yl)methyl methanesulphonate [K. C. Iee et al., J. Org. Chem. 1999, 64 (23), 8576-8581].

| | | HPLC: | MS: m/z | LC/MS |
|---|---|---|---|---|
| Example | Structure | Rₜ [min] | [M + H]⁺ | method |
| 44A | 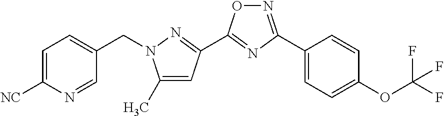 | 2.70 | 427 | C |
| | ¹H-NMR (400 MHz, CDCl₃, δ/ppm): 8.62 (d, 1 H), 8.24 (d, 2 H), 7.70 (d, 1 H), 7.62 (dd, 1 H), | | | |
| | 7.34 (d, 2 H), 6.88 (s, 1 H), 5.52 (s, 2 H), 2.34 (s, 3 H). | | | |
| 45A | 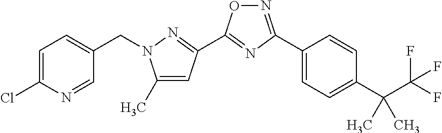 | 2.94 | 462 | C |
| | ¹H-NMR (400 MHz, CDCl₃, δ/ppm): 8.33 (d, 1 H), 8.18 (d, 2 H), 7.63 (d, 2 H), 7.52 (dd, 1 H), | | | |
| | 7.32 (d, 1 H), 6.84 (s, 1 H), 5.45 (s, 2 H), 2.32 (s, 3 H), 1.63 (s, 6 H). | | | |
| 46A | 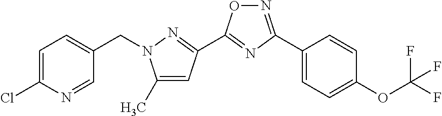 | 2.83 | 436 | C |
| | ¹H-NMR (400 MHz, CDCl₃, δ/ppm): 8.31 (d, 1 H), 8.25 (d, 2 H), 7.51 (dd, 1 H), 7.36- | | | |
| | 7.30 (m, 3 H), 6.82 (s, 1 H), 5.43 (s, 2 H), 2.32 (s, 3 H). | | | |
| 47A | 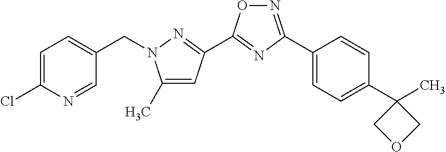 | 1.26 | 422 | F |
| | ¹H-NMR (400 MHz, CDCl₃, δ/ppm): 8.32 (d, 1 H), 8.20 (d, 2 H), 7.51 (dd, 1 H), 7.33 (d, 2 H), | | | |
| | 7.31 (d, 1 H), 6.83 (s, 1 H), 5.44 (s, 2 H), 5.00 (d, 2 H), 4.58 (d, 2 H), 2.33 (s, 3 H), 1.77 (s, 3 H). | | | |
| 48A | 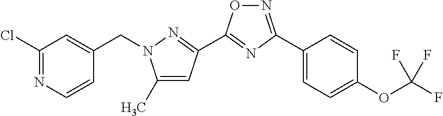 | 1.47 | 436 | F |
| | ¹H-NMR (400 MHz, CDCl₃, δ/ppm): 8.37 (d, 1 H), 8.28-8.22 (m, 2 H), 7.34 (d, 2 H), | | | |
| | 7.05 (s, 1 H), 6.97 (d, 1 H), 6.88 (s, 1 H), 5.43 (s, 2 H), 2.32 (s, 3 H). | | | |
| 49A | 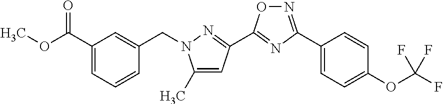 | 1.55 | 459 | F |
| | ¹H-NMR (400 MHz, CDCl₃, δ/ppm): 8.25 (d, 2 H), 7.99 (d, 1 H), 7.89 (s, 1 H), 7.42 (dd, 1 H), | | | |
| | 7.33 (d, 2 H and d, 1 H), 6.82 (s, 1 H), 5.50 (s, 2 H) 3.90 (s, 3 H), 2.29 (s, 3 H). | | | |
| 50A | 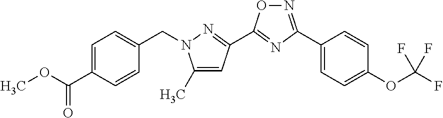 | 1.54 | 459 | F |
| | ¹H-NMR (400 MHz, CDCl₃, δ/ppm): 8.25 (d, 2 H), 8.01 (d, 2 H), 7.33 (d, 2 H), 7.21 (d, 2 H), | | | |
| | 6.83 (s, 1 H), 5.52 (s, 2 H), 3.91 (s, 3 H), 2.27 (s, 3 H). | | | |
| 51A | 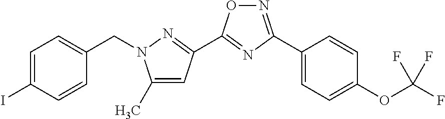 | 2.85 | 527 | E |
| | ¹H-NMR (400 MHz, CDCl₃, δ/ppm): 8.25 (d, 2 H), 7.67 (d, 2 H), 7.32 (d, 2 H), 6.91 (d, 2 H), | | | |
| | 6.81 (s, 1 H), 5.39 (s, 2 H), 2.27 (s, 3 H). | | | |
| 52A | 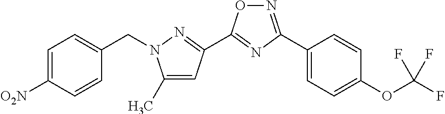 | 5.20 | 446 | B |
| | ¹H-NMR (400 MHz, CDCl₃, δ/ppm): 8.18 (d, 2 H), 8.14 (d, 2 H), 7.27 (d, 2 H), 7.24 (d, 2 H), | | | |
| | 6.80 (s, 1 H), 5.48 (s, 2 H), 2.23 (s, 3 H). | | | |
| 53A | 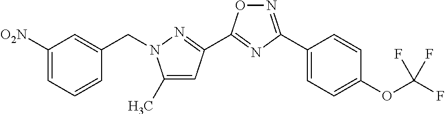 | 1.52 | 446 | F |

¹H-NMR (400 MHz, CDCl₃, δ/ppm): 8.25 (d, 2 H), 8.19 (d, 1 H), 8.04 (s, 1 H), 7.54 (dd, 1 H),
7.49 (d, 1 H), 7.33 (d, 2 H), 6.84 (s, 1 H), 5.55 (s, 2 H), 2.33 (s, 3 H).

### Example 54A

### 5-[(5-Methyl-3-{3-[4-(trifluoromethoxy)phenyl]-1,2,4-oxadiazol-5-yl}-1H-pyrazol-1-yl)methyl]-pyridine-2-carbaldehyde

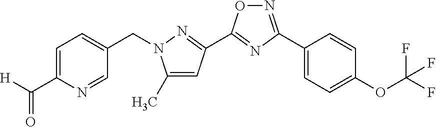

3.5 ml (3.5 mmol) of a 1 M solution of diisobutylaluminium hydride (DIBAL-H) in heptane was added to a solution of 980 mg (2.30 mmol) of the compound from Example 44A in 30 ml of anhydrous THF under inert conditions and at −78° C. After the reaction mixture had been stirred at −78° C. for 3 h, 22 ml of 1 M hydrochloric acid were added. The mixture was allowed to warm to RT, while stirring. It was then extracted with ethyl acetate. The organic extract was washed successively with water and saturated sodium chloride solution. After drying over anhydrous magnesium sulphate, the mixture was filtered and the solvent was removed on a rotary evaporator. The crude product was purified by MPLC (silica gel, cyclohexane/ethyl acetate 1:1). 300 mg (30% of theory) of the title compound were obtained.

¹H-NMR (400 MHz, CDCl₃, δ/ppm): 10.07 (s, 1H), 8.67 (d, 1H), 8.25 (d, 2H), 7.95 (d, 1H), 7.67 (dd, 1H), 7.34 (d, 2H), 6.87 (s, 1H), 5.57 (s, 2H), 2.35 (s, 3H).

MS (DCI, NH₃): m/z=430 [M+H]⁺.

LC/MS (method C, ESIpos): Rₜ=2.66 min, m/z=430 [M+H]⁺.

### Example 55A

### 5-[(5-Methyl-3-{3-[4-(trifluoromethoxy)phenyl]-1,2,4-oxadiazol-5-yl}-1H-pyrazol-1-yl)methyl]-pyridine-2-carboxylic acid

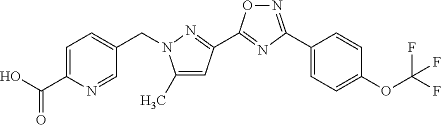

5 ml of a 30% strength potassium hydroxide solution in water were added to a solution of 500 mg (1.17 mmol) of the compound from Example 44A in 5 ml of ethanol and the mixture was heated at reflux for 1 h. After cooling to RT, approx. 20 ml of water were added and the product was precipitated out with concentrated hydrochloric acid. This was filtered off, washed neutral with water and dried under high vacuum. 448 mg (86% of theory) of the title compound were obtained.

¹H-NMR (400 MHz, CDCl₃, δ/ppm): 8.52 (d, 1H), 8.24 (d, 2H), 8.22 (d, 1H), 7.75 (dd, 1H), 7.33 (d, 2H), 6.88 (s, 1H), 5.57 (s, 2H), 2.36 (s, 3H).

MS (DCI, NH₃): m/z=446 [M+H]⁺.

LC/MS (method F, ESIpos): Rₜ=1.22 min, m/z=446 [M+H]⁺.

### Example 56A

### 3-[(5-Methyl-3-{3-[4-(trifluoromethoxy)phenyl]-1,2,4-oxadiazol-5-yl}-1H-pyrazol-1-yl)methyl]-benzenecarboxylic acid

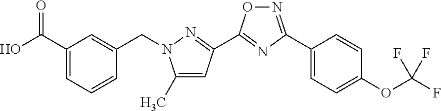

89 ml (88.7 mmol) of a 1 M sodium hydroxide solution were added to a suspension of 8.13 g (17.7 mmol) of the compound from Example 49A in 120 ml of methanol and the mixture was heated at reflux for 1 h. The methanol was then mostly removed on a rotary evaporator. The aqueous solution which remained was acidified with 100 ml of 1 M hydrochloric acid, while stirring. The product thereby precipitated out, and was filtered off with suction, washed with water and dried under high vacuum. 7.51 g (95% of theory) of the title compound were obtained.

¹H-NMR (400 MHz, DMSO-d₆, δ/ppm): 13.07 (s, broad, 1H), 8.20 (d, 2H), 7.40 (d, 1H), 7.78 (s, 1H), 7.59 (d, 2H), 7.51 (dd, 1H), 7.46 (d, 1H), 6.97 (s, 1H), 5.60 (s, 2H), 2.34 (s, 3H).

LC/MS (method C, ESIpos): Rₜ=2.68 min, m/z=445 [M+H]⁺.

Analogously to the process described in Example 56A, the compound in the following table was obtained by hydrolysis of the corresponding ester:

| | | HPLC: | MS: m/z | LC/MS |
|---|---|---|---|---|
| Example | Structure | Rₜ [min] | [M + H]⁺ | method |
| 57A | 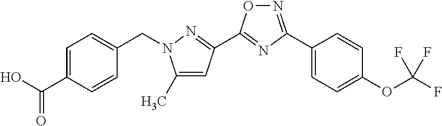 | 2.56 | 445 | D |

¹H-NMR (400 MHz, DMSO-d₆, δ/ppm): 13.00 (broad, 1 H), 8.20 (d, 2 H), 7.94 (d, 2 H),
7.59 (d, 2 H), 7.29 (d, 2 H), 6.96 (s, 1 H), 5.60 (s, 2 H), 2.33 (s, 3 H).

### Example 58A

### 3-[(5-Methyl-3-{3-[4-(trifluoromethoxy)phenyl]-1,2,4-oxadiazol-5-yl}-1H-pyrazol-1-yl)methyl]-phenol

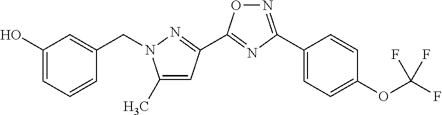

199 mg (1.77 mmol) of solid potassium tert-butylate were added to a solution of 500 mg (1.61 mmol) of the compound from Example 17A and 719 mg (2.10 mmol) of the compound from Example 33A in 10 ml of anhydrous THF at 0° C. The reaction mixture was subsequently allowed to come to RT. After 15 h, approx. 100 ml of water were added and the mixture was extracted three times with approx. 100 ml of ethyl acetate each time. The combined organic extracts were washed with saturated sodium chloride solution and dried over anhydrous magnesium sulphate. After filtration, the solvent was removed on a rotary evaporator. The residue obtained in this way was dissolved again in 20 ml of THF, and 3.2 ml (3.2 mmol) of a 1 M solution of tetra-n-butylammonium fluoride in THF were added at 0° C. After the mixture had been stirred at RT for 1 h, the batch was diluted with a few ml of methanol and separated into its components directly by preparative HPLC (method K). 218 mg (32% of theory) of the title compound were obtained.

¹H-NMR (400 MHz, CDCl₃, δ/ppm): 8.11 (d, 2H), 7.29 (d, 2H), 7.20 (t, 1H), 6.80 (d, 1H), 6.79 (s, 1H), 6.73 (d, 1H), 6.62 (s, 1H), 6.50 (s, 1H), 5.33 (s, 2H), 2.06 (s, 3H).

HPLC (method A): Rₜ=4.81 min.

MS (DCI, NH₃): m/z=417 [M+H]⁺.

LC/MS (method E, ESIpos): Rₜ=2.34 min, m/z=417 [M+H]⁺.

Analogously to the process described in Example 58A, the compound in the following table was obtained from the corresponding starting materials:

| | | HPLC: | MS: m/z | LC/MS |
|---|---|---|---|---|
| Example | Structure | Rₜ [min] | [M + H]⁺ | method |
| 59A | 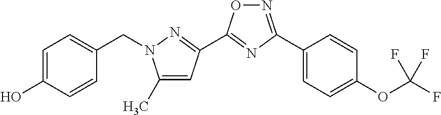 | 2.55 | 417 | D |

¹H-NMR (400 MHz, CDCl₃, δ/ppm): 8.24 (d, 2 H), 7.32 (d, 2 H), 7.07 (d, 2 H), 6.80 (s, 1 H),
6.79 (d, 2 H), 5.37 (s, 2 H), 5.31 (s, broad, 1 H), 2.28 (s, 3 H).

### Example 60A

### 4-[(5-Methyl-3-{3-[4-(trifluoromethoxy)phenyl]-1,2,4-oxadiazol-5-yl}-1H-pyrazol-1-yl)methyl]-aniline

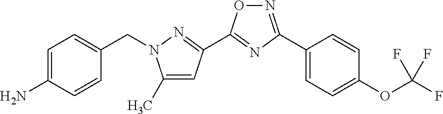

A solution of 400 mg (0.898 mmol) of the compound from Example 52A in a mixture of 25 ml of ethanol and 25 ml of ethyl acetate was hydrogenated in a flow-through hydrogenation apparatus (“H-Cube” from ThalesNano, Budapest, Hungary) (conditions: 10% Pd/C catalyst, “full H₂” mode, 1 ml/min, 25° C.). After removal of the solvent, the residue was taken up in a few ml of ethanol and the undissolved material was filtered off. This undissolved material was educt material, which was subsequently hydrogenated once more, as described above. The crude product obtained from the two hydrogenations was combined and purified by preparative HPLC (method K). 229 mg (62% of theory) of the title compound were obtained.

¹H-NMR (400 MHz, CDCl₃, δ/ppm): 8.25 (d, 2H), 7.33 (d, 2H), 7.01 (d, 2H), 6.87 (s, 1H), 6.63 (d, 2H), 5.33 (s, 2H), 3.69 (broad, 2H), 2.27 (s, 3H).

LC/MS (method C, ESIpos): Rₜ=2.57 min, m/z=416 [M+H]⁺.

The compound in the following table was prepared from the corresponding nitro compound by hydrogenation analogously to the process described in Example 60A:

| | | HPLC: | MS: m/z | LC/MS |
|---|---|---|---|---|
| Example | Structure | Rₜ [min] | [M + H]⁺ | method |
| 61A | 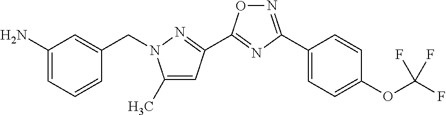 | 2.63 | 416 | D |

¹H-NMR (400 MHz, CDCl₃, δ/ppm): 8.26 (d, 2 H), 7.33 (d, 2 H), 7.10 (dd, 1 H), 6.80 (s, 1 H),
6.60 (dd, 1 H), 6.55 (dd, 1 H), 6.44 (dd, 1 H), 5.36 (s, 2 H), 3.67 (s, broad, 2 H), 2.27 (s, 3 H).

### Example 62A

### tert-Butyl {4-[(5-methyl-3-{3-[4-(trifluoromethoxy)phenyl]-1,2,4-oxadiazol-5-yl}-1H-pyrazol-1-yl)methyl]phenyl}carbamate

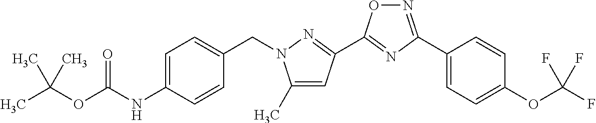

134 μl (0.963 mmol) of triethylamine and 3 mg (0.024 mmol) of DMAP were added to a solution of 200 mg (0.481 mmol) of the compound from Example 60A in 10 ml of anhydrous THF. The reaction mixture was cooled to 0° C. and 132 mg (0.602 mmol) of di-tert-butyl dicarbonate were added. The reaction mixture was stirred at 0° C. for 1 h and then at RT for a further 16 h. Thereafter, it was diluted with 5 ml of methanol and the product was isolated in two portions by preparative HPLC (method K). 74 mg (30% of theory) of the title compound were obtained.

¹H-NMR (400 MHz, CDCl₃, δ/ppm): 8.26 (d, 2H), 7.33 (2 d, tog. 4H), 7.12 (d, 2H), 6.79 (s, 1H), 6.49 (s, broad, 1H), 5.39 (s, 2H), 2.26 (s, 3H), 1.50 (s, 9H).

LC/MS (method E, ESIpos): Rₜ=2.74 min, m/z=516 [M+H]⁺.

### Example 63A

### 5-{1-[4-(2-Chloroethoxy)benzyl]-5-methyl-1H-pyrazol-3-yl}-3-[4-(trifluoromethoxy)phenyl]-1,2,4-oxadiazole

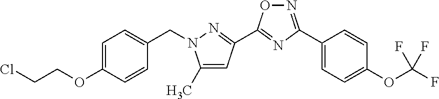

208 mg (0.50 mmol) of the compound from Example 59A together with 358 mg (1.10 mmol) of caesium carbonate were initially charged in 10 ml of DMF, 258 mg (1.10 mmol) of 2-chloroethyl 4-methylbenzenesulphonate were added and the reaction mixture was stirred at RT overnight. The mixture was then concentrated on a rotary evaporator, the residue was triturated with water and the solid formed was filtered off. The solid was purified by preparative HPLC (method L). The combined product-containing fractions were concentrated and the resulting residue was triturated with pentane. The resulting solid was filtered off with suction and dried under reduced pressure. 178 mg (74% of theory) of the title compound were obtained.

¹H-NMR (400 MHz, CDCl₃, δ/ppm): 8.25 (d, 2H), 7.32 (d, 2H), 7.13 (d, 2H), 6.88 (d, 2H), 6.79 (s, 1H), 5.39 (s, 2H), 4.20 (t, 2H), 3.80 (t, 2H), 2.28 (s, 3H).

LC/MS (method E, ESIpos): Rₜ=2.72 min, m/z=479 [M+H]⁺.

### Example 64A

### 2-(Pyrrolidin-1-yl)ethanethiol

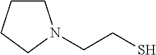

4.95 ml of ethylene sulphide were added dropwise to a solution of 6.94 ml (83.2 mmol) of pyrrolidine in 80 ml of toluene. The reaction mixture was then stirred initially at RT for 15 h and then at 50° C. for 4 h. The toluene was removed on a rotary evaporator and the residue was distilled under reduced pressure. The product had a boiling point of 59-60° C. at a pressure of about 7 mbar. 5.93 g (54% of theory) of the title compound were obtained.

¹H-NMR (400 MHz, CDCl₃, δ/ppm): 2.67-2.65 (m, 4H), 2.53-2.50 (m, 4H), 1.80-1.76 (m, 4H), 1.70 (s, broad, 1H).

GC/MS (method I, EIpos): Rₜ=2.67 min, m/z=131 [M]⁺.

### Example 65A

### 3-[4-(Chloromethyl)phenyl]propan-1-ol

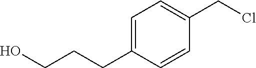

At RT, 483 μl (6.62 mmol) of thionyl chloride and 717 mg (6.02 mmol) of HOBt were added to a solution of 1.0 g (6.02 mmol) of 3-[4-(hydroxymethyl)phenyl]propan-1-ol [K. Tanaka et al., Org. Lett. 2007, 9 (7), 1215-1218] in 12 ml of anhydrous dichloromethane. After 5 min, a solution of 999 mg (6.02 mmol) of potassium iodide in 12 ml of DMF was added. After the reaction mixture had been stirred at RT for 16 h, it was diluted with 36 ml of water and the mixture was extracted three times with about 25 ml of diethyl ether each time. The combined organic extracts were washed successively with 5% strength aqueous sodium thiosulphate solution, water and saturated sodium chloride solution. Drying over anhydrous magnesium sulphate, filtration and subsequent evaporation of the solvent on a rotary evaporator gave a crude product which was purified by MPLC (silica gel, cyclohexane/ethyl acetate 2:1). 236 mg (21% of theory) of the title compound were obtained.

¹H-NMR (400 MHz, CDCl₃, δ/ppm): 7.31 (d, 2H), 7.20 (d, 2H), 4.57 (s, 2H), 3.68 (t, 2H), 2.71 (t, 2H), 1.89 (quint, 2H), 1.31 (s, broad, 1H).

MS (DCI, NH₃): m/z=202 [M+NH₄]⁺.

GC/MS (method I, EIpos): Rₜ=5.51 min, m/z=184 [M]⁺.

### Example 66A

### 3-{4-[(5-Methyl-3-{3-[4-(trifluoromethoxy)phenyl]-1,2,4-oxadiazol-5-yl}-1H-pyrazol-1-yl)-methyl]phenyl}propan-1-ol

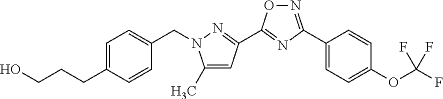

92 mg (0.820 mmol) of solid potassium tert-butylate were added to a solution of 231 mg (0.746 mmol) of the compound from Example 17A and 179 mg (0.969 mmol) of the compound from Example 65A in 7 ml of anhydrous THF at 0° C. The reaction mixture was stirred at RT for 16 h. Methanol was then added in the quantity required to dissolve the entire reaction mixture. This solution was separated into its components by preparative HPLC (method K). 138 mg (40% of theory) of the title compound were obtained.

¹H-NMR (400 MHz, CDCl₃, δ/ppm): 8.25 (d, 2H), 7.33 (d, 2H), 7.17 (d, 2H), 7.10 (d, 2H), 6.80 (s, 1H), 5.42 (s, 2H), 3.67 (quart, 2H), 2.70 (t, 2H), 2.28 (s, 3H), 1.87 (quint, 2H), 1.29 (t, 1H).

HPLC (method B): Rₜ=4.87 min.

MS (DCI, NH₃): m/z=459 [M+H]⁺.

### Example 67A

### 2-Bromo-5-[(5-methyl-3-{3-[4-(trifluoromethoxy)phenyl]-1,2,4-oxadiazol-5-yl}-1H-pyrazol-1-yl)-methyl]pyridine

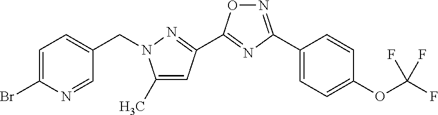

A mixture of 1.95 g (4.47 mmol) of the compound from Example 46A and 1.37 g (8.95 mmol) of bromo(trimethyl)silane in 0.5 ml of propionitrile was heated at 120° C. in a microwave apparatus for 70 min, while stirring (CEM Discover, initial irradiation power 250 W). During this operation a relatively marked increase in pressure and temperature was to be observed in the first 10 min. After cooling to RT, a further 350 mg (2.29 mmol) of bromo(trimethyl)silane were added and the mixture was heated at 120° C. in the microwave oven for a further 60 min. During this operation a relatively marked increase in pressure and temperature was again to be observed in the first 10 min. After cooling to RT, the mixture was diluted with 100 ml of water and 100 ml of ethyl acetate and the phases were separated. The organic phase was washed once with 100 ml of water, dried over sodium sulphate, filtered and concentrated on a rotary evaporator. The residue was purified by column chromatography on silica gel (mobile phase: cyclohexane/ethyl acetate 3:2). 1.45 g (65% of theory) of the title compound were obtained in a purity of 86% according to LC-MS. Approx. 10% of the educt (compound from Example 46A) was obtained as an impurity.

¹H-NMR (400 MHz, CDCl₃, δ/ppm): 8.31 (d, 1H), 8.23 (d, 2H), 7.47 (d, 1H), 7.40 (dd, 1H), 7.33 (d, 2H), 6.82 (s, 1H), 5.41 (s, 2H), 2.32 (s, 3H).

LC/MS (method E, ESIpos): Rₜ=2.54 min, m/z=480 [M+H]⁺.

### Example 68A

### 3-{5-[(5-Methyl-3-{3-[4-(trifluoromethoxy)phenyl]-1,2,4-oxadiazol-5-yl}-1H-pyrazol-1-yl)-methyl]pyridin-2-yl}prop-2-yn-1-ol

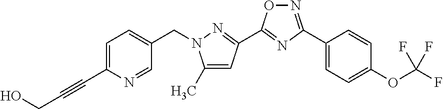

Under argon and at RT, 11.4 ml of triethylamine, 385 mg (6.87 mmol) of prop-2-yn-1-ol, 132 mg (0.115 mmol) of tetrakis(triphenylphosphine)palladium(0) and 44 mg (0.229 mmol) of copper(I)-iodide were added to a solution of 1.10 g (2.29 mmol) of the compound from Example 67A in 22 ml of degassed THF, and the mixture was stirred at RT for 16 h. The mixture was then concentrated on a rotary evaporator, the residue was dissolved in 8 ml of acetonitrile and 20 ml of water were added. After 30 min of stirring at RT, the solid formed was filtered off and washed in each case twice with water and ethyl acetate. Both the solid and the residue from the combined concentrated wash phases were purified separately by preparative HPLC (method L). The product-containing fractions were in each case combined, saturated aqueous sodium bicarbonate solution was added, the fractions were concentrated so that only a small residual volume of solvent remained and the mixture was extracted three times with ethyl acetate. The combined organic phases were in each case dried over sodium sulphate and concentrated. 634 mg (59% of theory) of the title compound were obtained from the two purifications.

¹H-NMR (400 MHz, CDCl₃, δ/ppm): 8.25 (d, 2H), 7.48-7.45 (dd, 1H), 7.40 (d, 1H), 7.33 (d, 2H), 6.82 (s, 1H), 5.46 (s, 2H), 4.51 (d, 2H), 2.31 (s, 3H), 1.96-1.91 (t, 1H).

LC/MS (method E, ESIpos): Rₜ=2.08 min, m/z=456 [M+H]⁺.

### Example 69A

### 3-{5-[(5-Methyl-3-{3-[4-(trifluoromethoxy)phenyl]-1,2,4-oxadiazol-5-yl}-1H-pyrazol-1-yl)-methyl]pyridin-2-yl}propan-1-ol

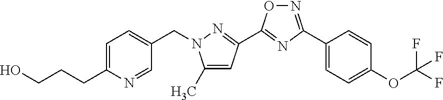

633 mg (1.39 mmol) of the compound from Example 68A were dissolved in a mixture of 7.5 ml of ethanol and 7.5 ml of THF, 358 μl (2.57 mmol) of triethylamine and 32 mg (0.139 mmol) of platinum (IV) oxide were added and the mixture was hydrogenated at RT and atmospheric pressure for 4 h. The reaction mixture was then filtered and the filtrate was purified by preparative HPLC (method L). The product-containing fractions were combined, and saturated aqueous sodium bicarbonate solution was added. Some of the solvent was removed on a rotary evaporator, and the remainder was then extracted three times with in each case 40 ml of ethyl acetate, the combined organic phases were dried over sodium sulphate and filtered and the solvent was removed. 390 mg (61% of theory) of the title compound were obtained.

¹H-NMR (400 MHz, CDCl₃, δ/ppm): 8.41 (s, 1H), 8.24 (d, 2H), 7.48 (dd, 1H), 7.32 (d, 2H), 7.18 (d, 1H), 6.82 (s, 1H), 5.42 (s, 2H), 3.70 (t, 2H), 2.96 (t, 2H), 2.31 (s, 3H), 2.01-1.93 (m, 2H).

LC/MS (method D, ESIpos): Rₜ=2.04 min, m/z=460 [M+H]⁺.

### Example 70A

### 3-{5-[(5-Methyl-3-{3-[4-(trifluoromethoxy)phenyl]-1,2,4-oxadiazol-5-yl}-1H-pyrazol-1-yl)-methyl]pyridine-2-yl}propanal

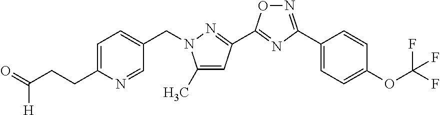

185 mg (0.402 mmol) of the compound from Example 69A were dissolved in 7.5 ml of dichloromethane, 256 mg (0.603 mmol) of 1,1-dihydro-1,1,1-triacetoxy-1,2-benziodoxol-3(1H)-one (Dess-Martin reagent) were added and the mixture was stirred at RT for 5 h. 40 ml of dilute aqueous sodium bicarbonate solution and 30 ml of dichloromethane were then added, the mixture was shaken and the phases were separated. The aqueous phase was extracted once with 20 ml of dichloromethane, the combined organic phases were dried over sodium sulphate, the solvent was removed and the residue was dried under reduced pressure. 180 mg (82% of theory) of the title compound were obtained in a purity of 73% according to LC-MS.

¹H-NMR (400 MHz, CDCl₃, δ/ppm): 9.85 (s, 1H), 8.42 (s, 1H), 8.24 (d, 2H), 7.44 (dd, 1H), 7.33 (d, 2H), 7.19 (d, 1H), 6.81 (s, 1H), 5.42 (s, 2H), 3.12 (t, 2H), 2.94 (t, 2H), 2.31 (s, 3H).

LC/MS (method M, ESIpos): Rₜ=0.91 min, m/z=458 [M+H]⁺.

### Example 71A

### 5-({4-[3-(4-tert-Butylphenyl)-1,2,4-oxadiazol-5-yl]-2-methyl-1H-pyrrol-1-yl}methyl)-2-chloro-pyridine

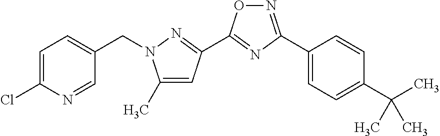

104 μl (1.20 mmol) of oxalyl chloride were added to a solution of 100 mg (0.399 mmol) of the compound from Example 37A in 3 ml of anhydrous dichloromethane at 0° C. under inert conditions. The reaction mixture was stirred at RT for 2 h. All volatile constituents were then removed on a rotary evaporator and the residue obtained in this way was dried under high vacuum for 20 min and then once more dissolved in 2 ml of dichloromethane. At 0° C., this solution was added dropwise to a solution of 92 mg (0.479 mmol) of 4-tert-butyl-N′-hydroxybenzenecarboximidamide and 111 μl (0.798 mmol) of triethylamine in 1 ml of dichloromethane. After the reaction mixture had been stirred at RT for 1 h, all the volatile constituents were again removed on a rotary evaporator and the residue obtained was dissolved in 4 ml of DMSO. This solution was then heated at 120° C. in a microwave oven for 30 min (CEM Discover, initial irradiation power 250 W). After cooling to RT, the reaction mixture was purified directly by preparative HPLC (method K). 71 mg (44% of theory) of the title compound were obtained.

¹H-NMR (400 MHz, CDCl₃, δ/ppm): 8.25 (d, 1H), 8.03 (d, 2H), 7.50 (d, 2H), 7.47 (d, 1H), 7.33 (d, 1H), 7.30 (dd, 1H), 6.60 (d, 1H), 5.10 (s, 2H), 2.20 (s, 3H), 1.37 (s, 9H).

HPLC (method A): Rₜ=5.20 min.

MS (DCI, NH₃): m/z=407 [M+H]⁺.

Analogously to the process described in Example 1A/step 5, the N′-hydroxycarboximidamides (hydroxyamidines) in the following table were prepared from the corresponding acrylonitriles. The acrylonitriles were either commercially available or described in the literature, or their preparation is described further above.

| | | HPLC: | MS: m/z | LC/MS |
|---|---|---|---|---|
| Example | Structure | Rₜ [min] | [M + H]⁺ | method |
| 72A | 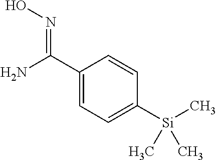 | 0.75 | 209 | M |
| | ¹H-NMR (400 MHz, CDCl₃, δ/ppm): 7.95 (s, broad, 1 H), 7.60 (d, 2 H), 7.55 (d, 2 H), | | | |
| | 4.86 (s, broad, 2 H), 0.27 (s, 9 H). | | | |
| 73A | 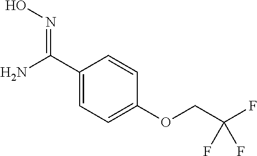 | 3.08 | 235 | A |

¹H-NMR (400 MHz, DMSO-d₆, δ/ppm): 9.51 (s, 1 H), 7.64 (d, 2 H), 7.06 (d, 2 H),
5.77 (s, broad, 2 H), 4.79 (quart, 2 H).

The compounds in the table below were prepared analogously to the process described in Example 17A. The time during which stirring was carried out at RT was 0.5 to 4 h, depending on the size of the batch. The mixture was heated at 140° C. for 1 to 15 h. Depending on the polarity of the product obtained, it precipitated even on addition of water after the reaction had ended, and it was then washed and dried under high vacuum, or it was extracted as described above and then purified chromatographically (silica gel MPLC or preparative HPLC). Various mobile phases were used for the chromatography on silica gel. In some cases it was possible to omit the chromatography and to purify the product directly by trituration with dichloromethane, ethyl acetate, acetonitrile or tert-butyl methyl ether.

| | | HPLC: | MS: m/z | LC/MS |
|---|---|---|---|---|
| Example | Structure | Rₜ [min] | [M + H]⁺ | method |
| 74A | 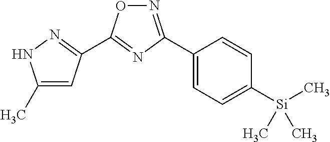 | 2.39 | 299 | E |
| | ¹H-NMR (400 MHz, CDCl₃, δ/ppm): 11.3 (s, broad, 1 H), 8.12 (d, 2 H), 7.63 (d, 2 H), | | | |
| | 6.81 (s, 1 H), 2.43 (s, 3 H), 0.31 (s, 9 H). | | | |
| 75A | 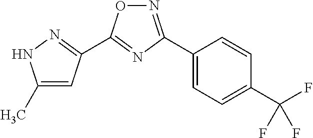 | 1.11 | 295 | M |
| | ¹H-NMR (400 MHz, CDCl₃, δ/ppm): 10.5 (broad, 1 H), 8.32 (d, 2 H), 7.77 (d, 2 H), | | | |
| | 6.82 (s, 1 H), 2.63 (s, 3 H). | | | |
| 76A | 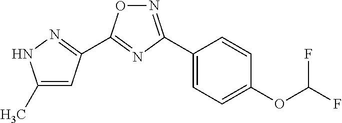 | 1.02 | 293 | M |

¹H-NMR (400 MHz, CDCl₃, δ/ppm): 10.85 (broad, 1 H), 8.20 (d, 2 H), 7.23 (d, 2 H),
6.81 (s, 1 H), 6.60 (t, 1 H), 2.46 (s, 3 H).

The compounds in the following table were prepared from the corresponding starting materials analogously to the processes described in Examples 42A and 43A. Depending on the polarity of the compounds, they were either isolated by trituration with dichloromethane, ethyl acetate, acetonitrile or diethyl ether, or they were purified by preparative HPLC or by MPLC on silica gel using cyclohexane/ethyl acetate mixtures as mobile phase. The arylmethyl chlorides, bromides or methanesulphonates used as starting materials were either commercially obtainable, or they were prepared as described above, or their preparation is described in the literature.

| | | HPLC: | MS: m/z | LC/MS |
|---|---|---|---|---|
| Example | Structure | Rₜ [min] | [M + H]⁺ | method |
| 77A | 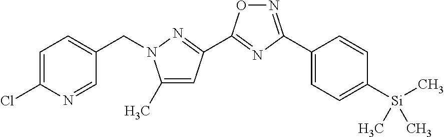 | 1.45 | 424 | M |
| | ¹H-NMR (400 MHz, DMSO-d₆, δ/ppm): 8.32 (dd, 1 H), 8.16 (d, 2 H), 7.63 (d, 2 H), | | | |
| | 7.52-7.49 (dd, 1 H), 7.31 (d, 1 H), 6.82 (s, 1 H), 5.42 (s, 2 H), 2.32 (s, 3 H), 0.31 (s, 9 H). | | | |
| 78A | 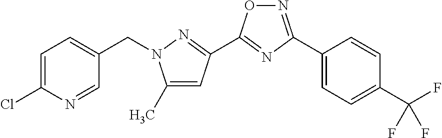 | 1.30 | 420 | M |
| | ¹H-NMR (400 MHz, DMSO-d₆, δ/ppm): 8.34-8.30 (m, 3 H), 7.77 (d, 2 H), 7.52 (dd, 1 H), | | | |
| | 7.31 (d, 1 H), 6.84 (s, 1 H), 5.44 (s, 2 H), 2.32 (s, 3 H). | | | |
| 79A | 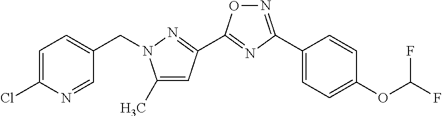 | 1.21 | 418 | M |

¹H-NMR (400 MHz, CDCl₃, δ/ppm): 8.32 (d, 1 H), 8.21 (d, 2 H), 7.51 (dd, 1 H), 7.31 (d, 1 H),
7.22 (d, 2 H), 6.82 (s, 1 H), 6.60 (t, 1 H), 5.43 (s, 2 H), 2.32 (s, 3 H).

The compounds in the following table were prepared from the corresponding precursors analogously to one of the processes described in Example 38A and 39A. The preparation of most of the N′-hydroxycarboximidamides (hydroxyamidines) employed has been described above; a very few were commercially obtainable or their preparation is described in the literature.

| | | HPLC: | MS: m/z | LC/MS |
|---|---|---|---|---|
| Example | Structure | Rₜ [min] | [M + H]⁺ | method |
| 80A | 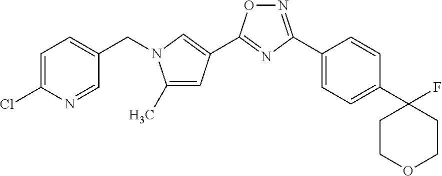 | 4.74 | 453 | A |
| | ¹H-NMR (400 MHz, CDCl₃, δ/ppm): 8.25 (d, 1 H), 8.14 (d, 2 H), 7.51 (d, 2 H), 7.48 (d, 1 H), | | | |
| | 7.33 (d, 1 H), 7.28 (dd, 1 H), 6.60 (d, 1 H), 5.11 (s, 2 H), 4.00-3.87 (m, 4 H), 2.29- | | | |
| | 2.11 (m, 2 H), 2.21 (s, 3 H), 1.98-1.91 (m, 2 H). | | | |
| 81A | 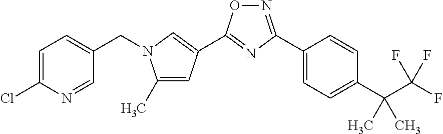 | 1.40 | 461 | M |
| | ¹H-NMR (400 MHz, CDCl₃, δ/ppm): 8.24 (d, 1 H), 8.10 (d, 2 H), 7.61 (d, 2 H), 7.50 (d, 1 H), | | | |
| | 7.34-7.30 (m, 2 H), 6.60 (d, 1 H), 5.13 (s, 2 H), 2.31 (s, 6 H), 2.21 (s, 3 H). | | | |
| 82A | 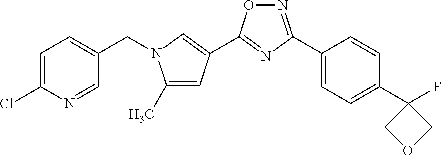 | 1.21 | 425 | M |
| | ¹H-NMR (400 MHz, CDCl₃, δ/ppm): 8.25 (d, 1 H), 8.20 (d, 2 H), 7.70 (d, 2 H), 7.49 (d, 1 H), | | | |
| | 7.33 (d, 1 H), 7.28 (dd, 1 H), 6.61 (d, 1 H), 5.11 (s, 2 H), 5.05 (dd, 2 H), 5.00 (dd, 2 H), 2.21 (s, 3 H). | | | |
| 83A | 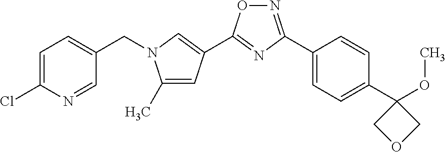 | 1.17 | 437 | M |
| | ¹H-NMR (400 MHz, CDCl₃, δ/ppm): 8.25 (d, 1 H), 8.18 (d, 2 H), 7.59 (d, 2 H), 7.48 (d, 1 H), | | | |
| | 7.33 (d, 1 H), 7.29 (dd, 1 H), 6.61 (d, 1 H), 5.11 (s, 2 H), 4.96 (d, 2 H), 4.85 (d, 2 H), | | | |
| | 3.17 (s, 3 H), 2.21 (s, 3 H). | | | |
| 84A | 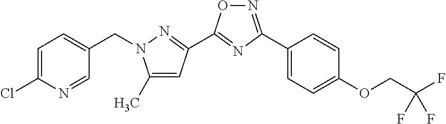 | 1.23 | 450 | M |
| | ¹H-NMR (400 MHz, CDCl₃, δ/ppm): 8.32 (d, 1 H), 8.18 (d, 2 H), 7.51 (dd, 1 H), 7.32 (d, 1 H), | | | |
| | 7.05 (d, 2 H), 6.82 (s, 1 H), 5.44 (s, 2 H), 4.43 (quart, 2 H), 2.33 (s, 3 H). | | | |
| 85A | 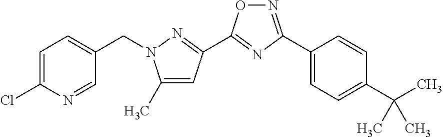 | 5.10 | 408 | A |
| | ¹H-NMR (400 MHz, CDCl₃, δ/ppm): 8.31 (d, 1 H), 8.13 (d, 2 H), 7.51 (d, 2 H), 7.51 (dd, 1 H), | | | |
| | 7.32 (d, 1 H), 6.83 (s, 1 H), 5.44 (s, 2 H), 2.32 (s, 3 H), 1.36 (s, 9 H). | | | |
| 86A | 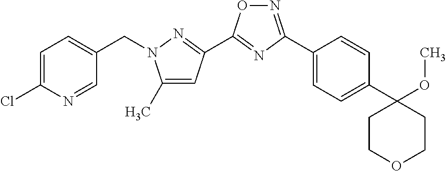 | 1.18 | 466 | A |
| | ¹H-NMR (400 MHz, CDCl₃, δ/ppm): 8.32 (d, 1 H), 8.21 (d, 2 H), 7.53 (d, 2 H), 7.52 (dd, 1 H), | | | |
| | 7.32 (d, 1 H), 6.84 (s, 1 H), 5.44 (s, 2 H), 3.93-3.83 (m, 4 H), 3.01 (s, 3 H), 2.33 (s, 3 H), | | | |
| | 2.11-1.98 (m, 4 H). | | | |
| 87A | 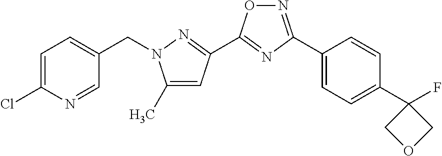 | 1.14 | 426 | M |
| | ¹H-NMR (400 MHz, CDCl₃, δ/ppm): 8.32 (d, 1 H), 8.28 (d, 2 H), 7.72 (d, 2 H), 7.52 (dd, 1 H), | | | |
| | 7.33 (d, 1 H), 6.84 (s, 1 H), 5.45 (s, 2 H), 5.05 (dd, 2 H), 5.00 (dd, 2 H), 2.33 (s, 3 H). | | | |
| 88A | 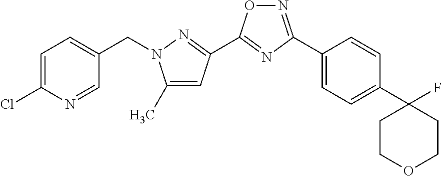 | 4.60 | 454 | A |
| | ¹H-NMR (400 MHz, CDCl₃, δ/ppm): 8.32 (d, 1 H), 8.23 (d, 2 H), 7.53 (d, 2 H), 7.51 (dd, 1 H), | | | |
| | 7.32 (d, 1 H), 6.83 (s, 1 H), 5.44 (s, 2 H), 4.00-3.86 (m, 4 H), 2.33 (s, 3 H), 2.29- | | | |
| | 2.12 (m, 2 H), 1.98-1.92 (m, 2 H). | | | |
| 89A | 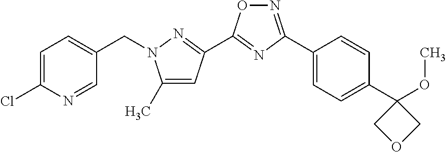 | 1.11 | 438 | M |
| | ¹H-NMR (400 MHz, CDCl₃, δ/ppm): 8.32 (d, 1 H), 8.26 (d, 2 H), 7.61 (d, 2 H), 7.52 (dd, 1 H), | | | |
| | 7.32 (d, 1 H), 6.84 (s, 1 H), 5.45 (s, 2 H), 4.97 (d, 2 H), 4.85 (d, 2 H), 3.19 (s, 3 H), | | | |
| | 2.33 (s, 3 H). | | | |
| 90A | 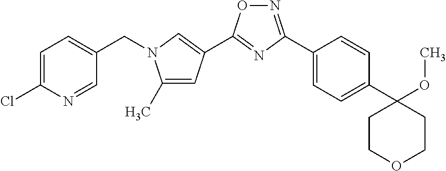 | 1.38 | 465 | F |
| | ¹H-NMR (400 MHz, CDCl₃, δ/ppm): 8.25 (d, 1 H), 8.12 (d, 2 H), 7.51 (d, 2 H), 7.48 (d, 1 H), | | | |
| | 7.33 (d, 1 H), 7.28 (dd, 1 H), 6.60 (d, 1 H), 5.11 (s, 2 H), 3.94-3.81 (m, 4 H), 3.01 (s, 3 H), | | | |
| | 2.20 (s, 3 H), 2.11-1.97 (m, 4 H). | | | |
| 91A | 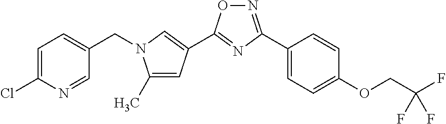 | 1.30 | 449 | M |

¹H-NMR (400 MHz, CDCl₃, δ/ppm): 8.25 (d, 1 H), 8.10 (d, 2 H), 7.46 (d, 1 H), 7.33 (d, 1 H),
7.27 (dd, 1 H), 7.04 (d, 2 H), 6.59 (d, 1 H), 5.10 (s, 2 H), 5.03 (s, 2 H), 4.42 (quart, 2 H),
2.20 (s, 3 H).

### Example 92A

N′-Hydroxy-4-(1-hydroxycyclobutyl)benzenecarboximidamide

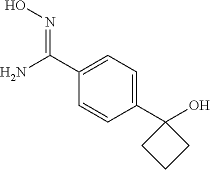

### Step 1: 4-(1-Hydroxycyclobutyl)benzenecarbonitrile

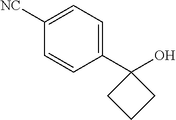

Analogously to the process described in Example 5A/step 1, 9.47 g (83% of theory) of the title compound were obtained from 15.0 g (65.5 mmol) of 4-iodobenzonitrile, 34.4 ml (68.8 mmol) of isopropylmagnesium chloride solution (2 M in diethyl ether) and 7.4 ml (98.2 mmol) of cyclobutanone. The purification of the product was carried out by MPLC (silica gel, mobile phase: cyclohexane/ethyl acetate 10:1→4:1).

¹H-NMR (400 MHz, CDCl₃, δ/ppm): 7.67 (d, 2H), 7.62 (d, 2H), 2.58-2.51 (m, 2H), 2.44-2.37 (m, 2H), 2.23-2.04 (m, 2H), 1.83-1.72 (m, 1H).

HPLC (method A): Rₜ=3.47 min.

MS (DCI, NH₃): m/z=191 [M+NH₄]⁺.

### Step 2: N′-Hydroxy-4-(1-hydroxycyclobutyl)benzenecarboximidamide

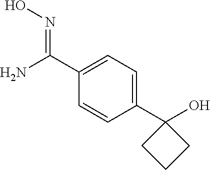

Analogously to the process described in Example 1A/step 5, 1.1 g of the title compound (92% of theory) were obtained starting from 1.0 g (5.77 mmol) of the compound from Example 92A/step 1. However, in variation to what is described in Example 1A/step 5, after removal of the solvent about 50 ml of water were added to the residue, and the mixture was extracted three times with in each case about 50 ml of ethyl acetate. The combined organic extracts were washed with saturated sodium chloride solution and dried over anhydrous magnesium sulphate. After filtration, the solvent was removed on a rotary evaporator and the residue obtained was purified by MPLC (silica gel, mobile phase: dichloromethane/methanol 50:1→10:1).

¹H-NMR (400 MHz, DMSO-d₆, δ/ppm): 9.57 (s, 1H), 7.63 (d, 2H), 7.47 (d, 2H), 5.79 (s, broad, 2H), 5.50 (s, 1H), 2.42-2.33 (m, 2H), 2.30-2.22 (m, 2H), 1.97-1.60 (m, 1H), 1.70-1.59 (m, 1H).

HPLC (method A): Rₜ=2.26 min.

MS (ESIpos): m/z=207 [M+H]⁺.

LC/MS (method M, ESIpos): Rₜ=0.25 min, m/z=207 [M+H]⁺.

### Example 93A

### N′-Hydroxy-4-(1-methoxycyclobutyl)benzenecarboximidamide

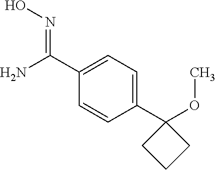

### Step 1: 4-(1-Methoxycyclobutyl)benzenecarbonitrile

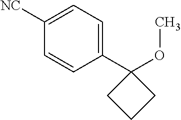

Analogously to the process described in Example 6A/step 1, 1.27 g (59% of theory) of the title compound were obtained from 2.0 g (11.5 mmol) of the compound from Example 92A/step 1, 508 mg (12.7 mmol) of a 60% strength dispersion of sodium hydride in mineral oil and 863 μl (13.9 mmol) of methyl iodide. The purification of the product was carried out by MPLC (silica gel, mobile phase: cyclohexane/ethyl acetate 20:1→4:1).

¹H-NMR (400 MHz, CDCl₃, δ/ppm): 7.68 (d, 2H), 7.54 (d, 2H), 2.95 (s, 3H), 2.46-2.32 (m, 4H), 2.03-1.93 (m, 1H), 1.76-1.63 (m, 1H).

MS (DCI, NH₃): m/z=205 [M+NH₄]⁺.

### Step 2: N′-Hydroxy-4-(1-methoxycyclobutyl)benzenecarboximidamide

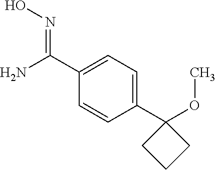

Analogously to the process described in Example 1A/step 5, 1.28 g of the title compound (98% of theory) were obtained starting from 1.1 g (5.87 mmol) of the compound from Example 93A/step 1.

¹H-NMR (400 MHz, DMSO-d₆, δ/ppm): 9.62 (s, 1H), 7.68 (d, 2H), 7.40 (d, 2H), 5.80 (s, broad, 2H), 2.83 (s, 3H), 2.37-2.24 (m, 4H), 1.91-1.81 (m, 1H), 1.65-1.53 (m, 1H).

HPLC (method A): Rₜ=3.02 min.

MS (DCI, NH₃): m/z=221 [M+H]⁺.

### Example 94A

### 4-(1-Fluorocyclobutyl)-N′-hydroxybenzenecarboximidamide

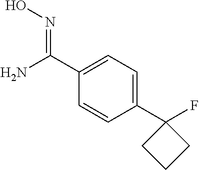

### Step 1: 4-(1-Fluorocyclobutyl)benzenecarbonitrile

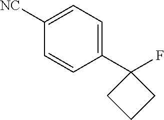

Analogously to the process described in Example 5A/step 2, 1.39 g (69% of theory) of the title compound were obtained from 2.0 g (11.5 mmol) of the compound from Example 92A/step 1 and 1.8 ml (13.9 mmol) of diethylaminosulphur trifluoride (DAST). The purification of the product was carried out by MPLC (silica gel, mobile phase: cyclohexane/ethyl acetate 10:1→5:1).

¹H-NMR (400 MHz, CDCl₃, δ/ppm): 7.69 (d, 2H), 7.57 (d, 2H), 2.78-2.62 (m, 2H), 2.58-2.48 (m, 2H), 2.20-2.09 (m, 1H), 1.87-1.75 (m, 1H).

GC/MS (method I, EIpos): Rₜ=4.71 min, m/z=155 [M−HF]⁺.

### Step 2: 4-(1-Fluorocyclobutyl)-N′-hydroxybenzenecarboximidamide

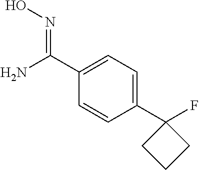

Analogously to the process described in Example 1A/step 5, 1.16 g of the title compound (78% of theory) were obtained starting from 1.25 g (7.13 mmol) of the compound from Example 94A/step 1.

¹H-NMR (400 MHz, CDCl₃, δ/ppm): 7.67 (d, 2H), 7.50 (d, 2H), 4.87 (s, broad, 2H), 2.72-2.52 (m, 5H), 2.16-2.05 (m, 1H), 1.82-1.71 (m, 1H).

HPLC (method A): Rₜ=3.17 min.

MS (DCI, NH₃): m/z=209 [M+H]⁺.

### Example 95A

### N′-Hydroxy-4-(1H-pyrrol-1-ylmethyl)benzenecarboximidamide

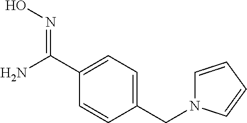

Analogously to the process described in Example 1A/step 5, 702 mg of the title compound (86% of theory) were obtained from 670 mg (3.68 mmol) of 4-(1H-pyrrol-1-ylmethyl)benzenecarbonitrile [M. Artico et al., Eur. J. Med. Chem. 1992, 27 (3), 219-228].

¹H-NMR (400 MHz, CDCl₃, δ/ppm): 7.76 (broad, 1H), 7.58 (d, 2H), 7.13 (d, 2H), 6.68 (dd, 2H), 6.20 (dd, 2H), 5.09 (s, 2H), 4.84 (s, broad, 2H).

LC/MS (method M, ESIpos): Rₜ=0.54 min, m/z=216 [M+H]⁺.

### Example 96A

### 4-[(Diisopropylamino)methyl]-N′-hydroxybenzenecarboximidamide

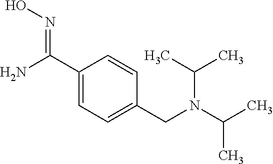

### Step 1: 4-[(Diisopropylamino)methyl]benzonitrile

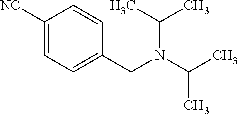

A mixture of 4.00 g (20.4 mmol) of 4-(bromomethyl)benzonitrile and 6.19 g (61.2 mmol) of diisopropylamine in 40 ml of toluene was heated in two portions at 150° C. in a microwave apparatus (CEM Discover, initial irradiation power 250 W) for in each case 3 h. After cooling to RT, the solid formed was filtered off and the filtrate was concentrated to obtain 4.52 g (92% of theory, purity 90%) of the title compound in this way.

LC/MS (method F, ESIpos): Rₜ=0.30 min, m/z=217 [M+H]⁺.

### Step 2: 4-[(Diisopropylamino)methyl]-N′-hydroxybenzenecarboximidamide

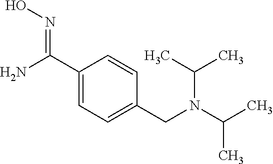

Analogously to the process described in Example 1A/step 5, 4.93 g (70% of theory) of the title compound were obtained from 6.80 g (28.29 mmol, purity 90%) of the compound from Example 96A/step 1.

¹H-NMR (400 MHz, CDCl₃, δ/ppm): 7.52 (d, 2H), 7.41 (d, 2H), 4.84 (s, broad, 2H), 3.64 (s, 2H), 3.05-2.95 (m, 2H), 1.01 (d, 12H).

LC/MS (method M, ESIpos): Rₜ=0.18 min, m/z=250 [M+H]⁺.

### Example 97A

### 4-[4-(Chloromethyl)benzyl]morpholine hydrochloride

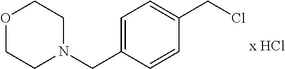

At RT, 0.219 ml (3.00 mmol) of thionyl chloride was added to a solution of 207 mg (1.00 mmol) of [4-(morpholin-4-ylmethyl)phenyl]methanol in 7.5 ml of dichloromethane, and the mixture was then stirred at this temperature for 3 h. The mixture was then concentrated on a rotary evaporator, 10 ml of dichloromethane were added to the residue and the mixture was concentrated again. Addition of dichloromethane and concentration were then repeated three more times. 259 mg (93% of theory, purity 94%) in total of the title compound were obtained.

¹H-NMR (400 MHz, CDCl₃, δ/ppm): 13.38 (s, broad, 1H), 7.71 (d, 2H), 7.49 (d, 2H), 4.60 (s, 2H), 4.38-4.26 (t, 2H), 4.16 (d, 2H), 3.98-3.90 (dd, 2H), 3.32 (d, 2H), 2.92-2.80 (m, 2H).

LC/MS (method D, ESIpos): Rₜ=0.96 min, m/z=226/228 [M+H]⁺.

### Example 98A

### 2-(1,1-Dioxidothiomorpholin-4-yl)ethanamine

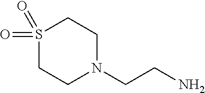

### Step 1: (1,1-Dioxidothiomorpholin-4-yl)acetonitrile

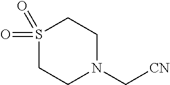

2.66 g (19.3 mmol) of potassium carbonate were added to a solution of 1.74 g (12.8 mmol) of thiomorpholine 1,1-dioxide [E. S. Lazer et al., J. Med. Chem. 2007, 37 (7), 913-923] and 1.69 g (14.1 mmol) of bromoacetonitrile in 30 ml of acetonitrile, and the mixture was stirred for 16 h at 60° C. After cooling, precipitated salts were filtered off and the filtrate was evaporated to dryness on a rotary evaporator. The residue obtained was purified by MPLC (silica gel, mobile phase: cyclohexane/ethyl acetate 1:1). 2.03 g (91% of theory) of the title compound were obtained.

¹H-NMR (400 MHz, CDCl₃, δ/ppm): 3.61 (s, 2H), 3.13 (s, 8H).

MS (DCI, NH₃): m/z=192 [M+NH₄]⁺.

GC/MS (method I, EIpos): Rₜ=6.66 min, m/z=174 [M]⁺.

### Step 2: 2-(1,1-Dioxidothiomorpholin-4-yl)ethanamine

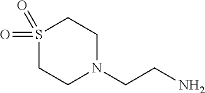

A solution of 310 mg (1.78 mmol) of the compound from Example 98A/step 1 in 40 ml of a 2 M solution of gaseous ammonia in methanol was hydrogenated in a flow-through hydrogenation apparatus (“H-Cube” from Thales Nano, Budapest, Hungary; conditions: Raney nickel cartridge, 70 bar of hydrogen, 70° C., flow rate 1 ml/min). After the solvent had been evaporated on a rotary evaporator, 311 mg (98% of theory) of the title compound were obtained.

¹H-NMR (400 MHz, CDCl₃, δ/ppm): 3.07-3.03 (m, 8H), 2.80 (t, 2H), 2.60 (t, 2H), 1.41 (broad, 2H).

MS (DCI, NH₃): m/z=179 [M+H]⁺.

### Example 99A

### 2-(4,4-Difluoropiperidin-1-yl)ethanamine

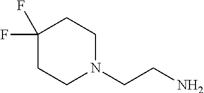

### Step 1: (4,4-Difluoropiperidin-1-yl)acetonitrile

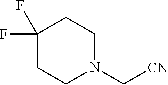

Analogously to the process described in Example 98A/step 1, 1.95 g (96% of theory) of the title compound were obtained from 2.00 g (12.7 mmol) of 4,4-difluoropiperidine hydrochloride and 1.67 g (14.0 mmol) of bromoacetonitrile.

¹H-NMR (400 MHz, CDCl₃, δ/ppm): 3.56 (s, 2H), 2.72-2.70 (m, 4H), 2.11-2.01 (m, 4H).

MS (DCI, NH₃): m/z=161 [M+H]⁺.

GC/MS (method I, EIpos): Rₜ=3.28 min, m/z=160 [M]⁺.

### Step 2: 2-(4,4-Difluoropiperidin-1-yl)ethanamine

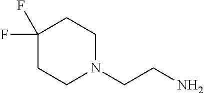

Analogously to the process described in Example 98A/step 2, 0.96 g (94% of theory) of the title compound was obtained from 1.00 g (6.24 mmol) of the compound from Example 99A/step 1.

¹H-NMR (400 MHz, CDCl₃, δ/ppm): 2.78 (t, 2H), 2.58-2.53 (m, 4H), 2.47 (t, 2H), 2.03-1.94 (m, 4H), 1.53 (broad, 2H).

MS (DCI, NH₃): m/z=165 [M+H]⁺.

GC/MS (method I, EIpos): Rₜ=2.82 min, m/z=164 [M]⁺.

### Example 100A

### 2-(2,2-Dimethylpyrrolidin-1-yl)ethanamine

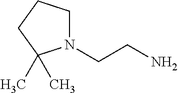

### Step 1: (2,2-Dimethylpyrrolidin-1-yl)acetonitrile

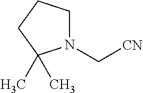

Analogously to the process described in Example 98A/step 1, 301 mg (59% of theory) of the title compound were obtained from 500 mg (3.69 mmol) of 2,2-dimethylpyrrolidine hydrochloride [Moffett, Org. Synth. Coll. Vol. IV, 354 (1963)] and 663 mg (5.53 mmol) of bromoacetonitrile.

¹H-NMR (400 MHz, CDCl₃, δ/ppm): 3.52 (s, 2H), 2.95 (t, 2H), 1.86-1.79 (m, 2H), 1.68 (t, 2H), 1.08 (s, 6H).

GC/MS (method I, EIpos): Rₜ=3.09 min, m/z=138 [M]⁺.

### Step 2: 2-(2,2-Dimethylpyrrolidin-1-yl)ethanamine

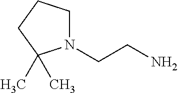

Analogously to the process described in Example 98A/step 2, 192 mg (62% of theory) of the title compound were obtained from 300 mg (2.17 mmol) of the compound from Example 100A/step 1.

¹H-NMR (400 MHz, CDCl₃, δ/ppm): 2.77-2.72 (m, 4H), 2.46 (t, 2H), 1.80-1.72 (m, 2H), 1.68-1.63 (m, 2H), 0.98 (s, 6H).

MS (DCI, NH₃): m/z=143 [M+H]⁺.

### Example 101A

### 2-(4-Fluoropiperidin-1-yl)ethanamine

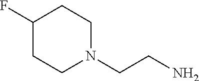

### Step 1: (4-Fluoropiperidin-1-yl)acetonitrile

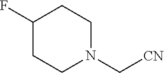

Analogously to the process described in Example 98A/step 1, 1.81 g (89% of theory) of the title compound were obtained from 2.0 g (14.3 mmol) of 4-fluoropiperidine hydrochloride and 2.06 g (17.2 mmol) of bromoacetonitrile.

¹H-NMR (400 MHz, CDCl₃, δ/ppm): 4.79-4.62 (m, 1H), 3.52 (s, 2H), 2.76-2.70 (m, 2H), 2.57-2.51 (m, 2H), 1.99-1.90 (m, 4H).

GC/MS (method I, EIpos): Rₜ=3.55 min, m/z=142 [M]⁺.

### Step 2: 2-(4-Fluoropiperidin-1-yl)ethanamine

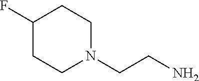

Analogously to the process described in Example 98A/step 2, 1.30 g (98% of theory) of the title compound were obtained from 1.30 g (9.14 mmol) of the compound from Example 101A/step 1.

¹H-NMR (400 MHz, CDCl₃, δ/ppm): 4.76-4.59 (m, 1H), 2.79 (t, 2H), 2.62-2.56 (m, 2H), 2.42 (t, 2H), 2.42-2.34 (m, 2H), 1.98-1.81 (m, 4H).

MS (DCI, NH₃): m/z=147 [M+H]⁺.

### Example 102A

### N-Isopropyl-N-{4-[5-(5-methyl-1H-pyrazol-3-yl)-1,2,4-oxadiazol-3-yl]benzyl}propan-2-amine

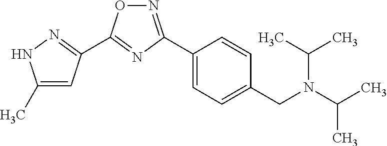

Analogously to the process described in Example 17A, 2.00 g (15.9 mmol) of 5-methyl-1H-pyrazole-3-carboxylic acid and 3.95 g (15.9 mmol) of the compound from Example 96A were reacted to give 1.49 g (26% of theory, purity 93%) of the title compound.

¹H-NMR (400 MHz, CDCl₃, δ/ppm): 11.50 (s, broad, 1H), 8.08 (d, 2H), 7.51 (d, 2H), 6.81 (s, 1H), 3.70 (s, 2H), 3.10-2.98 (m, 2H), 2.42 (s, 3H), 1.02 (d, 12H).

LC/MS (method F, ESIpos): Rₜ=0.73 min, m/z=340 [M+H]⁺.

### Example 103A

### 2-Chloro-5-[(3-{3-[4-(1-fluorocyclobutyl)phenyl]-1,2,4-oxadiazol-5-yl}-5-methyl-1H-pyrazol-1-yl)methyl]pyridine

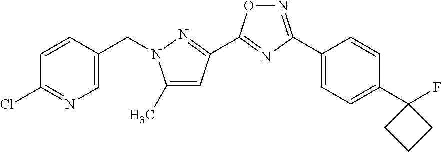

Analogously to the process described in Example 38A, 181 mg (72% of theory) of the title compound were obtained from 150 mg (0.596 mmol) of the compound from Example 36A and 137 mg (0.656 mmol) of the compound from Example 94A. The purification of the crude product was carried out by MPLC (silica gel, mobile phase: dichloromethane→cyclohexane/ethyl acetate 3:1).

¹H-NMR (400 MHz, CDCl₃, δ/ppm): 8.32 (d, 1H), 8.22 (d, 2H), 7.60 (d, 2H), 7.51 (dd, 1H), 7.32 (d, 1H), 6.84 (s, 1H), 5.44 (s, 2H), 2.77-2.55 (m, 4H), 2.33 (s, 3H), 2.20-2.08 (m, 1H), 1.87-1.75 (m, 1H).

HPLC (method A): Rₜ=4.84 min.

MS (DCI, NH₃): m/z=424 [M+H]⁺.

LC/MS (method M, ESIpos): Rₜ=1.32 min, m/z=424/426 [M+H]⁺.

### Example 104A

### 2-Chloro-5-[(3-{3-[4-(1-methoxycyclobutyl)phenyl]-1,2,4-oxadiazol-5-yl}-5-methyl-1H-pyrazol-1-yl)methyl]pyridine

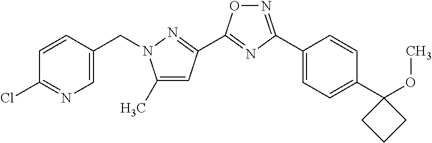

Analogously to the process described in Example 38A, 209 mg (80% of theory) of the title compound were obtained from 150 mg (0.596 mmol) of the compound from Example 36A and 144 mg (0.656 mmol) of the compound from Example 93A.

¹H-NMR (400 MHz, CDCl₃, δ/ppm): 8.32 (d, 1H), 8.20 (d, 2H), 7.56 (d, 2H), 7.52 (dd, 1H), 7.32 (d, 1H), 6.84 (s, 1H), 5.44 (s, 2H), 2.97 (s, 3H), 2.44-2.41 (m, 4H), 2.33 (s, 3H), 2.03-1.93 (m, 1H), 1.78-1.67 (m, 1H).

LC/MS (method M, ESIpos): Rₜ=1.30 min, m/z=436/438 [M+H]⁺.

### Example 105A

### 2-Chloro-5-[(5-methyl-3-{3-[4-(piperidin-1-yl)phenyl]-1,2,4-oxadiazol-5-yl}-1H-pyrazol-1-yl)-methyl]pyridine

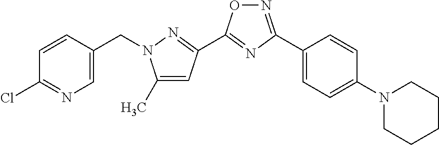

Analogously to the process described in Example 38A, 33 mg (14% of theory, purity 94%) of the title compound were obtained from 125 mg (0.497 mmol) of the compound from Example 36A and 184 mg (0.546 mmol) of the compound from Example 10A.

¹H-NMR (400 MHz, CDCl₃, δ/ppm): 8.31 (d, 1H), 8.03 (d, 2H), 7.50 (dd, 1H), 7.31 (d, 1H), 6.96 (d, 2H), 6.80 (s, 1H), 5.42 (s, 2H), 3.31-3.28 (m, 4H), 2.30 (s, 3H), 1.73-1.68 (m, 4H), 1.66-1.60 (m, 2H).

LC/MS (method M, ESIpos): Rₜ=1.33 min, m/z=435/437 [M+H]⁺.

### Example 106A

### 2-Chloro-5-[(5-methyl-3-{3-[4-(methylsulphonyl)phenyl]-1,2,4-oxadiazol-5-yl}-1H-pyrazol-1-yl)-methyl]pyridine

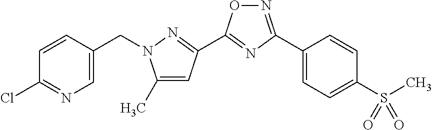

Analogously to the process described in Example 38A, 140 mg (66% of theory) of the title compound were obtained from 125 mg (0.497 mmol) of the compound from Example 36A and 117 mg (0.546 mmol) of the compound from Example 14A.

¹H-NMR (400 MHz, CDCl₃, δ/ppm): 8.42 (d, 2H), 8.31 (d, 1H), 8.08 (d, 2H), 7.52 (dd, 1H), 7.32 (d, 1H), 6.85 (s, 1H), 5.44 (s, 2H), 3.11 (s, 3H), 2.33 (s, 3H).

LC/MS (method F, ESIpos): Rₜ=1.13 min, m/z=430/432 [M+H]⁺.

### Example 107A

### 1-[4-(5-{1-[(6-Chloropyridin-3-yl)methyl]-5-methyl-1H-pyrazol-3-yl}-1,2,4-oxadiazol-3-yl)-phenyl]cyclobutanol

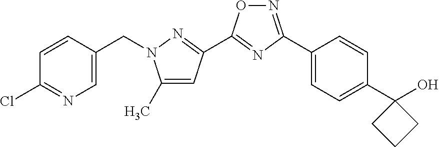

228 mg (1.19 mmol) of EDC and 182 mg (1.19 mmol) of HOBt were added to a solution of 250 mg (0.993 mmol) of the compound from Example 36A in 5 ml of anhydrous DMF at RT. After 30 min, 225 mg (1.09 mmol) of the compound from Example 92A, dissolved in 3 ml of DMF, were added. The mixture was then stirred initially at RT for 15 h and then at 120° C. in a microwave oven for 45 min (CEM Discover, initial irradiation power 250 W). After cooling to RT, the reaction mixture was purified directly by preparative HPLC (method K). 135 mg (32% of theory) of the title compound were obtained.

¹H-NMR (400 MHz, CDCl₃, δ/ppm): 8.32 (d, 1H), 8.20 (d, 2H), 7.63 (d, 2H), 7.51 (dd, 1H), 7.32 (d, 1H), 6.83 (s, 1H), 5.43 (s, 2H), 2.64-2.58 (m, 2H), 2.45-2.38 (m, 2H), 2.13-2.04 (m, 2H), 1.82-1.72 (m, 1H).

LC/MS (method M, ESIpos): Rₜ=1.11 min, m/z=422/424 [M+H]⁺.

### Example 108A

### 2-Chloro-5-{[5-methyl-3-(3-{4-[(trifluoromethyl)sulphonyl]phenyl}-1,2,4-oxadiazol-5-yl)-1H-pyrazol-1-yl]methyl}pyridine 1-oxide

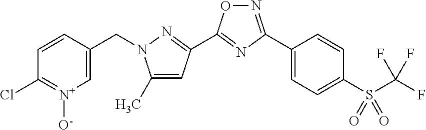

500 mg (1.03 mmol) of the compound from Example 40A were initially charged in 10 ml of dichloromethane. 951 mg (4.13 mmol, purity 75%) of 3-chloroperbenzoic acid were added, and the mixture was stirred at RT for 42 h. The mixture was then diluted with 50 ml of dichloromethane, and the solution was washed successively with in each case 50 ml of 1 N aqueous sodium hydroxide solution, water and saturated sodium chloride solution. The organic phase was then dried over magnesium sulphate, filtered and concentrated. The residue was triturated with a 1:1 mixture of pentane and tert-butyl methyl ether and then filtered. 463 mg (82% of theory, purity 92%) of the title compound were obtained.

LC/MS (method M, ESIpos): Rₜ=1.06 min, m/z=500/502 [M+H]⁺.

### Example 109A

### 2-Chloro-5-[(5-methyl-3-{3-[4-(pentafluoro-λ6-sulphanyl)phenyl]-1,2,4-oxadiazol-5-yl}-1H-pyrazol-1-yl)methyl]pyridine

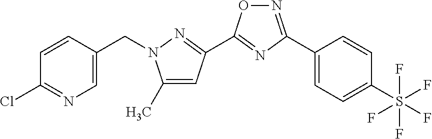

Analogously to the process described in Example 38A, 300 mg (1.19 mmol) of the compound from Example 36A and 313 mg (1.192 mmol) of the compound from Example 11A were reacted to give 310 mg (54% of theory) of the title compound.

¹H-NMR (400 MHz, CDCl₃, δ/ppm): 8.33-8.28 (m, 3H), 7.89 (d, 2H), 7.52 (dd, 1H), 7.32 (d, 1H), 6.84 (s, 1H), 5.44 (s, 2H), 2.32 (s, 3H).

LC/MS (method F, ESIpos): Rₜ=1.50 min, m/z=478/480 [M+H]⁺.

### Example 110A

### 2-Chloro-5-[(5-methyl-3-{3-[4-(1H-pyrrol-1-ylmethyl)phenyl]-1,2,4-oxadiazol-5-yl}-1H-pyrazol-1-yl)methyl]pyridine

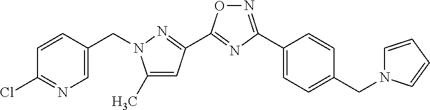

Analogously to the process described in Example 38A, 200 mg (0.795 mmol) of the compound from Example 36A and 171 mg (0.795 mmol) of the compound from Example 95A were reacted to give 157 mg (46% of theory) of the title compound.

¹H-NMR (400 MHz, CDCl₃, δ/ppm): 8.31 (d, 1H), 8.13 (d, 2H), 7.50 (dd, 1H), 7.31 (d, 1H), 7.22 (d, 2H), 6.82 (s, 1H), 6.72 (s, 2H), 6.22 (s, 2H), 5.42 (s, 2H), 5.13 (s, 2H), 2.31 (s, 3H).

LC/MS (method M, ESIpos): Rₜ=1.24 min, m/z=431/433 [M+H]⁺.

### Example 111A

### N-[4-(5-{1-[(6-Chloropyridin-3-yl)methyl]-5-methyl-1H-pyrazol-3-yl}-1,2,4-oxadiazol-3-yl)-benzyl]-N-isopropylpropane-2-amine

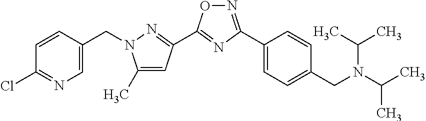

Analogously to the process described in Example 43A, 679 mg (2.00 mmol) of the compound from Example 102A and 421 mg (2.60 mmol) of 2-chloro-5-(chloromethyl)pyridine were reacted to give 387 mg (40% of theory, purity 96%) of the title compound. In this case, 400 mg (3.50 mmol) of potassium tert-butoxide were employed, and the reaction mixture was heated under reflux for 24 h.

¹H-NMR (400 MHz, CDCl₃, δ/ppm): 8.31 (d, 1H), 8.23 (d, 2H), 7.76 (d, 2H), 7.53-7.49 (dd, 1H), 7.31 (d, 1H), 6.82 (s, 1H), 5.42 (s, 2H), 4.30 (s, 2H), 3.85-3.76 (m, 2H), 2.32 (s, 3H), 1.44 (d, 12H).

LC/MS (method F, ESIpos): Rₜ=0.93 min, m/z=465/467 [M+H]⁺.

### Example 112A

### N-[4-(5-{1-[(6-Chloropyridin-3-yl)methyl]-5-methyl-1H-pyrrol-3-yl}-1,2,4-oxadiazol-3-yl)-benzyl]-N-isopropylpropane-2-amine

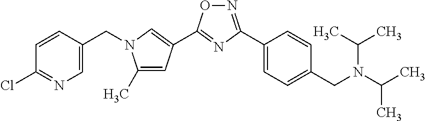

Analogously to the process described in Example 71A, 200 mg (0.798 mmol) of the compound from Example 37A and 199 mg (0.798 mmol) of the compound from Example 96A were reacted to give 80 mg (22% of theory) of the title compound.

¹H-NMR (400 MHz, CDCl₃, δ/ppm): 8.25 (d, 1H), 8.02 (d, 2H), 7.52-7.47 (m, 3H), 7.33-7.27 (m, 2H), 6.60 (s, 1H), 5.10 (s, 2H), 3.70 (s, 2H), 3.08-2.98 (m, 2H), 2.20 (s, 3H), 1.02 (d, 12H).

LC/MS (method F, ESIpos): Rₜ=1.03 min, m/z=464/466 [M+H]⁺.

### Example 113A

### 2-Chloro-5-[(2-methyl-4-{3-[4-(1H-pyrrol-1-ylmethyl)phenyl]-1,2,4-oxadiazol-5-yl}-1H-pyrrol-1-yl)methyl]pyridine

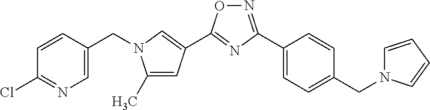

Analogously to the process described in Example 71A, 200 mg (0.798 mmol) of the compound from Example 37A and 172 mg (0.798 mmol) of the compound from Example 95A were reacted to give 57 mg (16% of theory) of the title compound.

¹H-NMR (400 MHz, CDCl₃, δ/ppm): 8.25 (d, 1H), 8.08 (d, 2H), 7.46 (s, 1H), 7.32-7.24 (m, 2H), 7.21 (d, 2H), 6.72-6.70 (t, 2H), 6.59 (s, 1H), 6.22-6.20 (t, 2H), 5.13 (s, 2H), 5.11 (s, 2H), 2.20 (s, 3H).

LC/MS (method D, ESIpos): Rₜ=2.70 min, m/z=430/432 [M+H]⁺.

### Example 114A

### 2-Chloro-5-[(4-{3-[4-(2-fluoropropan-2-yl)phenyl]-1,2,4-oxadiazol-5-yl}-2-methyl-1H-pyrrol-1-yl)methyl]pyridine

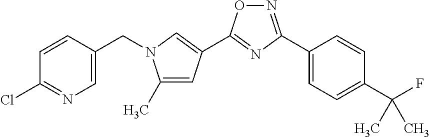

Analogously to the process described in Example 71A, 200 mg (0.798 mmol) of the compound from Example 37A and 157 mg (0.798 mmol) of the compound from Example 2A were reacted to give 78 mg (24% of theory) of the title compound.

¹H-NMR (400 MHz, CDCl₃, δ/ppm): 8.25 (d, 1H), 8.11 (d, 2H), 7.52-7.47 (m, 3H), 7.33-7.28 (m, 2H), 6.60 (s, 1H), 5.11 (s, 2H), 2.20 (s, 3H), 1.72 (s, 3H), 1.70 (s, 3H).

LC/MS (method F, ESIpos): Rₜ=1.50 min, m/z=411/413 [M+H]⁺.

### Example 115A

### 2-Chloro-5-[(2-methyl-4-{3-[4-(trifluoromethyl)phenyl]-1,2,4-oxadiazol-5-yl}-1H-pyrrol-1-yl)-methyl]pyridine

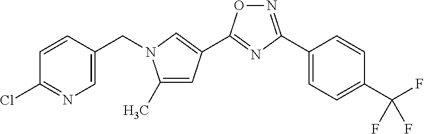

Analogously to the process described in Example 71A, 200 mg (0.798 mmol) of the compound from Example 37A and 163 mg (0.798 mmol) of N′-hydroxy-4-(trifluoromethyl)-benzenecarboximidamide were reacted to give 102 mg (30% of theory) of the title compound.

¹H-NMR (400 MHz, CDCl₃, δ/ppm): 8.28-8.23 (m, 3H), 7.75 (d, 2H), 7.49 (s, 1H), 7.33-7.28 (m, 2H), 6.61 (s, 1H), 5.11 (s, 2H), 2.21 (s, 3H).

LC/MS (method F, ESIpos): Rₜ=1.55 min, m/z=419/421 [M+H]⁺.

### Example 116A

### 2-Chloro-5-[(2-methyl-4-{3-[4-(trimethylsilyl)phenyl]-1,2,4-oxadiazol-5-yl}-1H-pyrrol-1-yl)-methyl]pyridine

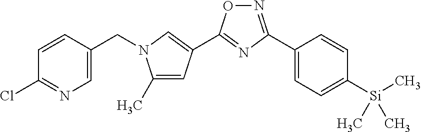

Analogously to the process described in Example 71A, 200 mg (0.798 mmol) of the compound from Example 37A and 166 mg (0.798 mmol) of the compound from Example 72A were reacted to give 83 mg (25% of theory) of the title compound.

¹H-NMR (400 MHz, CDCl₃, δ/ppm): 8.25 (s, 1H), 8.08 (d, 2H), 7.63 (d, 2H), 7.48 (d, 1H), 7.35-7.27 (m, 2H), 6.60 (s, 1H), 5.11 (s, 2H), 2.20 (s, 3H), 0.30 (s, 9H).

LC/MS (method F, ESIpos): Rₜ=1.71 min, m/z=423/425 [M+H]⁺.

### Example 117A

### 2-Chloro-5-[(2-methyl-4-{3-[4-(pentafluoro-λ⁶-sulphanyl)phenyl]-1,2,4-oxadiazol-5-yl}-1H-pyrrol-1-yl)methyl]pyridine

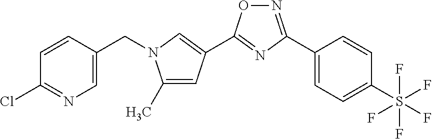

Analogously to the process described in Example 71A, 200 mg (0.798 mmol) of the compound from Example 37A and 209 mg (0.798 mmol) of the compound from Example 11A were reacted to give 178 mg (47% of theory) of the title compound.

¹H-NMR (400 MHz, CDCl₃, δ/ppm): 8.25-8.20 (m, 3H), 7.87 (d, 2H), 7.49 (d, 1H), 7.33 (d, 1H), 7.29 (dd, 1H), 6.60 (d, 1H), 5.12 (s, 2H), 2.21 (s, 3H).

LC/MS (method M, ESIpos): Rₜ=1.38 min, m/z=477/479 [M+H]⁺.

### Working Examples

### Example 1

### 6-[(5-Methyl-3-{3-[4-(trifluoromethoxy)phenyl]-1,2,4-oxadiazol-5-yl}-1H-pyrazol-1-yl)methyl]-N-[2-(pyrrolidin-1-yl)ethyl]pyridine-2-amine

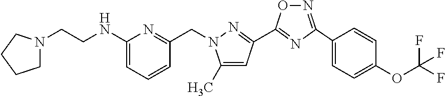

A solution of 150 mg (0.312 mmol) of the compound from Example 42A and 792 μl (6.25 mmol) of N-(2-aminoethyl)pyrrolidine in 1.5 ml of diethylene glycol dimethyl ether was heated at 180° C. in a microwave oven (CEM Discover) at an initial irradiation power of 250 W for 3 h. After cooling, the mixture was diluted with about 1 ml of acetonitrile and 1 ml of water and then purified by preparative HPLC (method L). The product fractions were concentrated, made basic with aqueous sodium bicarbonate solution and extracted with ethyl acetate. The organic extract was dried over anhydrous sodium sulphate and filtered, and the solvent was removed on a rotary evaporator. 66 mg (40% of theory) of the title compound were obtained.

¹H-NMR (400 MHz, CDCl₃, δ/ppm): 8.24 (d, 2H), 7.35-7.28 (m, 3H), 6.80 (s, 1H), 6.29 (d, 1H), 6.18 (d, 1H), 5.35 (s, 2H), 5.05 (s, broad, 1H), 3.35 (dd, 2H), 2.70 (t, 2H), 2.56 (s, 4H), 2.39 (s, 3H), 1.82-1.75 (m, 4H).

HPLC (method B): Rₜ=4.36 min.

LC/MS (method E, ESIpos): Rₜ=1.56 min, m/z=514 [M+H]⁺.

The compounds in the following table were obtained in an analogous manner, as described for Example 1, from the compound from Example 42A and the corresponding amine components.

| | | HPLC: | MS: m/z | LC/MS |
|---|---|---|---|---|
| Example | Structure | Rₜ [min] | [M + H]⁺ | method |
| 2 | 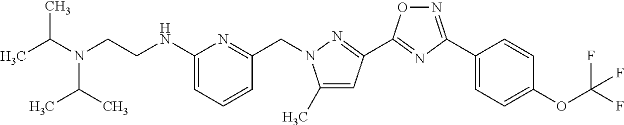 | 1.61 | 544 | E |
| | ¹H-NMR (400 MHz, CDCl₃, δ/ppm): 8.24 (d, 2H), 7.35-7.28 (m, 3H), 6.80 (s, 1H), 6.28 | | | |
| | (d, 1H), 6.18 (d, 1H), 5.35 (s, 2H), 5.05 (t, 1H), 3.25-3.19 (m, 2H), 3.08-2.99 (m, 2H), | | | |
| | 2.70-2.65 (t, 2H), 2.39 (s, 3H), 1.01 (s, 12H). | | | |
| 3 | 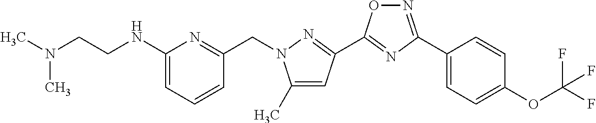 | 1.51 | 488 | E |

¹H-NMR (400 MHz, CDCl₃, δ/ppm): 8.24 (d, 2H), 7.35-7.28 (m, 3H), 6.80 (s, 1H), 6.29
(d, 1H), 6.19 (d, 1H), 5.35 (s, 2H), 5.02 (t, 1H), 3.33-3.28 (quart, 2H), 2.53-2.48 (t, 2H),
2.39 (s, 3H), 2.24 (s, 6H).

### Example 4

### 1-[2-({6-[(5-Methyl-3-{3-[4-(trifluoromethoxy)phenyl]-1,2,4-oxadiazol-5-yl}-1H-pyrazol-1-yl)-methyl]pyridin-3-yl}amino)ethyl]piperidin-4-ol

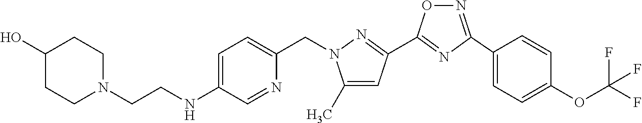

Under inert conditions, a mixture of 100 mg (0.190 mmol) of the compound from Example 43A, 28 mg (0.190 mmol) of 1-(2-aminoethyl)piperidin-4-ol [K. Pors et al., J. Med. Chem. 2005, 48 (21), 6690-6695], 1.3 mg (0.006 mmol) of palladium(II) acetate, 3.5 mg (0.006 mmol) of racemic 2,2′-bis(diphenylphosphino)-1,1′-binaphthyl, 309 mg (0.948 mmol) caesium carbonate and 1.6 μl (0.011 mmol) of triethylamine in 4 ml of toluene was heated at reflux. After 16 h, the mixture was allowed to cool to RT, and the same amounts of 1-(2-aminoethyl)piperidin-4-ol, palladium(II) acetate, 2,2′-bis(diphenylphosphino)-1,1′-binaphthyl, caesium carbonate and triethylamine were added again. The mixture was then once more heated under reflux for 16 h. After cooling, approx. 50 ml of water were added and the mixture was extracted three times with approx. 50 ml of ethyl acetate each time. The combined organic extracts were washed with saturated sodium chloride solution and dried over anhydrous magnesium sulphate. After filtration, the solvent was removed on a rotary evaporator. The crude product was purified by preparative HPLC (method K). The product fractions were combined, dissolved in methanol and freed via a bicarbonate cartridge (Polymerlabs, Stratospheres SPE, PL-HCO₃ MP SPE, capacity 0.9 mmol) from the formic acid originating from the HPLC mobile phase. After the solvent had been removed on a rotary evaporator, 30 mg (30% of theory) of the title compound were obtained.

¹H-NMR (400 MHz, CDCl₃, δ/ppm): 8.25 (d, 2H), 7.97 (d, 1H), 7.33 (d, 2H), 6.94 (d, 1H), 6.82 (dd, 1H), 6.79 (s, 1H), 5.44 (s, 2H), 4.47 (t, 1H), 3.77-3.70 (m, 1H), 3.12 (dt, 2H), 2.80-2.71 (m, 2H), 2.61 (t, 2H), 2.35 (s, 3H), 2.21-2.13 (m, 2H), 1.93-1.86 (m, 2H), 1.61-1.53 (m, 2H), 1.43 (d, 1H).

HPLC (method B): Rₜ=4.15 min.

MS (ESIpos): m/z=544 [M+H]⁺.

The compounds in the following table were prepared from the compound from Example 43A and the corresponding amine components analogously to the process described in Example 4.

| | | HPLC: | MS: m/z | LC/MS |
|---|---|---|---|---|
| Example | Structure | Rₜ [min] | [M + H]⁺ | method |
| 5 | 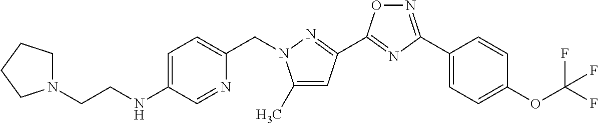 | 1.76 | 514 | C |
| | ¹H-NMR (400 MHz, CDCl₃, δ/ppm): 8.25 (d, 2H), 7.97 (d, 1H), 7.33 (d, 2H), 6.93 (d, | | | |
| | 1H), 6.83 (dd, 1H), 6.79 (s, 1H), 5.45 (s, 2H), 4.43 (t, 1H), 3.14 (dt, 2H), 2.72 (t, 2H), | | | |
| | 2.53-2.50 (m, 4H), 2.35 (s, 3H), 1.80-1.77 (m, 4H). | | | |
| 6 | 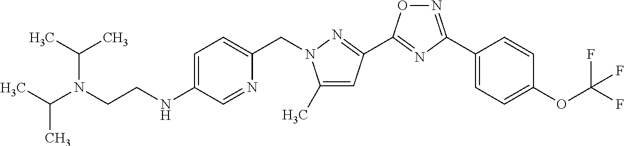 | 1.13 | 544 | F |
| | ¹H-NMR (400 MHz, CDCl₃, δ/ppm): 8.25 (d, 2H), 7.94 (d, 1H), 7.33 (d, 2H), 6.92 (d, | | | |
| | 1H), 6.80 (dd, 1H), 6.79 (s, 1H), 5.44 (s, 2H), 4.54 (t, 1H), 3.05-2.99 (m, 4H), 2.72 (t, | | | |
| | 2H), 2.35 (s, 3H), 1.01 (d, 12H). | | | |
| 7 | 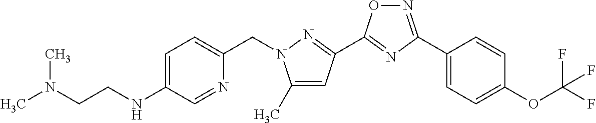 | 4.17 | 488 | B |

¹H-NMR (400 MHz, CDCl₃, δ/ppm): 8.24 (d, 2H), 7.98 (d, 1H), 7.33 (d, 2H), 6.93 (d,
1H), 6.82 (dd, 1H), 6.79 (s, 1H), 5.45 (s, 2H), 4.43 (t, 1H), 3.10 (dt, 2H), 2.55 (t, 2H),
2.35 (s, 3H), 2.23 (s, 6H).

### Example 8

### 2-Methoxy-N-({5-[(5-methyl-3-{3-[4-(trifluoromethoxy)phenyl]-1,2,4-oxadiazol-5-yl}-1H-pyrazol-1-yl)methyl]pyridin-2-yl}methyl)ethanamine

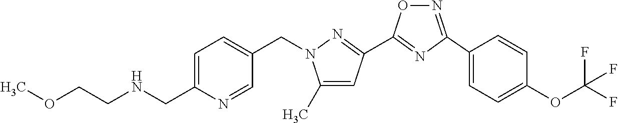

Initially 15 μl (0.171 mmol) of 2-methoxyethylamine and then 49 mg (0.228 mmol) of sodium triacetoxyborohydride were added to a solution of 70 mg (0.163 mmol) of the compound from Example 54A in 2.5 ml of 1,2-dichloroethane. After the reaction mixture had been stirred at RT for 16 h, approx. 20 ml of water were added and the mixture was extracted three times with approx. 20 ml of ethyl acetate each time. The organic extract was washed with saturated sodium chloride solution, dried over anhydrous magnesium sulphate and filtered. After removal of the solvent on a rotary evaporator, the crude product obtained was purified by preparative HPLC (method K). The product fractions were concentrated and then dissolved in methanol, and the adhering formic acid was removed via a bicarbonate cartridge (Polymerlabs, Stratospheres SPE, PL-HCO₃ MP SPE, capacity 0.9 mmol). 36 mg (41% of theory) of the title compound were obtained.

¹H-NMR (400 MHz, CDCl₃, δ/ppm): 8.47 (d, 1H), 8.25 (d, 2H), 7.49 (dd, 1H), 7.33 (d, 2H), 7.31 (d, 1H), 6.81 (s, 1H), 5.45 (s, 2H), 3.93 (d, 2H), 3.52 (t, 2H), 3.35 (s, 3H), 2.82 (dt, 2H), 2.31 (s, 3H), 2.09-2.00 (m, 1H).

HPLC (method B): Rₜ=4.46 min.

MS (ESIpos): m/z=489 [M+H]⁺.

LC/MS (method E, ESIpos): Rₜ=1.43 min, m/z=489 [M+H]⁺.

The compounds in the following table were prepared analogously to the process described in Example 8 from the compound from Example 54A and the corresponding amines

| | | HPLC: | MS: m/z | LC/MS |
|---|---|---|---|---|
| Example | Structure | Rₜ [min] | [M + H]⁺ | method |
| 9 | 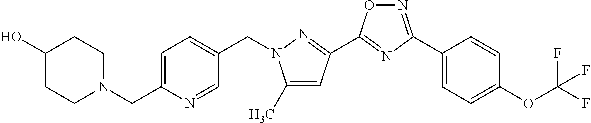 | 1.06 | 515 | F |
| | ¹H-NMR (400 MHz, CDCl₃, δ/ppm): 8.48 (d, 1H), 8.25 (d, 2H), 7.50 (dd, 1H), 7.40 (d, | | | |
| | 1H), 7.33 (d, 2H), 6.81 (s, 1H), 5.45 (s, 2H), 3.75-3.68 (m, 1H), 3.63 (s, 2H), 2.77 (dt, | | | |
| | 2H), 2.32 (s, 3H), 2.23 (dt, 2H), 1.88 (dt, 2H), 1.62 (dt, 2H), 1.37 (d, 1H). | | | |
| 10 | 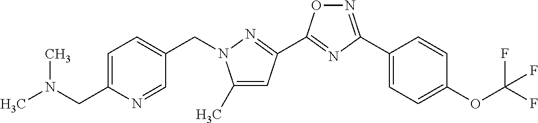 | 1.09 | 459 | F |
| | ¹H-NMR (400 MHz, CDCl₃, δ/ppm): 8.47 (d, 1H), 8.25 (d, 2H), 7.50 (dd, 1H), 7.37 (d, | | | |
| | 1H), 7.33 (d, 2H), 6.82 (s, 1H), 5.45 (s, 2H), 3.67 (s, 2H), 2.32 (s, 3H), 2.28 (s, 6H). | | | |
| 11 | 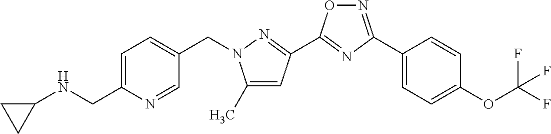 | 1.68 | 471 | C |
| | ¹H-NMR (400 MHz, CDCl₃, δ/ppm): 8.48 (d, 1H), 8.25 (d, 2H), 7.48 (dd, 1H), 7.33 (d, | | | |
| | 2H), 7.27 (d, 1H), 6.81 (s, 1H), 5.45 (s, 2H), 3.96 (s, 2H), 2.31 (s, 3H), 2.18-2.13 (m, | | | |
| | 1H), 0.47-0.38 (m, 4H). | | | |
| 12 | 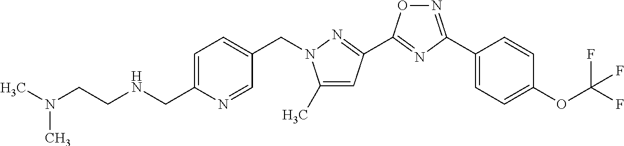 | 1.21 | 502 | E |

¹H-NMR (400 MHz, CDCl₃, δ/ppm): 8.47 (d, 1H), 8.25 (d, 2H), 7.49 (dd, 1H), 7.34-7.31
(m, 3H), 6.81 (s, 1H), 5.44 (s, 2H), 3.92 (d, 2H), 2.72 (dt, 2H), 2.43 (t, 2H), 2.31
(s, 3H), 2.20 (s, 6H).

### Example 13

### N-[2-(4-Hydroxypiperidin-1-yl)ethyl]-5-[(5-methyl-3-{3-[4-(trifluoromethoxy)phenyl]-1,2,4-oxadiazol-5-yl}-1H-pyrazol-1-yl)methyl]pyridine-2-carboxamide

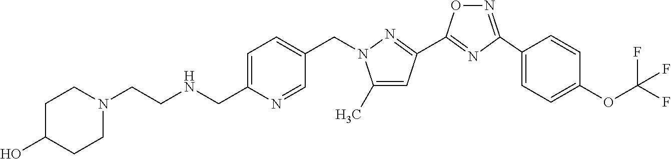

Under inert conditions, a solution of 85 mg (0.191 mmol) of the compound from Example 55A was initially charged in 3 ml of anhydrous dichloromethane, and 83 μl (0.954 mmol) of oxalyl chloride and a small drop of DMF were added at RT. After 2 h, the reaction mixture was freed from all volatile components on a rotary evaporator, and the crude product was dried under high vacuum for 1 h. The residue was then dissolved in 2 ml of anhydrous THF and added dropwise to a solution of 41 mg (0.286 mmol) of 1-(2-aminoethyl)piperidin-4-ol [K. Pors et al., J. Med. Chem. 2005, 48 (21), 6690-6695] and 67 μl (0.382 mmol) of N,N-diisopropylethylamine in 1 ml of anhydrous THF. The reaction mixture was stirred at RT for 16 h. Approx. 1 ml of water was then added and the reaction mixture was separated directly by preparative HPLC (method K). The product fractions were concentrated and then once more dissolved in methanol, and the adhering formic acid was removed via a bicarbonate cartridge (Polymerlabs, Stratospheres SPE, PL-HCO₃ MP SPE, capacity 0.9 mmol). 87 mg (80% of theory) of the title compound were obtained.

¹H-NMR (400 MHz, CDCl₃, δ/ppm): 8.43 (d, 1H), 8.30 (t, 1H), 8.25 (d, 2H), 8.18 (d, 1H), 7.65 (dd, 1H), 7.33 (d, 2H), 6.85 (s, 1H), 5.53 (s, 2H), 3.76-3.69 (m, 1H), 3.54 (quart, 2H), 2.81 (dt, 2H), 2.58 (t, 2H), 2.33 (s, 3H), 2.21 (dt, 2H), 1.90 (dt, 2H), 1.60 (dt, 2H), 1.46 (broad, 1H).

HPLC (method B): Rₜ=4.36 min.

MS (ESIpos): m/z=572 [M+H]⁺.

LC/MS (method E, ESIpos): Rₜ=1.42 min, m/z=572 [M+H]⁺.

### Example 14

### 5-[(5-Methyl-3-{3-[4-(1,1,1-trifluoro-2-methylpropan-2-yl)phenyl]-1,2,4-oxadiazol-5-yl}-1H-pyrazol-1-yl)methyl]-N-[2-(pyrrolidin-1-yl)ethyl]pyridine-2-amine

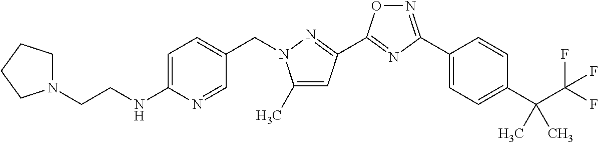

A solution of 120 mg (0.223 mmol) of the compound from Example 45A and 283 μl (2.23 mmol) of 2-(pyrrolidin-1-yl)ethanamine in 1 ml of diethylene glycol dimethyl ether was heated in a microwave oven (CEM Discover, initial irradiation power 250 W) at 180° C. Since, after 3 h, the reaction had not yet gone to completion, a further 283 μl (2.23 mmol) of 2-(pyrrolidin-1-yl)ethanamine were added and the reaction was continued under identical conditions. After 2 h, the reaction mixture was cooled to RT and purified directly by preparative HPLC (method L). The product fractions were combined and concentrated on a rotary evaporator, so that only water remained as solvent. Solid sodium bicarbonate was added, and the mixture was stirred for a number of minutes. The solid product was then filtered off with suction, washed with water and dried under high vacuum. 64 mg (53% of theory) of the title compound were obtained.

¹H-NMR (400 MHz, CDCl₃, δ/ppm): 8.20 (d, 2H), 8.02 (s, 1H), 7.62 (d, 2H), 7.33 (d, 1H), 6.77 (s, 1H), 6.37 (d, 1H), 5.28 (s, 2H), 5.19 (s, broad, 1H), 3.40-3.33 (m, 2H), 2.75-2.69 (m, 2H), 2.59-2.52 (m, 4H), 2.31 (s, 3H), 1.80-1.74 (m, 4H), 1.65-1.60 (m, 6H).

LC/MS (method C, ESIpos): Rₜ=1.73 min, m/z=540 [M+H]⁺.

The compounds in the following table were prepared by the process described in Example 14 from the corresponding chloropyridine derivatives and the corresponding amine components. Here, in each case 20 equivalents of the amine compound were used. The amine compounds were either commercially available, or they were prepared according to the following literature procedure: N-methyl-2-(morpholin-4-yl)ethanamine [T. Hayashi et al., Tetrahedron 1992, 48 (11), 1999-2012], 1-(2-aminoethyl)piperidin-4-ol [K. Pors et al., J. Med. Chem. 2005, 48 (21), 6690-6695]. Depending on the batch size, the product was, after purification by preparative HPLC, either stirred with aqueous sodium bicarbonate solution or dissolved in methanol and passed through a bi-carbonate cartridge (Polymerlabs, Stratospheres SPE, PL-HCO₃ MP SPE, capacity 0.9 mmol).

| | | HPLC: | MS: m/z | LC/MS |
|---|---|---|---|---|
| Example | Structure | Rₜ [min] | [M + H]⁺ | method |
| 15 | 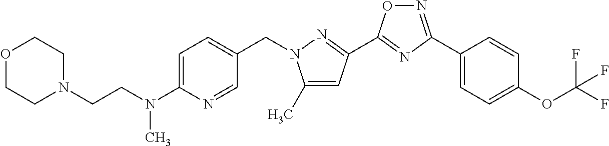 | 1.14 | 544 | F |
| | ¹H-NMR (400 MHz, CDCl₃, δ/ppm): 8.25 (d, 2H), 8.08 (s, 1H), 7.39 (d, 1H), 7.33 (d, | | | |
| | 2H), 6.76 (s, 1H), 6.45 (d, 1H), 5.30 (s, 2H), 3.71-3.63 (m, 6H), 3.05 (s, 3H), 2.58-2.46 | | | |
| | (m, 6H), 2.32 (s, 3H). | | | |
| 16 | 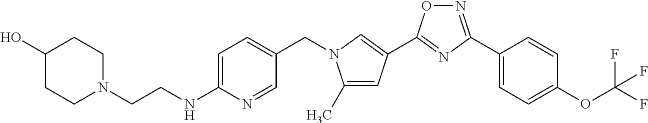 | 1.72 | 543 | C |
| | ¹H-NMR (400 MHz, CDCl₃, δ/ppm): 8.16 (d, 2H), 7.98 (d, 1H), 7.41 (d, 1H), 7.31 (d, | | | |
| | 2H), 7.18 (dd, 1H), 6.52 (d, 1H), 6.40 (d, 1H), 5.21 (t, 1H), 4.92 (s, 2H), 3.75-3.69 (m, | | | |
| | 1H), 3.33 (quart, 2H), 2.81-2.75 (m, 2H), 2.60 (t, 2H), 2.26 (s, 3H), 2.20 (dt, 2H), 1.93-1.87 | | | |
| | (m, 2H), 1.64-1.54 (m, 3H). | | | |
| 17 | 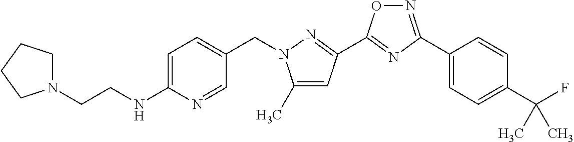 | 1.35 | 489 | E |
| | ¹H-NMR (400 MHz, DMSO-d₆, δ/ppm): 8.08 (d, 2H), 7.98 (d, 1H), 7.62 (d, 2H), 7.29 | | | |
| | (dd, 1H), 6.38 (s, 1H), 6.51 (t, 1H), 6.47 (d, 1H), 5.27 (s, 2H), 3.35-3.29 (m, 2H), 2.58-2.40 | | | |
| | (m, 6H), 2.38 (s, 3H), 1.72 (s, 3H), 1.70-1.65 (m, 7H). | | | |
| 18 | 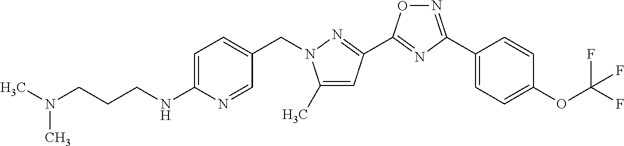 | 1.23 | 502 | E |
| | ¹H-NMR (400 MHz, DMSO-d₆, δ/ppm): 8.20 (d, 2H), 7.97 (d, 1H), 7.59 (d, 2H), 7.28 | | | |
| | (dd, 1H), 6.88 (s, 1H), 6.62 (t, 1H), 6.42 (d, 1H), 5.27 (s, 2H), 3.25-3.18 (m, 2H), 2.38 | | | |
| | (s, 3H), 2.24 (t, 2H), 2.10 (s, 6H), 1.65-1.58 (m, 2H). | | | |
| 19 | 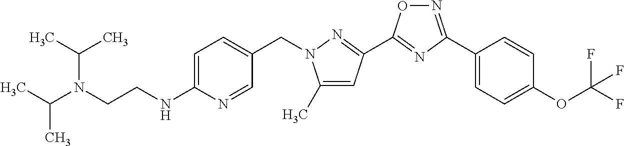 | 1.60 | 543 | E |
| | ¹H-NMR (400 MHz, CDCl₃, δ/ppm): 8.25 (d, 2H), 8.02 (s, 1H), 7.37-7.31 (m, 3H), 6.76 | | | |
| | (s, 1H), 6.32 (d, 1H), 5.29 (s, 2H), 5.21 (s, broad, 1H), 3.25-3.18 (m, 2H), 3.09-2.98 (m, | | | |
| | 2H), 2.72-2.67 (m, 2H), 2.31 (s, 3H), 1.01 (d, 12H). | | | |
| 20 | 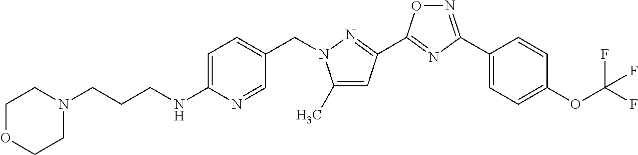 | 1.18 | 544 | E |
| | ¹H-NMR (400 MHz, CDCl₃, δ/ppm): 8.25 (d, 2H), 8.01 (s, 1H), 7.36-7.31 (m, 3H), 6.76 | | | |
| | (s, 1H), 6.32 (d, 1H), 5.42 (t, 1H), 5.29 (s, 2H), 3.72 (t, 4H), 3.39-3.32 (m, 2H), 2.50-2.40 | | | |
| | (m, 6H), 2.31 (s, 3H), 1.81-1.75 (m, 2H). | | | |
| 21 | 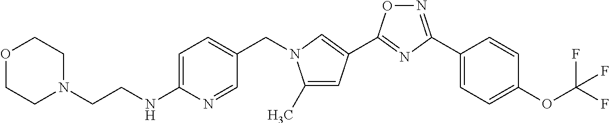 | 1.49 | 529 | E |
| | ¹H-NMR (400 MHz, CDCl₃, δ/ppm): 8.16 (d, 2H), 7.98 (d, 1H), 7.41 (d, 1H), 7.31 (d, | | | |
| | 2H), 7.18 (dd, 1H), 6.52 (d, 1H), 6.40 (d, 1H), 5.18 (t, 1H), 4.92 (s, 2H), 3.73-3.70 (m, | | | |
| | 4H), 3.36 (quart, 2H), 2.62 (t, 2H), 2.50-2.47 (m, 4H), 2.23 (s, 3H). | | | |
| 22 | 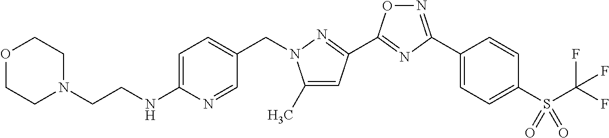 | 1.45 | 578 | E |
| | ¹H-NMR (400 MHz, DMSO-d₆, δ/ppm): 8.49 (d, 2H), 8.36 (d, 2H), 7.98 (s, 1H), 7.30 | | | |
| | (d, 1H), 6.90 (s, 1H), 6.52-6.45 (m, 2H), 5.29 (s, 2H), 3.60-3.52 (m, 4H), 3.38-3.28 (m, | | | |
| | 2H), 2.46-2.33 (m, 9H). | | | |
| 23 | 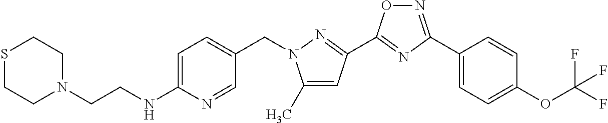 | 1.51 | 546 | E |
| | ¹H-NMR (400 MHz, CDCl₃, δ/ppm): 8.25 (d, 2H), 8.02 (s, 1H), 7.38-7.31 (m, 3H), 6.76 | | | |
| | (s, 1H), 6.38 (d, 1H), 5.29 (s, 2H), 5.11 (s, broad, 1H), 3.34-3.28 (m, 2H), 2.77-2.70 (m, | | | |
| | 4H), 2.70-2.65 (m, 4H), 2.65-2.60 (m, 2H), 2.32 (s, 3H). | | | |
| 24 | 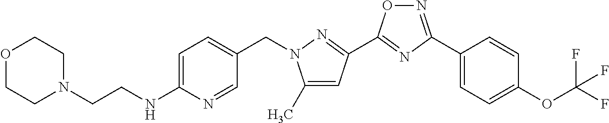 | 1.47 | 530 | E |
| | ¹H-NMR (400 MHz, CDCl₃, δ/ppm): 8.25 (d, 2H), 8.02 (s, 1H), 7.38-7.30 (m, 3H), 6.76 | | | |
| | (s, 1H), 6.38 (d, 1H), 5.30 (s, 2H), 5.19 (s, broad, 1H), 3.71 (t, 4H), 3.38-3.31 (m, 2H), | | | |
| | 2.65-2.58 (m, 2H), 2.48 (m, 4H), 2.31 (s, 3H). | | | |
| 25 | 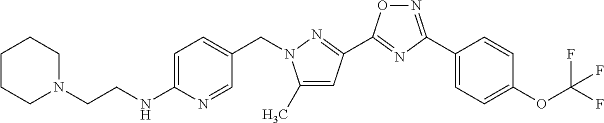 | 1.47 | 528 | E |
| | ¹H-NMR (400 MHz, CDCl₃, δ/ppm): 8.26 (d, 2H), 8.02 (s, 1H), 7.36-7.30 (m, 3H), 6.77 | | | |
| | (s, 1H), 6.38 (d, 1H), 5.29 (s, 2H), 3.35-3.28 (m, 2H), 2.59-2.52 (m, 2H), 2.45-2.30 (m, | | | |
| | broad, 4H), 2.32 (s, 3H), 1.62-1.38 (m, 6H). | | | |
| 26 | 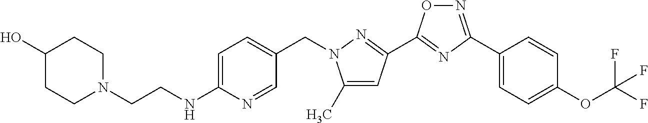 | 1.05 | 544 | F |
| | ¹H-NMR (400 MHz, CDCl₃, δ/ppm): 8.25 (d, 2H), 8.02 (d, 1H), 7.38-7.31 (m, 3H), 6.77 | | | |
| | (s, 1H), 6.37 (d, 1H), 5.29 (s, 2H), 5.20 (s, broad, 1H), 3.75-3.67 (m, 1H), 3.32 (quart, | | | |
| | 2H), 2.80-2.72 (m, 2H), 2.61-2.56 (m, 2H), 2.31 (s, 3H), 2.22-2.13 (m, 2H), 1.92-1.85 | | | |
| | (m, 2H), 1.64-1.52 (m, 3H). | | | |
| 27 | 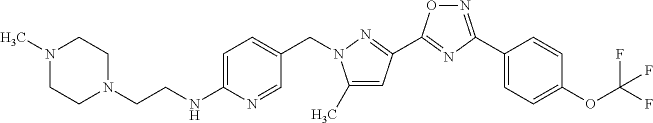 | 1.17 | 543 | E |
| | ¹H-NMR (400 MHz, CDCl₃, δ/ppm): 8.26 (d, 2H), 8.02 (s, 1H), 7.37-7.31 (m, 3H), 6.78 | | | |
| | (s, 1H), 6.37 (d, 1H), 5.30 (s, 2H), 5.17 (t, 1H), 3.35-3.29 (m, 2H), 2.62-2.58 (m, 2H), | | | |
| | 2.60-2.30 (m, 8H), 2.31 (s, 3H), 2.28 (s, 3H). | | | |
| 28 | 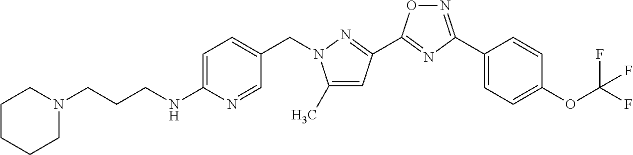 | 1.08 | 542 | F |
| | ¹H-NMR (400 MHz, DMSO-d₆, δ/ppm): 8.20 (d, 2H), 7.97 (d, 1H), 7.60 (d, 2H), 7.29 | | | |
| | (dd, 1H), 6.88 (s, 1H), 6.64 (t, 1H), 6.42 (d, 1H), 5.27 (s, 2H), 3.25-3.18 (m, 2H), 2.39 | | | |
| | (s, 3H), 2.39-2.20 (m, 6H), 1.69-1.60 (m, 2H), 1.52-1.42 (m, 4H), 1.42-1.32 (m, 2H). | | | |
| 29 | 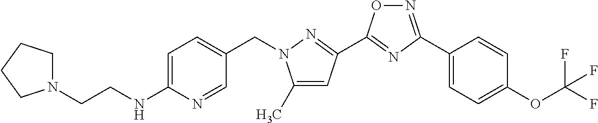 | 1.36 | 514 | E |
| | ¹H-NMR (400 MHz, DMSO-d₆, δ/ppm): 8.20 (d, 2H), 7.97 (d, 1H), 7.58 (d, 2H), 7.29 | | | |
| | (dd, 1H), 6.88 (s, 1H), 6.51 (t, 1H), 6.47 (d, 1H), 5.26 (s, 2H), 3.32 (quart, 2H), 2.53 (t, | | | |
| | 2H), 2.46-2.42 (m, 4H), 2.38 (s, 3H), 1.68-1.64 (m, 4H). | | | |
| 30 | 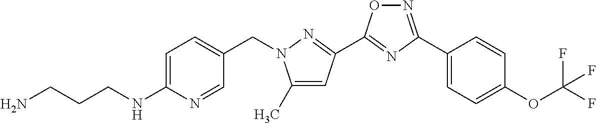 | 1.12 | 474 | E |
| | ¹H-NMR (400 MHz, CDCl₃, δ/ppm): 8.25 (d, 2H), 8.01 (s, 1H), 7.36-7.32 (m, 3H), 6.78 | | | |
| | (s, 1H), 6.35 (d, 1H), 5.29 (s, 2H), 5.01 (t, 1H), 3.41-3.35 (m, 2H), 2.84 (t, 2H), 2.32 (s, | | | |
| | 3H), 1.80-1.72 (m, 2H). | | | |
| 31 | 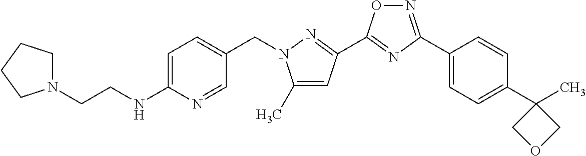 | 1.54 | 500 | D |
| | ¹H-NMR (400 MHz, DMSO-d₆, δ/ppm): 8.06 (d, 2H), 7.97 (d, 1H), 7.47 (d, 2H), 7.29 | | | |
| | (dd, 1H), 6.87 (s, 1H), 6.51 (t, 1H), 6.47 (d, 1H), 5.25 (s, 2H), 4.83 (d, 2H), 4.59 (d, | | | |
| | 2H), 3.31 (quart, 2H), 2.53 (t, 2H), 2.46-2.43 (m, 4H), 2.38 (s, 3H), 1.67 (s, 3H), 1.69-1.64 | | | |
| | (m, 4H). | | | |
| 32 | 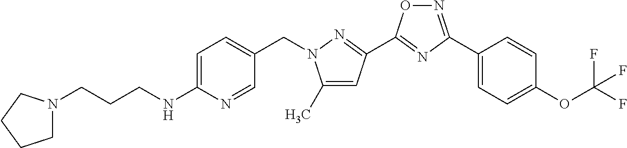 | 0.99 | 528 | F |
| | ¹H-NMR (400 MHz, CDCl₃, δ/ppm): 8.25 (d, 2H), 8.01 (s, 1H), 7.32-7.30 (m, 3H), 6.77 | | | |
| | (s, 1H), 6.32 (d, 1H), 5.43 (t, 1H), 5.27 (s, 2H), 3.36-3.32 (m, 2H), 2.58 (t, 2H), 2.52-2.45 | | | |
| | (m, 4H), 2.31 (s, 3H), 1.85-1.75 (m, 6H). | | | |
| 33 | 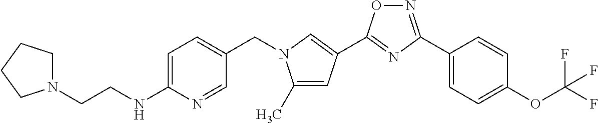 | 1.50 | 513 | E |
| | ¹H-NMR (400 MHz, CDCl₃, δ/ppm): 8.16 (d, 2H), 7.98 (d, 1H), 7.41 (d, 1H), 7.31 (d, | | | |
| | 2H), 7.17 (dd, 1H), 6.52 (d, 1H), 6.40 (d, 1H), 5.17 (t, 1H), 4.90 (s, 2H), 3.38 (quart, | | | |
| | 2H), 2.71 (t, 2H), 2.56-2.51 (m, 4H), 2.25 (s, 3H), 1.80-1.75 (m, 4H). | | | |
| 34 | 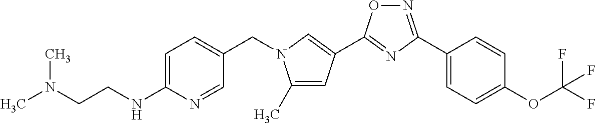 | 1.69 | 487 | C |
| | ¹H-NMR (400 MHz, CDCl₃, δ/ppm): 8.16 (d, 2H), 7.98 (d, 1H), 7.41 (d, 1H), 7.31 (d, | | | |
| | 2H), 7.16 (dd, 1H), 6.53 (d, 1H), 6.40 (d, 1H), 5.14 (t, 1H), 4.91 (s, 2H), 3.35 (quart, | | | |
| | 2H), 2.55 (t, 2H), 2.25 (s, 6H), 2.24 (s, 3H). | | | |
| 35 | 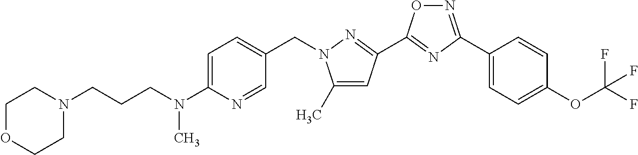 | 1.59 | 558 | C |
| | ¹H-NMR (400 MHz, CDCl₃, δ/ppm): 8.25 (d, 2H), 8.07 (d, 1H), 7.38 (dd, 1H), 7.34 (d, | | | |
| | 2H), 6.77 (s, 1H), 6.49 (d, 1H), 5.29 (s, 2H), 3.70 (t, 4H), 3.55 (t, 2H), 3.03 (s, 3H), | | | |
| | 2.45-2.32 (m, 9H), 1.81-1.71 (m, 2H). | | | |
| 36 | 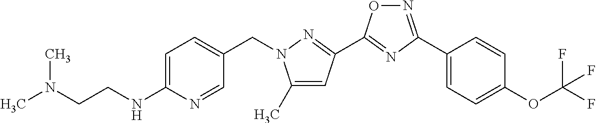 | 1.67 | 488 | C |
| | ¹H-NMR (400 MHz, CDCl₃, δ/ppm): 8.22 (d, 2H), 7.82-7.74 (m, 2H), 7.32 (d, 2H), 7.03 | | | |
| | (d, 1H), 6.82 (s, 1H), 5.80 (s, 2H), 3.98-3.92 (t, 2H), 3.40-3.35 (t, 2H), 2.90 (s, 6H), | | | |
| | 2.38 (s, 3H). | | | |
| 37 | 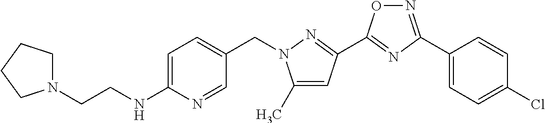 | 1.01 | 464 | F |

¹H-NMR (400 MHz, CDCl₃, δ/ppm): 8.14 (d, 2H), 8.01 (d, 1H), 7.47 (d, 2H), 7.33 (dd,
1H), 6.77 (s, 1H), 6.38 (d, 1H), 5.28 (s, 2H), 5.21 (t, 1H), 3.37 (quart, 2H), 2.71 (t, 2H),
2.57-2.52 (m, 4H), 2.31 (s, 3H), 1.81-1.74 (m, 4H).

### Example 38

### N-{5-[(5-Methyl-3-{3-[4-(trifluoromethoxy)phenyl]-1,2,4-oxadiazol-5-yl}-1H-pyrazol-1-yl)-methyl]pyridin-2-yl}ethane-1,2-diamine

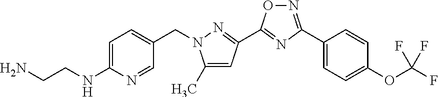

A mixture of 500 mg (1.15 mmol) of the compound from Example 46A and 7.7 ml (115 mmol) of 1,2-diaminoethane was heated at 120° C. for 15 h. After cooling to RT, approx. 50 ml of water were added and the mixture was extracted twice with approx. 50 ml of ethyl acetate each time. The solvent was removed from the combined organic extracts on a rotary evaporator and the residue was purified by preparative HPLC (method L). The product fractions were combined and concentrated on a rotary evaporator, so that only water remained as solvent. Solid sodium bicarbonate was added, and the mixture was stirred for a number of minutes. The precipitated product was filtered off with suction, washed with water and dried under high vacuum. 215 mg (41% of theory) of the title compound were obtained.

¹H-NMR (400 MHz, CDCl₃, δ/ppm): 8.25 (d, 2H), 8.02 (d, 1H), 7.35 (dd, 1H), 7.33 (d, 2H), 6.78 (s, 1H), 6.39 (d, 1H), 5.29 (s, 2H), 4.91 (t, 1H), 3.35 (quart, 2H), 2.93 (t, 2H), 2.32 (s, 3H), 1.47 (broad, 2H).

LC/MS (method E, ESIpos): Rₜ=1.24 min, m/z=460 [M+H]⁺.

### Example 39

### 2-{Methyl[2-({5-[(5-methyl-3-{3-[4-(trifluoromethoxy)phenyl]-1,2,4-oxadiazol-5-yl}-1H-pyrazol-1-yl)methyl]pyridin-2-yl}amino)ethyl]amino}ethanol

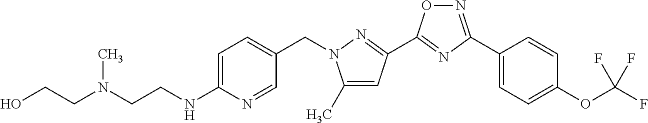

0.5 mg (0.002 mmol) of palladium(II) acetate and 1.3 mg (0.002 mmol) of (R)-(−)-1-[(S)-2-(dicyclohexylphosphino)ferrocenyl]ethyldi-tert-butylphosphine were added to a mixture of 100 mg (0.229 mmol) of the compound from Example 46A, 33 mg (0.275 mmol) of 2-[(2-aminoethyl)(methyl)amino]ethanol [G. Rewcastle et al., J. Med. Chem. 1998, 41 (5), 742-751] and 31 mg (0.321 mmol) of sodium tert-butoxide in 250 μl of DME, and the mixture was heated at 75° C. for 16 h. After cooling to RT, the mixture was diluted with a few ml of acetonitrile and separated into its components by preparative HPLC (method L). The product fractions were combined and substantially freed from the organic solvents on a rotary evaporator. By addition of saturated aqueous sodium bicarbonate solution, the aqueous solution obtained was alkaline, and the mixture was extracted twice with in each case about 10 ml of dichloromethane. The combined organic extracts were dried over anhydrous magnesium sulphate. Filtration and removal of the solvent on a rotary evaporator gave 40 mg (34% of theory) of the title compound.

¹H-NMR (400 MHz, CDCl₃, δ/ppm): 8.25 (d, 2H), 8.05 (d, 1H), 7.47 (dd, 1H), 7.33 (d, 2H), 6.80 (s, 1H), 6.74 (d, 1H), 5.37 (s, 2H), 4.38 (t, 2H), 2.78 (quart, 4H), 2.51 (t, 2H), 2.33 (s, 6H).

LC/MS (method E, ESIpos): Rₜ=1.18 min, m/z=518 [M+H]⁺.

### Example 40

### 5-[(5-Methyl-3-{3-[4-(trifluoromethoxy)phenyl]-1,2,4-oxadiazol-5-yl}-1H-pyrazol-1-yl)methyl]-2-[2-(pyrrolidin-1-yl)ethoxy]pyridine

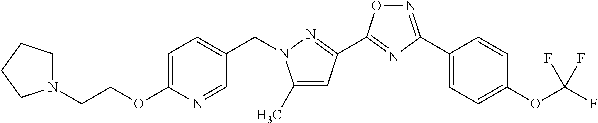

40 mg (0.344 mmol) of 2-(pyrrolidin-1-yl)ethanol were dissolved in 1 ml of NMP, 14 mg (0.344 mmol) of sodium hydride (60% dispersion in mineral oil) were added and the stirred mixture was heated at 90° C. for 30 min. 100 mg (0.229 mmol) of the compound from Example 46A were then added, and the mixture was stirred at 90° C. for a further 2 h. The reaction mixture was then cooled to RT, water was added slowly and the mixture was stirred at RT for 5 min. 1 ml of acetonitrile was then added, and the mixture was purified by preparative HPLC (method L). The product fractions were combined, some of the volume of liquid was removed on a rotary evaporator and a basic pH was established using solid sodium bicarbonate. Extraction was then carried out three times with in each case 15 ml of dichloromethane, the combined dichloromethane phases were dried over sodium sulphate and filtered and the solvent was removed on a rotary evaporator. After drying under reduced pressure, 39 mg (33% of theory) of the title compound were obtained.

¹H-NMR (400 MHz, CDCl₃, δ/ppm): 8.25 (d, 2H), 8.05 (d, 1H), 7.47 (dd, 1H), 7.32 (d, 2H), 6.81-6.73 (m, 2H), 5.37 (s, 2H), 4.43 (t, 2H), 2.90 (t, 2H), 2.62 (s, 6H), 2.32 (s, 3H), 1.83-1.77 (m, 2H).

LC/MS (method D, ESIpos): Rₜ=1.99 min, m/z=515 [M+H]⁺.

### Example 41

### 5-[(5-Methyl-3-{3-[4-(trifluoromethoxy)phenyl]-1,2,4-oxadiazol-5-yl}-1H-pyrazol-1-yl)methyl]-2-{[2-(pyrrolidin-1-yl)ethyl]sulphanyl}pyridine

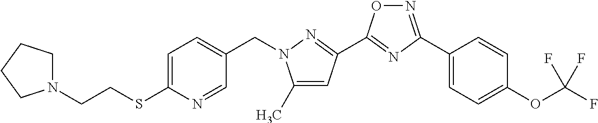

83 mg (2.07 mmol) of a 60% dispersion of sodium hydride in mineral oil were added to a solution of 271 mg (2.07 mmol) of 2-(pyrrolidin-1-yl)ethanethiol in 7.2 ml of diethylene glycol dimethyl ether. The mixture was stirred at RT for 15 min, and 200 mg (0.413 mmol) of the compound from Example 46A were then added. This reaction mixture was then heated at 100° C. in a microwave oven for 30 min (CEM Discover, initial irradiation power 250 W). After cooling to RT, the reaction mixture was diluted with a little acetonitrile and separated into its components by preparative HPLC (method K). The product fractions were combined and freed from the solvent on a rotary evaporator, and the residue was taken up in a little methanol. The solution was passed through a bicarbonate cartridge (Polymerlabs, Stratospheres SPE, PL-HCO₃ MP SPE, capacity 0.9 mmol). After the solvent had been removed on a rotary evaporator, 208 mg (95% of theory) of the title compound were obtained.

¹H-NMR (400 MHz, CDCl₃, δ/ppm): 8.31 (d, 1H), 8.24 (d, 2H), 7.33 (d, 2H and dd, 1H), 7.14 (d, 1H), 6.80 (s, 1H), 5.38 (s, 2H), 3.32 (t, 2H), 2.77 (t, 2H), 2.60-2.54 (m, 4H), 2.32 (s, 3H), 1.82-1.77 (m, 4H).

HPLC (method B): Rₜ=4.68 min.

MS (DCI, NH₃): m/z=531 [M+H]⁺.

LC/MS (method E, ESIpos): Rₜ=1.49 min, m/z=531 [M+H]⁺.

### Example 42

### 4-[(5-Methyl-3-{3-[4-(trifluoromethoxy)phenyl]-1,2,4-oxadiazol-5-yl}-1H-pyrazol-1-yl)methyl]-N-[2-(morpholin-4-yl)ethyl]pyridine-2-amine

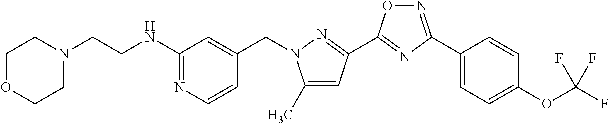

A solution of 100 mg (0.229 mmol) of the compound from Example 48A and 598 μl (4.59 mmol) of 2-(morpholin-4-yl)ethanamine in 1 ml of diethylene glycol dimethyl ether was heated in a microwave oven (CEM Discover, initial irradiation power 250 W) at 180° C. for 3 h. After cooling to RT, the reaction mixture was purified directly by preparative HPLC (method L). The product fractions were combined and concentrated on a rotary evaporator, so that only water remained as solvent. Solid sodium bicarbonate was added, and the mixture was stirred for a number of minutes. The precipitated product was then filtered off with suction, washed with water and dried under high vacuum. 82 mg (67% of theory) of the title compound were obtained.

¹H-NMR (400 MHz, CDCl₃, δ/ppm): 8.25 (d, 2H), 8.02 (d, 1H), 7.32 (d, 2H), 6.83 (s, 1H), 6.32 (d, 1H), 6.03 (s, 1H), 5.32 (s, 2H), 5.10 (s, broad, 1H), 3.70 (s, 4H), 3.35-3.26 (m, 2H), 2.62-2.53 (m, 2H), 2.42 (s, 4H), 2.29 (s, 3H).

LC/MS (method F, ESIpos): Rₜ=1.03 min, m/z=530 [M+H]⁺.

The compounds in the following table were prepared analogously to the process described in Example 42 from the compound from Example 48A and the corresponding amine compounds. These were either commercially available, or they were prepared according to the following literature procedure: N-methyl-2-(morpholin-4-yl)propanamine [The Wellcome Foundation Ltd., GB-Patent Specification 830,519], 1-(2-aminoethyl)piperidin-4-ol [K. Pors et al., J. Med. Chem. 2005, 48 (21), 6690-6695].

| | | HPLC: | MS: m/z | LC/MS |
|---|---|---|---|---|
| Example | Structure | Rₜ [min] | [M + H]⁺ | method |
| 43 | 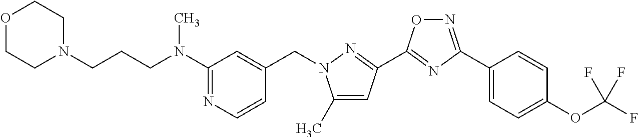 | 1.02 | 558 | F |
| | ¹H-NMR (400 MHz, CDCl₃, δ/ppm): 8.25 (d, 2H), 8.08 (d, 1H), 7.32 (d, 2H), 6.82 (s, | | | |
| | 1H), 6.24 (d, 1H), 6.15 (s, 1H), 5.32 (s, 2H), 3.70 (t, 4H), 3.53 (t, 2H), 2.99 (s, 3H), | | | |
| | 2.43-2.35 (m, 4H), 2.35-2.27 (m, 5H), 1.78-1.70 (m, 2H). | | | |
| 44 | 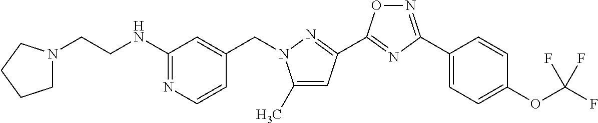 | 1.07 | 514 | F |
| | ¹H-NMR (400 MHz, CDCl₃, δ/ppm): 8.25 (d, 2H), 8.02 (d, 1H), 7.32 (d, 2H), 6.83 (s, | | | |
| | 1H), 6.31 (d, 1H), 6.02 (s, 1H), 5.32 (s, 2H), 5.10 (t, 1H), 3.33-3.29 (m, 2H), 2.69 (t, | | | |
| | 2H), 2.55-2.47 (m, 4H), 2.29 (s, 3H), 1.78-1.70 (m, 4H). | | | |
| 45 | 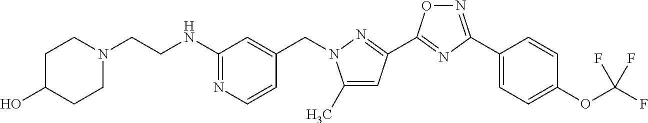 | 1.58 | 544 | C |
| | ¹H-NMR (400 MHz, CDCl₃, δ/ppm): 8.25 (d, 2H), 8.02 (d, 1H), 7.32 (d, 2H), 6.82 (s, | | | |
| | 1H), 6.31 (d, 1H), 6.02 (s, 1H), 5.32 (s, 2H), 5.12 (s, broad, 1H), 3.75-3.65 (s, broad, | | | |
| | 1H), 3.32-3.25 (m, 2H), 2.80-2.70 (m, 2H), 2.56 (t, 2H), 2.30 (s, 3H), 2.21-2.11 (m, | | | |
| | 2H), 1.91-1.82 (m, 2H), 1.62-1.40 (m, 3H). | | | |
| 46 | 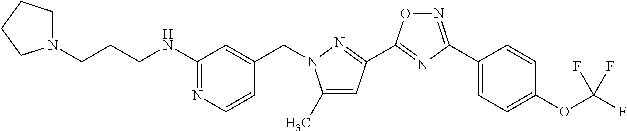 | 0.96 | 528 | F |
| | ¹H-NMR (400 MHz, CDCl₃, δ/ppm): 8.25 (d, 2H), 8.01 (d, 1H), 7.32 (d, 2H), 6.83 (s, | | | |
| | 1H), 6.28 (d, 1H), 5.97 (s, 1H), 5.38 (t, 1H), 5.32 (s, 2H), 3.35-3.29 (m, 2H), 2.55 (t, | | | |
| | 2H), 2.52-2.43 (m, 4H), 2.29 (s, 3H), 1.85-1.70 (m, 6H). | | | |
| 47 | 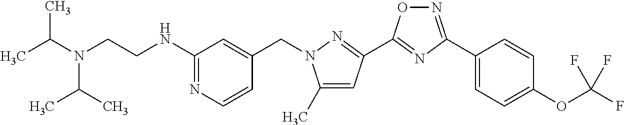 | 1.81 | 544 | C |

¹H-NMR (400 MHz, CDCl₃, δ/ppm): 8.25 (d, 2H), 8.02 (d, 1H), 7.32 (d, 2H), 6.82 (s,
1H), 6.27 (d, 1H), 6.02 (s, 1H), 5.32 (s, 2H), 5.13 (s, broad, 1H), 3.20-3.13 (m, 2H),
3.08-2.95 (m, 2H), 2.70-2.65 (m, 2H), 2.29 (s, 3H), 0.99 (d, 12H).

### Example 48

### N-[2-(Dimethylamino)ethyl]-N-methyl-3-[(5-methyl-3-{3-[4-(trifluoromethoxy)phenyl]-1,2,4-oxa-diazol-5-yl}-1H-pyrazol-1-yl)methyl]benzenecarboxamide

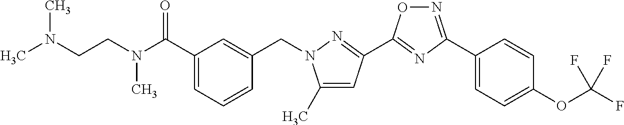

Under inert conditions, a solution of 55 mg (0.124 mmol) of the compound from Example 56A was initially charged in 2 ml of anhydrous dichloromethane, and 54 μl (0.619 mmol) of oxalyl chloride and a small drop of DMF were added at RT. After 2 h, the reaction mixture was freed from all volatile components on a rotary evaporator, and the intermediate was dried under high vacuum for 1 h. The residue was then dissolved in 1 ml of anhydrous THF and added dropwise to a solution of 19 mg (0.186 mmol) of N,N,N′-trimethylethane-1,2-diamine and 65 μl (0.371 mmol) of N,N-diisopropylethylamine in 1 ml of anhydrous THF. The reaction mixture was stirred at RT for 16 h. Approx. 1 ml of DMF and 1 ml of methanol were then added and the reaction mixture was separated directly by preparative HPLC (method K). The product fractions were concentrated and then once more dissolved in methanol, and the adhering formic acid was removed via a bicarbonate cartridge (Polymerlabs, Stratospheres SPE, PL-HCO₃ MP SPE, capacity 0.9 mmol). 31 mg (48% of theory) of the title compound were obtained.

¹H-NMR (400 MHz, CDCl₃, δ/ppm): 8.25 (d, 2H), 7.40-7.32 (m, 4H), 7.21 (s, 1H), 7.17 (d, 1H), 6.82 (s, 1H), 5.47 (s, 2H), 3.61 (broad, 1H), 3.27 (broad, 1H), 3.06 and 2.93 (broad, tog. 3H), 2.55 (broad, 1H), 2.33 (broad, 1H), 2.29 (s, 3H), 2.28 (broad, 3H), 2.03 (broad, 3H).

HPLC (method A): Rₜ=4.51 min.

LC/MS (method F, ESIpos): Rₜ=1.12 min, m/z=529 [M+H]⁺.

The compounds in the following table were prepared analogously to the process described in Example 48 from the compound from Example 56A and the corresponding amine compounds.

| Ex- | | HPLC: | | |
|---|---|---|---|---|
| am- | | Rₜ | MS: m/z | LC/MS |
| ple | Structure | [min] | [M + H]⁺ | method |
| 49 | 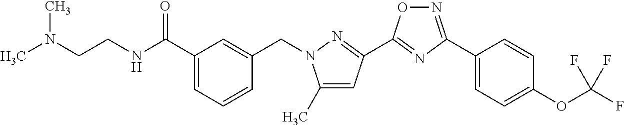 | 1.74 | 515 | C |
| | ¹H-NMR (400 MHz, CDCl₃, δ/ppm): 8.25 (d, 2H), 7.70 (d, 1H), 7.65 (s, 1H), 7.40 (dd, | | | |
| | 1H), 7.33 (d, 2H), 7.26 (d, 1H), 6.82 (s, 1H), 6.81 (t, broad, 1H), 5.50 (s, 2H), 3.49 | | | |
| | (quart, 2H), 2.50 (t, 2H), 2.29 (s, 3H), 2.23 (s, 6H). | | | |
| 50 | 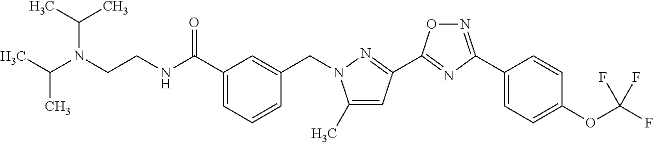 | 1.85 | 571 | C |
| | ¹H-NMR (400 MHz, CDCl₃, δ/ppm): 8.25 (d, 2H), 7.69 (d, 1H), 7.60 (s, 1H), 7.41 (dd, | | | |
| | 1H), 7.33 (d, 2H), 7.29 (d, 1H), 6.93 (t, broad, 1H), 6.81 (s, 1H), 5.50 (s, 2H), 3.40 | | | |
| | (quart, 2H), 3.00 (m, 2H), 2.67 (t, 2H), 2.28 (s, 3H), 0.99 (d, 12H). | | | |
| 51 | 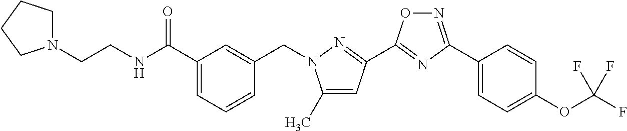 | 1.77 | 541 | C |
| | ¹H-NMR (400 MHz, CDCl₃, δ/ppm): 8.25 (d, 2H), 7.69 (d, 1H), 7.65 (s, 1H), 7.40 (dd, | | | |
| | 1H), 7.33 (d, 2H), 7.27 (d, 1H), 6.86 (t, broad, 1H), 6.82 (s, 1H), 5.49 (s, 2H), 3.53 | | | |
| | (quart, 2H), 2.69 (t, 2H), 2.55-2.51 (m, 4H), 2.29 (s, 3H), 1.78-1.73 (m, 4H). | | | |
| 52 | 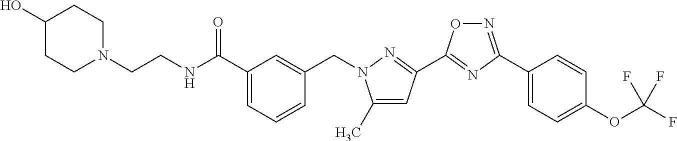 | 1.71 | 571 | C |

¹H-NMR (400 MHz, CDCl₃, δ/ppm): 8.24 (d, 2H), 7.70 (d, 1H), 7.62 (s, 1H), 7.42 (dd,
1H), 7.33 (d, 2H), 7.30 (d, 1H), 6.84 (s, 1H), 6.83 (t, broad, 1H), 5.50 (s, 2H), 3.73-3.65
(m, 1H), 3.50 (quart, 2H), 2.81-2.74 (m, 2H), 2.55 (t, 2H), 2.31 (s, 3H), 2.20-2.13 (m,
2H), 1.92-1.85 (m, 2H), 1.70-1.67 (m, 1H), 1.58-1.49 (m, 2H).

### Example 53

### N-(2-Aminoethyl)-3-[(5-methyl-3-{3-[4-(trifluoromethoxy)phenyl]-1,2,4-oxadiazol-5-yl}-1H-pyrazol-1-yl)methyl]benzenecarboxamide hydrochloride

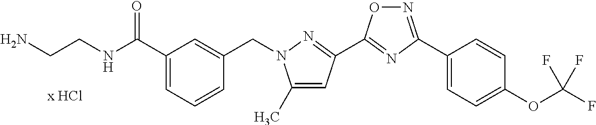

### Step 1: tert-Butyl{2-[({3-[(5-methyl-3-{3-[4-(trifluoromethoxy)phenyl]-1,2,4-oxadiazol-5-yl}-1H-pyrazol-1-yl)methyl]phenyl}carbonyl)amino]ethyl}carbamate

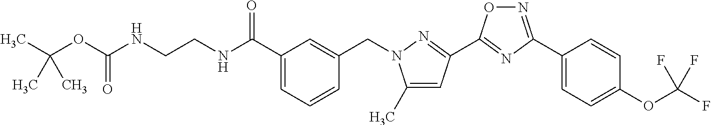

Analogously to the process described in Example 48, 99 mg (92% of theory) of the title compound were obtained from 80 mg (0.180 mmol) of the compound from Example 56A and 58 mg (0.360 mmol) of tert-butyl (2-aminoethyl)carbamate.

¹H-NMR (400 MHz, CDCl₃, δ/ppm): 8.25 (d, 2H), 7.75 (s, 1H), 7.71 (d, 2H), 7.38 (t, 1H), 7.33 (d, 2H), 7.29 (t, broad, 1H), 7.23 (d, 1H), 6.82 (s, 1H), 5.50 (s, 2H), 5.50 (t, broad, 1H), 3.54 (quart, 2H), 3.40 (quart, 2H), 2.29 (s, 3H), 1.41 (s, 9H).

LC/MS (method E, ESIpos): Rₜ=2.43 min, m/z=587 [M+H]⁺.

### Step 2: N-(2-Aminoethyl)-3-[(5-methyl-3-{3-[4-(trifluoromethoxy)phenyl]-1,2,4-oxadiazol-5-yl}-1H-pyrazol-1-yl)methyl]benzenecarboxamide hydrochloride

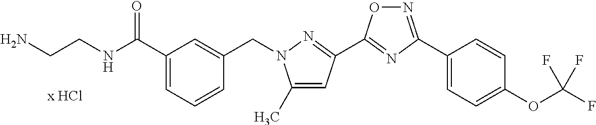

5 ml of a 4 M solution of hydrogen chloride in dioxane were added to 80 mg (0.136 mmol) of the compound from Example 53/step 1. The reaction mixture was stirred at RT for 30 min, 10 ml of diethyl ether were then added and the mixture was stirred at RT for 10 min. The precipitated product was filtered off with suction, washed with diethyl ether and dried under high vacuum. 63 mg (86% of theory) of the title compound were obtained.

¹H-NMR (400 MHz, DMSO-d₆, δ/ppm): 8.77 (t, 1H), 8.20 (d, 2H), 7.95 (broad, 3H), 7.87 (d, 1H), 7.78 (s, 1H), 7.60 (d, 2H), 7.50 (t, 1H), 7.37 (d, 1H), 6.96 (s, 1H), 5.57 (s, 2H), 3.50 (quart, 2H), 3.00-2.94 (m, 2H), 2.36 (s, 3H).

LC/MS (method E, ESIpos): Rₜ=1.43 min, m/z=487 [M+H]⁺.

### Example 54

### N-(2-Aminoethyl)-3-[(5-methyl-3-{3-[4-(trifluoromethoxy)phenyl]-1,2,4-oxadiazol-5-yl}-1H-pyrazol-1-yl)methyl]benzenecarboxamide

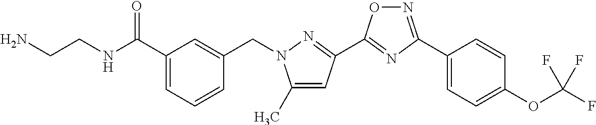

40 mg (0.076 mmol) of the compound from Example 53 were dissolved in 10 ml of methanol and passed through a bicarbonate filter cartridge (Polymerlabs, Stratospheres SPE, PL-HCO₃ MP SPE, capacity 0.9 mmol). The mixture was then evaporated to dryness on a rotary evaporator and the residue obtained was dried under high vacuum. 36 mg (97% of theory) of the title compound were obtained.

¹H-NMR (400 MHz, CDCl₃, δ/ppm): 8.25 (d, 2H), 7.70 (d, 1H), 7.69 (s, 1H), 7.40 (t, 1H), 7.33 (d, 2H), 7.27 (d, 1H), 6.82 (s, 1H), 6.73 (t, broad, 1H), 5.49 (s, 2H), 3.48 (quart, 2H), 2.96-2.92 (m, 2H), 2.29 (s, 3H), 1.39 (broad, 2H).

LC/MS (method C, ESIpos): Rₜ=1.72 min, m/z=487 [M+H]⁺.

### Example 55

### N-{3-[(5-Methyl-3-{3-[4-(trifluoromethoxy)phenyl]-1,2,4-oxadiazol-5-yl}-1H-pyrazol-1-yl)-methyl]phenyl}-3-(pyrrolidin-1-yl)propanamide

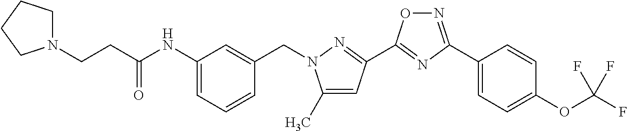

At RT, 98 μl (1.13 mmol) of oxalyl chloride and a small drop of DMF were added to a solution of 32 mg (0.216 mmol) of 3-(pyrrolidin-1-yl)propanoic acid [Z. Dega-Szafran et al., J. Mol. Struct. 1997, 436 (1), 107-122] in 5 ml of anhydrous dichloromethane. After 1 h at RT, the mixture was evaporated to dryness on a rotary evaporator and the residue was once more dissolved in 2.5 ml of anhydrous dichloromethane. This solution was added to a solution of 75 mg (0.181 mmol) of the compound from Example 61A and 63 μl (0.451 mmol) of triethylamine in 2.5 ml of dichloromethane. The reaction mixture was stirred at RT for 16 h. The mixture was then diluted with about 2 ml of methanol and the complete mixture was then separated into its components by preparative HPLC (method K). The product fractions were combined and concentrated to dryness on a rotary evaporator. The residue was taken up in approx. 5 ml of methanol and the solution was passed through a bicarbonate cartridge (Polymerlabs, Stratospheres SPE, PL-HCO₃ MP SPE, capacity 0.9 mmol) in order to remove adhering formic acid originating from the HPLC purification. 53 mg (54% of theory) of the title compound were obtained.

¹H-NMR (400 MHz, CDCl₃, δ/ppm): 11.41 (s, broad, 1H), 8.25 (d, 2H), 7.42 (d, 1H), 7.33 (d, 2H), 7.28 (s, 1H), 7.25 (dd, 1H), 6.84 (d, 1H), 6.81 (s, 1H), 5.43 (s, 2H), 2.81 (dd, 2H), 2.63-2.60 (m, 4H), 2.50 (dd, 2H), 2.30 (s, 3H), 1.30-1.27 (m, 4H).

HPLC (method B): Rₜ=4.53 min.

MS (ESIpos): m/z=541 [M+H]⁺.

LC/MS (method F, ESIpos): Rₜ=1.17 min, m/z=541 [M+H]⁺.

### Example 56

### N,N-Dimethyl-3-{3-[(5-methyl-3-{3-[4-(trifluoromethoxy)phenyl]-1,2,4-oxadiazol-5-yl}-1H-pyrazol-1-yl)methyl]phenoxy}propane-1-amine

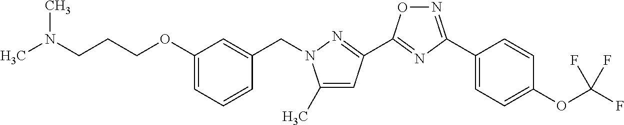

At 0° C., 181 mg (1.61 mmol) of solid potassium tert-butoxide were added to a solution of 200 mg (0.645 mmol) of the compound from Example 17A and 221 mg (0.838 mmol) of 3-[3-(chloromethyl)phenoxy]-N,N-dimethylpropane-1-amine hydrochloride in 7.5 ml of anhydrous THF. After warming to RT, the reaction mixture was stirred at this temperature for 16 h. About 2 ml of water were then added, and the mixture was separated into its components by preparative HPLC (method K). The product fractions were combined and freed from the solvent on a rotary evaporator. The residue obtained was re-purified by MPLC (silica gel, cyclohexane/ethyl acetate/triethylamine 5:1:0.5). 113 mg (35% of theory) of the title compound were obtained.

¹H-NMR (400 MHz, CDCl₃, δ/ppm): 8.26 (d, 2H), 7.33 (d, 2H), 7.23 (t, 1H), 6.82 (dd, 1H), 6.81 (s, 1H), 6.72 (d, 1H), 6.70 (d, 1H), 5.42 (s, 2H), 3.97 (t, 2H), 2.41 (t, 2H), 2.28 (s, 3H), 2.23 (s, 6H), 1.91 (quint, 2H).

LC/MS (method E, ESIpos): Rₜ=1.60 min, m/z=502 [M+H]⁺.

### Example 57

### 5-(5-Methyl-1-{3-[2-(pyrrolidin-1-yl)ethoxy]benzyl}-1H-pyrazol-3-yl)-3-[4-(trifluoromethoxy)-phenyl]-1,2,4-oxadiazole

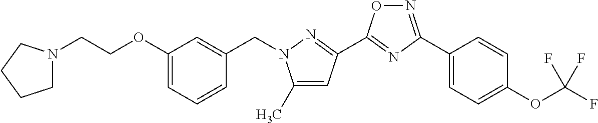

At RT, 19 mg (0.468 mmol) of a 60% dispersion of sodium hydride in mineral oil were added to a solution of 75 mg (0.180 mmol) of the compound from Example 58A in 2 ml of anhydrous DMF. After 10 min, 46 mg (0.270 mmol) of 1-(2-chloroethyl)pyrrolidine hydrochloride were added and the reaction mixture was stirred at RT for 15 h. 1 ml of water was then added and the reaction mixture was separated by preparative HPLC (method K). The product fractions were combined and the solvent was removed on a rotary evaporator. The residue obtained was dissolved in 5 ml of methanol and freed via a bicarbonate cartridge (Polymerlabs, Stratospheres SPE, PL-HCO₃ MP SPE, capacity 0.9 mmol) from the adhering formic acid originating from the HPLC purification. 24 mg (26% of theory) of the title compound were obtained.

¹H-NMR (400 MHz, CDCl₃, δ/ppm): 8.25 (d, 2H), 7.33 (d, 2H), 7.23 (t, 1H), 6.84 (dd, 1H), 6.80 (s, 1H), 6.73 (d, 1H), 6.70 (d, 1H), 5.42 (s, 2H), 4.05 (t, 2H), 2.86 (t, 2H), 2.61-2.58 (m, 4H), 2.27 (s, 3H), 1.80-1.76 (m, 4H).

LC/MS (method C, ESIpos): Rₜ=1.82 min, m/z=514 [M+H]⁺.

### Example 58

### 1-(3-{4-[(5-Methyl-3-{3-[4-(trifluoromethoxy)phenyl]-1,2,4-oxadiazol-5-yl}-1H-pyrazol-1-yl)-methyl]phenyl}propyl)piperidin-4-ol

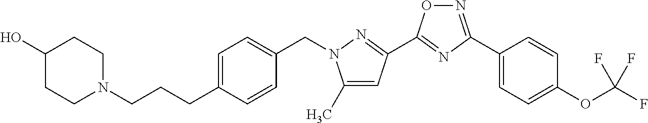

At −78° C., 24 μl (0.144 mmol) of trifluoromethanesulphonic anhydride and 42 μl (0.360 mmol) of 2,6-dimethylpyridine were added under inert conditions to a solution of 55 mg (0.120 mmol) of the compound from Example 66A in 4 ml of anhydrous dichloromethane. After 1 h, still at −78° C., 121 mg (1.20 mmol) of 4-hydroxypiperidine were added. The reaction mixture was then allowed to warm to RT and stirred for another 16 h. The mixture was then evaporated to complete dryness on a rotary evaporator. The residue was taken up in a few ml of DMSO, and the product was isolated by preparative HPLC (method K). 51 mg (79% of theory) of the title compound were obtained.

¹H-NMR (400 MHz, CDCl₃, δ/ppm): 8.24 (d, 2H), 7.33 (d, 2H), 7.15 (d, 2H), 7.08 (d, 2H), 6.80 (s, 1H), 5.41 (s, 2H), 3.71-3.64 (m, 1H), 2.78-2.71 (m, 2H), 2.60 (t, 2H), 2.32 (dd, 2H), 2.13-2.06 (m, 2H), 1.92-1.84 (m, 2H), 1.78 (quint, 2H), 1.64-1.53 (m, 3H).

HPLC (method B): Rₜ=4.52 min.

MS (ESIpos): m/z=542 [M+H]⁺.

LC/MS (method F, ESIpos): Rₜ=1.19 min, m/z=542 [M+H]⁺.

The compound in the following table was prepared from the compound from Example 66A and the corresponding amine analogously to the process described in Example 58:

| | | HPLC: | MS: m/z | LC/MS |
|---|---|---|---|---|
| Example | Structure | Rₜ [min] | [M + H]⁺ | method |
| 59 | 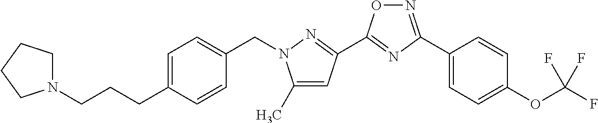 | 1.67 | 512 | E |

¹H-NMR (400 MHz, CDCl₃, δ/ppm): 8.25 (d, 2H), 7.33 (d, 2H), 7.16 (d, 2H), 7.09 (d,
2H), 6.80 (s, 1H), 5.42 (s, 2H), 2.62 (t, 2H), 2.50-2.46 (m, 4H), 2.44 (dd, 2H), 2.27 (s,
3H), 1.81 (quint, 2H), 1.78-1.75 (m, 4H).

### Example 60

### 4-[(5-Methyl-3-{3-[4-(trifluoromethoxy)phenyl]-1,2,4-oxadiazol-5-yl}-1H-pyrazol-1-yl)methyl]-N-[2-(morpholin-4-yl)ethyl]aniline

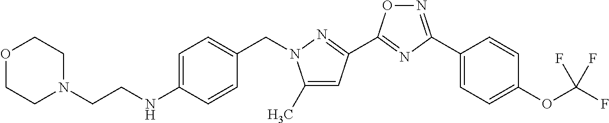

200 mg (0.380 mmol) of the compound from Example 51A and 59 mg (0.456 mmol) of 2-(morpholin-4-yl)ethanamine were initially charged in 2 ml of toluene, and 51 mg (0.532 mmol) of sodium tert-butoxide, 17 mg (0.019 mmol) of tris(dibenzylideneacetone)dipalladium(0) and 21 mg (0.038 mmol) of 1,1′-bis(diphenylphosphino)ferrocene were added at RT. The mixture was then stirred at reflux temperature overnight. After cooling to RT, the reaction mixture was filtered through kieselguhr, the filter cake was washed with ethyl acetate and the filtrate was concentrated on a rotary evaporator. The residue was purified by preparative HPLC (method L). The combined product-containing fractions were concentrated on a rotary evaporator until only a small residual volume of liquid remained. A weakly basic pH was established with sodium bicarbonate, and the mixture was extracted twice with ethyl acetate. The combined ethyl acetate phases were dried over magnesium sulphate, filtered and concentrated. The residue was re-purified by silica gel thick-layer chromatography (mobile phase: dichloromethane/methanol 95:5). The product zone was extracted with dichloromethane/methanol 9:1. The solvent was removed and the residue was dried under reduced pressure. 41 mg (20% of theory) of the title compound were obtained.

¹H-NMR (400 MHz, CDCl₃, δ/ppm): 8.25 (d, 2H), 7.33 (d, 2H), 7.06 (d, 2H), 6.77 (s, 1H), 6.58 (d, 2H), 5.33 (s, 2H), 4.38 (s, broad, 1H), 3.74-3.68 (m, 4H), 3.14 (t, 2H), 2.64-2.59 (m, 2H), 2.49-2.43 (m, 4H), 2.28 (s, 3H).

LC/MS (method F, ESIpos): Rₜ=1.16 min, m/z=529 [M+H]⁺.

### Example 61

### 4-[(5-Methyl-3-{3-[4-(trifluoromethoxy)phenyl]-1,2,4-oxadiazol-5-yl}-1H-pyrazol-1-yl)methyl]-N-[2-(pyrrolidin-1-yl)ethyl]aniline

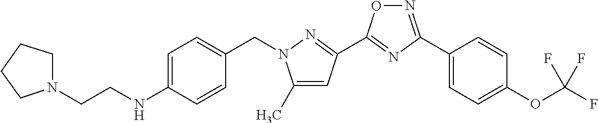

### Step 1: tert-Butyl{4-[(5-methyl-3-{3-[4-(trifluoromethoxy)phenyl]-1,2,4-oxadiazol-5-yl}-1H-pyrazol-1-yl)methyl]phenyl}[2-(pyrrolidin-1-yl)ethyl]carbamate

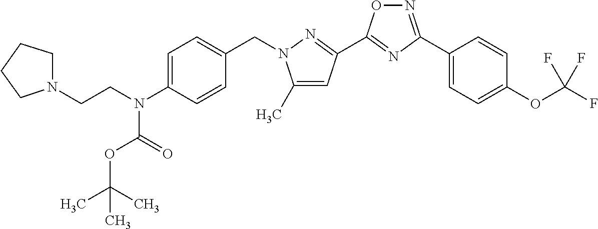

7 mg (0.179 mmol) of a 60% dispersion of sodium hydride in mineral oil were added to a solution of 60 mg (0.090 mmol) of the compound from Example 62A in 1 ml of anhydrous DMF. After 10 min, a solution of 15 mg (0.090 mmol) of 1-(2-chloroethyl)pyrrolidine hydrochloride in 1 ml of anhydrous DMF was added. The reaction mixture was initially stirred at RT for 1 h. A further 1.8 mg of the sodium hydride dispersion were then added, and stirring at RT was continued. After 24 h, a further 1.8 mg of the sodium hydride dispersion were added. After a further 15 h of stirring, the reaction mixture was then diluted with about 3 ml of methanol and separated directly into its components by preparative HPLC (method K). The product fractions were combined and freed from the solvent on a rotary evaporator, and the residue was triturated with pentane/diethyl ether (10:1). The solid was filtered off and dried under high vacuum. 41 mg (62% of theory, purity 83%) of the title compound were obtained.

¹H-NMR (400 MHz, CDCl₃, δ/ppm): 8.26 (d, 2H), 7.34 (d, 2H), 7.20 (d, 2H), 7.11 (d, 2H), 6.81 (s, 1H), 5.43 (s, 2H), 3.75 (t, 2H), 2.65 (t, 2H), 2.56-2.50 (m, 4H), 2.28 (s, 3H), 1.72-1.68 (m, 4H), 1.42 (s, 9H).

LC/MS (method E, ESIpos): Rₜ=1.72 min, m/z=613 [M+H]⁺.

### Step 2: 4-[(5-Methyl-3-{3-[4-(trifluoromethoxy)phenyl]-1,2,4-oxadiazol-5-yl}-1H-pyrazol-1-yl)methyl]-N-[2-(pyrrolidin-1-yl)ethyl]aniline

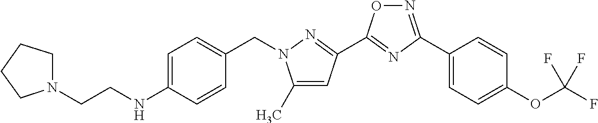

1.5 ml of a 4 M solution of hydrogen chloride in dioxane were added to 32 mg (0.061 mmol) of the compound from Example 61/step 1. The mixture was stirred at RT for 15 min and then evaporated to complete dryness on a rotary evaporator. The residue obtained was dissolved in approx. 5 ml of methanol and the solution was passed through a bicarbonate cartridge (Polymerlabs, Stratospheres SPE, PL-HCO₃ MP SPE, capacity 0.9 mmol). Since the product was still contaminated it was re-purified initially by preparative thick-layer chromatography (silica gel; mobile phase: 25 ml of cyclohexane/ethyl acetate 1:3 with 1 ml of triethylamine) and then by preparative HPLC (method K). The product fractions were combined and concentrated to dryness on a rotary evaporator. The residue was dissolved in about 5 ml of methanol and freed from adhering formic acid via a bicarbonate cartridge (see above). 13 mg (59% of theory) of the title compound were obtained in this manner.

¹H-NMR (400 MHz, CDCl₃, δ/ppm): 8.25 (d, 2H), 7.33 (d, 2H), 7.04 (d, 2H), 6.77 (s, 1H), 6.58 (d, 2H), 5.33 (s, 2H), 4.34 (t, broad, 1H), 3.15 (quart, 2H), 2.71 (t, 2H), 2.53-2.49 (m, 4H), 2.29 (s, 3H), 1.80-1.74 (m, 4H).

LC/MS (method F, ESIpos): Rₜ=1.18 min, m/z=513 [M+H]⁺.

### Example 62

### N-{4-[(5-Methyl-3-{3-[4-(trifluoromethoxy)phenyl]-1,2,4-oxadiazol-5-yl}-1H-pyrazol-1-yl)-methyl]phenyl}-2-(pyrrolidin-1-yl)acetamide

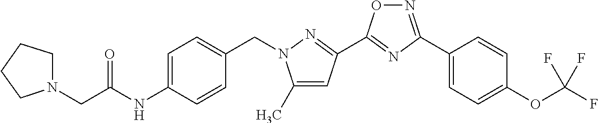

60 μl (0.434 mmol) of triethylamine, 36 mg (0.188 mmol) of EDC and 29 mg (0.188 mmol) of HOBt were added successively to a solution of 30 mg (0.144 mmol) of pyrrolidin-1-ylacetic acid hydrochloride in 2 ml of DMF. After 5 min, 60 mg (0.144 mmol) of the compound from Example 60A were added. The reaction mixture was stirred at RT for 16 h. The mixture was then diluted with about 2 ml of methanol and the complete mixture was then separated into its components by preparative HPLC (method K). The product fractions were combined and concentrated to dryness on a rotary evaporator. The residue was taken up in approx. 5 ml of methanol and the solution was passed through a bicarbonate cartridge (Polymerlabs, Stratospheres SPE, PL-HCO₃ MP SPE, capacity 0.9 mmol) in order to remove adhering formic acid originating from the HPLC purification. 37 mg (50% of theory) of the title compound were obtained.

¹H-NMR (400 MHz, CDCl₃, δ/ppm): 9.12 (s, broad, 1H), 8.25 (d, 2H), 7.56 (d, 2H), 7.33 (d, 2H), 7.17 (d, 2H), 6.80 (s, 1H), 5.41 (s, 2H), 3.28 (s, 2H), 2.70-2.66 (m, 4H), 2.27 (s, 3H), 1.87-1.82 (m, 4H).

HPLC (method B): Rₜ=4.56 min.

MS (DCI, NH₃): m/z=527 [M+H]⁺.

The compound in the following table was prepared from the compound from Example 60A and the corresponding amino acid analogously to the process described in Example 62:

| | | HPLC: | MS: m/z | LC/MS |
|---|---|---|---|---|
| Example | Structure | Rₜ [min] | [M + H]⁺ | method |
| 63 | 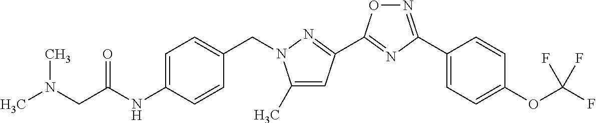 | 1.51 | 501 | E |

¹H-NMR (400 MHz, CDCl₃, δ/ppm): 9.12 (s, broad, 1H), 8.25 (d, 2H), 7.57 (d, 2H),
7.33 (d, 2H), 7.17 (d, 2H), 6.80 (s, 1H), 5.42 (s, 2H), 3.07 (s, 2H), 2.36 (s, 6H), 2.27 (s,
3H).

### Example 64

### N-{4-[(5-Methyl-3-{3-[4-(trifluoromethoxy)phenyl]-1,2,4-oxadiazol-5-yl}-1H-pyrazol-1-yl)-methyl]phenyl}-3-(pyrrolidin-1-yl)propanamide

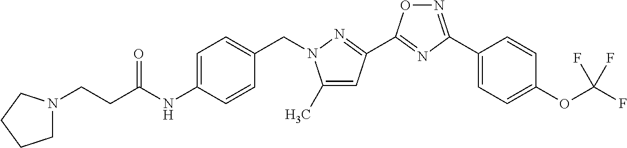

At RT, 92 μl (1.05 mmol) of oxalyl chloride and a small drop of DMF were added to a solution of 30 mg (0.211 mmol) of 3-(pyrrolidin-1-yl)propanoic acid [Z. Dega-Szafran et al., J. Mol. Struct. 1997, 436 (1), 107-122] in 1 ml of anhydrous dichloromethane. After 1 h at RT, the mixture was evaporated to dryness on a rotary evaporator and the residue was once more dissolved in 1 ml of anhydrous dichloromethane. This solution was added to a solution of 70 mg (0.169 mmol) of the compound from Example 60A and 59 μl (0.421 mmol) of triethylamine in 2 ml of dichloromethane. The reaction mixture was stirred at RT for 16 h. The mixture was then diluted with about 2 ml of methanol and the complete mixture was then separated into its components by preparative HPLC (method K). The product fractions were combined and concentrated to dryness on a rotary evaporator. The residue was taken up in approx. 5 ml of methanol and the solution was passed through a bicarbonate cartridge (Polymerlabs, Stratospheres SPE, PL-HCO₃ MP SPE, capacity 0.9 mmol) in order to remove adhering formic acid originating from the HPLC purification. 56 mg (62% of theory) of the title compound were obtained.

¹H-NMR (400 MHz, CDCl₃, δ/ppm): 11.37 (s, broad, 1H), 8.25 (d, 2H), 7.46 (d, 2H), 7.33 (d, 2H), 7.13 (d, 2H), 6.79 (s, 1H), 5.40 (s, 2H), 2.83 (dd, 2H), 2.67-2.63 (m, 4H), 2.52 (dd, 2H), 2.26 (s, 3H), 1.88-1.84 (m, 4H).

LC/MS (method F, ESIpos): Rₜ=1.16 min, m/z=541 [M+H]⁺.

### Example 65

### 1-(2-{4-[(5-Methyl-3-{3-[4-(trifluoromethoxy)phenyl]-1,2,4-oxadiazol-5-yl}-1H-pyrazol-1-yl)-methyl]phenoxy}ethyl)piperidin-4-ol

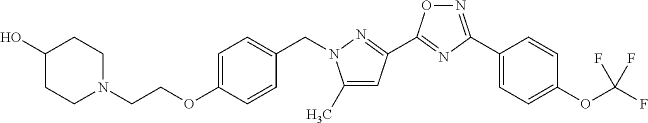

A mixture of 100 mg (0.209 mmol) of the compound from Example 63A and 42 mg (0.418 mmol) of piperidin-4-ol in 5 ml of DMF was heated at 80° C. overnight. The temperature was then increased to 100° C., and the mixture was stirred for another night. After cooling to RT, the mixture was purified directly by preparative HPLC (method L). The combined product-containing fractions were concentrated on a rotary evaporator until only a small residual volume of liquid remained. Sodium bicarbonate was added, the mixture was stirred for a few minutes and the solid formed was then filtered off and washed twice with water. After drying under reduced pressure, 84 mg (74% of theory) of the title compound were obtained.

¹H-NMR (400 MHz, CDCl₃, δ/ppm): 8.24 (d, 2H), 7.32 (d, 2H), 7.11 (d, 2H), 6.85 (d, 2H), 6.79 (s, 1H), 5.38 (s, 2H), 4.12-4.00 (m, 2H), 3.76-3.65 (m, 1H), 2.90-2.80 (m, 2H), 2.80-2.75 (m, 2H), 2.36-2.20 (m, 5H), 1.96-1.83 (m, 2H), 1.76-1.40 (m, 3H).

LC/MS (method C, ESIpos): Rₜ=1.76 min, m/z=544 [M+H]⁺.

### Example 66

### 5-(5-Methyl-1-{4-[2-(pyrrolidin-1-yl)ethoxy]benzyl}-1H-pyrazol-3-yl)-3-[4-(trifluoromethoxy)-phenyl]-1,2,4-oxadiazole

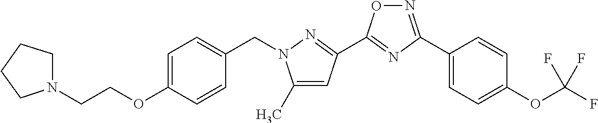

100 mg (0.240 mmol) of the compound from Example 59A together with 172 mg (0.528 mmol) of caesium carbonate were initially charged in 2 ml of DMF, 49 mg (0.288 mmol) of 1-(2-chloroethyl)pyrrolidine hydrochloride were added and the mixture was stirred at 150° C. for 2 h. After cooling to RT, the reaction mixture was filtered, the solid which had been filtered off was washed with a little DMF and the filtrate obtained in this manner was purified by preparative HPLC (method L). The combined product-containing fractions were concentrated on a rotary evaporator until only a small residual volume of liquid remained. Sodium bicarbonate was added, the mixture was stirred for a few minutes and the solid formed was then filtered off and washed twice with water. After drying under reduced pressure, 83 mg (67% of theory) of the title compound were obtained.

¹H-NMR (400 MHz, CDCl₃, δ/ppm): 8.25 (d, 2H), 7.32 (d, 2H), 7.12 (d, 2H), 6.88 (d, 2H), 6.79 (s, 1H), 5.38 (s, 2H), 4.08 (t, 2H), 2.88 (t, 2H), 2.63-2.58 (m, 4H), 2.27 (s, 3H), 1.82-1.77 (m, 4H).

LC/MS (method C, ESIpos): Rₜ=1.85 min, m/z=514 [M+H]⁺.

### Example 67

### 5-[5-Methyl-1-(4-{[2-(pyrrolidin-1-yl)ethyl]sulphanyl}benzyl)-1H-pyrazol-3-yl]-3-[4-(trifluoromethoxy)phenyl]-1,2,4-oxadiazole

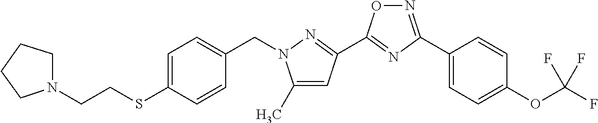

Under inert conditions, 17 mg (0.019 mmol) of tris(dibenzylideneacetone)dipalladium(0), 42 mg (0.076 mmol) of 1,1′-bis(diphenylphosphino)ferrocene and 265 μl (1.90 mmol) of triethylamine were added to a solution of 500 mg (0.95 mmol) of the compound from Example 51A in 6 ml of NMP. After 15 min of stirring at RT, 125 mg (0.950 mmol) of the compound from Example 64A were added. The reaction mixture was then stirred at 60° C. for 48 h. Since after this period the reaction had not yet gone to completion, the same amounts of the compound from Example 64A and tris(dibenzylideneacetone)dipalladium(0), 1,1′-bis(diphenylphosphino)ferrocene and tri-ethylamine were added and stirring was continued at 80° C. After a further 20 h, the reaction mixture was allowed to cool to RT, about 50 ml of water were added and the mixture was extracted three times with in each case about 50 ml of ethyl acetate. The combined organic extracts were washed with saturated sodium chloride solution and dried over anhydrous magnesium sulphate. After filtration and removal of the solvent on a rotary evaporator, the residue was purified by preparative HPLC (method K). The product fractions were combined and evaporated to dryness. The residue was dissolved in approx. 5 ml of methanol and the solution was passed through a bicarbonate cartridge (Polymerlabs, Stratospheres SPE, PL-HCO₃ MP SPE, capacity 0.9 mmol) in order to remove adhering formic acid originating from the HPLC purification. After the mixture had been concentrated and the residue had been dried under high vacuum, 90 mg (18% of theory) of the title compound were obtained.

¹H-NMR (400 MHz, CDCl₃, δ/ppm): 8.25 (d, 2H), 7.32 (d, 2H), 7.30 (d, 2H), 7.09 (d, 2H), 6.81 (s, 1H), 5.41 (s, 2H), 3.05 (dd, 2H), 2.71 (dd, 2H), 2.55-2.51 (m, 4H), 2.29 (s, 3H), 1.80-1.76 (m, 4H).

HPLC (method B): Rₜ=4.76 min.

MS (ESIpos): m/z=530 [M+H]⁺.

### Example 68

### 5-[(5-Methyl-3-{3-[4-(trifluoromethoxy)phenyl]-1,2,4-oxadiazol-5-yl}-1H-pyrazol-1-yl)methyl]-2-[3-(pyrrolidin-1-yl)propyl]pyridine

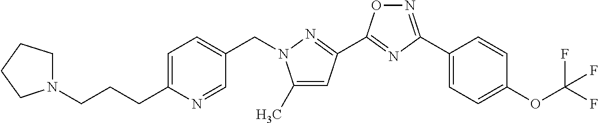

27 mg (0.059 mmol) of the compound from Example 70A were dissolved in 1 ml of ethanol, 25 μl (0.295 mmol) of pyrrolidine and 19 mg (0.089 mmol) of sodium triacetoxyborohydride were added and the mixture was stirred at RT for 4 h. 1 ml of acetic acid and 20 ml of dilute aqueous sodium chloride solution were then added, and the reaction mixture was extracted three times with in each case 20 ml of ethyl acetate. The combined organic phases were dried over sodium sulphate, filtered and concentrated on a rotary evaporator. The residue was purified by column chromatography on silica gel (mobile phase: dichloromethane/ethanol 10:1 dichloromethane/ethanol 10:1 with 0.5% concentrated aqueous ammonia solution dichloromethane/ethanol 5:1 with 0.5% concentrated aqueous ammonia solution). 12 mg (38% of theory) of the title compound were obtained.

¹H-NMR (400 MHz, CDCl₃, δ/ppm): 8.42 (d, 1H), 8.24 (d, 2H), 7.43 (dd, 1H), 7.32 (d, 2H), 7.14 (d, 1H), 6.81 (s, 1H), 5.42 (s, 2H), 2.83 (t, 2H), 2.65-2.55 (m, 6H), 2.04-1.93 (m, 2H), 1.90-1.70 (m, 4H).

LC/MS (method M, ESIpos): Rₜ=0.98 min, m/z=513 [M+H]⁺.

The compounds in the following table were prepared analogously to the process described in Example 68 from the compound from Example 70A and the corresponding amines:

| | | HPLC: | MS: m/z | LC/MS |
|---|---|---|---|---|
| Example | Structure | Rₜ [min] | [M + H]⁺ | method |
| 69 | 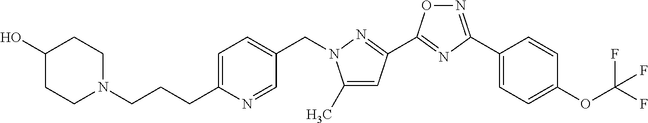 | 0.93 | 543 | M |
| | ¹H-NMR (400 MHz, CDCl₃, δ/ppm): 8.42 (s, 1H), 8.24 (d, 2H), 7.43 (dd, 1H), 7.32 (d, | | | |
| | 2H), 7.12 (d, 1H), 6.80 (s, 1H), 5.42 (s, 2H), 3.71 (s, broad, 1H), 2.82-2.74 (m, 4H), | | | |
| | 2.48-2.37 (m, 2H), 2.31 (s, 3H), 2.28-2.12 (m, 2H), 1.98-1.87 (m, 5H), 1.65-1.52 (m, | | | |
| | 2H). | | | |
| 70 | 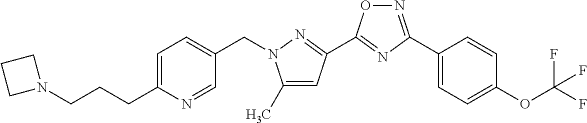 | 0.95 | 499 | M |

¹H-NMR (400 MHz, CDCl₃, δ/ppm): 8.42 (d, 1H), 8.24 (d, 2H), 7.44 (dd, 1H), 7.32 (d,
2H), 7.13 (d, 1H), 6.81 (s, 1H), 5.42 (s, 2H), 3.21 (t, 4H), 2.79 (t, 2H), 2.46 (t, 2H),
2.32 (s, 3H), 2.12-2.04 (m, 2H), 1.80-1.65 (m, 2H).

The compounds in the following table were prepared analogously to the process described in Example 14 from the corresponding chloropyridine derivatives and the corresponding amine compounds. In each case 20 equivalents of the amine compound were used. The amine compounds were either commercially available, or they were prepared according to literature procedures. Depending on the batch size, the product was, after purification by preparative HPLC, either stirred as described above with aqueous sodium bicarbonate solution or dissolved in methanol and passed through a bicarbonate cartridge (Polymerlabs, Stratospheres SPE, PL-HCO₃ MP SPE, capacity 0.9 mmol).

| | | HPLC: | MS: m/z | LC/MS |
|---|---|---|---|---|
| Example | Structure | Rₜ [min] | [M + H]⁺ | method |
| 71 | 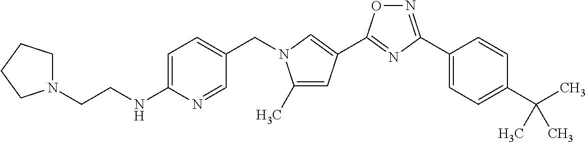 | 4.33 | 485 | A |
| | ¹H-NMR (400 MHz, CDCl₃, δ/ppm): 8.04 (d, 2H), 7.98 (s, 1H), 7.49 (d, 2H), 7.42 (s, | | | |
| | 1H), 7.15 (d, 1H), 6.54 (s, 1H), 6.39 (d, 1H), 5.20 (t, broad, 1H), 4.91 (s, 2H), 3.38 | | | |
| | (quart, 2H), 2.72 (t, 2H), 2.56-2.53 (m, 4H), 2.24 (s, 3H), 1.81-1.76 (m, 4H), 1.35 (s, | | | |
| | 9H). | | | |
| 72 | 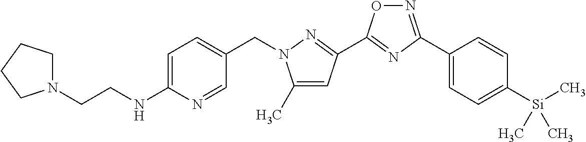 | 1.19 | 502 | F |
| | ¹H-NMR (400 MHz, CDCl₃, δ/ppm): 8.18 (d, 2H), 8.01 (d, 1H), 7.63 (d, 2H), 7.33 (dd, | | | |
| | 1H), 6.78 (s, 1H), 6.39 (d, 1H), 5.32-5.22 (m, 3H), 3.39 (dd, 2H), 2.77 (t, 2H), 2.62-2.52 | | | |
| | (m, 4H), 2.31 (s, 3H), 1.79 (s, 4H), 0.31 (s, 9H). | | | |
| 73 | 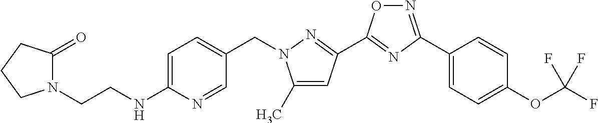 | 1.51 | 528 | E |
| | ¹H-NMR (400 MHz, CDCl₃, δ/ppm): 8.25 (d, 2H), 8.01 (s, 1H), 7.35-7.29 (m, 3H), | | | |
| | 6.76 (s, 1H), 6.38 (d, 1H), 5.28 (s, 2H), 4.96 (s, broad, 1H), 3.53-3.50 (m, 4H), 3.45 (t, | | | |
| | 2H), 2.36-2.30 (m, 5H), 2.01-1.94 (m, 2H). | | | |
| 74 | 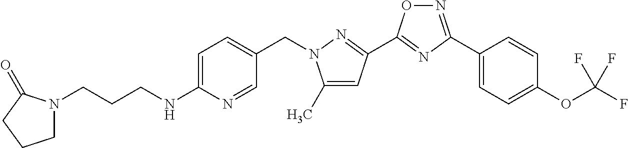 | 1.09 | 542 | F |
| | ¹H-NMR (400 MHz, CDCl₃, δ/ppm): 8.28 (d, 2H), 8.01 (s, 1H), 7.38-7.31 (m, 3H), | | | |
| | 6.79 (s, 1H), 6.41 (d, 1H), 5.32-5.28 (m, 3H), 3.42-3.31 (m, 6H), 2.41 (t, 2H), 2.34 (s, | | | |
| | 3H), 2.09-2.00 (m, 2H), 1.82-1.75 (m, 2H). | | | |
| 75 | 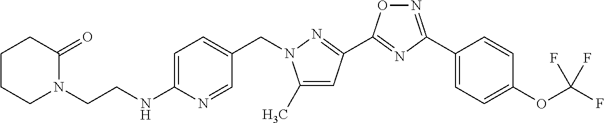 | 1.13 | 542 | F |
| | ¹H-NMR (400 MHz, CDCl₃, δ/ppm): 8.25 (d, 2H), 8.00 (s, 1H), 7.35-7.26 (m, 3H), | | | |
| | 6.76 (s, 1H), 6.39 (d, 1H), 5.31 (s, broad, 1H), 5.29 (s, 2H), 3.62-3.57 (m, 2H), 3.55-3.48 | | | |
| | (m, 2H), 3.35-3.30 (m, 2H), 2.36-2.26 (m, 5H), 1.78-1.65 (m 4H). | | | |
| 76 | 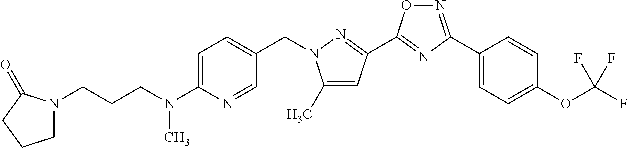 | 1.13 | 556 | F |
| | ¹H-NMR (400 MHz, CDCl₃, δ/ppm): 8.25 (d, 2H), 8.06 (s, 1H), 7.40-7.31 (m, 3H), | | | |
| | 6.76 (s, 1H), 6.42 (d, 1H), 5.30 (s, 2H), 3.55 (t, 2H), 3.38 (t, 2H), 3.32 (t, 2H), 3.01 (s, | | | |
| | 3H), 2.38 (t, 2H), 2.32 (s, 3H), 2.05-1.98 (m, 2H), 1.85-1.76 (m, 2H). | | | |
| 77 | 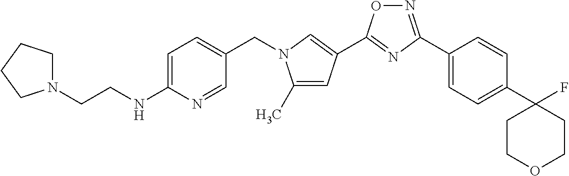 | 4.00 | 531 | A |
| | ¹H-NMR (400 MHz, CDCl₃, δ/ppm): 8.13 (d, 2H), 7.98 (s, 1H), 7.50 (d, 2H), 7.42 (s, | | | |
| | 1H), 7.16 (d, 1H), 6.54 (s, 1H), 6.40 (d, 1H), 5.19 (t, broad, 1H), 4.91 (s, 2H), 3.99-3.87 | | | |
| | (m, 4H), 3.38 (quart, 2H), 2.72 (t, 2H), 2.56-2.11 (m, 4H), 2.99-2.11 (m, 2H), | | | |
| | 2.25 (s, 3H), 1.97-1.91 (m, 2H), 1.80-1.76 (m, 4H). | | | |
| 78 | 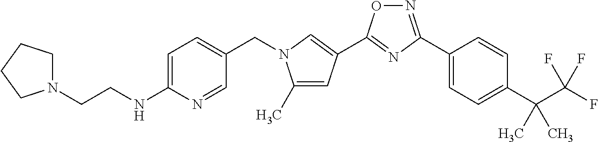 | 4.33 | 539 | B |
| | ¹H-NMR (400 MHz, CDCl₃, δ/ppm): 8.10 (d, 2H), 7.98 (d, 1H), 7.60 (d, 2H), 7.42 (d, | | | |
| | 1H), 7.16 (dd, 1H), 6.54 (d, 1H), 6.40 (d, 1H), 5.19 (t, broad, 1H), 4.91 (s, 2H), 3.38 | | | |
| | (quart, 2H), 2.72 (t, 2H), 2.56-2.52 (m, 4H), 2.25 (s, 3H), 1.80-1.76 (m, 4H), 1.61 (s, | | | |
| | 6H). | | | |
| 79 | 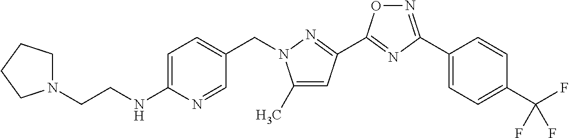 | 1.00 | 498 | M |
| | ¹H-NMR (400 MHz, CDCl₃, δ/ppm): 8.32 (d, 2H), 8.02 (d, 1H), 7.76 (d, 2H), 7.34 (dd, | | | |
| | 1H), 6.78 (s, 1H), 6.38 (d, 1H), 5.29 (s, 2H), 5.22 (t, broad, 1H), 3.38 (quart, 2H), 2.72 | | | |
| | (t, 2H), 2.59-2.52 (m, 4H), 2.32 (s, 3H), 1.85-1.69 (m, 4H). | | | |
| 80 | 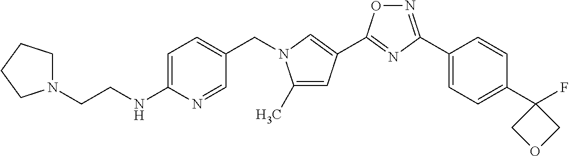 | 1.67 | 503 | D |
| | ¹H-NMR (400 MHz, CDCl₃, δ/ppm): 8.19 (d, 2H), 7.98 (d, 1H), 7.68 (d, 2H), 7.43 (d, | | | |
| | 1H), 7.16 (dd, 1H), 6.54 (d, 1H), 6.40 (d, 1H), 5.20 (t, broad, 1H), 5.05 (dd, 2H), 5.00 | | | |
| | (dd, 2H), 4.91 (s, 2H), 3.38 (quart, 2H), 2.72 (t, 2H), 2.56-2.52 (m, 4H), 2.25 (s, 3H), | | | |
| | 1.81-1.76 (m, 4H). | | | |
| 81 | 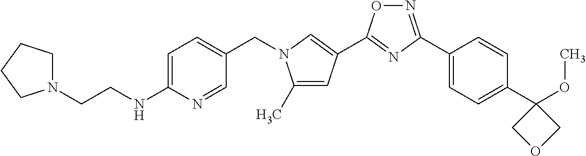 | 1.60 | 515 | D |
| | ¹H-NMR (400 MHz, CDCl₃, δ/ppm): 8.17 (d, 2H), 7.98 (d, 1H), 7.57 (d, 2H), 7.43 (d, | | | |
| | 1H), 7.16 (dd, 1H), 6.54 (d, 1H), 6.40 (d, 1H), 5.21 (t, broad, 1H), 4.95 (d, 2H), 4.91 (s, | | | |
| | 2H), 4.85 (d, 2H), 3.39 (quart, 2H), 3.17 (s, 3H), 2.73 (t, 2H), 2.58-2.53 (m, 4H), 2.25 | | | |
| | (s, 3H), 1.82-1.75 (m, 4H). | | | |
| 82 | 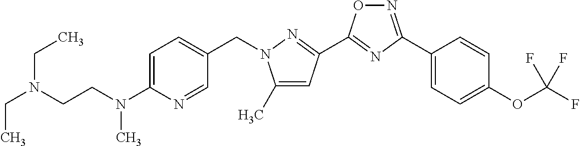 | 1.02 | 531 | O |
| 83 | 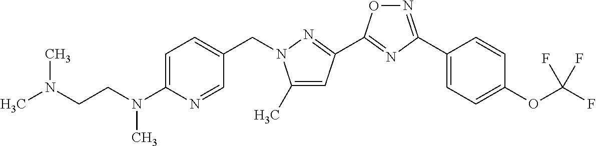 | 0.98 | 502 | O |
| 84 | 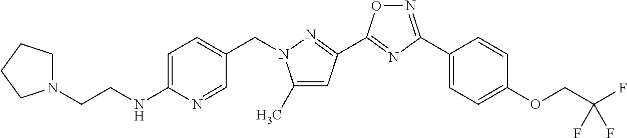 | 0.93 | 528 | M |
| | ¹H-NMR (400 MHz, CDCl₃, δ/ppm): 8.18 (d, 2H), 8.02 (d, 1H), 7.34 (dd, 1H), 7.05 (d, | | | |
| | 2H), 7.76 (s, 1H), 6.37 (d, 1H), 5.28 (s, 2H), 5.18 (t, broad, 1H), 4.43 (quart, 2H), 3.36 | | | |
| | (quart, 2H), 2.70 (t, 2H), 2.55-2.50 (m, 4H), 2.31 (s, 3H), 1.81-1.74 (m, 4H). | | | |
| 85 | 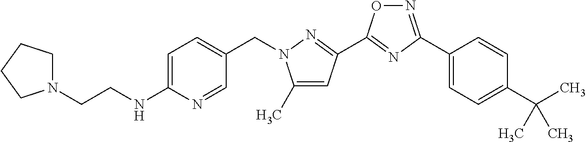 | 4.29 | 486 | A |
| | ¹H-NMR (400 MHz, CDCl₃, δ/ppm): 8.13 (d, 2H), 8.03 (d, 1H), 7.51 (d, 2H), 7.34 (dd, | | | |
| | 1H), 6.77 (s, 1H), 6.37 (d, 1H), 5.29 (s, 2H), 5.17 (t, broad, 1H), 3.36 (quart, 2H), 2.71 | | | |
| | (t, 2H), 2.56-2.50 (m, 4H), 2.31 (s, 3H), 1.81-1.74 (m, 4H), 1.36 (s, 9H). | | | |
| 86 | 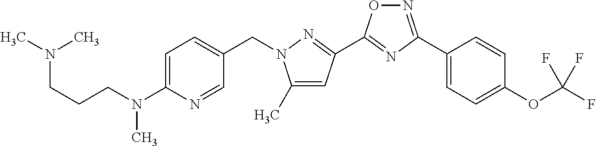 | 0.90 | 516 | O |
| 87 | 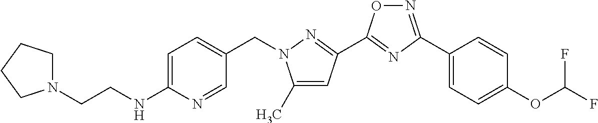 | 0.88 | 496 | M |
| | ¹H-NMR (400 MHz, CDCl₃, δ/ppm): 8.22 (d, 2H), 8.02 (d, 1H), 7.33 (dd, 1H), 7.22 (d, | | | |
| | 2H), 6.59 (t, 1H), 6.77 (s, 1H), 6.38 (d, 1H), 5.29 (s, 2H), 5.23 (t, broad, 1H), 3.38 | | | |
| | (quart, 2H), 2.72 (t, 2H), 2.60-2.52 (m, 4H), 2.31 (s, 3H), 1.82-1.72 (m, 4H). | | | |
| 88 | 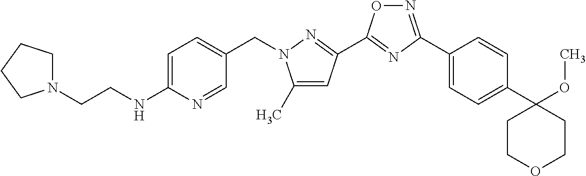 | 0.85 | 544 | M |
| | ¹H-NMR (400 MHz, CDCl₃, δ/ppm): 8.21 (d, 2H), 8.03 (d, 1H), 7.53 (d, 2H), 7.35 (dd, | | | |
| | 1H), 6.78 (s, 1H), 6.37 (d, 1H), 5.29 (s, 2H), 5.19 (t, broad, 1H), 3.93-3.82 (m, 4H), | | | |
| | 3.36 (quart, 2H), 3.01 (s, 3H), 2.71 (t, 2H), 2.56-2.51 (m, 4H), 2.32 (s, 3H), 2.11-1.98 | | | |
| | (m, 4H), 1.81-1.74 (m, 4H). | | | |
| 89 | 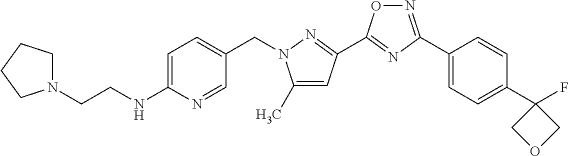 | 3.81 | 504 | A |
| | ¹H-NMR (400 MHz, CDCl₃, δ/ppm): 8.29 (d, 2H), 8.03 (d, 1H), 7.71 (d, 2H), 7.35 (dd, | | | |
| | 1H), 6.78 (s, 1H), 6.37 (d, 1H), 5.29 (s, 2H), 5.17 (t, broad, 1H), 5.06 (dd, 2H), 5.00 | | | |
| | (dd, 2H), 3.36 (quart, 2H), 2.70 (t, 2H), 2.55-2.50 (m, 4H), 2.32 (s, 3H), 1.80-1.74 (m, | | | |
| | 4H). | | | |
| 90 | 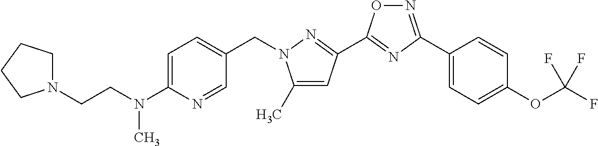 | 1.14 | 528 | F |
| | ¹H-NMR (400 MHz, CDCl₃, δ/ppm): 8.25 (d, 2H), 8.07 (s, 1H), 7.35 (dd, 1H), 7.32 (d, | | | |
| | 2H), 6.76 (s, 1H), 6.47 (d, 1H), 5.29 (s, 2H), 3.67 (t, 2H), 3.06 (s, 3H), 2.68 (t, 2H), | | | |
| | 2.59 (s, 4H), 2.32 (s, 3H), 1.79 (s, 4H). | | | |
| 91 | 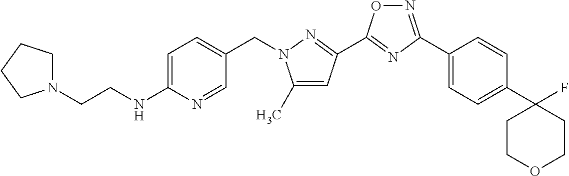 | 0.89 | 531 | M |
| | ¹H-NMR (400 MHz, CDCl₃, δ/ppm): 8.22 (d, 2H), 8.03 (d, 1H), 7.52 (d, 2H), 7.34 (dd, | | | |
| | 1H), 6.77 (s, 1H), 6.36 (d, 1H), 5.29 (s, 2H), 5.16 (t, broad, 1H), 4.00-3.87 (m, 4H), | | | |
| | 3.36 (quart, 2H), 2.70 (t, 2H), 2.55-2.50 (m, 4H), 2.32 (s, 3H), 2.30-2.11 (m, 2H), | | | |
| | 1.98-1.92 (m, 2H), 1.80-1.74 (m, 4H). | | | |
| 92 | 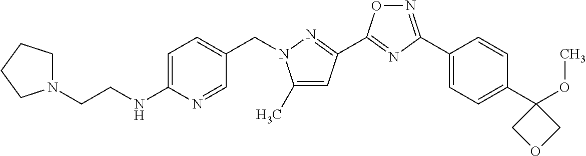 | 0.78 | 516 | M |
| | ¹H-NMR (400 MHz, CDCl₃, δ/ppm): 8.26 (d, 2H), 8.03 (d, 1H), 7.60 (d, 2H), 7.34 (dd, | | | |
| | 1H), 6.78 (s, 1H), 6.37 (d, 1H), 5.29 (s, 2H), 5.20 (t, broad, 1H), 4.96 (d, 2H), 4.85 (d, | | | |
| | 2H), 3.37 (quart, 2H), 3.18 (s, 3H), 2.71 (t, 2H), 2.57-2.52 (m, 4H), 2.32 (s, 3H), 1.81-1.74 | | | |
| | (m, 4H). | | | |
| 93 | 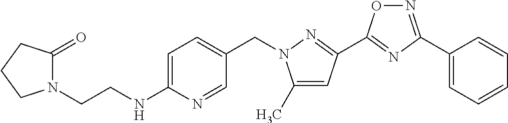 | 1.48 | 444 | C |
| | ¹H-NMR (400 MHz, CDCl₃, δ/ppm): 8.08 (dd, 2H), 7.99 (d, 1H), 7.65-7.57 (m, 3H), | | | |
| | 7.31 (dd, 1H), 6.88 (s, 1H), 6.67 (t, 1H), 6.44 (d, 1H), 5.29 (s, 2H), 3.44-3.28 (m, 6H), | | | |
| | 2.48 (s, 3H), 2.15 (t, 2H), 1.90-1.82 (m, 2H). | | | |
| 94 | 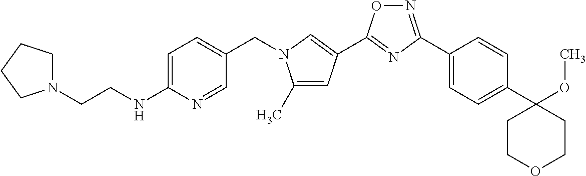 | 0.88 | 543 | M |
| | ¹H-NMR (400 MHz, CDCl₃, δ/ppm): 8.11 (d, 2H), 7.98 (d, 1H), 7.51 (d, 2H), 7.42 (d, | | | |
| | 1H), 7.16 (dd, 1H), 6.54 (d, 1H), 6.40 (d, 1H), 5.19 (t, broad, 1H), 4.91 (s, 2H), 3.92-3.82 | | | |
| | (m, 4H), 3.38 (quart, 2H), 3.00 (s, 3H), 2.72 (t, 2H), 2.57-2.51 (m, 4H), 2.25 (s, | | | |
| | 3H), 2.11-1.97 (m, 4H), 1.82-1.75 (m, 4H). | | | |
| 95 | 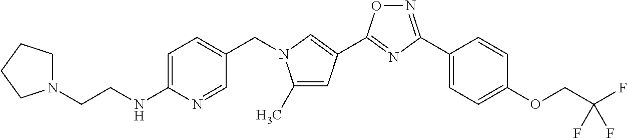 | 0.97 | 527 | M |
| | ¹H-NMR (400 MHz, CDCl₃, δ/ppm): 8.09 (d, 2H), 7.97 (d, 1H), 7 41 (d, 1H), 7.16 (dd, | | | |
| | 1H), 7.03 (d, 2H), 6.52 (d, 1H), 6.39 (d, 1H), 5.19 (t, broad, 1H), 4.92 (s, 2H), 4.41 | | | |
| | (quart, 2H), 3.38 (quart, 2H), 2.72 (t, 2H), 2.57-2.52 (m, 4H), 2.25 (s, 3H), 1.82-1.75 | | | |
| | (m, 4H). | | | |
| 96 | 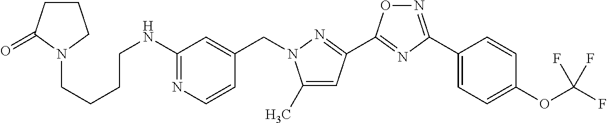 | 1.67 | 556 | N |
| 97 | 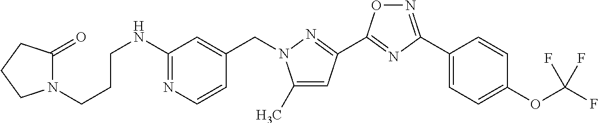 | 1.66 | 542 | N |
| 98 | 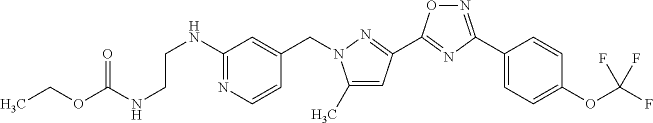 | 1.70 | 532 | N |
| 99 | 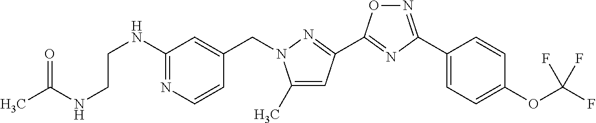 | 1.62 | 502 | N |

### Example 100

### 3-[(5-Methyl-3-{3-[4-(trifluoromethoxy)phenyl]-1,2,4-oxadiazol-5-yl}-1H-pyrazol-1-yl)methyl]-N-[2-(pyrrolidin-1-yl)ethyl]aniline

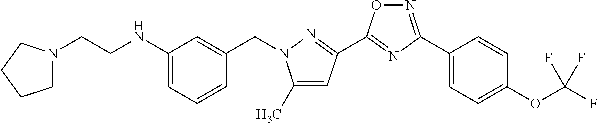

### Step 1: Benzyl {3-[(5-methyl-3-{3-[4-(trifluoromethoxy)phenyl]-1,2,4-oxadiazol-5-yl}-1H-pyrazol-1-yl)methyl]phenyl}carbamate

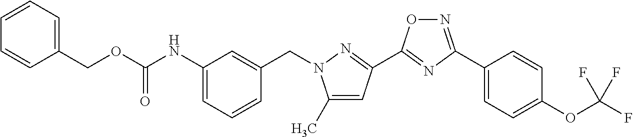

At 0° C., 130 μl (0.867 mmol) of benzyl chloroformate were added under inert conditions to a solution of 300 mg (0.722 mmol) of the compound from Example 61A and 252 μl (1.44 mmol) of N,N-diisopropylethylamine in 15 ml of anhydrous dichloromethane. The reaction mixture was then stirred at RT for 4 h. The mixture was then diluted with a little methanol and the complete reaction mixture was separated directly into its components by preparative HPLC (method K). 286 mg (72% of theory) of the title compound were obtained.

¹H-NMR (400 MHz, CDCl₃, δ/ppm): 8.23 (d, 2H), 7.37-7.27 (m, 9H), 7.21 (s, 1H), 6.84-6.79 (m, 3H), 5.41 (s, 2H), 5.18 (s, 2H), 2.26 (s, 3H).

HPLC (method B): Rₜ=5.26 min.

MS (ESIpos): m/z=550 [M+H]⁺.

### Step 2: Benzyl {3-[(5-methyl-3-{3-[4-(trifluoromethoxy)phenyl]-1,2,4-oxadiazol-5-yl}-1H-pyrazol-1-yl)methyl]phenyl}[2-(pyrrolidin-1-yl)ethyl]carbamate

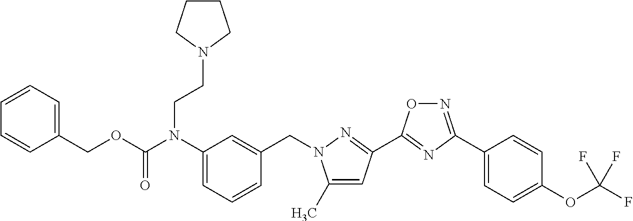

Under inert conditions, 47 mg (1.17 mmol) of a 60% suspension of sodium hydride in mineral oil were added to a solution of 160 mg (0.291 mmol) of the compound from Example 100/step 1 in 2.4 ml of anhydrous DMF. After 10 min of stirring at RT, a solution of 74.3 mg (0.437 mmol) of 1-(2-chloroethyl)pyrrolidine hydrochloride in 2.4 ml of anhydrous DMF was added. The reaction mixture was then heated at 80° C. for 4 h. After cooling to RT, the reaction mixture was diluted with a little methanol and the entire reaction mixture was separated directly into its components by preparative HPLC (method K). 119 mg (64% of theory) of the title compound were obtained.

¹H-NMR (400 MHz, CDCl₃, δ/ppm): 8.25 (d, 2H), 7.34-7.18 (m, 9H), 7.05-7.00 (m, 2H), 6.79 (s, 1H), 5.42 (s, 2H), 5.12 (s, 2H), 3.78 (t, 2H), 2.59 (t, 2H), 2.47-2.41 (m, 4H), 2.20 (s, 3H), 1.71-1.66 (m, 4H).

LC/MS (method C, ESIpos): Rₜ=1.98 min, m/z=647 [M+H]⁺.

### Step 3: 3-[(5-Methyl-3-{3-[4-(trifluoromethoxy)phenyl]-1,2,4-oxadiazol-5-yl}-1H-pyrazol-1-yl)methyl]-N-[2-(pyrrolidin-1-yl)ethyl]aniline

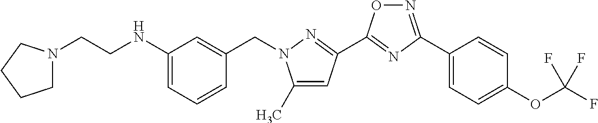

A suspension of 84 mg (0.130 mmol) of the compound from Example 100/step 2 in 4 ml of 6 M hydrochloric acid was stirred at a temperature of 50-60° C. for 7 days. 1 ml of saturated aqueous sodium bicarbonate solution was then added, the reaction mixture was stirred vigorously and the aqueous phase was then removed via an Extrelut NT3 cartridge. The filtrate was freed from the solvent on a rotary evaporator and the residue was chromatographed on a Pasteur pipette filled with silica gel (mobile phase: dichloromethane→cyclohexane/ethyl acetate 1:1→cyclohexane/ethyl acetate/triethylamine 30:30:1). 24 mg (36% of theory) of the title compound were obtained.

¹H-NMR (400 MHz, CDCl₃, δ/ppm): 8.26 (d, 2H), 7.33 (d, 2H), 7.12 (t, 1H), 6.80 (s, 1H), 6.54 (d, 1H), 6.48 (d, 2H), 6.40 (s, 1H), 5.37 (s, 2H), 4.31 (t, broad, 1H), 3.13 (quart, 2H), 2.69 (t, 2H), 2.52-2.47 (m, 4H), 2.29 (s, 3H), 1.78-1.74 (m, 4H).

LC/MS (method M, ESIpos): Rₜ=0.99 min, m/z=513 [M+H]⁺.

### Example 101

### N-(2-{4-[(5-Methyl-3-{3-[4-(trifluoromethoxy)phenyl]-1,2,4-oxadiazol-5-yl}-1H-pyrazol-1-yl)-methyl]phenoxy}ethyl)-N-(propan-2-yl)propane-2-amine

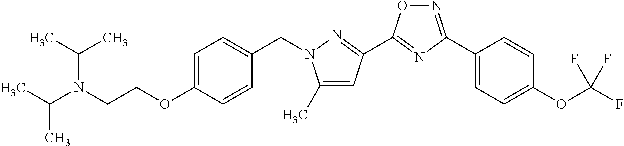

100 mg (0.240 mmol) of the compound from Example 59A and 172 mg (0.528 mmol) of caesium carbonate were initially charged in 2 ml of DMF. 58 mg (0.288 mmol) of N-(2-chloroethyl)-N-(propan-2-yl)propane-2-amine were added at RT and the stirred mixture was then heated at 150° C. for 2 h. After cooling to RT, the solid present was filtered off and the filtrate was purified by preparative HPLC (method L). The combined product-containing fractions were concentrated so that only a small residual volume of solvent remained. A little sodium bicarbonate was added, whereupon a solid precipitated out. This was filtered off, washed twice with water and dried under reduced pressure. 39 mg (30% of theory) of the title compound were obtained.

¹H-NMR (400 MHz, CDCl₃, δ/ppm): 8.26 (d, 2H), 7.32 (d, 2H), 7.12 (d, 2H), 6.83 (d, 2H), 6.79 (s, 1H), 5.38 (s, 2H), 3.86 (t, 2H), 3.09-2.98 (m, 2H), 2.79 (t, 2H), 2.28 (s, 3H), 1.01 (d, 12H).

LC/MS (method C, ESIpos): Rₜ=1.92 min, m/z=544 [M+H]⁺.

### Example 102

### N-[2-(1,1-Dioxidothiomorpholin-4-yl)ethyl]-5-[(5-methyl-3-{3-[4-(trifluoromethoxy)phenyl]-1,2,4-oxadiazol-5-yl}-1H-pyrazol-1-yl)methyl]pyridine-2-amine

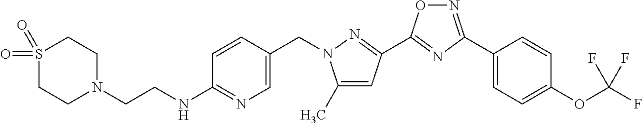

A solution of 74 mg (0.169 mmol) of the compound from Example 46A and 302 mg (1.69 mmol) of the compound from Example 98A in 1 ml of diethylene glycol dimethyl ether was heated in a microwave oven (CEM Discover, initial irradiation power 250 W) at 180° C. After 3 h, the reaction mixture was cooled to RT and purified directly by preparative HPLC (method K). The product fractions were combined and concentrated to dryness on a rotary evaporator. The residue was then dissolved in approx. 5 ml of methanol and the solution was passed through a bicarbonate cartridge (Polymerlabs, Stratospheres SPE, PL-HCO₃ MP SPE, capacity 0.9 mmol) in order to remove adhering formic acid originating from the HPLC purification. 30 mg (31% of theory) of the title compound were obtained.

¹H-NMR (400 MHz, CDCl₃, δ/ppm): 8.24 (d, 2H), 8.03 (d, 1H), 7.37 (dd, 1H), 7.33 (d, 2H), 6.78 (s, 1H), 6.37 (d, 1H), 5.30 (s, 2H), 4.89 (t, broad, 1H), 3.39 (quart, 2H), 3.06 (s, 8H), 2.78 (t, 2H), 2.33 (s, 3H).

HPLC (method A): Rₜ=4.12 min.

LC/MS (method M, ESIpos): Rₜ=0.98 min, m/z=578 [M+H]⁺.

### Example 103

### N-[2-(4,4-Difluoropiperidin-1-yl)ethyl]-5-[(5-methyl-3-{3-[4-(trifluoromethoxy)phenyl]-1,2,4-oxa-diazol-5-yl}-1H-pyrazol-1-yl)methyl]pyridine-2-amine

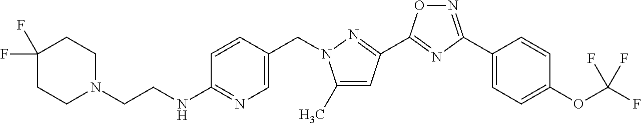

Analogously to the process described in Example 102, 89 mg (69% of theory) of the title compound were obtained from 100 mg (0.229 mmol) of the compound from Example 46A and 377 mg (2.29 mmol) of the compound from Example 99A.

¹H-NMR (400 MHz, CDCl₃, δ/ppm): 8.25 (d, 2H), 8.03 (d, 1H), 7.37 (dd, 1H), 7.33 (d, 2H), 6.77 (s, 1H), 6.37 (d, 1H), 5.30 (s, 2H), 5.08 (t, broad, 1H), 3.33 (quart, 2H), 2.65 (t, 2H), 2.60-2.55 (m, 4H), 2.33 (s, 3H), 2.03-1.94 (m, 4H).

HPLC (method A): Rₜ=4.13 min.

LC/MS (method F, ESIpos): Rₜ=1.19 min, m/z=564 [M+H]⁺.

### Example 104

### N-[2-(2,2-Dimethylpyrrolidin-1-yl)ethyl]-5-[(5-methyl-3-{3-[4-(trifluoromethoxy)phenyl]-1,2,4-oxadiazol-5-yl}-1H-pyrazol-1-yl)methyl]pyridine-2-amine

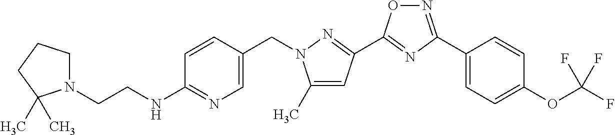

100 mg (0.229 mmol) of the compound from Example 46A and 163 mg (1.15 mmol) of the compound from Example 100A were stirred at 150° C. (oil bath temperature) for 15 h. After cooling, the reaction mixture was diluted with about 4 ml of acetonitrile and then purified directly by preparative HPLC (method K). The product fractions were combined and concentrated to dryness on a rotary evaporator. The residue was then dissolved in approx. 5 ml of methanol and the solution was passed through a bicarbonate cartridge (Polymerlabs, Stratospheres SPE, PL-HCO₃ MP SPE, capacity 0.9 mmol) in order to remove adhering formic acid originating from the HPLC purification. 13 mg (10% of theory) of the title compound were obtained.

¹H-NMR (400 MHz, CDCl₃, δ/ppm): 8.24 (d, 2H), 8.02 (d, 1H), 7.34 (dd, 1H), 7.33 (d, 2H), 6.77 (s, 1H), 6.35 (d, 1H), 5.28 (s, 2H), 5.23 (t, broad, 1H), 3.26 (quart, 2H), 2.71 (t, 2H), 2.62 (t, 2H), 2.31 (s, 3H), 1.78-1.69 (m, 2H), 1.64-1.61 (m, 2H), 0.98 (s, 6H).

LC/MS (method F, ESIpos): Rₜ=1.22 min, m/z=542 [M+H]⁺.

### Example 105

### N-[2-(4-Fluoropiperidin-1-yl)ethyl]-5-[(5-methyl-3-{3-[4-(trifluoromethoxy)phenyl]-1,2,4-oxa-diazol-5-yl}-1H-pyrazol-1-yl)methyl]pyridine-2-amine

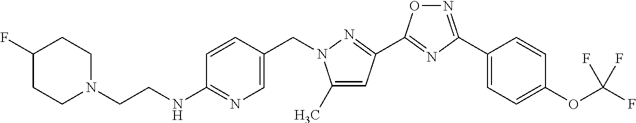

Analogously to the process described in Example 102, 135 mg (72% of theory) of the title compound were obtained from 150 mg (0.344 mmol) of the compound from Example 46A and 503 mg (3.44 mmol) of the compound from Example 101A.

¹H-NMR (400 MHz, CDCl₃, δ/ppm): 8.25 (d, 2H), 8.03 (d, 1H), 7.36 (dd, 1H), 7.33 (d, 2H), 6.78 (s, 1H), 6.37 (d, 1H), 5.29 (s, 2H), 5.15 (t, broad, 1H), 4.77-4.60 (m, 1H), 3.32 (quart, 2H), 2.62-2.57 (m, 4H), 2.42-2.37 (m, 2H), 2.32 (s, 3H), 1.96-1.83 (m, 4H).

HPLC (method A): Rₜ=4.10 min.

MS (DCI, NH₃): m/z=546 [M+H]⁺.

LC/MS (method D, ESIpos): Rₜ=1.95 min, m/z=546 [M+H]⁺.

### Example 106

### 5-[(3-{3-[4-(1-Fluorocyclobutyl)phenyl]-1,2,4-oxadiazol-5-yl}-5-methyl-1H-pyrazol-1-yl)methyl]-N-[2-(pyrrolidin-1-yl)ethyl]pyridine-2-amine

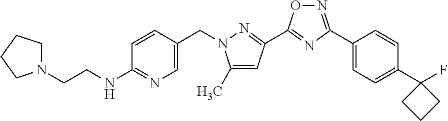

Analogously to the process described in Example 102, 32 mg (46% of theory) of the title compound were obtained from 60 mg (0.142 mmol) of the compound from Example 103A and 323 mg (2.83 mmol) of 1-(2-aminoethyl)pyrrolidine.

¹H-NMR (400 MHz, CDCl₃, δ/ppm): 8.23 (d, 2H), 8.03 (d, 1H), 7.59 (d, 2H), 7.34 (dd, 1H), 6.78 (s, 1H), 6.37 (d, 1H), 5.29 (s, 2H), 5.16 (t, broad, 1H), 3.37 (quart, 2H), 2.70 (t, 2H), 2.73-2.57 (m, 4H), 2.53-2.50 (m, 4H), 2.32 (s, 3H), 2.19-2.08 (m, 1H), 1.87-1.77 (m, 1H), 1.80-1.75 (m, 4H).

LC/MS (method M, ESIpos): Rₜ=0.98 min, m/z=502 [M+H]⁺.

### Example 107

### 5-[(3-{3-[4-(1-Methoxycyclobutyl)phenyl]-1,2,4-oxadiazol-5-yl}-5-methyl-1H-pyrazol-1-yl)-methyl]-N-[2-(pyrrolidin-1-yl)ethyl]pyridine-2-amine

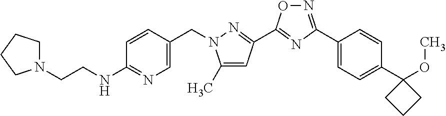

Analogously to the process described in Example 102, 80 mg (85% of theory) of the title compound were obtained from 80 mg (0.184 mmol) of the compound from Example 104A and 419 mg (3.67 mmol) of 1-(2-aminoethyl)pyrrolidine.

¹H-NMR (400 MHz, CDCl₃, δ/ppm): 8.21 (d, 2H), 8.02 (d, 1H), 7.55 (d, 2H), 7.35 (dd, 1H), 6.78 (s, 1H), 6.38 (d, 1H), 5.29 (s, 2H), 5.17 (t, broad, 1H), 3.35 (quart, 2H), 2.97 (s, 3H), 2.70 (t, 2H), 2.55-2.50 (m, 4H), 2.44-2.40 (m, 4H), 2.32 (s, 3H), 2.02-1.94 (m, 1H), 1.79-1.75 (m, 4H), 1.79-1.69 (m, 1H).

HPLC (method A): Rₜ=3.98 min.

LC/MS (method M, ESIpos): Rₜ=0.93 min, m/z=514 [M+H]⁺.

### Example 108

### 5-[(5-Methyl-3-{3-[4-(piperidin-1-yl)phenyl]-1,2,4-oxadiazol-5-yl}-1H-pyrazol-1-yl)methyl]-N-[2-(pyrrolidin-1-yl)ethyl]pyridine-2-amine

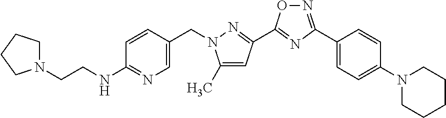

Analogously to the process described in Example 102, 19 mg (63% of theory) of the title compound were obtained from 26 mg (0.060 mmol) of the compound from Example 105A and 136 mg (1.20 mmol) of 1-(2-aminoethyl)pyrrolidine.

¹H-NMR (400 MHz, CDCl₃, δ/ppm): 8.05 (d, 2H), 8.02 (d, 1H), 7.33 (dd, 1H), 6.97 (d, 2H), 6.74 (s, 1H), 6.37 (d, 1H), 5.27 (s, 2H), 5.13 (t, broad, 1H), 3.34 (quart, 2H), 3.31-3.29 (m, 4H), 2.70 (t, 2H), 2.54-2.50 (m, 4H), 2.30 (s, 3H), 1.78-1.74 (m, 4H), 1.72-1.67 (m, 4H), 1.64-1.61 (m, 2H).

HPLC (method A): Rₜ=3.98 min.

LC/MS (method F, ESIpos): Rₜ=1.12 min, m/z=513 [M+H]⁺.

### Example 109

### 5-[(5-Methyl-3-{3-[4-(methylsulphonyl)phenyl]-1,2,4-oxadiazol-5-yl}-1H-pyrazol-1-yl)methyl]-N-[2-(pyrrolidin-1-yl)ethyl]pyridine-2-amine

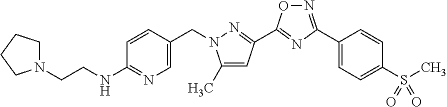

Analogously to the process described in Example 102, 54 mg (57% of theory) of the title compound were obtained from 80 mg (0.186 mmol) of the compound from Example 106A and 425 mg (3.72 mmol) of 1-(2-aminoethyl)pyrrolidine.

¹H-NMR (400 MHz, CDCl₃, δ/ppm): 8.43 (d, 2H), 8.08 (d, 2H), 8.03 (d, 1H), 7.33 (dd, 1H), 6.89 (s, 1H), 6.38 (d, 1H), 5.29 (s, 2H), 5.21 (t, broad, 1H), 3.37 (quart, 2H), 3.31 (s, 3H), 2.70 (t, 2H), 2.55-2.51 (m, 4H), 2.33 (s, 3H), 1.79-1.75 (m, 4H).

LC/MS (method M, ESIpos): Rₜ=0.73 min, m/z=508 [M+H]⁺.

### Example 110

### 1-[4-(5-{5-Methyl-1-[(6-{[2-(pyrrolidin-1-yl)ethyl]amino}pyridin-3-yl)methyl]-1H-pyrazol-3-yl}-1,2,4-oxadiazol-3-yl)phenyl]cyclobutanol

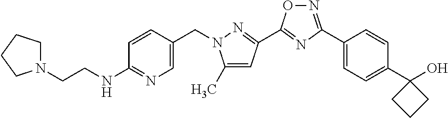

Analogously to the process described in Example 102, 63 mg (91% of theory) of the title compound were obtained from 58 mg (0.137 mmol) of the compound from Example 107A and 314 mg (2.75 mmol) of 1-(2-aminoethyl)pyrrolidine.

¹H-NMR (400 MHz, CDCl₃, δ/ppm): 8.21 (d, 2H), 8.02 (d, 1H), 7.63 (d, 2H), 7.33 (dd, 1H), 6.77 (s, 1H), 6.37 (d, 1H), 5.28 (s, 2H), 5.19 (t, broad, 1H), 3.36 (quart, 2H), 2.70 (t, 2H), 2.63-2.57 (m, 2H), 2.54-2.50 (m, 4H), 2.45-2.37 (m, 2H), 2.31 (s, 3H), 2.15-2.03 (m, 1H), 1.82-1.70 (m, 5H).

LC/MS (method M, ESIpos): Rₜ=0.81 min, m/z=500 [M+H]⁺.

### Example 111

### 5-{[5-Methyl-3-(3-{4-[(trifluoromethyl)sulphonyl]phenyl}-1,2,4-oxadiazol-5-yl)-1H-pyrazol-1-yl]methyl}-N-[2-(pyrrolidin-1-yl)ethyl]pyridine-2-amine1-oxide

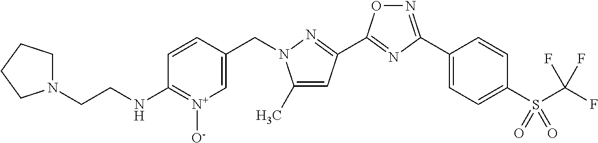

Under argon, a mixture of 200 mg (0.40 mmol) of the compound from Example 108A and 1.0 ml (8.00 mmol) of 2-(pyrrolidin-1-yl)ethanamine was initially stirred at 40° C. for 24 h and then at 50° C. for a further 24 h. The reaction mixture was then purified directly by preparative HPLC (method L). The combined product fractions were concentrated on a rotary evaporator until only a small residual volume of liquid remained. Saturated aqueous sodium bicarbonate solution was added and the mixture was extracted twice with ethyl acetate. The combined ethyl acetate phases were dried over magnesium sulphate, filtered and concentrated. 42 mg (17% of theory, purity 90%) of the title compound were obtained.

LC/MS (method M, ESIpos): Rₜ=0.88 min, m/z=578 [M+H]⁺.

### Example 112

### 5-{[5-Methyl-3-(3-{4-[(trifluoromethyl)sulphonyl]phenyl}-1,2,4-oxadiazol-5-yl)-1H-pyrazol-1-yl]methyl}-N-[2-(pyrrolidin-1-yl)ethyl]pyridine-2-amine

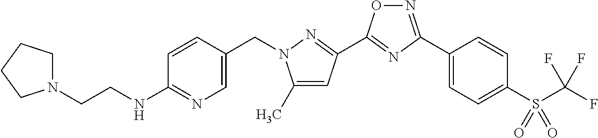

42 mg (0.065 mmol, purity 90%) of the compound from Example 111 were initially charged in 1 ml of dichloromethane, and the mixture was cooled to 0° C. 49 μl (0.098 mmol) of a 2 M solution of phosphorus trichloride in dichloromethane were then added, and the mixture was stirred initially at 0° C. for 10 min and then at RT for 30 min. A further 25 μl (0.049 mmol) of the 2 M solution of phosphorus trichloride in dichloromethane were then added, and the mixture was stirred at RT overnight. 1 ml of water was added, and the mixture was stirred for a few minutes, made weakly alkaline using saturated aqueous sodium bicarbonate solution and extracted three times with in each case 4 ml of ethyl acetate. The combined organic phases were washed once with saturated sodium chloride solution, dried over magnesium sulphate, filtered and concentrated. After the residue had been dried in vacuo, 24 mg (66% of theory) of the title compound were obtained.

¹H-NMR (400 MHz, CDCl₃, δ/ppm): 8.53 (d, 2H), 8.18 (d, 2H), 8.02 (s, 1H), 7.35 (dd, 1H), 6.80 (s, 1H), 6.40 (d, 1H), 5.41 (s, broad, 1H), 5.30 (s, 2H), 3.48-3.39 (m, 2H), 2.87-2.77 (m, 2H), 2.67 (s, broad, 4H), 2.35 (s, 3H), 1.83 (s, broad, 4H).

LC/MS (method M, ESIpos): Rₜ=0.94 min, m/z=562 [M+H]⁺.

### Example 113

### 5-[(5-Methyl-3-{3-[4-(pentafluoro-λ⁶-sulphanyl)phenyl]-1,2,4-oxadiazol-5-yl}-1H-pyrazol-1-yl)-methyl]-N-[2-(pyrrolidin-1-yl)ethyl]pyridine-2-amine

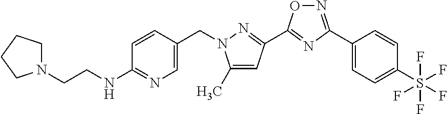

Analogously to the process described in Example 38, 200 mg (0.419 mmol) of the compound from Example 109A and 478 mg (4.18 mmol) of 2-(pyrrolidin-1-yl)ethanamine were reacted to give 122 mg (52% of theory) of the title compound. In this case, the reaction time was 90 h at a bath temperature of 100° C.

¹H-NMR (400 MHz, CDCl₃, δ/ppm): 8.31 (d, 2H), 8.02 (s, 1H), 7.89 (d, 2H), 7.32 (d, 1H), 6.78 (s, 1H), 6.39 (d, 1H), 5.29 (s, 3H), 3.42-3.37 (dd, 2H), 2.77-2.73 (t, 2H), 2.59 (s, 4H), 2.32 (s, 3H), 1.80 (s, 4H).

LC/MS (method F, ESIpos): Rₜ=1.14 min, m/z=556 [M+H]⁺.

### Example 114

### 5-[(5-Methyl-3-{3-[4-(1H-pyrrol-1-ylmethyl)phenyl]-1,2,4-oxadiazol-5-yl}-1H-pyrazol-1-yl)-methyl]-N-[2-(pyrrolidin-1-yl)ethyl]pyridine-2-amine

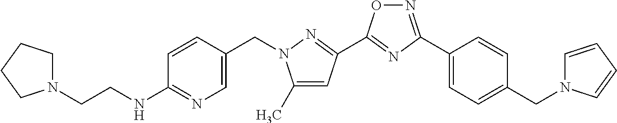

Analogously to the process described in Example 38, 155 mg (0.360 mmol) of the compound from Example 110A and 205 mg (1.80 mmol) of 2-(pyrrolidin-1-yl)ethanamine were reacted to give 127 mg (69% of theory) of the title compound. In this case, the reaction time was 15 h at a bath temperature of 150° C.

¹H-NMR (400 MHz, CDCl₃, δ/ppm): 8.15 (d, 2H), 8.01 (d, 1H), 7.34-7.31 (dd, 1H), 7.22 (d, 2H), 6.75 (s, 1H), 6.72-6.71 (t, 2H), 6.39 (d, 1H), 6.23-6.21 (dd, 2H), 5.28 (s, 2H), 5.12 (s, 2H), 3.42-3.38 (m, 2H), 2.78-2.72 (m, 2H), 2.64-2.52 (m, 4H), 2.31 (s, 3H), 1.83-1.78 (m, 4H).

LC/MS (method M, ESIpos): Rₜ=0.91 min, m/z=509 [M+H]⁺.

### Example 115

### 5-{[3-(3-{4-[(Diisopropylamino)methyl]phenyl}-1,2,4-oxadiazol-5-yl)-5-methyl-1H-pyrazol-1-yl]-methyl}-N-[2-(pyrrolidin-1-yl)ethyl]pyridine-2-amine

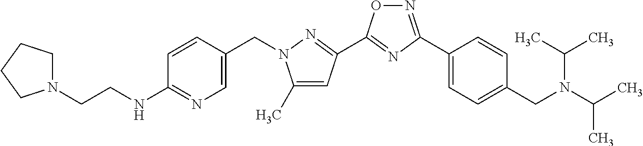

Analogously to the process described in Example 38, 210 mg (0.452 mmol) of the compound from Example 111A and 260 mg (2.26 mmol) of 2-(pyrrolidin-1-yl)ethanamine were reacted to give 156 mg (64% of theory) of the title compound. In this case, the reaction time was 15 h at a bath temperature of 150° C.

¹H-NMR (400 MHz, CDCl₃, δ/ppm): 8.11 (d, 2H), 8.01 (d, 1H), 7.50 (d, 2H), 7.36-7.32 (dd, 1H), 6.76 (s, 1H), 6.38 (d, 1H), 5.29 (s, 2H), 5.28 (s, broad, 1H), 3.70 (s, 2H), 3.42-3.38 (quart, 2H), 3.09-2.99 (m, 2H), 2.78-2.73 (t, 2H), 2.59 (s, 4H), 2.31 (s, 3H), 1.82-1.78 (m, 4H), 1.02 (d, 12H).

HPLC (method P): Rₜ=4.59 min.

MS (DCI, NH₃): m/z=543 [M+H]⁺.

LC/MS (method D, ESIpos): Rₜ=1.14/1.20 min, m/z=543 [M+H]⁺.

### Example 116

### 5-{[4-(3-{4-[(Diisopropylamino)methyl]phenyl}-1,2,4-oxadiazol-5-yl)-2-methyl-1H-pyrrol-1-yl]-methyl}-N-[2-(pyrrolidin-1-yl)ethyl]pyridine-2-amine

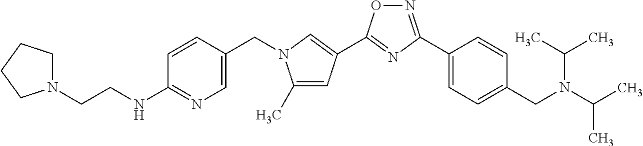

Analogously to the process described in Example 38, 60 mg (0.129 mmol) of the compound from Example 112A and 75 mg (0.647 mmol) of 2-(pyrrolidin-1-yl)ethanamine were reacted to give 37 mg (53% of theory) of the title compound. In this case, the reaction time was 15 h at a bath temperature of 150° C.

¹H-NMR (400 MHz, CDCl₃, δ/ppm): 8.01 (d, 2H), 7.93 (d, 1H), 7.48 (d, 2H), 7.42 (d, 1H), 7.18-7.14 (dd, 1H), 6.52 (s, 1H), 6.42 (d, 1H), 5.50 (s, broad, 1H), 4.90 (s, 2H), 3.68 (s, 2H), 3.52-3.46 (m, 2H), 3.07-2.98 (m, 2H), 2.88-2.83 (t, 2H), 2.72 (s, broad, 4H), 2.23 (s, 3H), 1.90-1.82 (m, 4H), 1.02 (d, 12H).

LC/MS (method D, ESIpos): Rₜ=1.24/1.26 min, m/z=542 [M+H]⁺.

### Example 117

### 5-[(2-Methyl-4-{3-[4-(1H-pyrrol-1-ylmethyl)phenyl]-1,2,4-oxadiazol-5-yl}-1H-pyrrol-1-yl)-methyl]-N-[2-(pyrrolidin-1-yl)ethyl]pyridine-2-amine

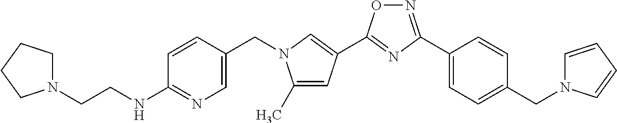

Analogously to the process described in Example 38, 45 mg (0.105 mmol) of the compound from Example 113A and 60 mg (0.523 mmol) of 2-(pyrrolidin-1-yl)ethanamine were reacted to give 26 mg (49% of theory) of the title compound. In this case, the reaction time was 15 h at a bath temperature of 150° C.

¹H-NMR (400 MHz, CDCl₃, δ/ppm): 8.06 (d, 2H), 7.96 (d, 1H), 7.40 (d, 1H), 7.21 (d, 2H), 7.18-7.12 (dd, 1H), 6.70 (s, 2H), 6.52 (s, 1H), 6.41 (d, 1H), 6.21 (s, 2H), 5.32 (s, broad, 1H), 5.12 (s, 2H), 4.90 (s, 2H), 3.48-3.40 (m, 2H), 2.82-2.77 (t, 2H), 2.70-2.60 (s, broad, 4H), 2.22 (s, 3H), 1.81 (s, broad, 4H).

LC/MS (method M, ESIpos): Rₜ=0.96 min, m/z=508 [M+H]⁺.

### Example 118

### 5-[(4-{3-[4-(2-Fluoropropan-2-yl)phenyl]-1,2,4-oxadiazol-5-yl}-2-methyl-1H-pyrrol-1-yl)methyl]-N-[2-(pyrrolidin-1-yl)ethyl]pyridine-2-amine

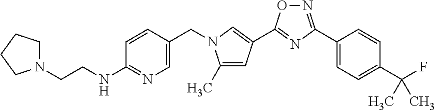

Analogously to the process described in Example 38, 63 mg (0.153 mmol) of the compound from Example 114A and 88 mg (0.767 mmol) of 2-(pyrrolidin-1-yl)ethanamine were reacted to give 22 mg (29% of theory) of the title compound. In this case, the reaction time was 15 h at a bath temperature of 150° C.

¹H-NMR (400 MHz, CDCl₃, δ/ppm): 8.10 (d, 2H), 7.97 (s, 1H), 7.48 (d, 2H), 7.42 (d, 1H), 7.16 (dd, 1H), 6.54 (s, 1H), 6.42 (d, 1H), 5.38 (s, broad, 1H), 4.91 (s, 2H), 3.47-3.39 (m, 2H), 2.80 (t, 2H), 2.64 (s, broad, 4H), 2.25 (s, 3H), 1.82 (s, broad, 4H), 1.74 (s, 3H), 1.69 (s, 3H).

LC/MS (method M, ESIpos): Rₜ=0.93 min, m/z=489 [M+H]⁺.

### Example 119

### 5-[(2-Methyl-4-{3-[4-(trifluoromethyl)phenyl]-1,2,4-oxadiazol-5-yl}-1H-pyrrol-1-yl)methyl]-N-[2-(pyrrolidin-1-yl)ethyl]pyridine-2-amine

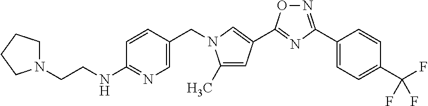

Analogously to the process described in Example 38, 76 mg (0.181 mmol) of the compound from Example 115A and 105 mg (0.907 mmol) of 2-(pyrrolidin-1-yl)ethanamine were reacted to give 60 mg (67% of theory) of the title compound. In this case, the reaction time was 15 h at a bath temperature of 150° C.

¹H-NMR (400 MHz, CDCl₃, δ/ppm): 8.24 (d, 2H), 7.97 (d, 1H), 7.78-7.64 (d, 2H), 7.43 (d, 1H), 7.17 (dd, 1H), 6.54 (d, 1H), 6.41 (d, 1H), 5.30 (s, broad, 1H), 4.92 (s, 2H), 3.42 (quart, 2H), 2.78 (t, 2H), 2.26 (s, 3H), 1.86-1.71 (m, 4H).

LC/MS (method M, ESIpos): Rₜ=0.99 min, m/z=497 [M+H]⁺.

### Example 120

### 5-[(2-Methyl-4-{3-[4-(trimethylsilyl)phenyl]-1,2,4-oxadiazol-5-yl}-1H-pyrrol-1-yl)methyl]-N-[2-(pyrrolidin-1-yl)ethyl]pyridine-2-amine

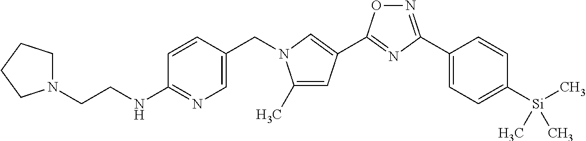

Analogously to the process described in Example 38, 68 mg (0.160 mmol) of the compound from Example 116A and 92 mg (0.798 mmol) of 2-(pyrrolidin-1-yl)ethanamine were reacted to give 53 mg (66% of theory) of the title compound. In this case, the reaction time was 15 h at a bath temperature of 150° C.

¹H-NMR (400 MHz, CDCl₃, δ/ppm): 8.08 (d, 2H), 7.97 (d, 1H), 7.62 (d, 2H), 7.42 (d, 1H), 7.16 (dd, 1H), 6.54 (s, 1H), 6.41 (d, 1H), 5.27 (s, broad, 1H), 4.91 (s, 2H), 3.41 (quart, 2H), 2.76 (t, 2H), 2.59 (s, broad, 4H), 2.25 (s, 3H), 1.80 (s, broad, 4H), 0.30 (s, 9H).

LC/MS (method M, ESIpos): Rₜ=1.12 min, m/z=501 [M+H]⁺.

### Example 121

### 5-[(2-Methyl-4-{3-[4-(pentafluoro-λ⁶-sulphanyl)phenyl]-1,2,4-oxadiazol-5-yl}-1H-pyrrol-1-yl)-methyl]-N-[2-(pyrrolidin-1-yl)ethyl]pyridine-2-amine

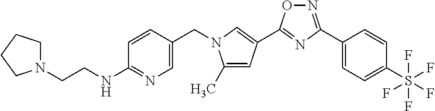

Analogously to the process described in Example 38, 178 mg (0.373 mmol) of the compound from Example 117A and 426 mg (3.73 mmol) of 2-(pyrrolidin-1-yl)ethanamine were reacted to give 86 mg (41% of theory) of the title compound. In this case, the reaction time was 90 h at a bath temperature of 100° C.

¹H-NMR (400 MHz, CDCl₃, δ/ppm): 8.22 (d, 2H), 7.96 (d, 1H), 7.86 (d, 2H), 7.43 (d, 1H), 7.17 (dd, 1H), 6.53 (s, 1H), 6.42 (d, 1H), 5.36 (s, broad, 1H), 4.91 (s, 2H), 3.44 (quart, 2H), 2.80 (t, 2H), 2.64 (s, broad, 4H), 2.26 (s, 3H), 1.88-1.76 (m, 4H).

LC/MS (method M, ESIpos): Rₜ=0.98 min, m/z=555 [M+H]⁺.

### Example 122

### N-[2-(2,4-Dioxo-1,3-thiazolidin-3-yl)ethyl]-3-[(5-methyl-3-{3-[4-(trifluoromethoxy)phenyl]-1,2,4-oxadiazol-5-yl}-1H-pyrazol-1-yl)methyl]benzamide

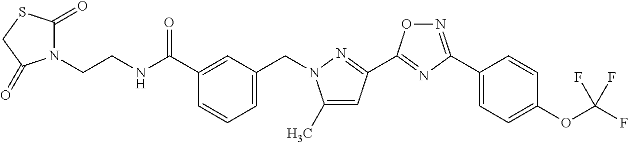

Analogously to the process described in Example 48, 80 mg (0.180 mmol) of the compound from Example 56A and 71 mg (0.360 mmol) of 3-(2-aminoethyl)-1,3-thiazolidine-2,4-dione hydrochloride [P. M. Kushakova et al., Chem. Heterocycl. Comp. 2006, 42 (2), 221-226] gave 67 mg (63% of theory) of the title compound.

¹H-NMR (400 MHz, CDCl₃, δ/ppm): 8.25 (d, 2H), 7.67 (dd, 1H), 7.55 (d, 1H), 7.41 (t, 1H), 7.33 (d, 2H), 7.30 (dd, 1H), 6.82 (s, 1H), 6.58 (t, broad, 1H), 5.49 (s, 2H), 3.93 (s, 2H), 3.92-3.90 (m, 2H), 3.70-3.66 (m, 2H), 2.30 (s, 3H).

LC/MS (method C, ESIpos): Rₜ=2.64 min, m/z=587 [M+H]⁺.

### Example 123

### 4-(2-{3-[(5-Methyl-3-{3-[4-(trifluoromethoxy)phenyl]-1,2,4-oxadiazol-5-yl}-1H-pyrazol-1-yl)-methyl]phenoxy}ethyl)morpholine

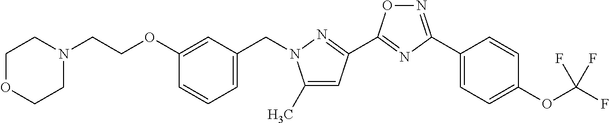

63 μl (0.317 mmol) of diisopropyl azodicarboxylate (DIAD) were added to a solution of 83 mg (0.317 mmol) of triphenylphosphine in 2 ml of anhydrous THF, and the mixture was stirred at RT for 5 min. A solution of 39 μl (0.317 mmol) of 4-(2-hydroxyethyl)morpholine and of 120 mg (0.288 mmol) of the compound from Example 58A, in each case in 1 ml of anhydrous THF, were then added in succession. The reaction mixture was stirred at RT for 3 days and then diluted with in each case 1 ml of methanol and DMF. The solution obtained in this manner was separated directly into its components by preparative HPLC (method K). The product fractions were combined, freed from the solvent on a rotary evaporator and purified again by MPLC (silica gel; mobile phase: dichloromethane/methanol 10:1). 31 mg (19% of theory) of the title compound were obtained.

¹H-NMR (400 MHz, CDCl₃, δ/ppm): 8.25 (d, 2H), 7.33 (d, 2H), 7.24 (t, 1H), 6.83 (dd, 1H), 6.81 (s, 1H), 6.76 (dd, 1H), 6.70 (d, 1H), 5.42 (s, 2H), 4.06 (t, 2H), 3.71 (m, 4H), 2.77 (t, 2H), 2.54 (m, 4H), 2.28 (s, 3H).

LC/MS (method M, ESIpos): Rₜ=0.99 min, m/z=530 [M+H]⁺.

### Example 124

### N,N-Dimethyl-2-{3-[(5-methyl-3-{3-[4-(trifluoromethoxy)phenyl]-1,2,4-oxadiazol-5-yl}-1H-pyrazol-1-yl)methyl]phenoxy}ethanamine

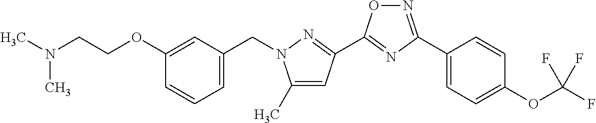

A mixture of 100 mg (0.240 mmol) of the compound from Example 58A, 42 mg (0.288 mmol) of 2-chloro-N,N-dimethylethanamine hydrochloride and 83 mg (0.600 mmol) of potassium carbonate in 3 ml of DMF was stirred at 80° C. for 2 days. After cooling, undissolved salts were filtered off. The filtrate was diluted with about 2 ml of methanol and separated into its components by preparative HPLC (method K). The product fractions were combined and concentrated to dryness on a rotary evaporator. The product obtained was dissolved in approx. 5 ml of methanol and the solution was passed through a bicarbonate cartridge (Polymerlabs, Stratospheres SPE, PL-HCO₃ MP SPE, capacity 0.9 mmol) in order to remove adhering formic acid originating from the HPLC purification. 44 mg (34% of theory) of the title compound were obtained.

¹H-NMR (400 MHz, CDCl₃, δ/ppm): 8.26 (d, 2H), 7.33 (d, 2H), 7.23 (t, 1H), 6.83 (dd, 1H), 6.81 (s, 1H), 6.74 (dd, 1H), 6.71 (d, 1H), 5.42 (s, 2H), 4.00 (t, 2H), 2.69 (t, 2H), 2.31 (s, 6H), 2.27 (s, 3H).

LC/MS (method M, ESIpos): Rₜ=0.98 min, m/z=488 [M+H]⁺.

### Example 125

### 4-{4-[(5-Methyl-3-{3-[4-(trifluoromethoxy)phenyl]-1,2,4-oxadiazol-5-yl}-1H-pyrazol-1-yl)-methyl]benzyl}morpholine

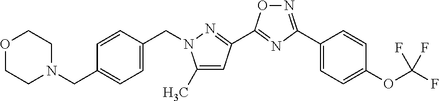

Analogously to the process described in Example 42A, 100 mg (0.322 mmol) of the compound from Example 17A and 110 mg (0.419 mmol) of the compound from Example 97A were reacted to give 102 mg (63% of theory) of the title compound. In this case, the reaction mixture was stirred at 50° C. for 2 days. The purification of the crude product was carried out by preparative HPLC (method L).

¹H-NMR (400 MHz, CDCl₃, δ/ppm): 8.25 (d, 2H), 7.36-7.28 (m, 4H), 7.12 (d, 2H), 6.80 (s, 1H), 5.42 (s, 2H), 3.72-3.68 (t, 4H), 3.48 (s, 2H), 2.43-2.38 (m, 4H), 2.28 (s, 3H).

LC/MS (method M, ESIpos): Rₜ=0.99 min, m/z=500 [M+H]⁺.

### B. EVALUATION OF THE PHARMACOLOGICAL ACTIVITY

The pharmacological activity of the compounds according to the invention can be demonstrated by in vitro and in vivo studies such as are known to the person skilled in the art. The usefulness of the substances according to the invention can be illustrated by way of example by in vitro (tumour) cell experiments and in vivo tumour models such as are described below. The connection between an inhibition of the HIF transcription activity and the inhibition of tumour growth is demonstrated by numerous studies described in the literature (cf. e.g. Warburg, 1956; Semenza, 2007).

### B-1. HIF-Luciferase Assay

HCT 116 cells were transfected in a stable manner with a plasmid which contained a luciferase reporter under the control of an HIF-responsive sequence. These cells were sown in microtitre plates [20 000 cells/cavity in RPMI 1640 medium with 10% foetal calf serum (FCS) and 100 μg/ml of hygromycin]. Incubation was carried out overnight under standard conditions (5% CO₂, 21% O₂, 37° C., moistened). The following morning the cells were incubated with various concentrations of the test substances (0-10 μmol/l) in a hypoxia chamber (1% O₂). After 24 h, Bright Glo reagent (Promega, Wisconsin, USA) was added in accordance with the manufacturer's instructions, and after 5 min the luminescence was measured. Cells which were incubated under normoxia served as background controls.

The IC₅₀ values from this assay for representative embodiment examples are listed in the following table:

| Example no. | IC₅₀ [nmol/l] |
|---|---|
| 14 | 7 |
| 16 | 8 |
| 22 | 10 |
| 24 | 10 |
| 48 | 10 |
| 57 | 10 |
| 60 | 9 |
| 67 | 10 |
| 71 | 6 |
| 73 | 8 |
| 74 | 8 |
| 102 | 2.5 |
| 103 | 3 |
| 123 | 1 |
| 124 | 5 |

### B-2. Suppression of HIF Target Genes In Vitro

Human bronchial carcinoma cells (H460 and A549) were incubated for 16 h with variable concentrations of the test substances (1 nM to 10 μM) under normoxic conditions and under a 1% oxygen partial pressure (see HIF-luciferase assay). The total RNA was isolated from the cells and transcribed into cDNA and the mRNA expression of HIF target genes was analysed in real time PCR. Active test substances already lower the mRNA expression of the HIF target genes compared with untreated cells under normoxic conditions, but above all under hypoxic conditions.

### B-3. Human Xenograft and Syngenic Tumour Models

Human tumour xenograft models in immunodeficient mice and syngenic tumour mouse models were used for evaluation of the substances. For this, tumour cells were cultured in vitro and implanted subcutaneously, or tumour xenotransplant pieces were transplanted further subcutaneously. The animals were treated by oral, subcutaneous or intraperitoneal therapy after the tumour was established. The activity of the test substances was analysed in monotherapy and in combination therapy with other pharmacological active substances. The tumour inhibitory potency of the test substances on tumours of advanced size (approx. 100 mm²) was moreover characterized. The state of health of the animals was checked daily, and the treatments were performed in accordance with animal protection regulations. The tumour area was measured with slide gauges (length L, breadth B=shorter dimension). The tumour volume was calculated from the formula (L×B²)/2. The inhibition in tumour growth was determined at the end of the study as the T/C ratio of the tumour areas and tumour weights and as the TGI value (tumour growth inhibition, calculated from the formula [1−(T/C)]×100) (T=tumour size in the treated group; C=tumour size in the untreated control group).

The influence of the test substances on the tumour vessel architecture and the blood flow within the tumour was identified with the aid of computer microtomography and ultrasound microstudies on treated and untreated tumour-carrying mice.

### C. DETERMINATION OF THE SOLUBILITY AND PHARMACOKINETIC PARAMETERS

The solubility of the compounds according to the invention in aqueous systems and the pharmacokinetic parameters following intravenous and/or oral administration can be determined in the assays described below:

### C-1. Determination of the Solubility

### Preparation of the Starting Solution (Original Solution):

At least 1.5 mg of the test substance are weighed accurately into a wide-necked 10 mm screw V vial (from Glastechnik Gräfenroda GmbH, Art. No. 8004-WM-H/V15μ with fitting screw cap and septum, DMSO is added to a concentration of 50 mg/ml and the mixture is vortexed for 30 minutes.

### Preparation of the Calibration Solutions:

The pipetting steps required are carried out in a 1.2 ml 96 deep well plate (DWP) using a liquid-handling robot. The solvent used is a mixture of acetonitrile and water (8:2).

Preparation of the starting solution for calibration solutions (stock solution): 833 μl of the solvent mixture are added to 10 μl of the original solution (concentration=600 μg/ml), and the mixture is homogenized. From each test substance, 1:100 dilutions are prepared in separate DWPs and homogenized for their part.

Calibration solution 5 (600 ng/ml): 270 μl of solvent mixture are added to 30 μl of the stock solution, and the mixture is homogenized.

Calibration solution 4 (60 ng/ml): 270 μl of solvent mixture are added to 30 μl of calibration solution 5, and the mixture is homogenized.

Calibration solution 3 (12 ng/ml): 400 μl of solvent mixture are added to 100 μl of calibration solution 4, and the mixture is homogenized.

Calibration solution 2 (1.2 ng/ml): 270 μl of solvent mixture are added to 30 μl of calibration solution 3, and the mixture is homogenized.

Calibration solution 1 (0.6 ng/ml): 150 μl of solvent mixture are added to 150 μl of calibration solution 2, and the mixture is homogenized.

### Preparation of the Sample Solutions:

The pipetting steps required are carried out in a 1.2 ml 96 deep well plate (DWP) using a liquid-handling robot. 1000 μl of PBS buffer pH 6.5 [PBS buffer pH 6.5: 61.86 g of sodium chloride, 39.54 g of sodium dihydrogenphosphate and 83.35 g of 1 N sodium hydroxide solution are weighed into a 1-litre measuring flask which is then filled with water and stirred for about 1 h; 500 ml of this solution are added to a 5-litre measuring flask which is then filled with water, and the pH is adjusted to 6.5 using 1 N sodium hydroxide solution] are added to 10.1 μl of the stock solution.

### Procedure:

The pipetting steps required are carried out in a 1.2 ml 96 deep well plate (DWP) using a liquid-handling robot. The sample solutions prepared in this manner are shaken at 1400 rpm in a temperature-adjustable shaker at 20° C. for 24 h. In each case 180 μl are taken from these solutions and transferred into Beckman Polyallomer centrifuge tubes. These solutions are centrifuged at about 223 000×g for 1 h. From each of the sample solutions, 100 μl of supernatant are removed and diluted 1:10 and 1:1000 with PBS buffer pH 6.5.

### Analysis:

The samples are analysed by HPLC-MS/MS. Quantification is carried out using a five-point calibration curve of the test compound. The solubility is expressed in mg/l.

Analysis sequence: 1) blank (solvent mixture); 2) calibration solution 0.6 ng/ml; 3) calibration solution 1.2 ng/ml; 4) calibration solution 12 ng/ml; 5) calibration solution 60 ng/ml; 6) calibration solution 600 ng/ml; 7) blank (solvent mixture); 8) sample solution 1:1000; 9) sample solution 1:10.

### HPLC-MS/MS Method:

HPLC: Agilent 1100, quat. pump (G1311A), autosampler CTC HTS PAL, degasser (G1322A) and column thermostat (G1316A); column: Oasis HLB 20 mm×2.1 mm, 25μ; temperature: 40° C.; mobile phase A: water+0.5 ml formic acid/1, mobile phase B: acetonitrile+0.5 ml formic acid/1; flow rate: 2.5 ml/min; stop time: 1.5 min; gradient: 0 min 95% A, 5% B; ramp: 0-0.5 min 5% A, 95% B; 0.5-0.84 min 5% A, 95% B; ramp: 0.84-0.85 min 95% A, 5% B; 0.85-1.5 min 95% A, 5% B.

MS/MS: Waters Quattro Micro Tandem MS/MS; Z-Spray API-Interface; HPLC-MS inlet splitter 1:20; measured in the ESI mode.

### C-2. Determination of Pharmacokinetic Parameters Following Intravenous and Oral Administration:

The substance to be investigated was administered to animals (e.g. mice or rats) intravenously as a solution (e.g. in corresponding plasma with a small addition of DMSO or in a PEG/ethanol/water mixture), and oral administration took place as a solution (e.g. in a Solutol/ethanol/water or PEG/ethanol/water mixture) or as a suspension (e.g. in tylose), in each case via a stomach tube. After administration of the substance, blood was taken from the animals at specified points in time. This was heparinized, and plasma was then obtained therefrom by centrifugation. The substance was quantified analytically in the plasma via LC-MS/MS. From the plasma concentration/time plots determined in this way, the pharmacokinetic parameters, such as AUC (area under the concentration/time curve), Cₘₐₓ (maximum plasma concentration), T_{1/2} (half life), V_{SS} (distribution volume) and CL (clearance), and the absolute and the relative bioavailability (i.v./p.o. comparison or comparison of suspension to solution after p.o. administration), were calculated using an internal standard and with the aid of a validated computer program.

### D. WORKING EXAMPLES OF PHARMACEUTICAL COMPOSITIONS

The compounds according to the invention can be converted into pharmaceutical formulations as follows.

### Tablet:

### Composition:

100 mg of the compound according to the invention, 50 mg of lactose (monohydrate), 50 mg of maize starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

### Preparation:

The mixture of compound according to the invention, lactose and starch is granulated with a 5% strength solution (w/w) of the PVP in water. After drying, the granules are mixed with the magnesium stearate for 5 minutes. This mixture is pressed with a conventional tablet press (for tablet format see above). A pressing force of 15 kN is used as the recommended value for the pressing.

### Suspension for Oral Administration:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to an individual dose of 100 mg of the compound according to the invention.

### Preparation:

The Rhodigel is suspended in ethanol and the compound according to the invention is added to the suspension. The water is added with stirring. The mixture is stirred for approx. 6 h until swelling of the Rhodigel has ended.

### Solution for Oral Administration:

### Composition:

500 mg of the compound according to the invention, 2.5 g of polysorbate and 97 g of polyethylene glycol 400.20 g of oral solution correspond to an individual dose of 100 mg of the compound according to the invention.

### Preparation:

The compound according to the invention is suspended in the mixture of polyethylene glycol and polysorbate, while stirring. The stirring operation is continued until dissolution of the compound according to the invention is complete.

i.v. Solution:

The compound according to the invention is dissolved in a concentration below the saturation solubility in a physiologically acceptable solvent (e.g. isotonic saline solution, glucose solution 5% and/or PEG 400 solution 30%). The solution is subjected to sterile filtration and is transferred into sterile and pyrogen-free injection containers.

### E LITERATURE REFERENCES

Globocan 2002 Report
IARC International Agency for Research on Cancer: Globocan 2002, http://www-dep.iarc.fr/globocan/downloads.htm
American Cancer Society, Cancer Facts and Figures 2005
American Cancer Society: Cancer Facts and Figures 2007, http://www.cancer.org/docroot/STT/content/STT_{—}1x_Cancer_Facts_Figures_{—}2007. asp
Gibbs J B, 2000
Gibbs J B: Mechanism-based target identification and drug discovery in cancer research, Science 2000, 287 (5460), 1969-1973.
Semenza and Wang, 1992
Semenza G L, Wang G L: A nuclear factor induced by hypoxia via de novo protein synthesis binds to the human erythropoietin gene enhancer at a site required for transcriptional activation, Mol. Cell. Biol. 1992, 12 (12), 5447-5454.
Wang and Semenza, 1995
Wang G L, Semenza G L: Purification and characterization of hypoxia-inducible factor 1, J. Biol. Chem. 1995, 270 (3), 1230-1237.
Wang, Jiang et al., 1995
Wang G L, Jiang B H, Rue E A, Semenza G L: Hypoxia-inducible factor 1 is a basic-helix-loop-helix-PAS heterodimer regulated by cellular O₂ tension, PNAS 1995, 92 (12), 5510-5514.
Jiang, Rue et al., 1996
Jiang B H, Rue E, Wang G L, Roe R, Semenza G L: Dimerization, DNA binding, and transactivation properties of hypoxia-inducible factor 1, J. Biol. Chem. 1996, 271 (30), 17771-17778.
Makino, Cao et al., 2001
Makino Y, Cao R, Svensson K, Bertilsson G, Asman M, Tanaka H, Cao Y, Poellinger L: Nature 2001, 414 (6863), 550-554.
Jiang, Semenza et al., 1996
Jiang B H, Semenza G L, Bauer C, Marti H H: Hypoxia-inducible factor 1 levels vary exponentially over a physiologically relevant range of O₂ tension, Am. J. Physiol. 1996, 271, 1172-1180.
Maxwell, Wiesener et al., 1999
Maxwell P H, Wiesener M S, Chang G W, Clifford S C, Vaux E C, Cockman M E, Wykoff C C, Ratcliffe P J: The tumour suppressor protein VHL targets hypoxia-inducible factors for oxygen-dependent proteolysis, Nature 1999, 399 (6733), 271-275.
Hirota and Semenza, 2006
Hirota K, Semenza G L: Regulation of angiogenesis by hypoxia-inducible factor 1, Crit. Rev. Oncol. Hematol. 2006, 59 (1), 15-26.
Chen, Zhao et al., 2003
Chen J, Zhao S, Nakada K, Kuge Y, Tamaki N, Okada F, Wang J, Shindo M, Higashino F, Takeda K, Asaka M, Katoh H, Sugiyama T, Hosokawa M, Kobayashi M: Dominant-negative hypoxia-inducible factor-1alpha reduces tumorigenicity of pancreatic cancer cells through the suppression of glucose metabolism, Am. J. Pathol. 2003, 162 (4), 1283-1291.
Stoeltzing, McCarty et al., 2004
Stoeltzing O, McCarty M F, Wey J S, Fan F, Liu W, Belcheva A, Bucana C D, Semenza G L, Ellis L M: Role of hypoxia-inducible factor-1alpha in gastric cancer cell growth, angiogenesis, and vessel maturation, J. Natl. Cancer Inst. 2004, 96 (12), 946-956.
Li, Lin et al., 2005
Li L, Lin X, Stayer M, Shoemaker A, Semizarov D, Fesik S W, Shen Y: Evaluating hypoxia-inducible factor-1alpha as a cancer therapeutic target via inducible RNA interference in vivo, Cancer Res. 2005, 65 (16), 7249-7258.
Mizukami, Jo et al., 2005
Mizukami Y, Jo W S, Duerr E M, Gala M, Li J, Zhang X, Zimmer M A, Iliopoulos O, Zukerberg L R, Kohgo Y, Lynch M P, Rueda B R, Chung D C: Induction of interleukin-8 preserves the angiogenic response in HIF-1alpha-deficient colon cancer cells, Nat. Med. 2005, 11 (9), 992-997.
Li, Shi et al., 2006
Li J, Shi M, Cao Y, Yuan W, Pang T, Li B, Sun Z, Chen L, Zhao R C: Knockdown of hypoxia-inducible factor-1 alpha in breast carcinoma MCF-7 cells results in reduced tumor growth and increased sensitivity to methotrexate, Biochem. Biophys. Res. Commun. 2006, 342, 1341-1351.
Semenza, 2007
Semenza G L: Drug Discov. Today 2007, 12 (19-20), 853-859.
Weidemann and Johnson, 2008
Weidemann A, Johnson R S: Cell Death and Differentiation 2008, 15, 621-627.
Aiello et al., 1994
Aiello et al.: New Engl. J. Med. 1994, 331, 1480.
Peer et al., 1995
Peer et al.: Lab. Invest. 1995, 72, 638.
Lopez et al., 1996
Lopez et al.: Invest. Ophthalmol. Vis. Sci. 1996, 37, 855.
Warburg, 1956
Warburg O: Science 1956, 123 (3191), 309-314.

1. Compound of the formula (I) 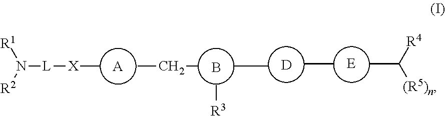
in which
the ring 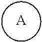
represents a phenyl or pyridyl ring,
the ring 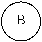
with the substituent R³ represents a heteroaryl ring of the formula 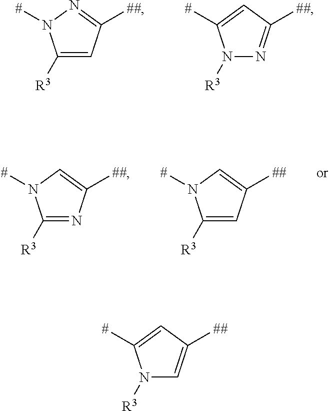
wherein
# designates the linkage point with the adjacent CH₂ group
and
## designates the linkage point with the ring 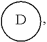
the ring 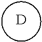
represents a heteroaryl ring of the formula 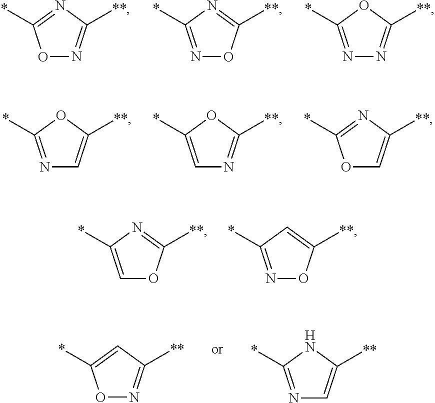
wherein
* designates the linkage point with the ring 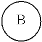
and
** designates the linkage point with the ring 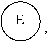
the ring 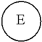
represents a phenyl or pyridyl ring,
X represents a bond or represents —N(R⁶)—, —O—, —S—, —S(═O)₂—, ♦-C(═O)—N(R⁶)-♦♦ or ♦-N(R⁶)—C(═O)-♦♦, wherein
♦ designates the linkage point with the group L
and
♦♦ designates the linkage point with the ring 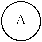
and
R⁶ denotes hydrogen, (C₁-C₆)-alkyl or (C₃-C₆)-cycloalkyl,
wherein (C₁-C₆)-alkyl and (C₃-C₆)-cycloalkyl can each be substituted by hydroxyl or (C₁-C₄)-alkoxy,
L represents straight-chain (C₁-C₄)-alkanediyl if X denotes a bond or the group —S(═O)₂— or ♦-C(═O)—N(R⁶)-♦♦,
and
represents straight-chain (C₂-C₄)-alkanediyl if X denotes the group —N(R⁶)—, —O—, —S— or ♦-N(R⁶)—C(═O)-♦♦,
R¹ represents hydrogen, (C₁-C₆)-alkyl, (C₁-C₆)-alkylcarbonyl, (C₁-C₆)-alkoxycarbonyl, (C₁-C₆)-alkylsulphonyl or (C₃-C₆)-cycloalkyl,
wherein the alkyl group in (C₁-C₆)-alkyl, (C₁-C₆)-alkylcarbonyl, (C₁-C₆)-alkoxycarbonyl and (C₁-C₆)-alkylsulphonyl can be substituted up to three times by fluorine and up to two times by identical or different radicals selected from the group consisting of hydroxyl, (C₁-C₄)-alkoxy, amino, mono-(C₁-C₄)-alkylamino and di-(C₁-C₄)-alkylamino
and
(C₃-C₆)-cycloalkyl can be substituted up to two times by identical or different radicals selected from the group consisting of fluorine, (C₁-C₄)-alkyl, hydroxyl, (C₁-C₄)-alkoxy, amino, mono-(C₁-C₄)-alkylamino and di-(C₁-C₄)-alkylamino,
R² represents hydrogen, (C₁-C₆)-alkyl or (C₃-C₆)-cycloalkyl,
where (C₁-C₆)-alkyl can be substituted up to three times by fluorine and up to two times by identical or different radicals selected from the group consisting of hydroxyl, (C₁-C₄)-alkoxy, amino, mono-(C₁-C₄)-alkylamino and di-(C₁-C₄)-alkylamino
and
(C₃-C₆)-cycloalkyl can be substituted up to two times by identical or different radicals selected from the group consisting of fluorine, (C₁-C₄)-alkyl, hydroxyl, (C₁-C₄)-alkoxy, amino, mono-(C₁-C₄)-alkylamino and di-(C₁-C₄)-alkylamino,
or
R¹ and R² together with the nitrogen atom to which they are attached form a saturated 4- to 7-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl, (C₁-C₄)-alkylsulphonyl and (C₃-C₆)-cycloalkyl,
wherein (C₁-C₄)-alkyl and (C₃-C₆)-cycloalkyl for their part can be substituted by hydroxyl or (C₁-C₄)-alkoxy,
R³ represents methyl, ethyl or trifluoromethyl,
R⁴ represents hydrogen or a substituent chosen from the group consisting of halogen, cyano, pentafluorothio, (C₁-C₆)-alkyl, tri-(C₁-C₄)-alkylsilyl, —OR⁷, —NR⁷R⁸, —N(R⁷)—C(═O)—R⁸, —N(R⁷)—C(═O)—OR⁸, —N(R⁷)—S(═O)₂—R⁸, —C(═O)—OR⁷, —C(═O)—NR⁷R⁸, —SR⁷, —S(═O)—R⁷, —S(═O)₂—R⁷, —S(═O)₂—NR⁷R⁸, (C₃-C₆)-cycloalkyl, 4- to 6-membered heterocyclyl and 5- or 6-membered heteroaryl,
where (C₁-C₆)-alkyl for its part can be substituted up to three times by fluorine and up to two times by identical or different radicals selected from the group consisting of —OR⁷, —NR⁷R⁸, —N(R⁷)—C(═O)—R⁸, —N(R⁷)—C(═O)—OR⁸, —C(═O)—OR⁷, —C(═O)—NR⁷R⁸, (C₃-C₆)-cycloalkyl, 4- to 6-membered heterocyclyl and 5- or 6-membered heteroaryl
and where
the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo, amino, mono-(C₁-C₄)-alkylamino, di-(C₁-C₄)-alkylamino, (C₁-C₄)-alkylcarbonylamino, (C₁-C₄)-alkoxycarbonylamino, (C₁-C₄)-alkylcarbonyl and (C₁-C₄)-alkoxycarbonyl
and
the heteroaryl groups mentioned for their part can be substituted up to two times by identical or different radicals selected from the group consisting of fluorine, chlorine, cyano, (C₁-C₄)-alkyl, trifluoromethyl, (C₁-C₄)-alkoxy and trifluoromethoxy,
and wherein
R⁷ and R⁸ independently of each other for each individual occurrence denote hydrogen, (C₁-C₆)-alkyl, (C₃-C₆)-cycloalkyl or 4- to 6-membered heterocyclyl,
where (C₁-C₆)-alkyl can be substituted up to three times by fluorine and up to two times by identical or different radicals selected from the group consisting of hydroxyl, (C₁-C₄)-alkoxy, trifluoromethoxy, amino, mono-(C₁-C₄)-alkylamino, di-(C₁-C₄)-alkylamino, (C₁-C₄)-alkoxycarbonyl, (C₃-C₆)-cycloalkyl and 4- to 6-membered heterocyclyl
and
the cycloalkyl and heterocyclyl groups mentioned can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
or
R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
R⁵ represents a substituent selected from the group consisting of fluorine, chlorine, cyano, methyl, trifluoromethyl and hydroxyl
and
n represents the number 0, 1 or 2,
where, if the substituent R⁵ occurs twice, its meanings can be identical or different,
or a salt thereof.

2. Compound of the formula (I) according to claim 1 in which
the ring 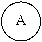
represents a phenyl or pyridyl ring and the adjacent groups X and CH₂ are bonded to ring carbon atoms of 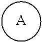
in 1,3 or 1,4 relation to one another
and
the ring 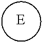
with the substituents R⁴ and R⁵ represents a phenyl ring of the formula 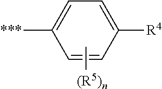
wherein
*** designates the linkage point with the ring 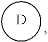
or a salt thereof.

3. Compound of the formula (I) according to claim 1 in which
the ring 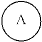
represents a pyridyl ring and the adjacent groups X and CH₂ are bonded to ring carbon atoms of this pyridyl ring in 1,3 or 1,4 relation to one another
and
the ring 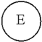
with the substituents R⁴ and R⁵ represents a phenyl ring of the formula 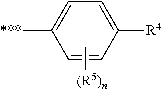
wherein
*** designates the linkage point with the ring 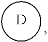
or a salt thereof.

4. Compound of the formula (I) according to claim 1 in which
the ring 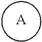
represents a phenyl ring and the adjacent groups X and CH₂ are bonded to this phenyl ring in 1,3 or 1,4 relation to one another,
the ring 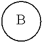
with the substituent R³ represents a heteroaryl ring of the formula 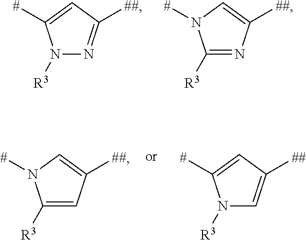
wherein
# designates the linkage point with the adjacent CH₂ group
and
## designates the linkage point with the ring 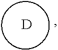
and
the ring 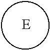
with the substituents R⁴ and R⁵ represents a phenyl ring of the formula 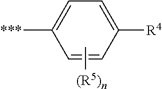
wherein
*** designates the linkage point with the ring 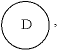
or a salt thereof.

5. Compound of the formula (I) according to claim 1 in which
the ring 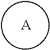
represents a phenyl ring and the adjacent groups X and CH₂ are bonded to this phenyl ring in 1,3 or 1,4 relation to one another,
the ring 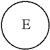
with the substituents R⁴ and R⁵ represents a phenyl ring of the formula 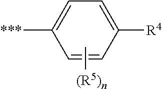
wherein
*** designates the linkage point with the ring 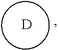
and
R¹ and R² together with the nitrogen atom to which they are attached form a saturated 4- to 7-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl, (C₁-C₄)-alkylsulphonyl and (C₃-C₆)-cycloalkyl,
wherein (C₁-C₄)-alkyl and (C₃-C₆)-cycloalkyl for their part can be substituted by hydroxyl or (C₁-C₄)-alkoxy,
or a salt thereof.

6. Compound of the formula (I) according to claim 1 in which
the ring 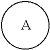
represents a pyridyl ring and the adjacent groups X and CH₂ are bonded to ring carbon atoms of this pyridyl ring in 1,3 or 1,4 relation to one another,
the ring 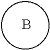
with the substituent R³ represents a heteroaryl ring of the formula 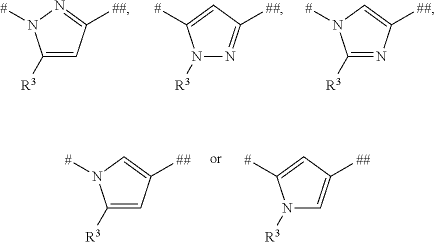
wherein
# designates the linkage point with the adjacent CH₂ group
and
## designates the linkage point with the ring 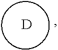
the ring 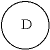
represents a heteroaryl ring of the formula 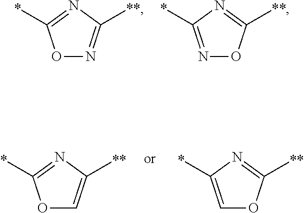
wherein
* designates the linkage point with the ring 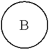
and
** designates the linkage point with the ring 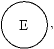
the ring 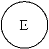
with the substituents R⁴ and R⁵ represents a phenyl ring of the formula 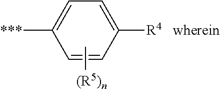
*** designates the linkage point with the ring 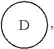
X represents a bond or represents —N(R⁶)—, —O—, —S—, ♦-C(═O)—N(R⁶)-♦♦ or ♦-N(R⁶)—C(═O)-♦♦, wherein
♦ designates the linkage point with the group L
and
♦♦ designates the linkage point with the ring 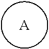
and
R⁶ denotes hydrogen, (C₁-C₄)-alkyl or (C₃-C₆)-cycloalkyl,
L represents straight-chain (C₁-C₄)-alkanediyl if X denotes a bond or the group ♦-C(═O)—N(R⁶)-♦♦,
and
represents straight-chain (C₂-C₄)-alkanediyl if X denotes the group —N(R⁶)—, —O—, —S— or ♦-N(R⁶)—C(═O)-♦♦,
R¹ represents hydrogen, (C₁-C₄)-alkyl, (C₁-C₄)-alkylcarbonyl, (C₁-C₄)-alkylsulphonyl or (C₃-C₆)-cycloalkyl,
where the alkyl group in (C₁-C₄)-alkyl, (C₁-C₄)-alkylcarbonyl and (C₁-C₄)-alkylsulphonyl can be substituted up to three times by fluorine and up to two times by identical or different radicals selected from the group consisting of hydroxyl and (C₁-C₄)-alkoxy
and
(C₃-C₆)-cycloalkyl can be substituted up to two times by identical or different radicals selected from the group consisting of fluorine, (C₁-C₄)-alkyl, hydroxyl and (C₁-C₄)-alkoxy,
R² represents hydrogen, (C₁-C₄)-alkyl or (C₃-C₆)-cycloalkyl,
where (C₁-C₄)-alkyl may be substituted up to three times by fluorine
and
(C₃-C₆)-cycloalkyl can be substituted up to two times by identical or different radicals selected from the group consisting of fluorine and (C₁-C₄)-alkyl,
or
R¹ and R² together with the nitrogen atom to which they are attached form a saturated 4- to 6-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl, (C₁-C₄)-alkylsulphonyl and (C₃-C₆)-cycloalkyl,
R³ represents methyl, ethyl or trifluoromethyl,
R⁴ represents a substituent selected from the group consisting of fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, (C₃-C₆)-cycloalkyl and 4- to 6-membered heterocyclyl,
where (C₁-C₆)-alkyl for its part can be substituted up to three times by fluorine and up to two times by identical or different radicals selected from the group consisting of —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
and where
the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
and
the heteroaryl groups mentioned for their part can be substituted up to two times by identical or different radicals selected from the group consisting of fluorine, chlorine, cyano, (C₁-C₄)-alkyl, trifluoromethyl, (C₁-C₄)-alkoxy and trifluoromethoxy,
and wherein
R⁷ and R⁸ independently of each other for each individual occurrence denote hydrogen, (C₁-C₄)-alkyl, (C₃-C₆)-cycloalkyl or 4- to 6-membered heterocyclyl,
where (C₁-C₄)-alkyl can be substituted up to three times by fluorine and up to two times by identical or different radicals selected from the group consisting of hydroxyl, (C₁-C₄)-alkoxy, trifluoromethoxy, (C₃-C₆)-cycloalkyl and 4- to 6-membered heterocyclyl
and
the cycloalkyl and heterocyclyl groups mentioned can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
or
R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents a substituent selected from the group consisting of fluorine, chlorine and methyl
and
n represents the number 0 or 1,
or a salt thereof.

7. Compound of the formula (I) according to claim 1 in which
the ring 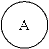
represents a phenyl ring and the adjacent groups X and CH₂ are bonded to this phenyl ring in 1,3 or 1,4 relation to one another,
the ring 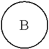
with the substituent R³ represents a heteroaryl ring of the formula 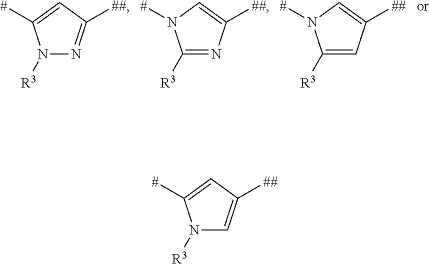
wherein
# designates the linkage point with the adjacent CH₂ group
and
## designates the linkage point with the ring 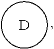
the ring 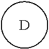
represents a heteroaryl ring of the formula 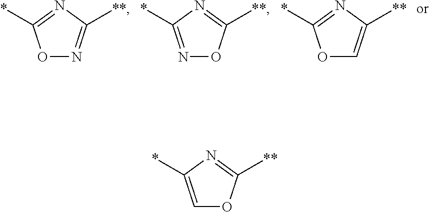
wherein
* designates the linkage point with the ring 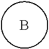
and
** designates the linkage point with the ring 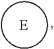
the ring 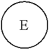
with the substituents R⁴ and R⁵ represents a phenyl ring of the formula 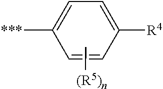
wherein
*** designates the linkage point with the ring 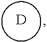
X represents a bond or represents —N(R⁶)—, —O—, —S—, ♦-C(═O)—N(R⁶)-♦♦ or ♦-N(R⁶)—C(═O)-♦♦, wherein
♦ designates the linkage point with the group L
and
♦♦ designates the linkage point with the ring 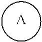
and
R⁶ denotes hydrogen, (C₁-C₄)-alkyl or (C₃-C₆)-cycloalkyl,
L represents straight-chain (C₁-C₄)-alkanediyl if X denotes a bond or the group ♦-C(═O)—N(R⁶)-♦♦,
and
represents straight-chain (C₂-C₄)-alkanediyl if X denotes the group —N(R⁶)—, —O—, —S— or ♦-N(R⁶)—C(═O)-♦♦,
R¹ represents hydrogen, (C₁-C₄)-alkyl, (C₁-C₄)-alkylcarbonyl, (C₁-C₄)-alkylsulphonyl or (C₃-C₆)-cycloalkyl,
where the alkyl group in (C₁-C₄)-alkyl, (C₁-C₄)-alkylcarbonyl and (C₁-C₄)-alkylsulphonyl can be substituted up to three times by fluorine and up to two times by identical or different radicals selected from the group consisting of hydroxyl and (C₁-C₄)-alkoxy
and
(C₃-C₆)-cycloalkyl can be substituted up to two times by identical or different radicals selected from the group consisting of fluorine, (C₁-C₄)-alkyl, hydroxyl and (C₁-C₄)-alkoxy,
R² represents hydrogen, (C₁-C₄)-alkyl or (C₃-C₆)-cycloalkyl,
where (C₁-C₄)-alkyl may be substituted up to three times by fluorine
and
(C₃-C₆)-cycloalkyl can be substituted up to two times by identical or different radicals selected from the group consisting of fluorine and (C₁-C₄)-alkyl,
R³ represents methyl, ethyl or trifluoromethyl,
R⁴ represents a substituent selected from the group consisting of fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, (C₃-C₆)-cycloalkyl and 4- to 6-membered heterocyclyl,
where (C₁-C₆)-alkyl for its part can be substituted up to three times by fluorine and up to two times by identical or different radicals selected from the group consisting of —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
and where
the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
and
the heteroaryl groups mentioned for their part can be substituted up to two times by identical or different radicals selected from the group consisting of fluorine, chlorine, cyano, (C₁-C₄)-alkyl, trifluoromethyl, (C₁-C₄)-alkoxy and trifluoromethoxy,
and wherein
R⁷ and R⁸ independently of each other for each individual occurrence denote hydrogen, (C₁-C₄)-alkyl, (C₃-C₆)-cycloalkyl or 4- to 6-membered heterocyclyl,
where (C₁-C₄)-alkyl can be substituted up to three times by fluorine and up to two times by identical or different radicals selected from the group consisting of hydroxyl, (C₁-C₄)-alkoxy, trifluoromethoxy, (C₃-C₆)-cycloalkyl and 4- to 6-membered heterocyclyl
and
the cycloalkyl and heterocyclyl groups mentioned can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
or
R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents a substituent selected from the group consisting of fluorine, chlorine and methyl
and
n represents the number 0 or 1,
or a salt thereof.

8. Compound of the formula (I) according to claim 1 in which
the ring 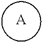
represents a phenyl ring and the adjacent groups X and CH₂ are bonded to this phenyl ring in 1,3 or 1,4 relation to one another,
the ring 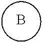
with the substituent R³ represents a heteroaryl ring of the formula 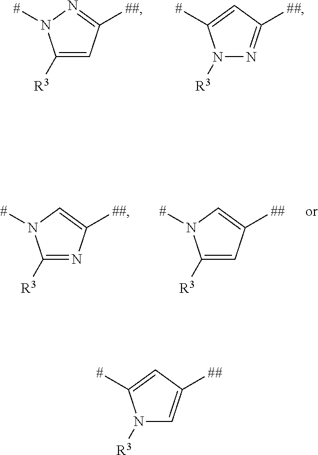
wherein
# designates the linkage point with the adjacent CH₂ group
and
## designates the linkage point with the ring 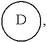
the ring 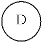
represents a heteroaryl ring of the formula 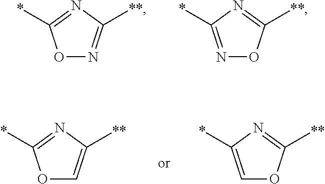
wherein
* designates the linkage point with the ring 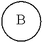
and
** designates the linkage point with the ring 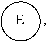
the ring 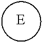
with the substituents R⁴ and R⁵ represents a phenyl ring of the formula 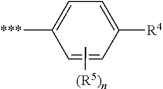
wherein
*** designates the linkage point with the ring 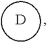
X represents a bond or represents —N(R⁶)—, —O—, —S—, ♦-C(═O)—N(R⁶)-♦♦ or ♦-N(R⁶)—C(═O)-♦♦, wherein
♦ designates the linkage point with the group L
and
♦♦ designates the linkage point with the ring 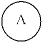
and
R⁶ denotes hydrogen, (C₁-C₄)-alkyl or (C₃-C₆)-cycloalkyl,
L represents straight-chain (C₁-C₄)-alkanediyl if X denotes a bond or the group ♦-C(═O)—N(R⁶)-♦♦,
and
represents straight-chain (C₂-C₄)-alkanediyl if X denotes the group —N(R⁶)—, —O—, —S— or ♦-N(R⁶)—C(═O)-♦♦,
R¹ and R² together with the nitrogen atom to which they are attached form a saturated 4- to 6-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl, (C₁-C₄)-alkylsulphonyl and (C₃-C₆)-cycloalkyl,
R³ represents methyl, ethyl or trifluoromethyl,
R⁴ represents a substituent selected from the group consisting of fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, (C₃-C₆)-cycloalkyl and 4- to 6-membered heterocyclyl,
where (C₁-C₆)-alkyl for its part can be substituted up to three times by fluorine and up to two times by identical or different radicals selected from the group consisting of —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
and where
the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
and
the heteroaryl groups mentioned for their part can be substituted up to two times by identical or different radicals selected from the group consisting of fluorine, chlorine, cyano, (C₁-C₄)-alkyl, trifluoromethyl, (C₁-C₄)-alkoxy and trifluoromethoxy,
and wherein
R⁷ and R⁸ independently of each other for each individual occurrence denote hydrogen, (C₁-C₄)-alkyl, (C₃-C₆)-cycloalkyl or 4- to 6-membered heterocyclyl,
where (C₁-C₄)-alkyl can be substituted up to three times by fluorine and up to two times by identical or different radicals selected from the group consisting of hydroxyl, (C₁-C₄)-alkoxy, trifluoromethoxy, (C₃-C₆)-cycloalkyl and 4- to 6-membered heterocyclyl
and
the cycloalkyl and heterocyclyl groups mentioned can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
or
R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents a substituent selected from the group consisting of fluorine, chlorine and methyl
and
n represents the number 0 or 1,
or a salt thereof.

9. Compound of the formula (I) according to claim 1 in which
the ring 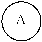
represents a phenyl ring and the adjacent groups X and CH₂ are bonded to this phenyl ring in 1,3 or 1,4 relation to one another,
the ring 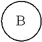
with the substituent R³ represents a heteroaryl ring of the formula 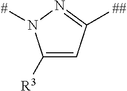
wherein
# designates the linkage point with the adjacent CH₂ group
and
## designates the linkage point with the ring 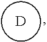
the ring 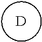
represents a heteroaryl ring of the formula 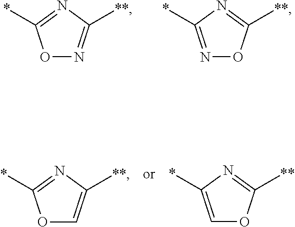
wherein
* designates the linkage point with the ring 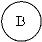
and
** designates the linkage point with the ring 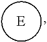
the ring 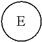
with the substituents R⁴ and R⁵ represents a phenyl ring of the formula 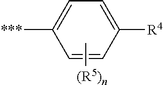
wherein
*** designates the linkage point with the ring 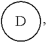
X represents a bond or represents —N(R⁶)—, —O—, —S—, ♦-C(═O)—N(R⁶)-♦♦ or ♦-N(R⁶)—C(═O)-♦♦, wherein
♦ designates the linkage point with the group L
and
♦♦ designates the linkage point with the ring 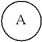
and
R⁶ denotes hydrogen, (C₁-C₄)-alkyl or (C₃-C₆)-cycloalkyl,
L represents straight-chain (C₁-C₄)-alkanediyl if X denotes a bond or the group ♦-C(═O)—N(R⁶)-♦♦,
and
represents straight-chain (C₂-C₄)-alkanediyl if X denotes the group —N(R⁶)—, —O—, —S— or ♦-N(R⁶)—C(═O)-♦♦,
R¹ represents hydrogen, (C₁-C₄)-alkyl or (C₃-C₆)-cycloalkyl,
where (C₁-C₄)-alkyl can be substituted up to three times by fluorine and up to two times by identical or different radicals selected from the group consisting of hydroxyl and (C₁-C₄)-alkoxy,
R² represents hydrogen, (C₁-C₄)-alkyl or (C₃-C₆)-cycloalkyl,
R³ represents methyl, ethyl or trifluoromethyl,
R⁴ represents a substituent selected from the group consisting of fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, (C₃-C₆)-cycloalkyl and 4- to 6-membered heterocyclyl,
where (C₁-C₆)-alkyl for its part can be substituted up to three times by fluorine and up to two times by identical or different radicals selected from the group consisting of —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
and where
the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
and
the heteroaryl groups mentioned for their part can be substituted up to two times by identical or different radicals selected from the group consisting of fluorine, chlorine, cyano, (C₁-C₄)-alkyl, trifluoromethyl, (C₁-C₄)-alkoxy and trifluoromethoxy,
and wherein
R⁷ and R⁸ independently of each other for each individual occurrence denote hydrogen, (C₁-C₄)-alkyl, (C₃-C₆)-cycloalkyl or 4- to 6-membered heterocyclyl,
where (C₁-C₄)-alkyl can be substituted up to three times by fluorine and up to two times by identical or different radicals selected from the group consisting of hydroxyl, (C₁-C₄)-alkoxy, trifluoromethoxy, (C₃-C₆)-cycloalkyl and 4- to 6-membered heterocyclyl
and
the cycloalkyl and heterocyclyl groups mentioned can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
or
R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents a substituent selected from the group consisting of fluorine, chlorine and methyl
and
n represents the number 0 or 1,
or a salt thereof.

10. Compound of the formula (I) according to claim 1 in which
the ring 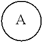
represents a pyridyl ring of the formula 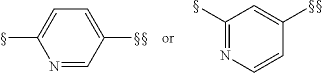
wherein
§ designates the linkage point with the adjacent group X
and
§§ designates the linkage point with the adjacent CH₂ group,
the ring 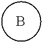
with the substituent R³ represents a heteroaryl ring of the formula 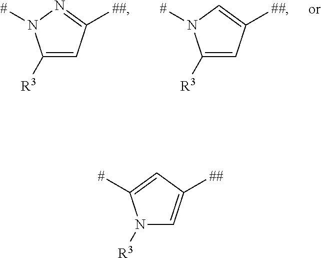
wherein
# designates the linkage point with the adjacent CH₂ group
and
## designates the linkage point with the ring 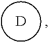
the ring 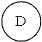
represents a heteroaryl ring of the formula 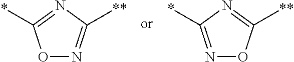
wherein
* designates the linkage point with the ring 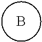
and
** designates the linkage point with the ring 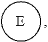
the ring 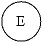
with the substituents R⁴ and R⁵ represents a phenyl ring of the formula 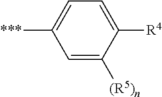
wherein
*** designates the linkage point with the ring 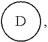
X represents —N(R⁶)—, —O—, —S— or ♦-N(R⁶)—C(═O)-♦♦ wherein
♦ designates the linkage point with the group L
and
♦♦ designates the linkage point with the ring 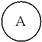
and
R⁶ denotes hydrogen, methyl, ethyl, isopropyl, cyclopropyl or cyclobutyl,
L represents ethane-1,2-diyl or propane-1,3-diyl,
R¹ represents hydrogen, (C₁-C₄)-alkyl, (C₁-C₄)-alkylcarbonyl, (C₁-C₄)-alkylsulphonyl or (C₃-C₆)-cycloalkyl,
where the alkyl group in (C₁-C₄)-alkyl, (C₁-C₄)-alkylcarbonyl and (C₁-C₄)-alkylsulphonyl may be substituted by hydroxyl or (C₁-C₄)-alkoxy or up to three times by fluorine
and
(C₃-C₆)-cycloalkyl can be substituted up to two times by identical or different radicals selected from the group consisting of fluorine, (C₁-C₄)-alkyl, hydroxyl and (C₁-C₄)-alkoxy,
R² represents hydrogen, (C₁-C₄)-alkyl or cyclopropyl,
where (C₁-C₄)-alkyl may be substituted up to three times by fluorine,
or
R¹ and R² together with the nitrogen atom to which they are attached form a saturated 4- to 6-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₃-C₆)-cycloalkyl,
R³ represents methyl,
R⁴ represents a substituent selected from the group consisting of chlorine, (C₁-C₆)-alkyl, trimethylsilyl, —OR⁷, —SR⁷, —S(═O)—R⁷, —S(═O)₂—R⁷, (C₃-C₆)-cycloalkyl and 4- to 6-membered heterocyclyl,
wherein (C₁-C₆)-alkyl for its part can be substituted by a radical selected from the group consisting of —OR⁷, —NR⁷R⁸, —C(═O)—NR⁷R⁸, (C₃-C₆)-cycloalkyl and 4- to 6-membered heterocyclyl and up to three time by fluorine
and
the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
and wherein
R⁷ and R⁸ independently of each other for each individual occurrence denote hydrogen, (C₁-C₄)-alkyl or (C₃-C₆)-cycloalkyl,
where (C₁-C₄)-alkyl can be substituted by hydroxyl, (C₁-C₄)-alkoxy, trifluoromethoxy or (C₃-C₆)-cycloalkyl and up to three times by fluorine
and
the cycloalkyl groups mentioned can be substituted up to two times by identical or different radicals selected from the group consisting of fluorine, (C₁-C₄)-alkyl, trifluoromethyl, hydroxyl, (C₁-C₄)-alkoxy and trifluoromethoxy,
or
R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
and
n represents the number 0 or 1,
or a salt thereof.

11. Compound of the formula (I) according to claim 1 in which
the ring 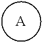
represents a phenyl ring and the adjacent groups X and CH₂ are bonded to this phenyl ring in 1,3 or 1,4 relation to one another,
the ring 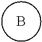
with the substituent R³ represents a heteroaryl ring of the formula 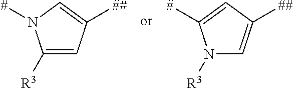
wherein
# designates the linkage point with the adjacent CH₂ group
and
## designates the linkage point with the ring 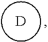
the ring 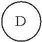
represents a heteroaryl ring of the formula 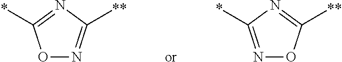
wherein
* designates the linkage point with the ring 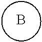
and
** designates the linkage point with the ring 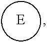
the ring 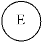
with the substituents R⁴ and R⁵ represents a phenyl ring of the formula 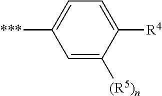
wherein
*** designates the linkage point with the ring 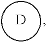
X represents —N(R⁶)—, —O—, —S— or ♦-N(R⁶)—C(═O)-♦♦ wherein
♦ designates the linkage point with the group L
and
♦♦ designates the linkage point with the ring 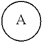
and
R⁶ denotes hydrogen, methyl, ethyl, isopropyl, cyclopropyl or cyclobutyl,
L represents ethane-1,2-diyl or propane-1,3-diyl,
R¹ represents hydrogen, (C₁-C₄)-alkyl, (C₁-C₄)-alkylcarbonyl, (C₁-C₄)-alkylsulphonyl or (C₃-C₆)-cycloalkyl,
where the alkyl group in (C₁-C₄)-alkyl, (C₁-C₄)-alkylcarbonyl and (C₁-C₄)-alkylsulphonyl may be substituted by hydroxyl or (C₁-C₄)-alkoxy or up to three times by fluorine
and
(C₃-C₆)-cycloalkyl can be substituted up to two times by identical or different radicals selected from the group consisting of fluorine, (C₁-C₄)-alkyl, hydroxyl and (C₁-C₄)-alkoxy,
R² represents hydrogen, (C₁-C₄)-alkyl or cyclopropyl,
where (C₁-C₄)-alkyl may be substituted up to three times by fluorine,
R³ represents methyl,
R⁴ represents a substituent selected from the group consisting of chlorine, (C₁-C₆)-alkyl, trimethylsilyl, —OR⁷, —SR⁷, —S(═O)—R⁷, —S(═O)₂R⁷, (C₃-C₆)-cycloalkyl and 4- to 6-membered heterocyclyl,
wherein (C₁-C₆)-alkyl for its part can be substituted by a radical selected from the group consisting of —OR⁷, —NR⁷R⁸, —C(═O)—NR⁷R⁸, (C₃-C₆)-cycloalkyl and 4- to 6-membered heterocyclyl and up to three times by fluorine
and
the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
and wherein
R⁷ and R⁸ independently of each other for each individual occurrence denote hydrogen, (C₁-C₄)-alkyl or (C₃-C₆)-cycloalkyl,
where (C₁-C₄)-alkyl can be substituted by hydroxyl, (C₁-C₄)-alkoxy, trifluoromethoxy or (C₃-C₆)-cycloalkyl and up to three times by fluorine
and
the cycloalkyl groups mentioned can be substituted up to two times by identical or different radicals selected from the group consisting of fluorine, (C₁-C₄)-alkyl, trifluoromethyl, hydroxyl, (C₁-C₄)-alkoxy and trifluoromethoxy,
or
R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
and
n represents the number 0 or 1,
or a salt thereof.

12. Compound of the formula (I) according to claim 1 in which
the ring 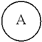
represents a phenyl ring and the adjacent groups X and CH₂ are bonded to this phenyl ring in 1, 3 or 1,4 relation to one another,
the ring 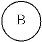
with the substituent R³ represents a heteroaryl ring of the formula 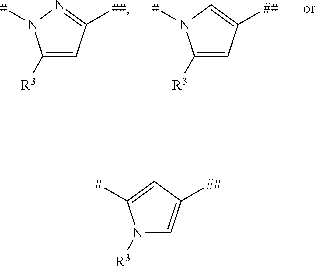
wherein
# designates the linkage point with the adjacent CH₂ group
and
## designates the linkage point with the ring 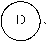
the ring 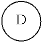
represents a heteroaryl ring of the formula 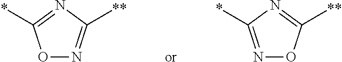
wherein
* designates the linkage point with the ring 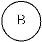
and
** designates the linkage point with the ring 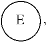
the ring 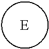
with the substituents R⁴ and R⁵ represents a phenyl ring of the formula 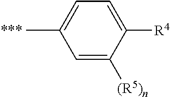
wherein
*** designates the linkage point with the ring 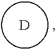
X represents —N(R⁶)—, —O—, —S— or ♦-N(R⁶)—C(═O)-♦♦ wherein
♦ designates the linkage point with the group L
and
♦♦ designates the linkage point with the ring 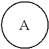
and
R⁶ denotes hydrogen, methyl, ethyl, isopropyl, cyclopropyl or cyclobutyl,
L represents ethane-1,2-diyl or propane-1,3-diyl,
R¹ and R² together with the nitrogen atom to which they are attached form a saturated 4- to 6-membered heterocycle which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₃-C₆)-cycloalkyl,
R³ represents methyl,
R⁴ represents a substituent selected from the group consisting of chlorine, (C₁-C₆)-alkyl, trimethylsilyl, —OR⁷, —SR⁷, —S(═O)—R⁷, —S(═O)₂—R⁷, (C₃-C₆)-cycloalkyl and 4- to 6-membered heterocyclyl,
wherein (C₁-C₆)-alkyl for its part can be substituted by a radical selected from the group consisting of —OR⁷, —NR⁷R⁸, —C(═O)—NR⁷R⁸, (C₃-C₆)-cycloalkyl and 4- to 6-membered heterocyclyl and up to three times by fluorine
and
the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
and wherein
R⁷ and R⁸ independently of each other for each individual occurrence denote hydrogen, (C₁-C₄)-alkyl or (C₃-C₆)-cycloalkyl,
where (C₁-C₄)-alkyl can be substituted by hydroxyl, (C₁-C₄)-alkoxy, trifluoromethoxy or (C₃-C₆)-cycloalkyl and up to three times by fluorine
and
the cycloalkyl groups mentioned can be substituted up to two times by identical or different radicals selected from the group consisting of fluorine, (C₁-C₄)-alkyl, trifluoromethyl, hydroxyl, (C₁-C₄)-alkoxy and trifluoromethoxy,
or
R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
and
n represents the number 0 or 1,
or a salt thereof.

13. Compound of the formula (I) according to claim 1 in which
the ring 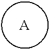
represents a phenyl ring and the adjacent groups X and CH₂ are bonded to this phenyl ring in 1,3 or 1,4 relation to one another,
the ring 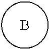
with the substituent R³ represents a heteroaryl ring of the formula 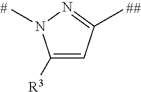
wherein
# designates the linkage point with the adjacent CH₂ group
and
## designates the linkage point with the ring 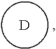
the ring 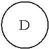
represents a heteroaryl ring of the formula 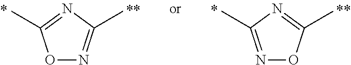
wherein
* designates the linkage point with the ring 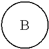
and
** designates the linkage point with the ring 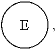
the ring 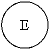
with the substituents R⁴ and R⁵ represents a phenyl ring of the formula 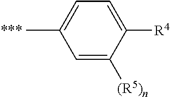
wherein
*** designates the linkage point with the ring 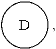
X represents —N(R⁶)—, —O—, —S— or ♦-N(R⁶)—C(═O)-♦♦ wherein
♦ designates the linkage point with the group L
and
♦♦ designates the linkage point with the ring 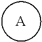
and
R⁶ denotes hydrogen, methyl, ethyl, isopropyl, cyclopropyl or cyclobutyl,
L represents ethane-1,2-diyl or propane-1,3-diyl,
R¹ represents hydrogen, methyl, ethyl, 2-hydroxyethyl, 2-methoxyethyl, isopropyl, cyclopropyl or cyclobutyl,
R² represents hydrogen, methyl or cyclopropyl,
R³ represents methyl,
R⁴ represents a substituent selected from the group consisting of chlorine, (C₁-C₆)-alkyl, trimethylsilyl, —OR⁷, —SR⁷, —S(═O)—R⁷, —S(═O)₂—R⁷, (C₃-C₆)-cycloalkyl and 4- to 6-membered heterocyclyl,
wherein (C₁-C₆)-alkyl for its part can be substituted by a radical selected from the group consisting of —OR⁷, —NR⁷R⁸, —C(═O)—NR⁷R⁸, (C₃-C₆)-cycloalkyl and 4- to 6-membered heterocyclyl and up to three times by fluorine
and
the cycloalkyl and heterocyclyl groups mentioned for their part can be substituted up to two times by identical or different radicals selected from the group consisting of fluorine, (C₁-C₄)-alkyl, trifluoromethyl, hydroxyl, (C₁-C₄)-alkoxy, trifluoromethoxy, oxo and (C₁-C₄)-alkylcarbonyl
and wherein
R⁷ and R⁸ independently of each other for each individual occurrence denote hydrogen, (C₁-C₄)-alkyl or (C₃-C₆)-cycloalkyl,
where (C₁-C₄)-alkyl can be substituted by hydroxyl, (C₁-C₄)-alkoxy, trifluoromethoxy or (C₃-C₆)-cycloalkyl and up to three times by fluorine
and
the cycloalkyl groups mentioned can be substituted up to two times by identical or different radicals selected from the group consisting of fluorine, (C₁-C₄)-alkyl, trifluoromethyl, hydroxyl, (C₁-C₄)-alkoxy and trifluoromethoxy,
or
R⁷ and R⁸ in the case where both are bonded to a nitrogen atom form a 4- to 6-membered heterocycle together with this nitrogen atom, which can contain a further ring heteroatom from the group consisting of N, O, S and S(O)₂ and which can be substituted up to two times by identical or different radicals selected from the group consisting of fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
and
n represents the number 0 or 1,
or a salt thereof.

14. (canceled)

15. (canceled)

16. (canceled)

17. (canceled)

18. (canceled)

19. A pharmaceutical composition comprising a compound as defined in claim 1 in combination with one or more inert, non-toxic, pharmaceutically suitable auxiliary substances.

20. A pharmaceutical composition comprising a compound as defined in claim 1 in combination with one or more other active compounds.

21. (canceled)

22. (canceled)

23. Method for the treatment and/or prevention of cancer diseases or tumour diseases comprising administering an effective amount of at least one compound as defined in claim 1 to a human or animal in need thereof.

24. Method for the treatment and/or prevention of ischaemic cardiovascular diseases, cardiac insufficiency, cardiac infarction, arrhythmia, stroke, pulmonary hypertension, fibrotic diseases of the kidney and lung, psoriasis, diabetic retinopathy, macular degeneration, rheumatic arthritis or Chugwash polycythaemia comprising administering an effective amount of at least one compound as defined in claim 1 to a human or animal in need thereof.

25. Process for the preparation of compounds of the formula (I-D) 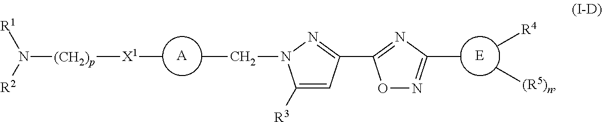
in which the rings A and E and R¹, R², R³, R⁴, R⁵ and n each have the meanings given in claim 1.
X¹ represents NH or O,
and
p represents the number 2, 3 or 4,
characterized in that initially an N′-hydroxyamidine of the formula (XI) 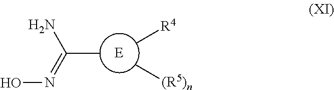
in which ring E and also R⁴, R⁵ and n have the meanings given above,
is condensed with a pyrazolecarboxylic acid of the formula (XXVIII) 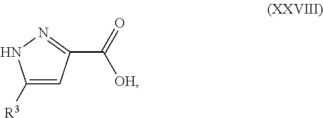
in which R³ has the meaning given above
to give a 1,2,4-oxadiazole derivative of the formula (XXIX) 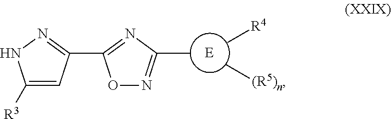
in which ring E and also R³, R⁴, R⁵ and n have the meanings given above,
and the compound (XXIX) is then alkylated in the presence of a base either
[A] with a compound of the formula (XXX) 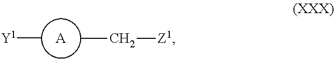
in which ring A has the meaning given above
Y¹ represents chlorine, bromine or iodine
and
Z¹ represents a leaving group such as, for example, chlorine, bromine, iodine, mesylate, triflate or tosylate,
to give a compound of the formula (XXXI) 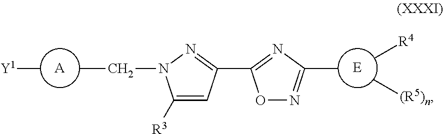
in which rings A and E and R³, R⁴, R⁵, n and Y¹ each have the meanings given above,
and then optionally reacted in the presence of a palladium catalyst and/or a base with a compound of the formula (XXXII) 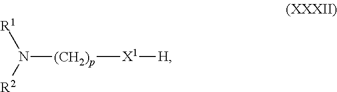
in which R¹, R², p and X¹ have the meanings given above,
to give the compound of the formula (I-D)
or
[B] in an alternative, if in compound (I-D) X¹ represents O and ring A represents a phenyl ring,
alkylated with a compound of the formula (XXXIII) 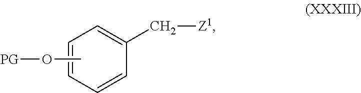
in which
PG represents a silyl protective group such as, for example trimethylsilyl, triisopropylsilyl or tert-butyldimethylsilyl
and
Z¹ represents a leaving group such as, for example, chlorine, bromine, iodine, mesylate, triflate or tosylate,
to give a compound of the formula (XXXIV) 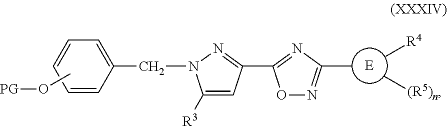
in which ring E and also R³, R⁴, R⁵, n and PG have the meanings given above,
and, after removal of the silyl protective group PG, reacting the resulting compound of the formula (XXXV) 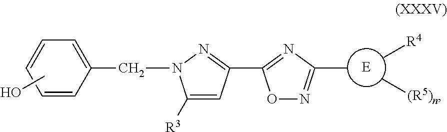
in which ring E and also R³, R⁴, R⁵ and n have the meanings given above,
if appropriate in the presence of a base with a compound of the formula (XXXVI) 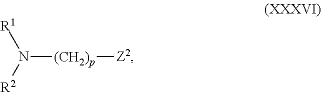
in which R¹, R² and p have the meanings given above,
and
Z² represents a leaving group such as, for example, chlorine, bromine, iodine, hydroxyl, mesylate, triflate or tosylate,
to give the compound of the formula (I-D′) 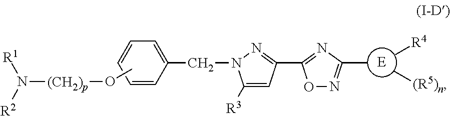
in which ring E and R¹, R², R³, R⁴, R⁵, n and p each have the meanings given above.
